(19) United States
(12) Patent Application Publication
(10) Pub. No.: US 2016/0208246 A1
(43) Pub. Date: Jul. 21, 2016

(54) COMPOSITIONS AND METHODS FOR TREATING A HEMATOLOGICAL MALIGNANCY ASSOCIATED WITH AN ALTERED RUNX1 ACTIVITY OR EXPRESSION

(71) Applicant: YEDA RESEARCH AND DEVELOPMENT CO. LTD., Rehovot (IL)

(72) Inventors: Yoram GRONER, Rehovot (IL); Oren BEN-AMI, Rehovot (IL)

(21) Appl. No.: 14/897,281

(22) PCT Filed: Jun. 10, 2014
(86) PCT No.: PCT/IL2014/050523

§ 371 Date: Dec. 10, 2015

Related U.S. Application Data
(60) Provisional application No. 61/833,006, filed on Jun. 10, 2013.

(51) Int. Cl. C12N 15/113

(52) U.S. Cl. CPC C12N 15/113

(57) ABSTRACT

A method of treating a hematological malignancy associated with an altered RUNX1 activity or expression is disclosed. The method comprising administering to a subject in need thereof a therapeutically effective amount of an agent which directly downregulates an activity or expression of RUNX1, thereby treating the hematological malignancy associated with the altered RUNX1 activity or expression.

### FIELD AND BACKGROUND OF THE INVENTION

The present invention, in some embodiments thereof, relates to compositions and methods for treating a hematological malignancy associated with an altered RUNX1 activity or expression.

Acute myeloid leukemia (AML) is characterized by a block in early progenitor differentiation leading to accumulation of immature, highly proliferative, leukemic stem cells (LSC) in bone marrow (BM) and blood. Genes coding for transcription factors (TFs) are frequently mutated or dysregulated in AML indicating their critical involvement in disease etiology. Chromosome-21-encoded TF RUNX1 (previously known as AML1) is a frequent target of various chromosomal translocations. The most prevalent translocation in AML is t(8;21), which creates a fused gene product designated AML1-ETO (A-E). It contains the DNA-binding domain of RUNX1 (the runt domain; RD), linked to the major part of the chromosome-8 encoded protein ETO, which by itself lacks DNA-binding capacity.

RUNX1 is a key hematopoietic gene-expression regulator in embryos and adults. Its major cofactor, the core-binding protein-β (CBFβ), is essential for RUNX1 function. On the other hand, ETO is a transcriptional repressor, known to interact with co-repressors such as NCoR/SMRT, mSin3a and HDACs. Of note, while the ETO gene is normally expressed in the gut and central nervous system, the t(8;21) translocation places it under transcription control of RUNX1 regulatory elements. This occurrence evokes expression of A-E in the myeloid cell lineage.

The prevailing notion is that A-E binds to RUNX1 target genes and acts as dominant-negative regulator thereby producing conditions that resemble the RUNX1^{-/-} phenotype. Consistent with this concept, mice expressing an A-E knock-in allele display early embryonic lethality and hematopoietic defects resembling the phenotype of Runx1^{-/-} mice. However, it has also been shown that A-E-mediated leukemogenicity involves other events that affect gene regulation, in addition to repression of RUNX1 targets. Reduction of A-E expression in leukemic cells by siRNA restores myeloid differentiation and delays in-vivo tumor formation. More recently Ptasinska et al. [Ptasinska, A. et al., Leukemia (2012) 26, 1829-1841] showed that depletion of A-E in t(8;21)⁺ AML cells causes genome-wide changes in chromatin structure leading to redistribution of RUNX1 genomic occupancy. These changes inhibited the leukemic cell self-renewal capacity and induced differentiation [Ptasinska et al. (2012), supra]. An additional AML subtype associated with altered RUNX1 activity involves the chromosomal aberrations inv(16)(p13q22) and t(16;16(p13;q22) [abbreviated as inv(16)]. This inversion fuses chromosome 16q22 encoded CBFβ gene with the MYH11 gene, which resides at the 16p13 region and encodes the smooth-muscle myosin-heavy chain (SMMHC). The resulting chimeric oncoprotein is known as CBFβ-SMMHC. Similar to A-E, CBFb-SMMHC (C-S) is a dominant inhibitor of RUNX1 activity which impairs myeloid differentiation and contributes to AML development.

Previous data have illustrated that RUNX1 is active in both t(8;21) and inv(16) AML patients, whereas RUNX1 is frequently inactivated in other forms of AML [Goyama, S. and Mulloy J C., Int J Hematol (2011) 94, 126-133].

U.S. Patent Application No. 20110217306 relates to a novel C-terminal exon of RUNX1/AML1, its nucleic acid sequence, its peptide and a full length amino acid sequence comprising same. U.S. 20110217306 teaches that the C-terminal exon (i.e. exon 5.4 at the C-terminus) comprises a dominant negative function which may be used for therapeutic and/or prophylactic treatment of diseases associated with RUNX1/AML1 target genes, as well as for the inhibition of cellular growth and/or induction of apoptosis. U.S. 20110217306 further provides an antibody against the C-terminal exon of RUNX1/AML1 and a pharmaceutical composition for the treatment of various diseases (e.g. tumors).

U.S. Patent Application No. 20090226956 relates to compounds for modulating the activity of Runx2 or Runx1 through inhibition by estrogen receptor α (ERα) or AR (androgen receptor) and the use of such compounds for treating bone diseases and cancer (e.g. leukemia).

### SUMMARY OF THE INVENTION

According to an aspect of some embodiments of the present invention there is provided a method of treating a hematological malignancy associated with an altered RUNX1 activity or expression, the method comprising administering to a subject in need thereof a therapeutically effective amount of an agent which directly downregulates an activity or expression of RUNX1, thereby treating the hematological malignancy associated with the altered RUNX1 activity or expression.

According to an aspect of some embodiments of the present invention there is provided a method of inducing apoptosis of hematopoietic cells associated with an altered RUNX1 activity or expression, the method comprising administering to the hematopoietic cells a therapeutically effective amount of an agent which directly downregulates an activity or expression of RUNX1, thereby inducing the apoptosis of the hematopoietic cells.

According to an aspect of some embodiments of the present invention there is provided a method of inducing apoptosis of hematopoietic cells of a subject having a hematological malignancy associated with an altered RUNX1 activity or expression, the method comprising administering to the subject a therapeutically effective amount of an agent which directly downregulates an activity or expression of RUNX1, thereby inducing apoptosis of the hematopoietic cells of the subject.

According to an aspect of some embodiments of the present invention there is provided an isolated polynucleotide which directly downregulates RUNX1 but not AML1-ETO (A-E), AML1-EVI1 or ETV6-RUNX1 (TEL/AML1).

According to an aspect of some embodiments of the present invention there is provided a nucleic acid construct comprising the isolated polynucleotide of some embodiments of the invention.

According to an aspect of some embodiments of the present invention there is provided a pharmaceutical composition comprising the isolated polynucleotide of some embodiments of the invention and a pharmaceutically acceptable carrier.

According to an aspect of some embodiments of the present invention there is provided a pharmaceutical composition comprising the isolated polynucleotide of some embodiments of the invention, a pro-apoptotic agent and a pharmaceutically acceptable carrier.

According to some embodiments of the invention, the RUNX1 is as set forth in SEQ ID NO: 44, 56 or 58.

According to some embodiments of the invention, the agent which downregulates the activity or expression of RUNX1 does not substantially affect an activity or expression of the altered RUNX1.

According to some embodiments of the invention, the hematological malignancy is a leukemia or lymphoma.

According to some embodiments of the invention, the leukemia is an acute myeloid leukemia (AML).

According to some embodiments of the invention, the AML is type t(8;21).

According to some embodiments of the invention, the AML is type inv(16).

According to some embodiments of the invention, the AML is type t(3;21).

According to some embodiments of the invention, the leukemia is an acute lymphoblastic leukemia (ALL).

According to some embodiments of the invention, the ALL is type t(12;21).

According to some embodiments of the invention, the agent is a polynucleotide agent.

According to some embodiments of the invention, the polynucleotide agent is selected from the group consisting of an antisense, a siRNA, a microRNA, a Ribozyme and a DNAzyme.

According to some embodiments of the invention, the polynucleotide agent is directed to a nucleic acid region selected from the group consisting of SEQ ID NO: 43, SEQ ID NO: 45, SEQ ID NO: 47, SEQ ID NO: 49, SEQ ID NO: 50, SEQ ID NO: 55 and SEQ ID NO: 57.

According to some embodiments of the invention, the polynucleotide agent comprises 15-25 nucleotides.

According to some embodiments of the invention, the polynucleotide agent is selected from the group consisting of SEQ ID NO: 52 and SEQ ID NO: 53.

According to some embodiments of the invention, the agent is a small molecule.

According to some embodiments of the invention, the RUNX1 is a wild-type RUNX1.

According to some embodiments of the invention, the therapeutically effective amount initiates apoptosis of hematopoietic cells of the hematological malignancy.

According to some embodiments of the invention, the apoptosis is caspase dependent.

According to some embodiments of the invention, the subject is a human subject.

According to some embodiments of the invention, the method further comprises administering to the subject a pro-apoptotic agent for targeted killing of the hematological malignancy.

According to some embodiments of the invention, the pro-apoptotic agent is caspase dependent.

According to some embodiments of the invention, the pro-apoptotic agent is administered prior to, concomitantly with or following administration of the agent which downregulates the activity or expression of the RUNX1.

According to some embodiments of the invention, the method is effected in-vivo.

According to some embodiments of the invention, the hematopoietic cells comprise myeloma cells or lymphocytes.

According to some embodiments of the invention, the leukemia is an acute myeloid leukemia (AML) selected from the group consisting of type t(8;21), t(3;21) and type inv(16).

According to some embodiments of the invention, the leukemia is an acute lymphoblastic leukemia (ALL) comprising type t(12;21).

According to some embodiments of the invention, the polynucleotide comprises a nucleic acid sequence as set forth in SEQ ID NO: 52 or SEQ ID NO: 53.

According to some embodiments of the invention, the pharmaceutical composition is formulated for penetrating a cell membrane.

According to some embodiments of the invention, the pharmaceutical composition comprises a nano-carrier.

According to some embodiments of the invention, the nano-carrier comprises a lipid vesicle.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1I depict that wild-type (WT) RUNX1 prevents apoptosis of t(8;21) Kasumi-1 leukemic cell line:

FIG. 1A, upper panel, is a schematic illustration of RUNX1 (blue) and RUNX1-ETO (A-E) (blue-red) transcripts indicating regions targeted by the siRNAs used to knock down (KD) expression of either RUNX1 (bars underneath RUNX1 marked in green and orange) or A-E (black bar underneath A-E fusion region). FIG. 1A, lower panel, illustrates a RT-qPCR analysis of siRNA mediated RUNX1 KD using the RUNX1-targeting siRNA (SEQ ID NO: 52) that matches the sequence: GACAUCGGCAGAAACUAGA (SEQ ID NO: 49) (as marked in green in the upper panel). Total RNA isolated 24 hrs post electroporation of RUNX1-targeting or non-targeting (NT) control siRNA. Data shown represent mean expression±SE. Shown are results from one of three experiments with the same findings. Primers used for RT-qPCR are presented in Table 1 (in the Examples section which follows).

FIGS. 1B and 1C illustrate cell cycle analysis 8 days post transfection with either RUNX1-targeting (SEQ ID NO: 52) or control non-targeting (NT) siRNA. FIG. 1B illustrates cells which were subjected to two successive transfections (at days 0 and 4) with either RUNX1-targeting or NT siRNA. Propidium iodide (PI) was used to assess cellular DNA content by FACS analysis. Bar numbers indicate the relative size (in %) of labeled population out of total cells. Indicated cell cycle phases: subG1; G1; S and G2M; and FIG. 1C are histograms summarizing the distribution of cell population as analyzed in FIG. 1B. Data represents mean±STDV values of five independent experiments.

FIG. 1D illustrates increased Kasumi-1^{RX1-KD} cell apoptosis. Cells were stained with Annexin-V following siRNA-mediated RUNX1 KD (SEQ ID NO: 52). Dead/late apoptotic cells were marked by staining with the eFluor780 viability dye. Results from one of two experiments with the same findings are shown (see also FIGS. 1J-1L).

FIG. 1E illustrates diminished Kasumi-1^{RX1-KD} cell viability. Eight days post transfection with either RUNX1-targeting (SEQ ID NO: 52) or NT siRNA total number of viable cells was assessed using standard hemocytometer cell counting excluding Trypan Blue stained cells. Data represents mean±STDV values of three independent experiments.

FIGS. 1F and 1G illustrate that RUNX1 KD induced apoptosis is associated with loss of mitochondrial membrane potential. FIG. 1F shows an ImageStream® System analysis of Kasumi-1 cells incubated for 4 days with RUNX1-targeting (SEQ ID NO: 52) or NT siRNA and stained for cell mitochondria and DNA content. Bright field visualizing indicates cell apoptotic morphology. Green-fluorescent dye (Mitogreen) stains mitochondria in both live and dead cells. Red-dye (MitoTracker Red CMXRos) stains mitochondria only in live cells, depends on mitochondrial membrane potential and indicates MPT. DNA was stained with DRAQ5. Cells with low Red/Green ratio and low DNA signal were defined as apoptotic. Results from one of two experiments with the same findings are shown; and FIG. 1G are histograms presenting quantitative data of ImageStream© System analysis for Kasumi-1^{RX1-KD} and Kasumi-1^{Cont} as mean±STDV of two biological repeats.

FIG. 1H illustrates that caspase inhibition rescues Kasumi-1^{RX1-KD} from apoptosis. Three days post siRNA-delivery cells were incubated with either Z-VAD-FMK (50 μM) or vehicle (DMSO) for additional 24 hrs. Histograms show the distribution of cells among cell cycle phases determined as detailed above. Data shown represent mean±STDV of four independent experiments.

FIG. 1I illustrates a western blot analysis demonstrating RUNX1 KD. Cells transfected with RUNX1-targeting (SEQ ID NO: 52) or NT siRNA were incubated for 72 hrs followed by additional 24 hrs incubation with Z-VAD-FMK (50 μM). Blots were reacted with an antibody (Ab) against RUNX1-N-terminus or Lamin. Results from one of two experiments with the same findings are shown.

FIGS. 1J-1L depict the efficacy of the alternative siRNA in causing RUNX1 KD-mediated Kasumi-1 cell apoptosis. An alternative siRNA (see FIG. 1A marked in orange) was used for KD of RUNX1 and analysis of consequent apoptosis of Kasumi-1^{RX1-KD} cells. This second siRNA (SEQ ID NO: 53) targets the following RUNX1 sequence: GGCGAUAGGUCUCACGCAA (SEQ ID NO: 50):

FIG. 1J illustrates a RT-qPCR analysis of RUNX1 KD by the siRNA set forth in SEQ ID NO: 53. Cells were incubated for 24 hrs with the specific siRNA or NT control siRNA prior to extraction of RNA.

FIG. 1K illustrates DNA content-based cell cycle analysis using PI-stained cells harvested 8 days after siRNA delivery. Results from one of four experiments with the same findings are shown.

FIG. 1L illustrates elevated Annexin-V⁺ among eFluor 780-negative viable cells indicating increased RUNX1 KD-dependent apoptosis of Kasumi-1 cells. Increased frequency of late apoptotic or dead Annexin V⁺eFluor 780⁺ cells was also observed in Kasumi-1^{RX1-KD} cell population. Results from one of two experiments with the same findings are shown.

FIGS. 2A-2G depict rescue of Kasumi-1^{RX1-KD} cells from apoptosis by KD of A-E:

FIGS. 2A-2B illustrate reduced expression of A-E in Kasumi-1^{AE-KD} cells. Expression of A-E following cell transfection with A-E-targeting siRNA (SEQ ID NO: 54, indicated by black bar in FIG. 1A, that matches the sequence: CCUCGAAAUCGUACUGAGA (SEQ ID NO: 51)) or NT siRNAs was analyzed by RT-qPCR (left panel) 24 h post transfection and by Western blotting (right panel) using anti ETO or lamin Abs 96 h post transfection (see also FIGS. 2H-1L).

FIGS. 2C-2G illustrate that KD of A-E rescues Kasumi-1 cells from RUNX1 KD-induced apoptosis. Cells were co-transfected with a 1:1 mixture of RUNX1 and A-E targeting siRNAs (SEQ ID NOs: 52 and 54, respectively) or separately with RUNX1 siRNA, A-E siRNA or NT siRNA. FIGS. 2C-2F, following incubation for 8 days, cells were stained with PI and analyzed by FACS for cell cycle; and FIG. 2G are histograms showing the distribution of cells among cell cycle phases. Data shown represent mean±STDV of four independent biological repeats.

FIGS. 2H-2L depict that KD of A-E expression diminished Kasumi-1 cell leukemogenic phenotype:

FIGS. 2H and 21 illustrate that A-E KD attenuates self-renewal and promotes myeloid differentiation of Kasumi-1 cells. FIG. 2H is a dye-dilution proliferation assay. To obtain prolonged A-E KD, cells were transfected with siRNA (SEQ ID NO: 54) twice. Four days following the initial siRNA delivery, cells were re-transfected with an additional amount of siRNA. After 24 hrs, cells were labeled with the membrane staining dye (Vybrant Dil cell-labeling solution; Life Technologies) and subjected to FACS analysis either immediately post-staining (Day 0) or following 6 days in culture (Day 6). Results from one of two experiments with the same findings are shown. Of note, Kasumi-1^{AE-KD} cells exhibit decreased proliferation compared to Kasumi-1^{Cont} cells, as evidenced by their higher staining intensity at Day 6. This observation corresponds with previously reported findings [Ptasinska et al. (2012), supra]; and FIG. 2I illustrates that KD of A-E in Kasumi-1 cells is associated with elevated expression of a gene subset characteristic of myeloid cell differentiation. RNA was isolated from Kasumi-1 cells 8 days post transfection with A-E targeting or NT siRNA and analyzed by RT-qPCR. Data shown represent mean±SE of two biological repeats.

FIGS. 2J and 2K illustrates that KD of A-E affects the expression of CD38 and CD34 genes that mark HSCs population playing role in AML etiology. FIG. 2J illustrates decreased expression of CD34 and CD38 genes in Kasumi-1^{AE-KD} cells. RT-qPCR of RNA isolated from cells incubated with either A-E-targeting siRNA (SEQ ID NO: 54) or control NT siRNA for 8 days. Data shown represent mean expression±SE of four biological repeats; and FIG. 2K illustrates a reduction in CD34⁺CD38⁻ leukemic cell population following A-E KD. FACS analysis of cells incubated with A-E targeting or control NT siRNAs for 8 days. Of note, the CD34⁺CD38⁻ cell population that initiates AML in severe combined immune-deficient (SCID) mice was markedly reduced. Results from one of four biological repeats with the same findings are shown.

FIG. 2L illustrate binding of RUNX1 and A-E to CD34 (upper panel) and CD38 (lower panel) genomic loci. Shown are ChIP-Seq readout wiggle files uploaded to UCSC Genome Browser hg18 genome assembly indicating that both RUNX1 and A-E bind to CD38 and CD34 genomic loci. Of note, this may suggest that A-E competitively inhibits the expression of genes normally regulated by RUNX1 and thereby promotes the CD34⁺CD38⁻ leukemogenic cell phenotype. The finding underscores the significant role of the interrelationships between A-E and WT RUNX1 in the etiology of t(8;21) hematopoietic malignancy.

FIGS. 3A-3G is a gene expression and ChIP-seq analysis of A-E and RUNX1 occupied genomic regions:

FIG. 3A is a gene expression profiling of Kasumi-1 following KD of either RUNX1 or A-E revealing a significant inverse gene expression response evidenced by negative Spearman correlation (R²=−0.33).

FIG. 3B is Venn diagram showing the number and relative proportion of genes whose expression significantly changed following KD of either RUNX1 or A-E. Differential expression cut-off was set to minimal absolute fold-change of 1.4, and maximal p-value of 0.05. See also Tables 2-5 (in the Examples section which follows).

FIG. 3C is a selective detection of RUNX1 or A-E proteins in Kasumi-1 cells. Western blotting of Kasumi-1 nuclear extracts using antibodies raised against RUNX1 C-terminus (left lane) or against ETO (right lane). The central lane was reacted with anti RUNX1-N-terminus antibody detecting both RUNX1 and A-E.

FIG. 3D is a Venn diagram of the number and relative proportion of RUNX1- and/or A-E-occupied genomic regions recorded by ChIP-Seq experiments using anti-RUNX1 C-terminus or anti-ETO antibodies.

FIG. 3E is a comparison of RUNX1 and/or A-E binding-affinity detected by ChIP-Seq analysis. Binding of A-E and RUNX1 strongly correlated (Pearson R²=0.72, p-value <2e⁻¹⁶).

FIGS. 3F and 3G illustrate enrichment of genes up- and down-regulated in response to KD of RUNX1 (FIG. 3F) and A-E (FIG. 3G), respectively. Data was compiled using integrated results of ChIP-seq and gene expression. Shown are enrichment ratios for up and down regulated genes computed as the fraction of bound regulated genes divided by the global fraction of bound genes.

FIGS. 4A-4D depicts a comparative sequence analysis of RUNX1 and A-E bound regions:

FIG. 4A illustrates the frequency of uniquely bound RUNX1 or A-E proximal to annotated TSS. Bound TF was defined as ‘proximal’ when distance to annotated TSS was less than 500 bp.

FIG. 4B illustrates enrichment of the canonical RUNX motif (left panel) and a RUNX-variant motif (right panel) in regions uniquely bound by RUNX1 or A-E. Level of motif enrichment is coded numerically (0=no to 10=high enrichment) and by color intensity in the Venn diagrams.

FIG. 4C illustrates that the ratio of ChIP-seq binding intensities of RUNX1 and A-E is positively correlated with the relative enrichment of the canonical and variant RUNX motifs. Shown are binding intensities, color-coded according to motif enrichments ratios: blue-high enrichment of canonical RUNX motif (observed mostly at upper left), and red-high enrichment of variant RUNX motif (observed mostly at lower right).

FIG. 4D illustrates enrichment of the ETS (upper) and AP4 (lower) TF motifs among unique and common RUNX1/A-E bound regions. Motifs were identified de-novo using A-E and RUNX1 ChIP-seq genomic bound regions. Level of enrichment is indicated both numerically and by color as in FIG. 4B. (see also FIGS. 4E-4F).

FIGS. 4E-4F depict genomic occupancy of the E-Box TF AP4 in Kasumi-1 cell line:

FIG. 4E illustrates that AP4 is highly expressed in Kasumi-1 cell line. Western blotting of Kasumi-1 nuclear extract using anti-AP4 antibodies revealed significant amount of AP4 protein. Emerin served as protein loading control.

FIG. 4F illustrates a genome wide co-occupancy of AP4 with A-E and/or RUNX1 in Kasumi-1 cell line. Venn diagram showing overlaps between genomic occupancy of AP4, A-E and RUNX1 as determined by ChIP-seq analysis. Anti-AP4 antibodies analyzed in (FIG. 4E) was used in AP4 ChIP-seq experiments. The frequencies of AP4/A-E or AP4/RUNX1 co-binding were found to be similar.

FIGS. 5A-5F depict a transcriptome analysis of Z-VAD-FMK treated Kasumi-1^{RX1-KD} cells highlighting a gene subset crucial for mitotic function: FIG. 5A illustrates a gene expression profile of Z-VAD-FMK treated Kasumi-1^{RX1-KD} cells. Scatter plot of differentially expressed genes in Kasumi-1 cells treated with control NT or RUNX1-targeting siRNA (SEQ ID NO: 52) for 96 hrs. During this time cells were incubated with Z-VAD-FMK (50 μM) for 40 hrs prior to FACS sorting of FITC⁺ cells for RNA isolation. Genes that were up- or down-regulated due to RUNX1 KD are marked by red or blue, respectively. Differential expression cut-off was set to minimal absolute fold-change of 1.4, and maximal p-value of 0.05 (see also Tables 6-7 in the Examples section which follows).

FIG. 5B illustrates a RT-qPCR analysis of mitotic genes scored by microarray gene expression. Results are presented as mean±SE of two biological repeats.

FIGS. 5C-5F illustrate that RUNX1 and A-E exhibit similar binding-pattern to the TOP2A, NEK6, SGOL1 and BUB1 genomic loci. Shown are ChIP-Seq tracing wiggle files uploaded to UCSC Genome Browser hg18 genome assembly.

FIGS. 6A-6N depict opposing effect of A-E and RUNX1 on Kasumi-1 cell SAC signaling and requirement of RUNX1 for survival of inv(16) ME-1 cell line and A-E-expressing CD34⁺ preleukemic cells.

SAC signaling is regulated by RUNX1 and A-E. Cells were transfected with the indicated siRNAs and incubated for 72 hrs prior to addition of vehicle (DMSO) (FIGS. 6A-6D) or Nocodazole (0.1 μg/ml) (FIGS. 6E-6H) for the subsequent 14 hrs. Cell cycle analysis was performed by FACS using PI labeling as described in FIG. 1B. Bar numbers indicate the relative population size (in %) out of total cell number. Results from one of three experiments with similar findings are shown.

FIG. 6I illustrates the relative activity of RUNX1 and A-E impact on SAC efficacy and thereby on cell tendency to undergo apoptosis. Histogram showing the ratio of % cells in G2/M vs. subG1. The ratio calculated for NT group was considered as 1.

FIGS. 6J and 6K illustrate that RUNX1 activity is essential for survival of inv(16) ME-1 cell line. FIG. 6J is a RT-qPCR demonstrating RUNX1 KD in ME-1 cells. RNA isolated from cells incubated for 24 hrs with RUNX1-targeting or NT siRNA was analyzed by RT-qPCR. Results are mean expression±SE values of two experiments with similar results; and FIG. 6K illustrates that KD of RUNX1 enhances apoptosis of ME-1 cell line. Cells were subjected to two successive rounds of electroporation (day 0 and 5) with either RUNX1-targeting (SEQ ID NO: 52) or NT siRNA. On Day 10, cell viability was determined by staining with viability dye and apoptosis was monitored by FACS analysis of Annexin V stained cells. Results from one of four experiments with similar findings are shown (see also FIGS. 6O-6P).

FIG. 6L illustrates qRT-PCR demonstrating RUNX1 KD in CD34+/A-E cells. RNA from CD34+/A-E cells 24 hrs posttransfection with RUNX1-targeting or NT siRNA was analyzed by qRT-PCR. Results are the mean expression±SE values of two experiments with similar results.

FIG. 6M and FIG. 6N illustrate KD of RUNX1 increased apoptosis of CD34+/A-E cells. Twelve days after transduction with A-E lentiviral vector, cells were transfected with either RUNX1-targeting or NT siRNA, and 4 days later GFP+ cells were assayed for Annexin-V staining by FACS. Histograms demonstrate a 2-fold increase in the proportion of Annexin-V-positive CD34+/A-E cells among RUNX1 KD in comparison to control cultures. Results from one of three experiments with similar findings are shown.

FIGS. 6O-6P depict that Inv(16) AML ME-1 cell line exhibits mixed population of diploid and tetraploid cells:

FIG. 6O illustrates untreated ME-1 cells stained with PI followed by FACS cell cycle analysis. Of note and as evidenced by PI-staining intensity, mixed populations of diploid and tetraploid cells are observed; and FIG. 6P illustrates that cellular DNA content is correlated with cell size as estimated by FACS forward scatter area parameter. Data shown represents one of two similar experiments.

FIG. 7 is a schematic model summarizing the role of RUNX1 in t(8;21)-mediated AML development. The 8;21 chromosomal translocation in HSC generates Pre-LSC, expressing A-E and WT RUNX1 that have acquired increased self-renewal, impaired differentiation, and compromised SAC. The combined expression of RUNX1 and A-E is essential for sustained viability and self-renewal that promotes acquisition of additional genetic alterations. The accumulation of genetic hits leads to further cell transformation, yielding LSC and consequently full-blown AML. Inactivation of RUNX1 in t(8;21) AML cells triggers A-E-mediated caspase-dependent apoptosis associated with further impairment of SAC activity and mitotic failure.

### DESCRIPTION OF SPECIFIC EMBODIMENTS OF THE INVENTION

The present invention, in some embodiments thereof, relates to compositions and methods for treating a hematological malignancy associated with an altered RUNX1 activity or expression.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Acute myeloid leukemia (AML) is characterized by a block in early progenitor differentiation leading to accumulation of immature, highly proliferative, leukemic stem cells in bone marrow and blood. The most prevalent translocation in AML is t(8;21), which creates a fused gene product designated AML1-ETO (A-E). A-E contains the DNA-binding domain of the chromosome-21-encoded transcription factor RUNX1 (the runt domain; RD), linked to the major part of the chromosome-8 encoded protein ETO (a transcriptional repressor). An additional AML subtype associated with altered RUNX1 activity involves the chromosomal aberrations inv(16)(p13q22) and t(16;16(p13;q22) [abbreviated as inv(16)], and results in an oncogenic fusion protein known as CBFβ-SMMHC (C-S).

While reducing the present invention to practice, the present inventors have surprisingly uncovered that the expression of wild-type (WT) RUNX1 is essential for survival and leukemogenesis of the t(8;21) and inv(16) leukemic cells. Specifically, the present inventors have uncovered a role of RUNX1 in regulation of mitotic checkpoint events through which it prevents the inherited apoptotic process in t(8;21) cells and facilitates leukemogenesis. Furthermore, the present inventors have shown that attenuation of RUNX1 activity or expression directs these cells to apoptosis.

As is shown hereinbelow and in the Examples section which follows, the present inventors have uncovered through laborious experimentation that WT RUNX1 is required for survival of t(8;21)-Kasumi-1 and inv(16)-ME-1 AML cell lines (see Examples 1 and 8, in the Examples section hereinbelow). RUNX1 knockdown (KD) in Kasumi-1 cells (Kasumi-1^{RX1-KD}) resulted in A-E-mediated caspase-dependent apoptosis. Specifically, RUNX1 KD in Kasumi-1 cells (Kasumi-1^{RX1-KD}) attenuated cell-cycle mitotic checkpoint, leading to apoptosis, whereas knocking-down the t(8;21)-onco-protein AML1-ETO in Kasumi-1^{RX1-KD} rescues these cells (see Examples 1, 2, 6 and 7). Moreover, malignant AML phenotype is sustained by a delicate AML1-ETO/RUNX1 balance that involves competition for common DNA binding sites regulating a subset of AML1-ETO/RUNX1 targets (see Examples 3 and 4). Thus, RUNX1 is a potential candidate for new therapeutic modalities.

Thus, according to one aspect of the present invention there is provided a method of treating a hematological malignancy associated with an altered RUNX1 activity or expression, the method comprising administering to a subject in need thereof a therapeutically effective amount of an agent which directly downregulates an activity or expression of RUNX1, thereby treating the hematological malignancy associated with the altered RUNX1 activity or expression.

As used herein the term “treating” refers to curing, reversing, attenuating, alleviating, minimizing, suppressing or halting the deleterious effects of a disorder or condition, e.g. hematological malignancy, associated with an altered RUNX1 activity or expression. According to a specific embodiment treating also refers to preventing.

As used herein the term “subject in need thereof” refers to a mammal, preferably a human being at any age which may benefit from the treatment modality of the present invention. According to a specific embodiment, the subject has a hematological malignancy associated with an altered RUNX1 activity or expression.

As used herein the term “RUNX1” relates to the wild-type Runt-related transcription factor 1, also known as acute myeloid leukemia 1 protein (AML1) or core-binding factor subunit alpha-2 (CBFA2). In humans, the gene RUNX1 is 260 kilobases (kb) in length, and is located on chromosome 21 (21q22.12). The protein RUNX1 typically acts as a transcription factor that regulates the differentiation of hematopoietic stem cells into mature blood cells. As a transcription factor, RUNX1's DNA binding ability is enabled by its runt domain. Exemplary protein accession numbers for human RUNX1 (wild-type RUNX1) include NP_001001890 (SEQ ID NO: 58), NP_001116079 (SEQ ID NO: 56) and NP_001745 (SEQ ID NO: 44). Exemplary nucleic acid accession numbers for human RUNX1 (wild-type RUNX1) mRNA include, but are not limited to, NM_001001890 (SEQ ID NO: 57), NM_001122607 (SEQ ID NO: 55) and NM_001754 (SEQ ID NO: 43).

As used herein the term “altered RUNX1 activity or expression” refers to a deviation in activity e.g., DNA binding activity, expression (e.g., over expression or under expression), localization (e.g., altered localization) as compared to that of the wild-type gene and its product.

Thus, the term “altered RUNX1 activity” encompasses altered DNA binding properties (i.e. increased or decreased DNA binding of about 10%, 20%, 30%, 40%, 50%, 60%, 70%, 80%, 90% or 100%, as compared to that of wild-type RUNX1) and/or altered localization and/or altered protein interaction such as with the core binding factor β (CBFβ). The altered RUNX1 activity may be a result of an indirect factor [e.g. alteration in the activity or expression of a RUNX1 cofactor e.g. core-binding protein-β (CBFβ)].

The term “altered RUNX1 expression” refers to disregulated expression i.e., over expression or under expression e.g., of about 10%, 20%, 30%, 40%, 50%, 60%, 70%, 80%, 90% or 100% as compared to that of wild-type transcription or protein product. The altered expression may also refer to structural alteration (e.g., mutation such as insertion, deletion, point mutation.

According to a specific embodiment, the altered RUNX1 results in a RUNX1 fusion protein, also known as a chimeric protein (i.e. a protein created through the joining of two or more genes which originally encode separate proteins). In numerous instances, a chromosomal translocation occurs between the RUNX1 gene [located on chromosome 21 (21q22.12)] with another gene (e.g. the ETO gene located on chromosome 8q22, or ETV6 gene located on chromosome 12p13) resulting in generation of a fusion protein [e.g., fusion protein AML-ETO or ETV6-RUNX1 (TEL/AML1), respectively].

Exemplary fusion proteins comprising RUNX1 include AML1-ETO (A-E) (as set forth in SEQ ID NO: 59) comprising the RUNX1 portion of the peptide as encoded by the mRNA sequence set forth in SEQ ID NO: 63; AML1-EVI1 (SEQ ID NO: 60) comprising the RUNX1 portion of the peptide as encoded by the mRNA sequence set forth in SEQ ID NO: 65; and ETV6-RUNX1 (also known as TEL/AML1) comprising the RUNX1 portion of the peptide as encoded by the mRNA sequence set forth in SEQ ID NO: 64.

Diseases and conditions, which involve altered RUNX1 activity or expression are those in which such an altered activity or expression of RUNX1 is evident.

Any measurement of RUNX1 activity or expression may be carried out in accordance with the present teachings in order to detect altered RUNX1, these include, but are not limited to Western blot analysis, ELISA, Immunofluorescent staining, gel-shift assays and transcription factor binding assays such as ChIP-Seq.

Detection of RUNX1 fusion proteins may be carried out using any method known in the art, including but not limited to, flow cytometric analysis, chromosome analysis, reverse transcriptase-PCR (RT-PCR) or fluorescence in situ hybridization (FISH) probes. Such FISH probes include, for example, the FISH Probe Kit for detection of the t(12;21)(p13;q22) translocation between the ETV6 gene and the RUNX1 gene, available e.g. from Abbott Molecular (Abbott Molecular/Vysis; Des Plaines, Ill., USA), and the FISH Probe Kit for detection of the t(8;21)(q21.3;q22) reciprocal translocation between the RUNX1 gene and the ETO gene, available e.g. from Abbott Molecular (Abbott Molecular/Vysis; Des Plaines, Ill., USA). Likewise, detection of t(3;21) leukemia may be carried out e.g. by the commercially available EVI1 three-color break-apart FISH probe (MetaSystems, Altlussheim, Germany) and AML1/ETO dual color dual fusion FISH probe (Abbott Molecular/Vysis; Des Plaines, Ill., USA).

Additionally, inversion 16 mutations which affect RUNX1 activity, as further detailed hereinbelow, may be detected, for example, using dual color fluorescence in situ hybridization (D-FISH) using a LSI CBFβ inv(16) break apart probe labeled by Spectrum red and Spectrum green, as taught by He Y X et al., Zhonghua Er Ke Za Zhi. (2012) 50(8):593-7, incorporated herein by reference.

A number of diseases and conditions, which involve altered RUNX1 activity or expression, can be treated using the present teachings. The most prevalent conditions involving altered RUNX1 activity or expression are hematological malignancies.

The term “hematological malignancies” (also named hematopoietic malignancies) as used herein refer to types of cancer that affect blood, bone marrow and lymph nodes. The hematological malignancies may comprise primary or secondary malignancies.

As used herein, the term “hematopoietic cells”, also termed hematopoietic stem cells (HSCs), refers to blood cells that give rise to all the other blood cells including e.g. myeloid cells (monocytes and macrophages, neutrophils, basophils, eosinophils, erythrocytes, megakaryocytes/platelets, dendritic cells) and lymphoid cells (T-cells, B-cells, NK-cells).

According to one embodiment, the hematological malignancy comprises a leukemia or lymphoma.

The term “lymphoma” means a type of cancer occurred in the lymphatic cells of the immune system and includes, but is not limited to, mature B-cell lymphomas, mature T-cell and natural killer cell lymphomas, Hodgkin's lymphomas, Non-Hodgkin lymphomas and immunodeficiency-associated lymphoproliferative disorders. The lymphoma can be relapsed, refractory or resistant to conventional therapy.

The term “leukemia” refers to malignant neoplasms of the blood-forming tissues. Leukemia of the present invention includes lymphocytic (lymphoblastic) leukemia and myelogenous (myeloid or nonlymphocytic) leukemia. Exemplary types of leukemia includes, but are not limited to, chronic lymphocytic leukemia, (CLL), chronic myelocytic leukemia (CML) [also known as chronic myelogenous leukemia (CML)], acute lymphoblastic leukemia (ALL) and acute myeloid leukemia (AML) [also known as acute myelogenous leukemia (AML), acute nonlymphocytic leukemia (ANLL) and acute myeloblastic leukemia (AML)]. The leukemia can be relapsed, refractory or resistant to conventional therapy.

The term “relapsed” refers to a situation where patients who have had a remission of leukemia/lymphoma after therapy have a return of leukemia/lymphoma cells in the marrow/lymph and a decrease in normal hematopoietic cells.

The term “refractory or resistant” refers to a circumstance where patients, even after intensive treatment, have residual leukemia/lymphoma cells in their marrow/lymph. The cancer may be resistant to treatment immediately or may develop a resistance during treatment.

The term “acute leukemia” means a disease that is characterized by a rapid increase in the numbers of immature blood cells that transform into malignant cells, rapid progression and accumulation of the malignant cells, which spill into the bloodstream and spread to other organs of the body.

The term “chronic leukemia” means a disease that is characterized by the excessive build up of relatively mature, but abnormal, white blood cells.

According to one embodiment, the leukemia is an acute myeloid leukemia (AML).

According to a specific embodiment, the leukemia (e.g. AML) is type t(8;21). AML type t(8;21) refers to an acute myeloid leukemia in which a translocations between chromosome 8 and 21 [t(8;21)] occurs. The 8;21 translocation (typically with breaks at 8q22 and 21q22.3) is a recurring translocation observed in approximately 20% of patients with acute myeloid leukemia [e.g. AML type M2, i.e. acute myeloblastic leukemia with granulocytic maturation]. This translocation results in the fusion of two genes, AML1 on chromosome 21, and ETO on chromosome 8, with the formation of a chimeric gene AML1/ETO (A-E) on the derivative 8 [der(8)] chromosome. The chimeric protein A-E contains the DNA-binding domain of RUNX1 (the runt domain) linked to the major part of ETO, which by itself lacks DNA-binding capacity. The chimeric protein A-E is involved in impaired activation (e g inhibition) of key hematopoietic transcription factors.

According to a specific embodiment, the leukemia (e.g. AML or CML) is type t(3;21). AML type t(3;21) refers to an acute myeloid leukemia in which a translocations between chromosome 3 and 21 [t(3;21)] occurs. The t(3;21)(q26;q22) translocation involving RUNX1 (AML1) occurs in a small number (approximately 1%) of AML or myelodysplastic syndrome (MDS), and in the blast phase (BP) of chronic myeloproliferative disorders (CMPD), particularly chronic myelogenous leukemia (CML). In this translocation, portions of the AML1 gene are variably fused to 3 genes located within the 3q26 region: EAP, MDS1, and/or EVI1. These fusion products, in cooperation with other genetic abnormalities, are capable of blocking myeloid differentiation possibly by interfering with the normal transcriptional regulatory functions of AML1.

According to a specific embodiment, the leukemia (e.g. AML) is type inv(16). AML type inv(16) refers to an acute myeloid leukemia with inversions in chromosome 16 [inv(16)]. This chromosomal aberrations includes both inv(16)(p13q22) and t(16;16(p13;q22). This inversion fuses chromosome 16q22 encoded core-binding factor subunit beta (CBFβ) gene with the MYH11 gene, which resides at the 16p13 region and encodes the smooth-muscle myosin-heavy chain (SMMHC). The resulting chimeric oncoprotein is known as CBFβ-SMMHC. CBFβ-SMMHC (C-S) is a dominant inhibitor of RUNX1 activity which impairs myeloid differentiation and contributes to AML development.

According to one embodiment, the leukemia is an acute lymphoblastic leukemia (ALL).

According to a specific embodiment, the leukemia (e.g. ALL) is type t(12;21). ALL type t(12;21) refers to an acute lymphoblastic leukemia in which a translocations between chromosome 12 and 21 [t(12;21)] occurs. The 12;21 translocation (typically p12;q22) is a recurring translocation in patients with B-cell lineage acute lymphoblastic leukemia (ALL) and is observed in approximately 30% of patients with childhood B-cell acute lymphoblastic leukemia. This translocation fuses the potential dimerization motif from the ets-related factor ETV6 (TEL) to the N terminus of RUNX1 (AML1), resulting in a fusion protein ETV6-RUNX1 (TEL/AML1). The t(12;21) fusion protein dominantly interferes with AML-1B-dependent transcription.

As illustrated in the Examples section which follows, the present inventors have shown that the expression of wild-type (WT) RUNX1 is essential for survival and leukemogenesis of leukemic cells. Furthermore, the present inventors have shown that attenuation of wild-type RUNX1 activity directs these cells to apoptosis.

As mentioned hereinabove, the methods of the present invention are performed by administering to a subject in need thereof a therapeutically effective amount of an agent which directly downregulates an activity or expression of RUNX1.

As used herein the term “directly” means that the agent acts upon the RUNX1 nucleic acid sequence or protein and not on a co-factor, an upstream activator or downstream effector of RUNX1.

According to one embodiment, the agent which downregulates an activity or expression of RUNX1 does not substantially affect an activity or expression of the altered RUNX1. According to an embodiment, the agent of the present invention affects the activity or expression of the altered RUNX1 by no more than 1%, 2%, 3%, 4%, 5%, 6%, 7%, 8%, 9% or 10%.

Thus, according to a specific embodiment such a RUNX1 inhibitor is designed to selectively bind the wild-type protein or nucleic acid sequence (e.g., RNA) but not the altered RUNX1 as defined above.

Downregulation of RUNX1 can be effected on the genomic and/or the transcript level using a variety of molecules which interfere with transcription and/or translation [e.g., RNA silencing agents (e.g., antisense, siRNA, shRNA, micro-RNA), Ribozyme and DNAzyme], or on the protein level using e.g., antagonists, enzymes that cleave the polypeptide and the like.

Following is a list of agents capable of downregulating expression level and/or activity of RUNX1. Measures are taken to direct the agent to the cellular localization where RUNX1 is active e.g., nucleus.

One example, of an agent capable of downregulating RUNX1 is an antibody or antibody fragment capable of specifically binding RUNX1. Preferably, the antibody specifically binds at least one epitope of RUNX1. The antibody is designed to interfere with RUNX1 activity as described above (e.g., interfere with DNA binding, localization, protein interaction). As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Exemplary RUNX1 targeting antibodies which may be used in accordance with the present teachings include those commercially available from Aviva Systems Biology, LifeSpan BioSciences and Zyagen Laboratories.

A suitable RUNX1 antibody can be an antibody which targets the wild-type RUNX1 and not the altered RUNX1. Thus, for example, for treatment of a subject who has type inv(16) leukemia (e.g. AML), the antibody may target a sequence (or portion thereof) as set forth in SEQ ID NO: 44, 56 or 58. For treatment of a subject who has type t(8;21) leukemia (e.g. AML), the antibody may target a sequence (or portion thereof) as set forth in SEQ ID NO: 48. For treatment of a subject who has type t(3;21) leukemia (e.g. AML or CML), the antibody may target a sequence (or portion thereof) as set forth in SEQ ID NO: 62. For treatment of a subject who has type t(12;21) leukemia (e.g. ALL), the antibody may target a sequence (or portion thereof) as set forth in SEQ ID NO: 46.

Any method known in the art may be used to target the anti-RUNX1 antibodies into live cells (e.g. hematological malignant cells). Thus, for example, efficient encapsulation and delivery of antibodies into live cells (e.g. malignant cells) may be carried out as taught by Marzia Massignani et al. (Marzia Massignani et al., Cellular delivery of antibodies: effective targeted subcellular imaging and new therapeutic tool, Nature Precedings, 10 May 2010) incorporated herein by reference. In brief, this delivery system is based on poly(2-(methacryloyloxy)ethyl phosphorylcholine)-block-(2-(diisopropylamino)ethyl methacrylate), (PMPC-PDPA), a pH sensitive diblock copolymer that self-assembles to form nanometer-sized vesicles, also known as polymersomes, at physiological pH. These polymersomes can successfully deliver relatively high antibody payloads within live cells. Once inside the cells, the antibodies can target their epitope by immune-labelling of cytoskeleton, Golgi, and transcription factor proteins in live cells.

Downregulation of RUNX1 can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., RUNX1) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003;13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the RUNX mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (wwwdotambiondotcom/techlib/tn/91/912dothtml).

Second, potential target sites are compared to an appropriate genomic database (e.g., human, mouse, rat etc.) using any sequence alignment software, such as the BLAST software available from the NCBI server (wwwdotncbidotnlmdotnihdotgov/BL AST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

A suitable RUNX1 siRNA can be an siRNA which targets the wild-type RUNX1 and not the altered RUNX1. Thus, for example, for treatment of a subject who has type inv(16) leukemia (e.g. AML), the siRNA may target a sequence (or portion thereof) as set forth in SEQ ID NO: 43, 55 or 57. For treatment of a subject who has type t(8;21) leukemia (e.g. AML), the siRNA may target a sequence (or portion thereof) as set forth in SEQ ID NO: 47. For treatment of a subject who has type t(3;21) leukemia (e.g. AML or CML), the siRNA may target a sequence (or portion thereof) as set forth in SEQ ID NO: 61. For treatment of a subject who has type t(12;21) leukemia (e.g. ALL), the siRNA may target a sequence (or portion thereof) as set forth in SEQ ID NO: 45.

For example, a suitable RUNX1 siRNA can be the siRNA as set forth in SEQ ID NO: 52, 53, 66, 67, 68, 69, 70, 71, 72 or 73.

Any method known in the art may be used to target the RUNX1 siRNA into live cells (e.g. hematological malignant cells). Thus, for example, efficient transport of siRNA into malignant cells may be carried out as taught by Ziv Raviv (Ziv Raviv, The Development of siRNA-Based Therapies for Cancer, Pharmaceutical Intelligence, May 9, 2013) incorporated herein by reference. In brief, for an efficient transport of the siRNA RUNX1, a delivery system can be formulated using liposome-based nanoparticles (NP) or other nanocarriers to facilitate the siRNA effective systemic distribution. Furthermore, PEGylation of the NPs carriers can be carried out to reduce non-specific tissue interactions, increase serum stability and half life, and reduce immunogenicity of the siRNA molecule. For site specific targeting of the RUNX1 siRNA (e.g. into hematological malignant cells), target tissue-specific distribution of the siRNA drug can be performed by attaching on the outer surface of the nanocarrier a ligand that directs the siRNA drug to the tumor site or tumor cell.

It will be appreciated that the RNA silencing agent of some embodiments of the invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specifity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Exemplary miRNA that may be used in accordance with the present invention to inhibit RUNX1 include those which inhibit RUNX1 function via binding to its 3′ untranslated region (3′UTR) such as miR-27a/b (as taught in Ben-Ami et al., Proc Natl Acad Sci USA. (2009) 106(1): 238-43, fully incorporated herein by reference) and miR-17-20-106 (Fontana et. al., Nat Cell Biol. (2007) (7):775-87, fully incorporated herein by reference).

It will be appreciated from the description provided herein above, that contacting hematological malignant cells (leukemia or lymphoma cells) with a miRNA may be affected in a number of ways:
1. Transiently transfecting the malignant cells with the mature double stranded miRNA;
2. Stably, or transiently transfecting the malignant cells with an expression vector which encodes the mature miRNA.
3. Stably, or transiently transfecting the malignant cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA.
4. Stably, or transiently transfecting the malignant cells with an expression vector which encodes the pri-miRNA. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. Preparation of miRNAs mimics can be effected by chemical synthesis methods or by recombinant methods.

Another agent capable of downregulating a RUNX1 is a DNAzyme molecule capable of specifically cleaving an mRNA transcript or DNA sequence of the RUNX1. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther wwwdotasgtdotorg). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a RUNX1 can also be effected by using an antisense polynucleotide capable of specifically hybridizing with an mRNA transcript encoding the RUNX1.

Design of antisense molecules which can be used to efficiently downregulate a RUNX1 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a RUNX1 is a ribozyme molecule capable of specifically cleaving an mRNA transcript encoding a RUNX1. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of an RUNX1 gene in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988;241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003;112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the RUNX1 regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003;31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003;112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Another agent capable of downregulating RUNX1 would be any molecule which binds to and/or cleaves RUNX1. Such molecules can be RUNX1 antagonists, or RUNX1 inhibitory peptide.

It will be appreciated that a non-functional analogue of at least a catalytic or binding portion of RUNX1 can be also used as an agent which downregulates RUNX1.

According to one embodiment, the agent which directly downregulates an activity or expression of RUNX1 is a polynucleotide agent directed to a nucleic acid region selected from the group consisting of SEQ ID NO: 43, SEQ ID NO: 45, SEQ ID NO: 47, SEQ ID NO: 49, SEQ ID NO: 50, SEQ ID NO: 55 or SEQ ID NO: 57.

According to one embodiment, the polynucleotide agent comprises 15-25 nucleotides.

According to an embodiment, there is provided an isolated polynucleotide which directly downregulates RUNX1 but not AML1-ETO (A-E), AML1-EVI1 or ETV6-RUNX1 (TEL/AML1).

According to one embodiment, the isolated polynucleotide comprises a nucleic acid sequence as set forth in SEQ ID NO: 52 and SEQ ID NO: 53.

According to another embodiment, there is provided a nucleic acid construct comprising the isolated polynucleotide of some embodiments of the invention.

Another agent which can be used along with some embodiments of the invention to downregulate RUNX1 is a small molecule.

Any small molecule which directly binds and downregulates RUNX1 may be used according to the present teachings. Preferably the small molecule of the present invention binds the RUNX1 runt domain and inhibits binding of RUNX1 to a DNA site.

It will be appreciated that each of the downregulating agents described hereinabove or the expression vector encoding the downregulating agents can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the RUNX1 downregulating agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to an embodiment of the present invention, the pharmaceutical composition is formulated for penetrating a cell membrane. Thus, for example, the pharmaceutical composition may comprise a lipid vesicle.

Alternately, one may administer the pharmaceutical composition in a local rather than systemic manner, for example, via injection of the pharmaceutical composition directly into a tissue region of a patient (e.g. necrotic tissue).

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. RUNX1 downregulating agent) effective to prevent, alleviate or ameliorate symptoms of a disorder (e.g., hematologic malignancy) or prolong the survival of the subject being treated.

According to an embodiment of the present invention, an effect amount of the agent of the present invention, is an amount selected to initiate apoptosis (i.e. cell apoptosis) of hematopoietic cells of the hematologic malignancy.

The term “cell apoptosis” as used herein refers to the cell process of programmed cell death. Apoptosis characterized by distinct morphologic alterations in the cytoplasm and nucleus, chromatin cleavage at regularly spaced sites, and endonucleolytic cleavage of genomic DNA at internucleosomal sites. These changes include blebbing, cell shrinkage, nuclear fragmentation, chromatin condensation, and chromosomal DNA fragmentation. Furthermore, apoptosis produces cell fragments called apoptotic bodies that phagocytic cells are able to engulf and quickly remove before the contents of the cell can spill out onto surrounding cells and cause damage.

According to one embodiment, the cell apoptosis is caspase dependent.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays (see e.g. Examples 1-8 in the Examples section which follows). Furthermore, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Animal models for hematologic malignancies include the humanized mouse model [see e.g. Inoue Y, Exp Hematol. (2007) 35(3):407-15] and the porcine animal model [see e.g. Cho P S et al. Blood. (2007) 1; 110(12): 3996-4004].

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In order to test treatment efficacy, the subject may be evaluated by physical examination as well as using any method known in the art for evaluating hematologic malignancies. Thus, for example, a bone marrow cell sample or lymph node tissue sample may be obtained (e.g. from a subject) and hematopoietic malignant cells may be identified, by light, fluorescence or electron microscopy techniques (e.g. by FACS analysis testing for specific cellular markers). Furthermore, the subject may undergo testing for hematological malignancies including e.g. blood tested, MRI, CT, pet-CT, ultrasound, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The agents of the invention can be suitably formulated as pharmaceutical compositions which can be suitably packaged as an article of manufacture. Such an article of manufacture comprises a label for use in treating a hematologic malignancy, the packaging material packaging a pharmaceutically effective amount of the RUNX1 downregulating agent.

It will be appreciated that each of the agents or compositions of the present invention may be administered in combination with other known treatments, including but not limited to, pro-apoptotic agents, chemotherapeutic agents (i.e., a cytotoxic drug), hormonal therapeutic agents, radiotherapeutic agents, anti-proliferative agents and/or any other compound with the ability to reduce or abrogate the uncontrolled growth of aberrant cells such as malignant hematologic cells.

According to one embodiment, the pro-apoptotic agent is for targeted killing of the hematologic malignancy.

According to a specific embodiment, the pro-apoptotic agent is caspase dependent (e.g. Gambogic acid).

Exemplary pro-apoptotic agents (i.e. apoptosis inducers) which may be used in accordance with the present invention include those which affect cellular apoptosis through a variety of mechanisms, including DNA cross-linking, inhibition of anti-apoptotic proteins and activation of caspases. Exemplary pro-apoptotic agents include, but are not limited to, Actinomycin D, Apicidin, Apoptosis Activator 2, AT 101, BAM 7, Bendamustine hydrochloride, Betulinic acid, C 75, Carboplatin, CHM 1, Cisplatin, Curcumin, Cyclophosphamide, 2,3-DCPE hydrochloride, Deguelin, Doxorubicin hydrochloride, Fludarabine, Gambogic acid, Kaempferol, 2-Methoxyestradiol, Mitomycin C, Narciclasine, Oncrasin 1, Oxaliplatin, Piperlongumine, Plumbagin, Streptozocin, Temozolomide and TW 37.

Non-limiting examples of chemotherapeutic agents include, but are not limited to, platinum-based drugs (e.g., oxaliplatin, cisplatin, carboplatin, spiroplatin, iproplatin, satraplatin, etc.), alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, 6-mercaptopurine, methotrexate, leucovorin, capecitabine, cytarabine, floxuridine, fludarabine, gemcitabine (Gemzar®), pemetrexed (ALIMTA®), raltitrexed, etc.), plant alkaloids (e.g., vincristine, vinblastine, vinorelbine, vindesine, podophyllotoxin, paclitaxel (Taxol®), docetaxel (Taxotere®), etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), pharmaceutically acceptable salts thereof, stereoisomers thereof, derivatives thereof, analogs thereof, and combinations thereof.

Examples of hormonal therapeutic agents include, but are not limited to, aromatase inhibitors (e.g., aminoglutethimide, anastrozole (Arimidex®), letrozole (Femora®), vorozole, exemestane (Aromasin®), 4-androstene-3,6,17-trione (6-OXO), 1,4,6-androstatrien-3,17-dione (ATD), formestane (Lentaron®), etc.), selective estrogen receptor modulators (e.g., bazedoxifene, clomifene, fulvestrant, lasofoxifene, raloxifene, tamoxifen, toremifene, etc.), steroids (e.g., dexamethasone), finasteride, and gonadotropin-releasing hormone agonists (GnRH) such as goserelin, pharmaceutically acceptable salts thereof, stereoisomers thereof, derivatives thereof, analogs thereof, and combinations thereof.

Examples of radiotherapeutic agents include, but are not limited to, radionuclides such as .sup.47Sc, .sup.64Cu, .sup.67Cu, .sup.89Sr, .sup.86Y, .sup.87Y, .sup.90Y, .sup.105Rh, .sup.111Ag, .sup.111In, .sup.117mSn, .sup.149Pm, .sup.153Sm, 166Ho, .sup.177Lu, .sup.186Re, .sup.188Re, .sup.211At, and .sup.212Bi, optionally conjugated to antibodies directed against tumor antigens.

Exemplary anti-proliferative agents include mTOR inhibitors such as sirolimus (rapamycin), temsirolimus (CCI-779), and everolimus (RAD001); Akt inhibitors such as IL6-hydroxymethyl-chiro-inositol-2-(R)-2-O-methyl-3-O-octadecyl-sn-glycer ocarbonate, 9-methoxy-2-methylellipticinium acetate, 1,3-dihydro-1-(1-44-(6-phenyl-1H-imidazo [4,5-g]quinoxalin-7-yl)phenyl)me-thyl)-4-piperidinyl)-2H-benzimidazol-2-one, 10-(4′-(N-diethylamino)butyl)-2-chlorophenoxazine, 3-formylchromone thiosemicarbazone (Cu(II)Cl.sub.2 complex), API-2, a 15-mer peptide derived from amino acids 10-24 of the proto-oncogene TCL1 (Hiromura et al., J. Biol. Chem., 279:53407-53418 (2004), KP372-1, and the compounds described in Kozikowski et al., J. Am. Chem. Soc., 125:1144-1145 (2003) and Kau et al., Cancer Cell, 4:463-476 (2003); and combinations thereof.

The agents or compositions of the present invention may be administered prior to, concomitantly with or following administration of the latter.

According to one embodiment, there is provided a method of inducing apoptosis of hematopoietic cells associated with an altered RUNX1 activity or expression, the method comprising administering to the hematopoietic cells a therapeutically effective amount of an agent which directly downregulates an activity or expression of RUNX1, thereby inducing the apoptosis of the hematopoietic cells.

According to an embodiment, the hematopoietic cells comprise myeloma cells or lymphocytes.

According to one embodiment, there is provided a method of inducing apoptosis of hematopoietic cells of a subject having a hematological malignancy associated with an altered RUNX1 activity or expression, the method comprising administering to the subject a therapeutically effective amount of an agent which directly downregulates an activity or expression of RUNX1, thereby inducing apoptosis of the hematopoietic cells of the subject.

According to an embodiment, the hematological malignancy is a leukemia or lymphoma.

According to one embodiment, the method of the present invention is effected in vivo.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

Cell Culture and Expression Analysis

Kasumi-1 cells were purchased from the ATCC (Manassas, Va.) and maintained in RPMI-1640 supplemented with 20% fetal bovine serum (FBS), 2 mM L-glutamine and 1% penicillin—streptomycin at 37° C. and 5% CO₂. ME-1 cells were obtained from DSMZ (Braunschweig, Germany) and grown in RPMI-1640 medium with 20% heat-inactivated FBS.

Knockdown (KD) of RUNX1 or A-E in Kasumi-1 and ME-1 Cell Lines (siRNA Transfection)

RUNX1-targeting, A-E-targeting or non-targeting control siRNA oligos (Thermo Scientific Dharmacon) were electroporated into Kasumi-1 or ME-1 leukemic cell lines.

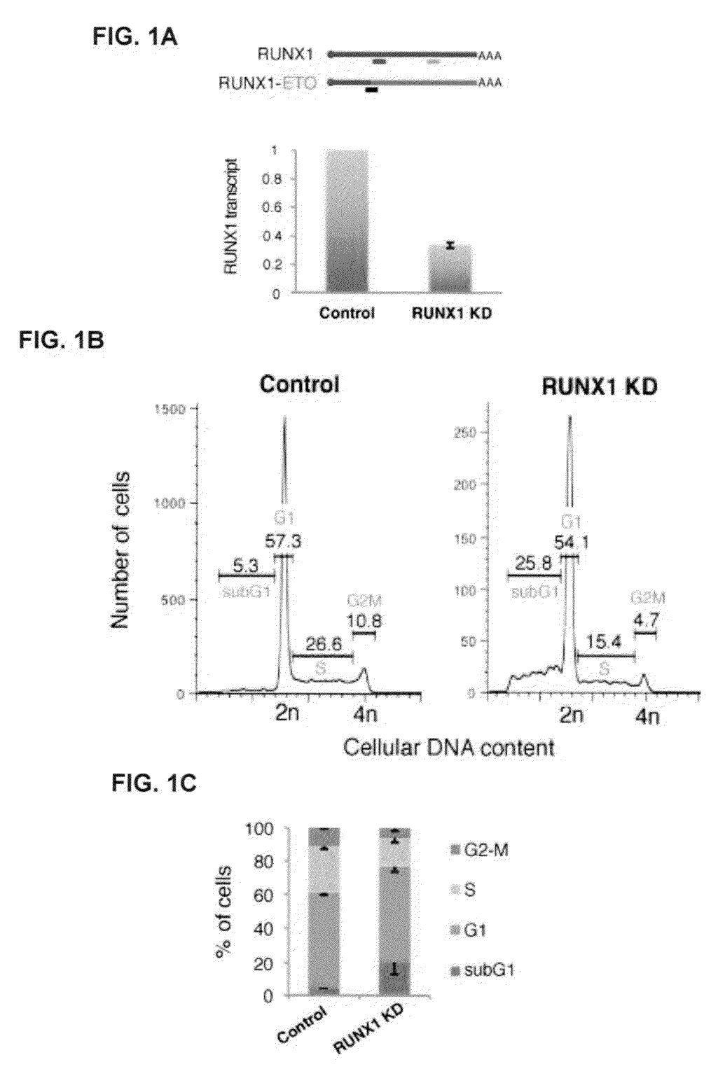

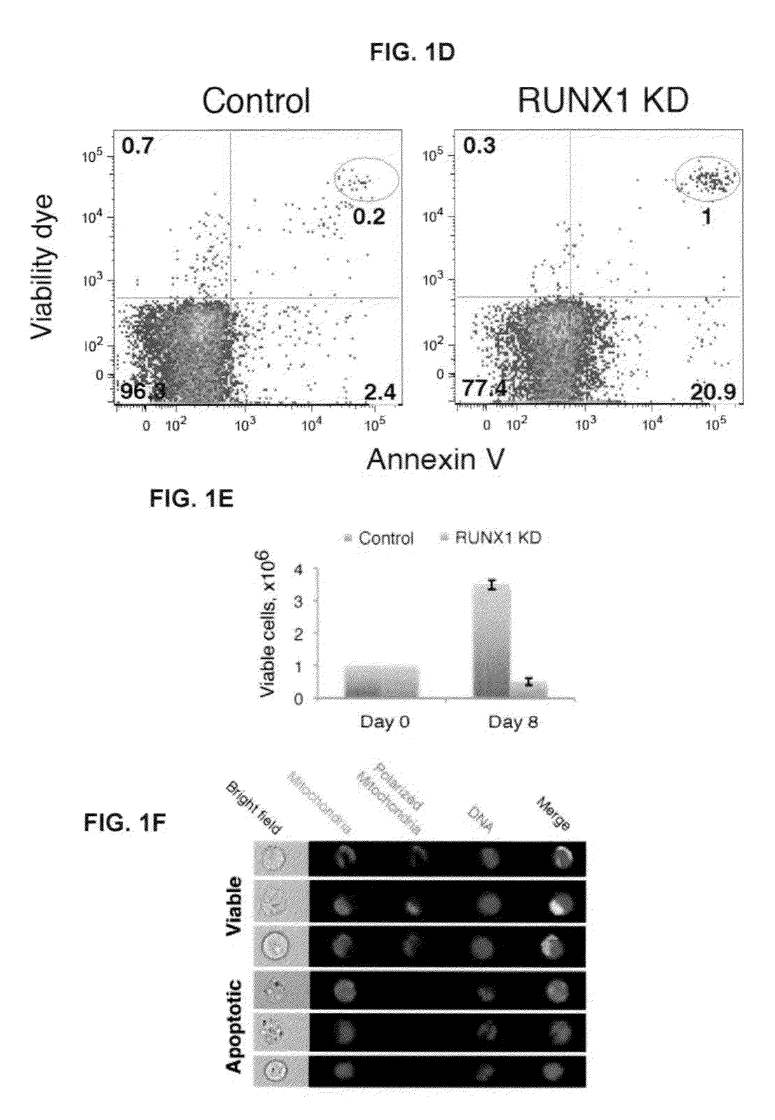

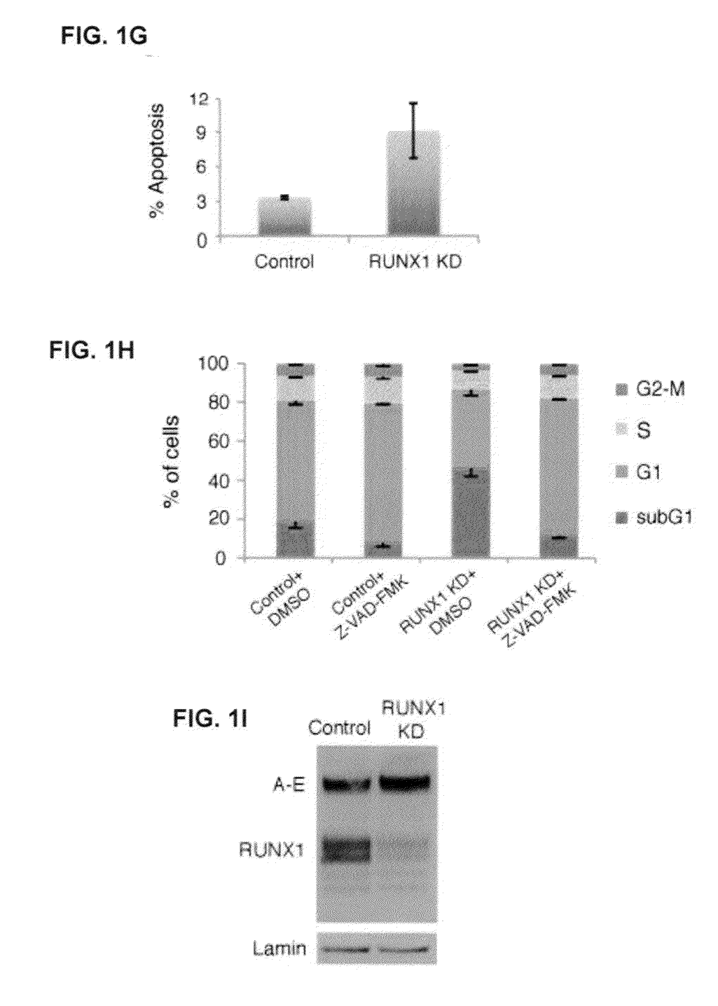

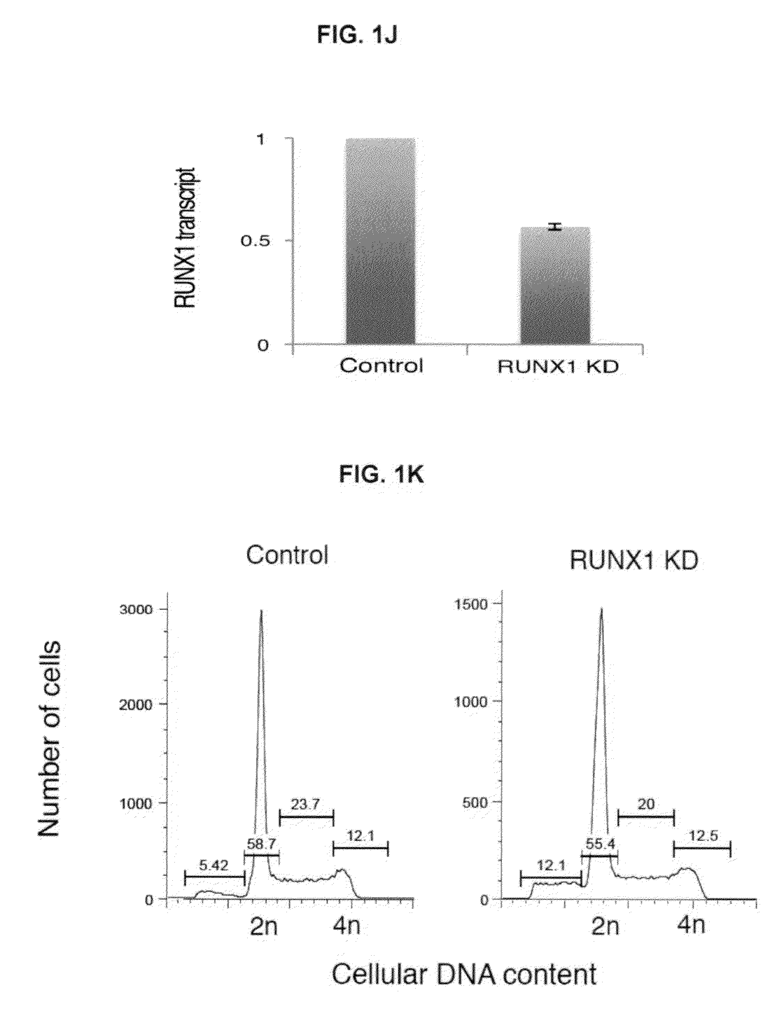

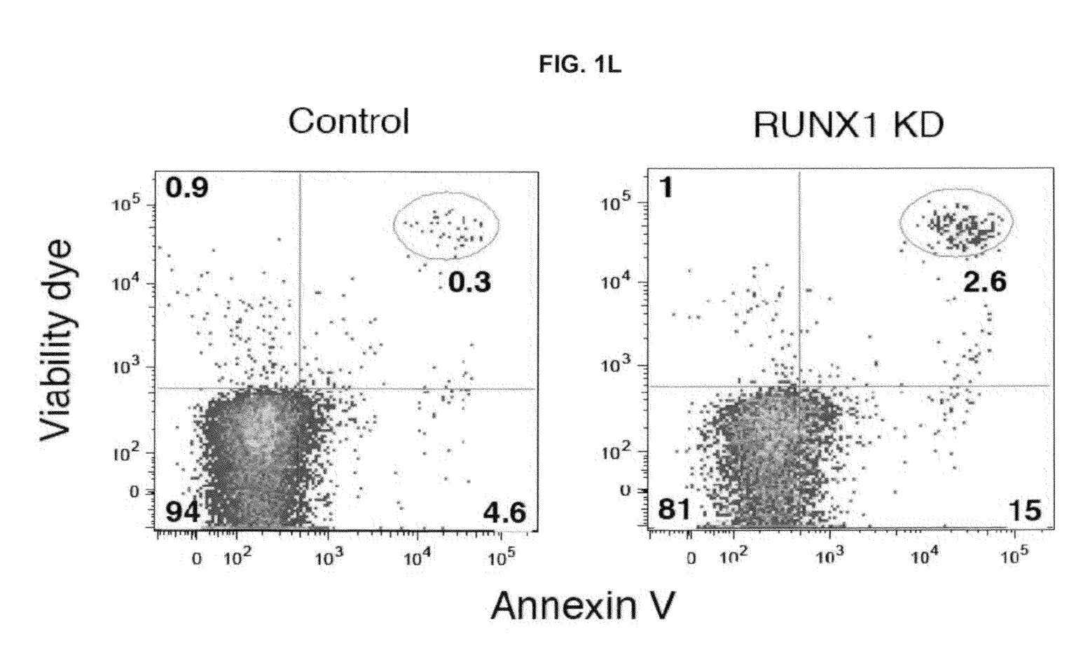

Specifically, Kasumi-1 cells were transfected with 2.5 μM of the relevant siRNA using the cell Line Nucleofector kit V and the P-019 protocol (Amaxa Nucleofector Technology, Lonza). Unless stated otherwise the RUNX1-targeting siRNA that matches the sequence: GACAUCGGCAGAAACUAGA (SEQ ID NO: 49, marked by green in FIG. 1A) was used. A-E KD was conducted using siRNA that targeted the following sequence: CCUCGAAAUCGUACUGAGA SEQ ID NO: 51 as previously taught by Heidenreich, O. et al., Blood (2003) 101, 3157-31631 For delivering siRNA into ME-1 cells the Super Electroporator NEPA21 (NEPAGENE, Japan) was used. KD efficiency was assessed both by RT-qPCR and immunoblotting. For extended (8 days) knockdown, cells were re-transfected with an additional dose of siRNA (2.5 μM), 96 hrs following the first siRNA delivery.

Western Blotting

Cells were collected, washed once in PBS, and nuclear proteins were extracted and analyzed by Western blotting as previously described [Aziz-Aloya, R. et al., Cell Death (1998) 5, 765-773]. Blots were probed with antibodies detecting either RUNX1 c-terminus (derived from sera of in-house rabbits immunized against a specific c-terminal peptide of RUNX1), RUNX1-N-terminus (#4334; Cell Signaling Technology) or ETO (PC283; Calbiochem). Lamin B was used as an internal loading control.

RT-qPCR

Total RNA was reverse-transcribed using miScript reverse transcription kit (QIAGEN) according to manufacturer's instructions. Quantitation of cDNAs was performed by qPCR using Roche LC480 LightCycler with sequence-specific primers (Table 1, below) and miScript SYBR Green PCR kit (QIAGEN). Target transcript quantification was calculated relative to ACTB mRNA, which served as an internal control. Standard errors were calculated using REST.

TABLE 1
List of primer sets used for RT-qPCR

| | Primer | |
|---|---|---|
| Gene name | orientation | Primer sequence (5′→3′) |
| RUNX1 | Forward | TCTGCAGAACTTTCCAGTCG (SEQ ID NO: 1) |
| | Reverse | AAGGCGCCTGGATAGTGCAT (SEQ ID NO: 2) |
| AML1-ETO | Forward | CACCTACCACAGAGCCATCAAA (SEQ ID NO: 3) |
| | Reverse | ATCCACAGGTGAGTCTGGCATT (SEQ ID NO: 4) |
| CEBPA | Forward | TGTATACCCCTGGTGGGAGA (SEQ ID NO: 5) |
| | Reverse | TCATAACTCCGGTCCCTCTG (SEQ ID NO: 6) |
| M-CSF-R | Forward | CTGCCCAGATCGTGTGCTC (SEQ ID NO: 7) |
| | Reverse | AGGTTGAGGGTCAGGACTTTTT (SEQ ID NO: 8) |
| RNASE2 | Forward | TTTACCTGGGCTCAATGGTTTG (SEQ ID NO: 9) |
| | Reverse | TGCATCGCCGTTGATAATTGT (SEQ ID NO: 10) |
| RNASE3 | Forward | GCAGACAGACCAGGAAGGAG (SEQ ID NO: 11) |
| | Reverse | AGGTGAACTGGAACCACAGG (SEQ ID NO: 12) |
| CTSG | Forward | CCACCCTCAATATAATCAGCGG (SEQ ID NO: 13) |
| | Reverse | GTTTCGATTCCGTCTGACTCTTC (SEQ ID NO: 14) |
| ITGB2 | Forward | TGCGTCCTCTCTCAGGAGTG (SEQ ID NO: 15) |
| | Reverse | GGTCCATGATGTCGTCAGCC (SEQ ID NO: 16) |
| CD34 | Forward | CTTTCAACCACTAGCACTAGCC (SEQ ID NO: 17) |
| | Reverse | TGCCCTGAGTCAATTTCACTTC (SEQ ID NO: 18) |
| CD38 | Forward | AGACTGCCAAAGTGTATGGGA (SEQ ID NO: 19) |
| | Reverse | GCAAGGTACGGTCTGAGTTCC (SEQ ID NO: 20) |
| NEK6 | Forward | CAGGACTGTGTCAAGGAGATCG (SEQ ID NO: 21) |
| | Reverse | ATGTTCAGCTCGTTGTCTTCG (SEQ ID NO: 22) |
| CCNA2 | Forward | TGGAAAGCAAACAGTAAACAGCC (SEQ ID NO: 23) |
| | Reverse | GGGCATCTTCACGCTCTATTT (SEQ ID NO: 24) |
| CCNB2 | Forward | CCGACGGTGTCCAGTGATTT (SEQ ID NO: 25) |
| | Reverse | TGTTGTTTTGGTGGGTTGAACT (SEQ ID NO: 26) |
| SPC25 | Forward | GACCCTAAGAATCCTGAGAGCC (SEQ ID NO: 27) |
| | Reverse | GGGGCACTATCTGACACTTCATA (SEQ ID NO: 28) |
| NDC80 | Forward | GTGCCCCTCATACGAACTTCC (SEQ ID NO: 29) |
| | Reverse | GTGCAAAAGGATACCCAAGGT (SEQ ID NO: 30) |
| SGOL1 | Forward | AACTCAGCAGTCACCTCATCT (SEQ ID NO: 31) |
| | Reverse | TGCACCTACGTTTAGGCAGAG (SEQ ID NO: 32) |
| BUB1B | Forward | GCACCGACAATTCCAAGCTC (SEQ ID NO: 33) |
| | Reverse | TGTGCTTCGTTGTGGTACAGA (SEQ ID NO: 34) |
| BUB1 | Forward | ACAATCAACGGAGAAAGCATGA (SEQ ID NO: 35) |
| | Reverse | CTCCACCACCTGATGCAACT (SEQ ID NO: 36) |
| TOP2A | Forward | TGGCTGTGGTATTGTAGAAAGC (SEQ ID NO: 37) |
| | Reverse | TTGGCATCATCGAGTTTGGGA (SEQ ID NO: 38) |
| NEK2 | Forward | TGCTTCGTGAACTGAAACATCC (SEQ ID NO: 39) |
| | Reverse | CCAGAGTCAACTGAGTCATCACT (SEQ ID NO: 40) |
| ACTB | Forward | GGACTTCGAGCAAGAGATGG (SEQ ID NO: 41) |
| | Reverse | AGCACTGTGTTGGCGTACAG (SEQ ID NO: 42) |

FACS Analyses

For cell cycle analysis, cells were stained with Propidium iodide (Sigma-Aldrich) according to standard procedure. For apoptosis assessment, Annexin V apoptosis detection kit was used (eBioscience) combined with the fixable viability dye eFluor 780 (eBioscience). For measurement of CD34/CD38 expression, cells were stained with PE-labeled CD38 (clone HB7; eBioscience) and PE-Cy7-labeled CD34 (Clone 4H11; eBioscience) antibodies. All data were collected using LSRII flow cytometer (BD Biosciences) and analyzed by FlowJo software.

Gene Expression Analysis

Gene expression analysis was performed using RNA isolated from FITC⁺ FACS sorted cells. Isolated RNA was reverse-transcribed, amplified and labeled (WT expression kit, Ambion). Labeled cDNA was analyzed using Human Gene 1.0 ST arrays (Affymetrix), according to the manufacturer's instructions. Arrays were scanned by Gene-Chip scanner 3000 7G. Collected data was summarized and normalized using the RMA method. For Z-VAD-FMK treated Kasumi-1^{RX1-KD} cell gene expression analysis cells were first transfected with control non-targeting (NT) or RUNX1-targeting siRNA and incubated for 60 hrs, Z-VAD-FMK (50 μM) was then added and incubation continued for additional 36 hrs prior to FACS sorting of FITC⁺ cells for RNA isolation.

Genome-Wide Chromatin Immunoprecipitation Sequencing (ChIP-Seq) Data Acquisition and Analysis

Two biological replicate ChIP-Seq experiments were done for the specific detection of either RUNX1- or AML1-ETO-bound genomic regions according to standard procedures previously summarized in Pencovich [Pencovich, N. et al., Blood (2011) 117, e1-14] including several modifications as detailed herein.

In short, cross-linked chromatin from approximately 5-10×10⁷ Kasumi-1 cells was prepared and fragmented to an average size of approximately 200 bp by 30-40 cycles of sonication (30 seconds each) in 15 ml tubes using the Bioruptor UCD-200 sonicator (Diagenode). For immunoprecipitation, the following antibodies were added to 12 mL of diluted, fragmented chromatin: 32 μL of anti-RUNX1 (Aziz-Aloya (1998), supra; Levanon, D. et al., EMBO Mol Med (2011) 3, 593-604) raised against the protein C-terminal fragment; 320 μl of anti-ETO (PC283; Calbiochem). Non-immunized rabbit serum served as control. DNA was purified using QIAquick spin columns (QIAGEN) and sequencing performed using Illumina genome analyzer IIx, according to the manufacturer's instructions. For ChIP-seq analysis, Illumina sequencing of short reads (40 bp) was conducted using the GAII system. ChIP-seq short read tags were mapped to the genome using bowtie. Mapped reads were then extended to 120 bp fragments in the appropriate strand and all fragments were piled up to generate a coverage track in 50 bp resolution.

The genome-wide distribution of coverage was computed on 50 bp bins for each track, and used to normalize piled-up chip-seq coverage by transforming coverage values v to log(1-quantile(v), defining the ChIP-seq binding intensity or binding enrichment. Binding intensities directly was preferably used, while using arbitrarily defined threshold on binding intensity to define binding sites was minimized. In cases where a threshold was needed (e.g. to report indicative statistics on binding, or to facilitate motif finding), genomic bins with normalized coverage >log(1-0.9985) (merging all sites that were within 250 bp of each other) were searched. A control non-immune serum (NIS) ChIP-seq experiment was used to filter spurious binding sites (defined as bins with NIS normalized intensity >log(1-0.9985)).

Definition of A-E and RUNX1 Target Genes

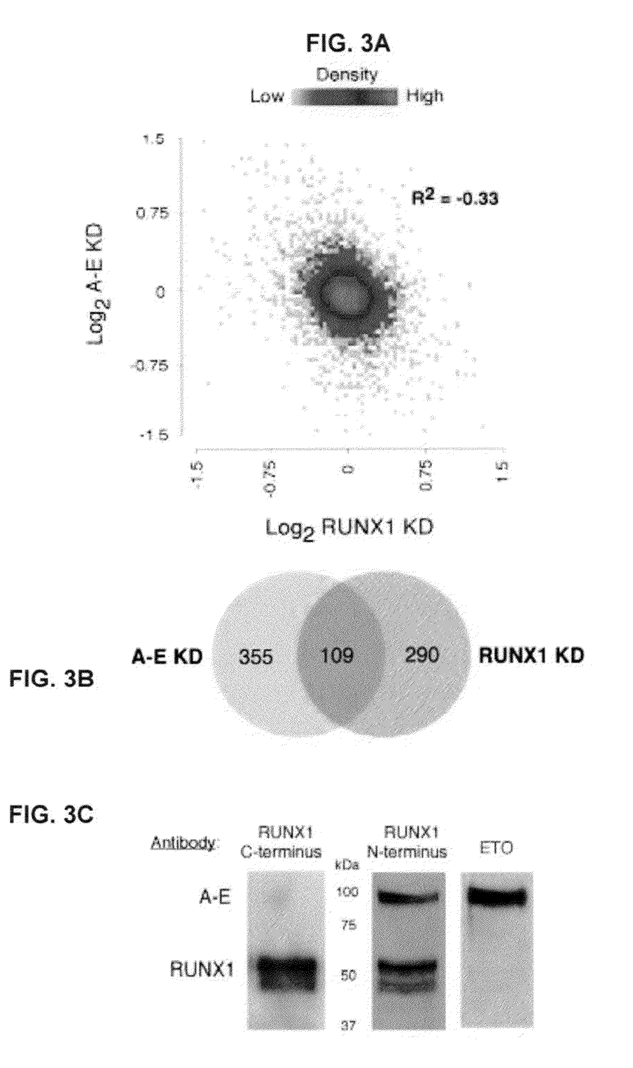

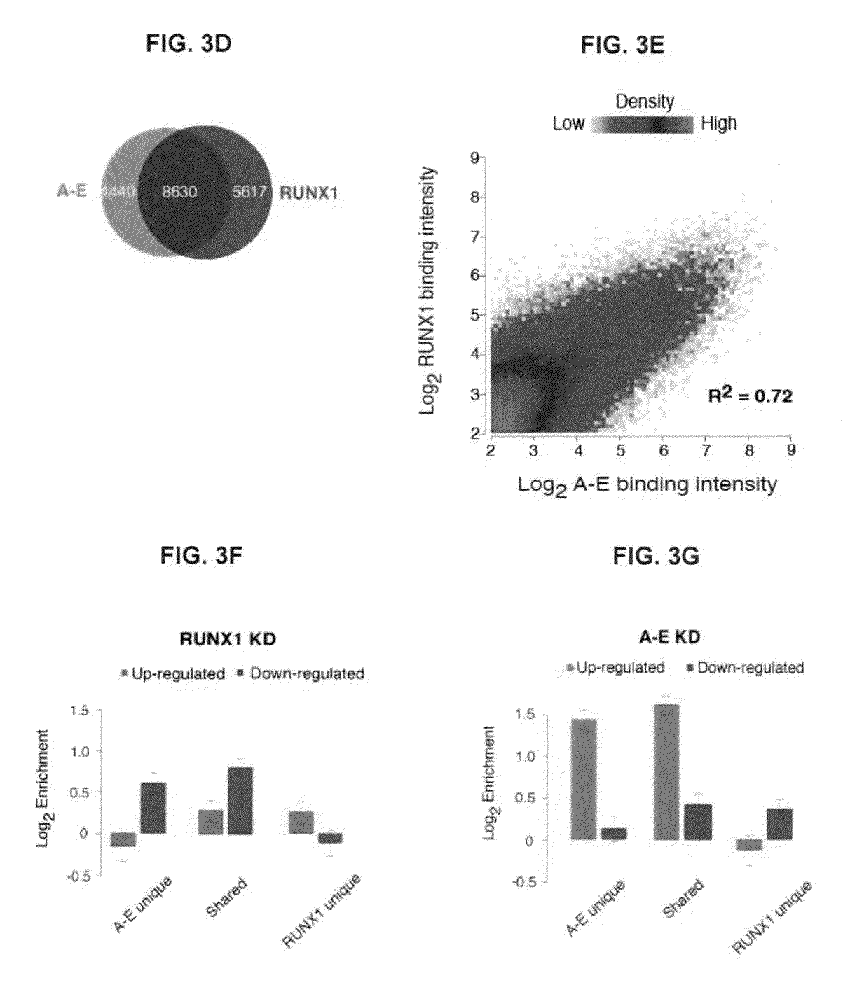

Genes were defined as differentially regulated in response to A-E and RUNX1 KD if the absolute fold difference in gene expression experiments comparing the expression before and after KD was >1.4 with p-value smaller than 0.05 (see “Gene expression analysis” section hereinabove). To derive enrichment data of genes up- and down-regulated in response to KD of RUNX1 and A-E (FIGS. 3F and 3G), genes were annotated according to the presence of RUNX1 or A-E ChIP-seq peak within 10 kb of TSS and the number of up- or down-regulated genes associated with unique or shared bound sites was determined.

Motif Finding

Motif finding on ChIP-seq peaks was performed through an adaptation of the MEME algorithm for usage of a mixture of 5′th order Markov models to describe background sequence distributions (available in A. Tanay website; www.compgenomics(dot)weizmann(dot)ac(dot)il/tanay/). Background model parameters were learned based on 117,000 human enhancer sequences showing H3K4mel ChIP-seq normalized binding intensity >log(1-0.9985) based on ENCODE H1 ES cells data (and using ChIP-seq processing as described above). Motif finding algorithm was performed on 2492 RUNX1, 3140 A-E, and 4652 common (RUNX1 and A-E) binding sites with default parameters.

Motif and Sequence Affinity

Motifs were represented using a positional weighted matrix (PWM) and were used to calculate approximate sequence affinity as was previously described in [Pencovich (2011), surpa]. A PWM model was used to approximate the local binding energy using the formula:

$${E}^{j} = - log ( P ( S ( [ j … j + L - 1 ] \vert j bound ) ) = - {\sum }_{k = 0}^{L - 1} log ( {W}_{k} ( {S}_{j + k} ) )$$

Where j is the position of the sequence S being analyzed, the W parameters define the nucleotide preferences of the motif probabilistically, and L is the motif length. It was noted that the motif consensus will be represented as the sequence with the highest weights and that the approximated binding affinity for a genomic region is derived by summing up motif probabilities over all possible binding positions—

$$E ( S ) = - log {\sum }_{j = 1}^{N - L} {\prod }_{k = 0}^{L - 1} {W}_{k} ( {S}_{j + k} )$$

According to this approach, it was accounted for multiple appearances of suboptimal sequences, while still considering the optimal binding sequences in the region as the most important.

Using this method, one can assess the correspondence between a set of sequences and the motif in a quantitative way by directly considering the affinity. It also enables to compute the PWM enrichment of a set of loci by estimating the distribution of sequence affinities in these loci and in background sequences (e.g. sampling sequences within 2 kb of the target loci). The enrichment value is than computed by testing the fraction of target loci that are within the top 5% of the background affinity distribution, and dividing this value by 0.05.

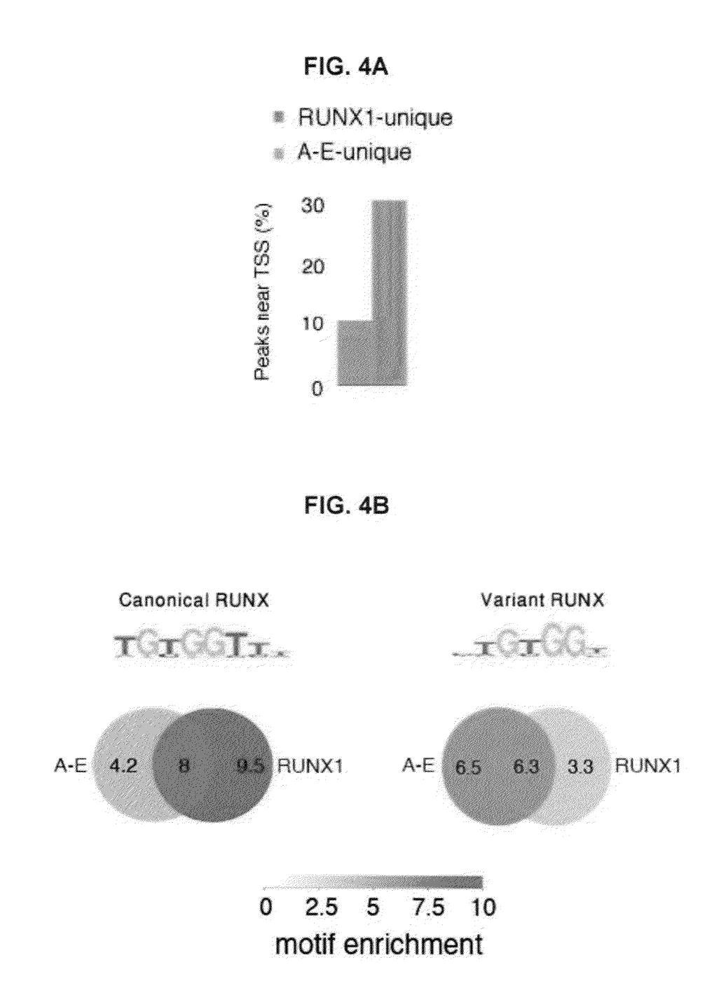

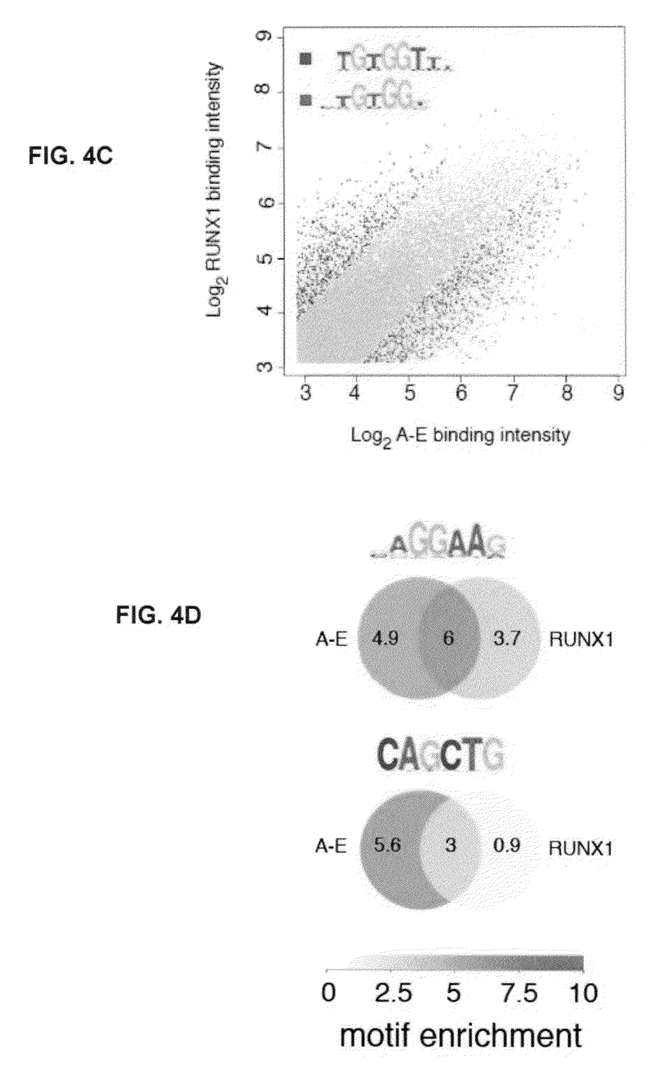

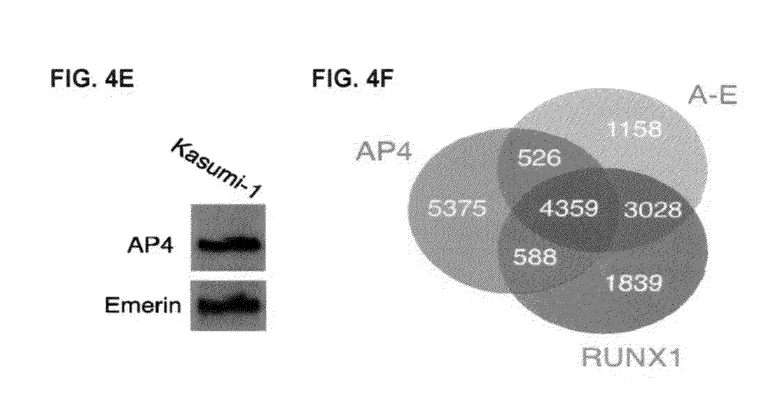

Sequence affinities were also used for quantitative comparison between motif variants enriched in A-E and RUNX1. This was done by computing the distribution of affinity values over all binding sites (separately for each PWM) and then transforming each affinity value e to log(1-quantile(e). The difference between the two normalized PWM affinities could now be used directly, e.g. color coding in FIG. 4C.

Multispectral Imaging Flow Cytometry (ImageStream© System Analysis)

For multispectral imaging flow cytometry, approximately 10⁴ siRNA-treated cells were collected per sample and data were analyzed using image analysis software (IDEAS 4.0; Amnis Corp).

Specifically, images were compensated for fluorescent dye overlap by using single-stain controls. Cells were first gated for single cells using the area and aspect-ratio features and then for focused cells using the Gradient RMS feature, as previously described [George, T. et al., J Immunol Methods (2006) 311, 117-129]. Apoptotic cells were determined using the following two parameters. First, the ratio between the staining-intensities of two mitochondrial probes; green dye which non-selectively stains mitochondria of live/apoptotic/dead cells (Mitogreen) and red-dye which selectively stains only mitochondria of live cells in a voltage-sensitive manner (MitoTracker Red CMXRos). Second, the area of the 50% highest intensity pixels of the DNA staining dye DRAQ5 (Cell Signaling Technology) calculated using the Threshold 50% mask. Cells exhibiting both low Red/Green mitochondrial-staining ratio and low DNA area were considered as apoptotic.

Transcriptome Data Acquisition and Analysis

For transcriptome data acquisition, FITC-labeled non-targeting siRNA oligos (#2013, Block-it fluorescent oligo, Life Technologies) were co-transfected with RUNX1-targeting, A-E-targeting or control NT siRNAs and FITC+ cells were FACS isolated following 96 hr incubation. RNA was obtained using miRNeasy (QIAGEN), its integrity assessed using Bioanalyzer (Agilent Technologies) and transcriptome analysis was conducted as previously described [Pencovich, (2011), supra].

Generation of A-E-Expressing Human Hematopoietic Progenitor

Human hematopoietic progenitor CD34+ cells were purchased from Invitrogen (Life Technologies) and cultured according to the manufacturer's instructions. These StemPro CD34+ cells are human cord blood hematopoietic progenitor cells derived from mixed donors. Human A-E cDNA was excised from Addgene (www(dot)addgene(dot)org) pUHD-A-E plasmid using Age I and subcloned into a modified Addgene pCSC lentiviral vector as previously described [Regev et al., Proc. Natl. Acad. Sci. USA (2010) 107: 4424-4429] downstream from the cytomegalovirus promoter and upstream from the internal ribosomal entry site (IRES)-GFP cassette. Recombinant pseudo-lentiviral particles were generated by cotransfection of the pCSC-A-E-IRES-GFP vector and packaging DNA plasmids into human embryonic kidney (HEK) 293T cells. Following isolation and purification of pCSC-A-E-IRES-GFP lentiviral particles, they were introduced into CD34⁺ cells as previously described [Millington et al., PLoS ONE (2009) 4: e6461]. Following lentiviral transduction, the cells were harvested and expression of A-E in HEK 293T and in GFP-expressing CD34+ cells was validated by western blotting and qRT-PCR, respectively.

### Example 1

### Expression of Wild-Type (WT) RUNX1 is Essential for t(8;21) AML Kasumi-1 Cell Survival

The cell phenotypic consequences of RUNX1 knockdown (KD) was assessed in Kasumi-1 cells to directly address the possibility that native RUNX1 function is required for the leukemogenic process in t(8;21) AML cells. Specific siRNA-oligo nucleotides targeting RUNX1 regions absent from the A-E transcript were used to attenuate the expression of RUNX1 (FIG. 1A). Cell cycle analysis of Kasumi-1^{RX1-KD} cells revealed a prominent increase in the proportion of cells bearing subG1 DNA-content (FIGS. 1B and 1C) and a significant decrease in the proportions of S and G2/M phases, as compared to cells transfected with control non-targeting (NT) siRNA (Kasumi-1^{Cont}) (FIGS. 1B and 1C). This abnormal Kasumi-1^{RX1-KD} cell cycle was associated with an elevated percentage of both Annexin-V⁺ viable and nonviable cells (FIG. 1D) and approximately 7 fold decrease in the total number of viable cells (FIG. 1E). These results indicated that KD of RUNX1 induces apoptotic cell death in Kasumi-1^{RX1-KD}. Transfection of siRNA oligo directed against a different RUNX1 region (orange bar in FIG. 1A) confirmed that the apoptosis resulted from decreased RUNX1 activity. This finding ruled out the possibility of a siRNA-specific off-target effect (FIGS. 1J, 1K and 1L).

Next, it was examined whether Kasumi-1^{RX1-KD} cell death involved mitochondrial permeability transition (MPT). Flow-cytometry imaging (ImageStream© System) analysis demonstrated that increased Kasumi-1^{RX1-KD} cell apoptosis was associated with loss of mitochondrial membrane potential (FIGS. 1F and 1G) suggesting involvement of MPT in inducing cell death. To assess whether this RUNX1 KD-triggered apoptosis involved caspase activation, Kasumi-1^{RX1-KD} and Kasumi-1^{Cont} cell cycle was analyzed in the presence of the broad-spectrum caspase inhibitor Z-VAD-FMK. Significantly, Z-VAD-FMK completely blocked apoptosis in Kasumi-1^{RX1-KD} cells, reflected in a profound decrease of the subG1 fraction to level similar to that of Kasumi-1^{Cont} cells (FIG. 1H). Of note, the majority of Z-VAD-FMK-rescued Kasumi-1^{RX1-KD} cells accumulated at cell-cycle G1 and G2/M phases (FIG. 1H), suggesting that RUNX1 KD-evoked apoptosis involved impaired G2/M->>G1 transition. Using Z-VAD-FMK treatment further reduced RUNX1 protein levels in Kasumi-1^{RX1-KD} cells (FIG. 1I). Taken together the results of cell-cycle analysis, Annexin-V staining, viability assay, ImageStream© analysis and Z-VAD-FMK experiments demonstrated that attenuation of wild-type (WT) RUNX1 expression in Kasumi-1 cells triggers pronounced caspase-dependent apoptosis associated with changes in mitochondrial permeability. The most likely implication of this data is that WT RUNX1 plays an anti-apoptotic role in t(8;21) AML cells and its activity is compromised by oncogenic chimeric proteins bearing the RUNX runt domain (RD). Therefore, the remaining WT RUNX1 activity is indispensable for the AML cell viability.

### Example 2

### A-E KD Rescues Kasumi-1^{RX1-KD} Cells from Apoptosis

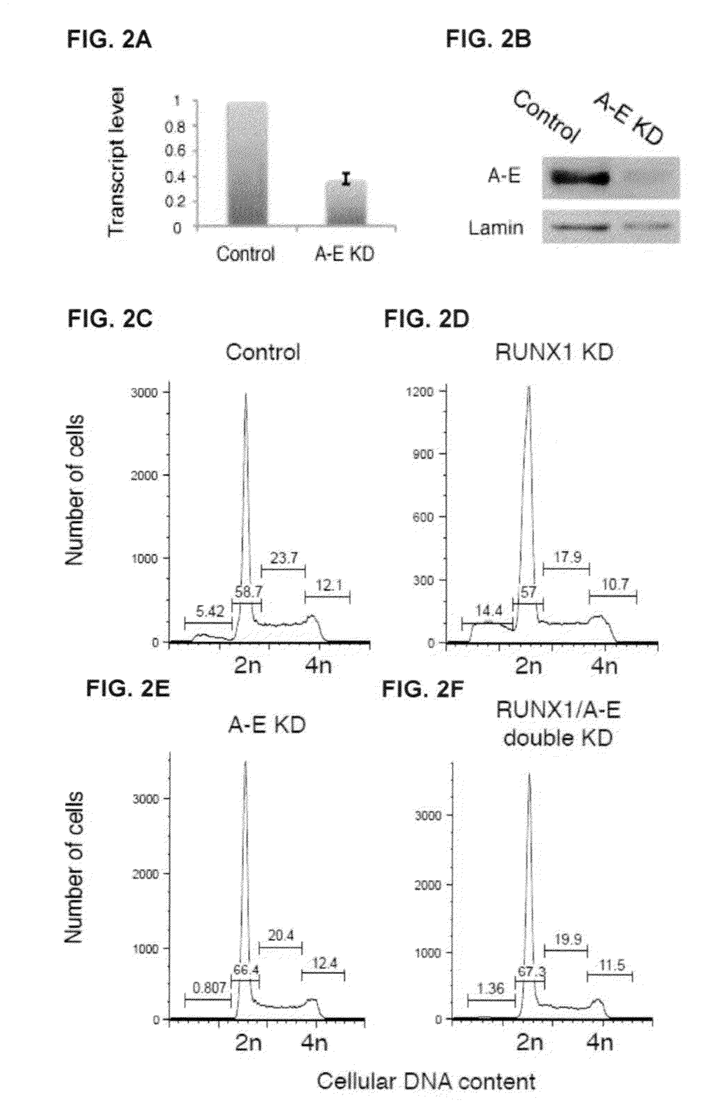

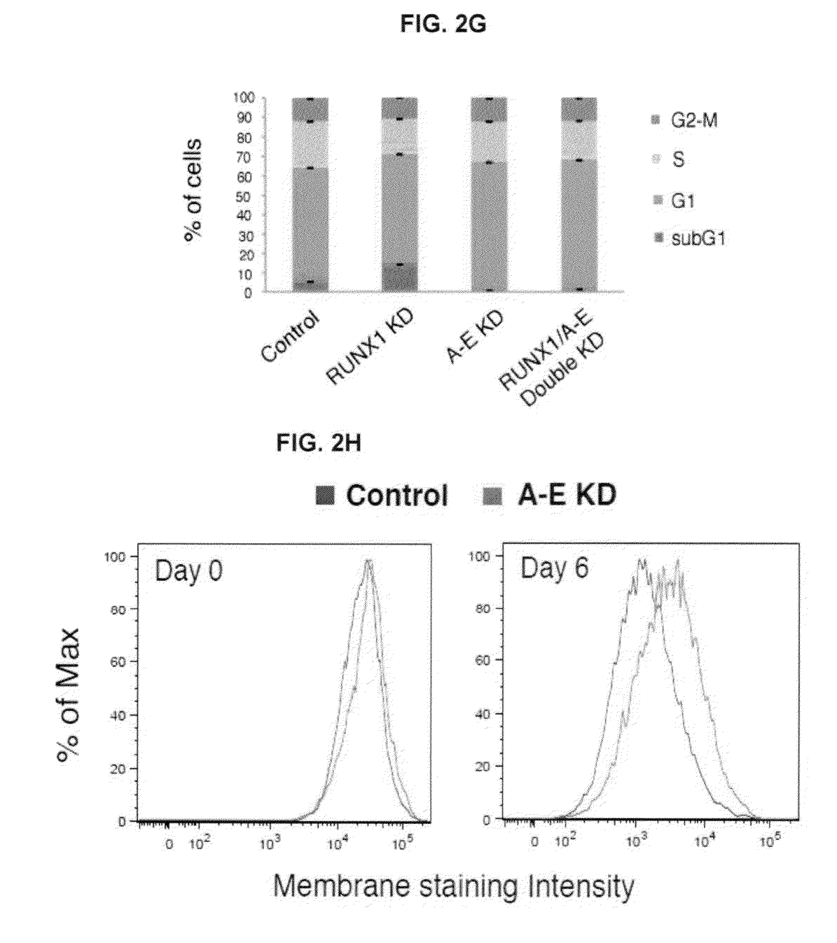

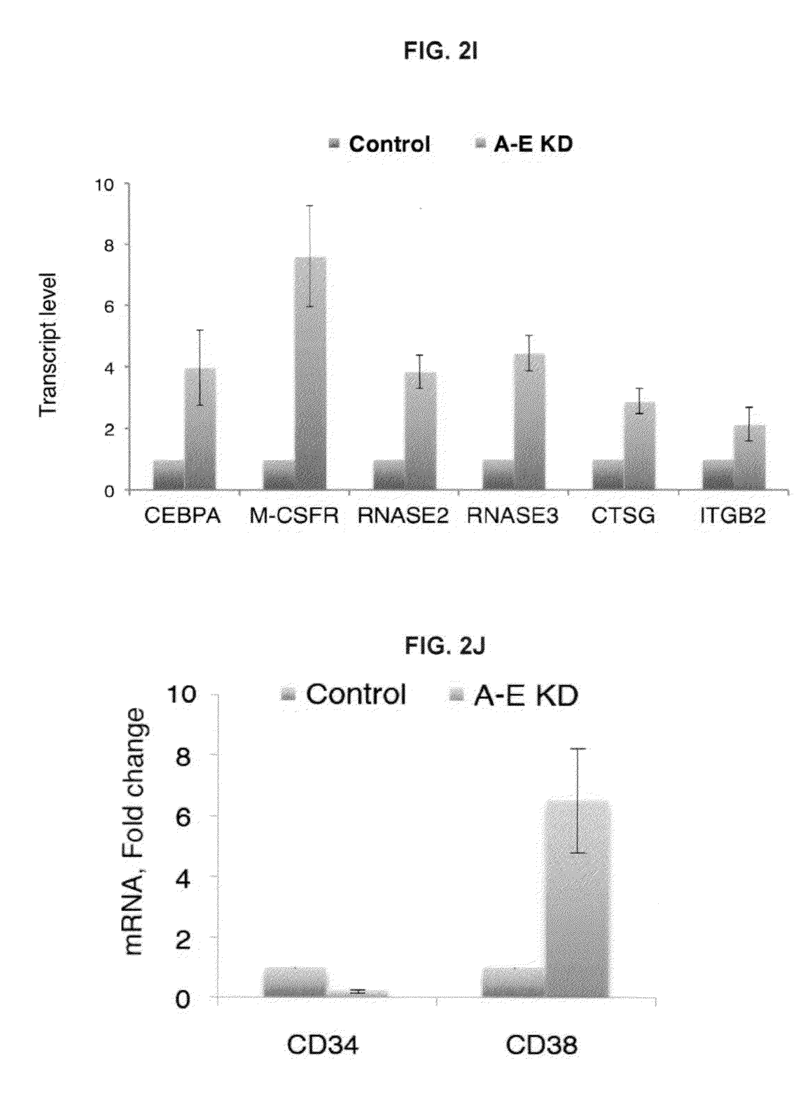

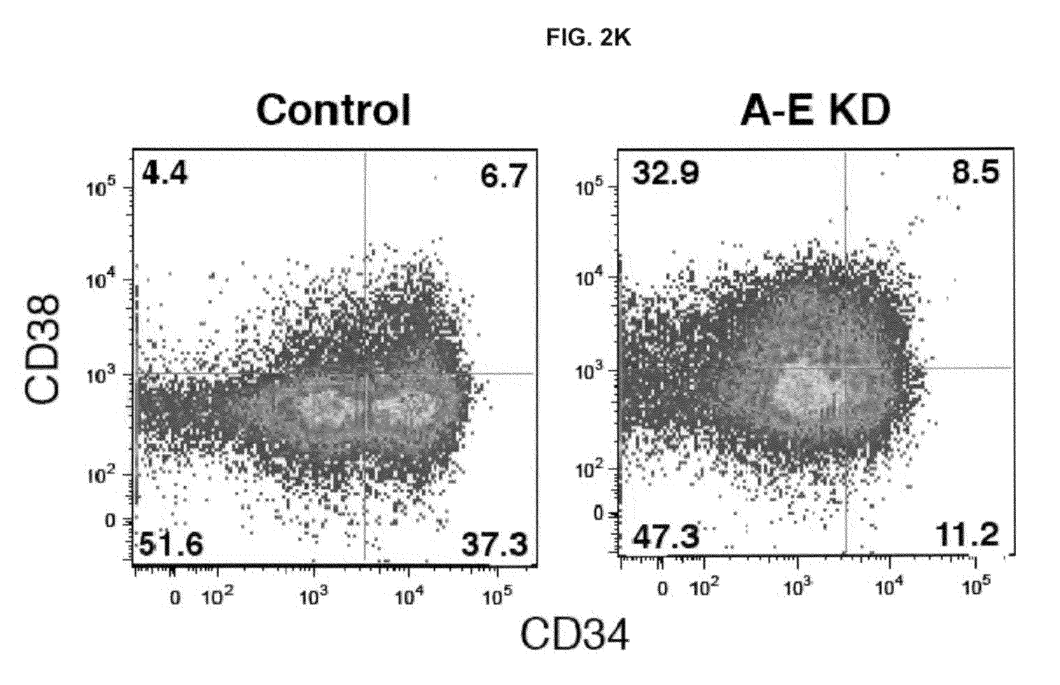

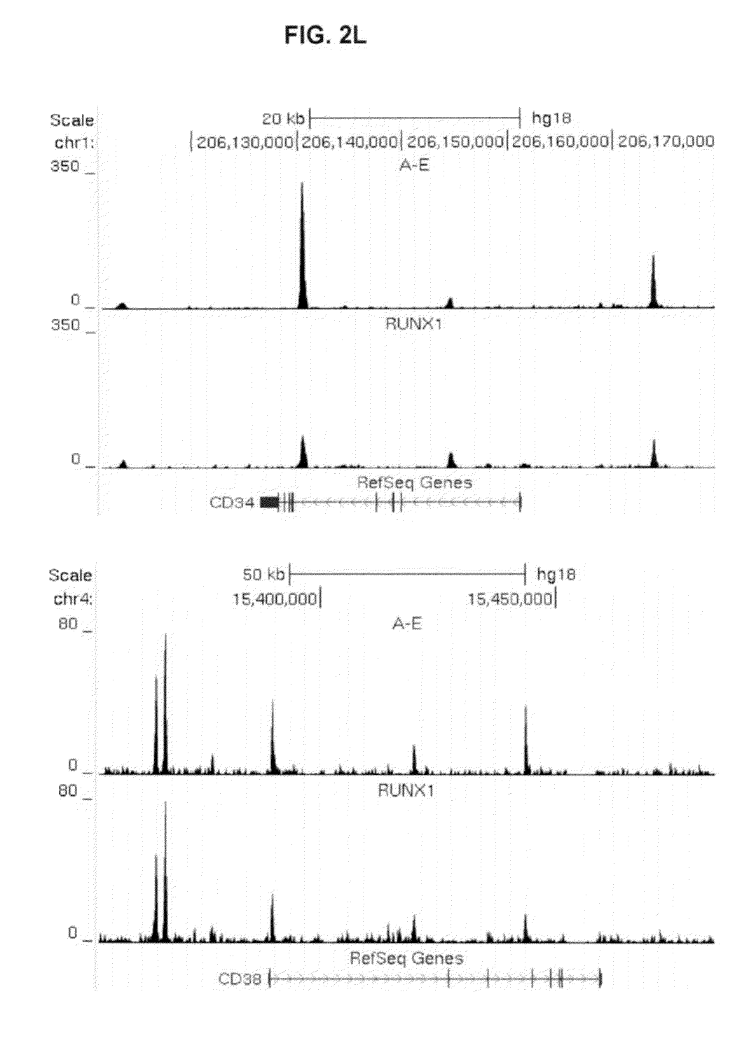

To further investigate the involvement of WT RUNX1 in the development of A-E-mediated t(8;21) AML, a siRNA specific for the translocated transcripts to KD A-E (Kasumi-1^{AE-KD} expression was used (FIGS. 2A and 2B). Kasumi-1^{AE-KD} cells displayed decreased proliferation and increased myeloid differentiation (FIGS. 2H and 2I), as was previously noted [Ptasinska et al. (2012), supra], as well as a marked reduction in the proportion of CD34⁺CD38⁻ leukemogenic cell-population (FIGS. 2J, 2K and 2L). Next, the impact of A-E KD on cell phenotype of Kasumi-1^{RX1-KD} was examined Interestingly, the double KD cells (Kasumi-1^{RX1/AE-KD} displayed an apoptotic level similar to, or even lower than, that of control cells (FIGS. 2B and 2C). This observation was consistent with the possibility that A-E activity contributed to Kasumi-1^{RX1-KD} cell apoptosis, underscoring the importance of the balance between A-E and RUNX1 activities for maintenance of leukemogenicity. It further suggested that A-E and RUNX1 are positive and negative apoptosis regulators by controlling the expression of their shared target genes in opposing manner.

### Example 3

### RUNX1- and A-E-Responsive Genes are Inversely Regulated

Next, the present inventors sought to identify RUNX1- and A-E-responsive genes that participate in the interplay between the two transcription factors (TFs) thereby affecting Kasumi-1 cell survival. First, the global gene-expression alterations in response to KD of either RUNX1 or A-E were assessed by analyzing the Kasumi-1^{RX1-KD} or Kasumi-1^{AE-KD} cell transcriptomes compared to that of Kasumi-1^{Cont} (Tables 2 and 3, hereinbelow) Importantly, the overall gene-expression profile in response to KD of A-E or RUNX1 was inversely correlated (FIG. 3A, R²=−0.33). Genes repressed following RUNX1 KD tended to be upregulated following A-E KD and vice-versa (FIG. 3A).

Of the 754 genes that responded to KD of either A-E or RUNX1, 109 were common and affected by KD of either one (FIG. 3B). The majority of these A-E/RUNX1 common genes (95 of 109) responded inversely to the KD of RUNX1 or A-E (Tables 4A-4B, hereinbelow). Interestingly, analysis of these inversely A-E/RUNX1-regulated genes (using Ingenuity® System IPA) revealed significant association with terms of cell death and/or apoptosis (Table 5, hereinbelow). Thus, the gene-expression data supported the idea that disruption of the cellular balance between RUNX1 and A-E activities is the underlying cause for Kasumi-1^{RX1-KD} cell apoptosis. Therefore, this regulatory interplay was further characterized by analyzing the genomic occupancy of the two TFs.

TABLE 2
Genes showing differential expression in Kasumi-1^{RX1-KD} versus Kasumi-1^{Cont}
measured by expression arrays (listed are genes that showed fold-change of at least
1.4 and p-value <0.05)

| | Fold | | | | | |
|---|---|---|---|---|---|---|
| | Change | | | | | |
| | relative to | | | | | |
| | Non- | | Non- | Non- | RUNX1 | RUNX1 |
| Gene Symbol | targeting | p-value | taregting_1 | taregting_2 | KD_1 | KD_2 |
| ACACB | −1.40763 | 0.00184072 | 7.61831 | 7.52981 | 7.09838 | 7.0632 |
| ACPP | −1.6384 | 0.00022972 | 6.57374 | 6.62035 | 5.84978 | 5.91972 |
| ADAMTS3 | 1.80447 | 0.00025414 | 7.5457 | 7.66217 | 8.45669 | 8.45433 |
| AIF1 | −1.50136 | 0.00178403 | 10.0478 | 10.0881 | 9.39191 | 9.57138 |
| ALDH1L2 | −2.30403 | 0.00454385 | 9.73728 | 9.20292 | 8.15744 | 8.37443 |
| ALDOC | 2.117 | 0.00182717 | 10.0601 | 10.0109 | 11.0603 | 11.1747 |
| ANPEP | 1.40783 | 0.0299307 | 7.19541 | 6.9524 | 7.69392 | 7.44083 |
| ANXA1 | −2.11624 | 0.00132635 | 6.74516 | 6.65924 | 5.71989 | 5.52151 |
| ARHGAP4 | −1.41471 | 0.00201766 | 9.07217 | 9.02455 | 8.63382 | 8.46189 |
| ARHGEF3 | 2.08274 | 4.50E−05 | 7.5762 | 7.52328 | 8.64067 | 8.57577 |
| ARRB2 | −1.75126 | 0.00044432 | 9.52019 | 9.35512 | 8.65117 | 8.60736 |
| ARRDC3 | 1.47535 | 0.0106114 | 9.05844 | 8.97947 | 9.53375 | 9.62629 |
| ASNS | −1.60616 | 0.0129201 | 9.5127 | 9.11703 | 8.59253 | 8.66997 |
| ATF4 | 1.42674 | 0.00274586 | 9.75879 | 9.58476 | 10.2261 | 10.1429 |
| ATP2B4 | −1.42073 | 0.0006802 | 8.37919 | 8.26548 | 7.83943 | 7.79198 |
| ATP5L | 1.40037 | 0.01694 | 10.9852 | 11.1045 | 11.5149 | 11.5463 |
| B3GALTL | 1.40306 | 0.00706544 | 7.18914 | 6.97544 | 7.54823 | 7.5935 |
| BCL2 | −1.72227 | 0.00154257 | 7.71963 | 7.61849 | 6.97341 | 6.7961 |
| BCL6 | 1.7364 | 0.0107196 | 5.14486 | 5.11468 | 6.0948 | 5.75694 |
| BMP4 | −1.49305 | 0.00729581 | 7.77767 | 7.60415 | 7.0522 | 7.1731 |
| BRI3BP | −1.81913 | 8.29E−07 | 10.1605 | 10.2048 | 9.32354 | 9.31531 |
| BVES | −1.47967 | 0.00668212 | 6.91308 | 6.90398 | 6.44672 | 6.23979 |
| C10orf114 | 1.7224 | 0.00170023 | 7.61435 | 7.69044 | 8.29985 | 8.57378 |
| C11orf17 | 1.47968 | 0.0009167 | 9.51608 | 9.59567 | 10.0999 | 10.1424 |
| C11orf24 | −1.68746 | 0.00046848 | 7.37018 | 7.4807 | 6.62772 | 6.71346 |
| C11orf67 | 1.42685 | 0.00893273 | 5.86616 | 6.04284 | 6.51411 | 6.42055 |
| C12orf23 | 1.40247 | 0.00757172 | 8.3435 | 8.3706 | 8.78115 | 8.90889 |
| C16orf54 | −1.47511 | 0.0104102 | 6.72924 | 6.51036 | 6.1588 | 5.95915 |
| C19orf51 | −1.51198 | 0.00196583 | 6.7297 | 6.71764 | 6.14685 | 6.10762 |
| C1orf96 | −1.40406 | 0.00135483 | 8.36727 | 8.40045 | 7.85511 | 7.9334 |
| C20orf43 | 1.42341 | 0.00860676 | 8.66106 | 8.86777 | 9.30392 | 9.24363 |
| C22orf9 | −1.58608 | 0.00069893 | 9.49356 | 9.40911 | 8.79368 | 8.77807 |
| C3AR1 | −1.69679 | 0.0245228 | 8.46407 | 8.41587 | 7.63553 | 7.71879 |
| C5 | −1.93866 | 0.00026536 | 8.74042 | 8.61672 | 7.65597 | 7.79105 |
| C5orf23 | 1.76542 | 0.00056469 | 7.03709 | 6.85503 | 7.82801 | 7.70414 |
| C8orf73 | −1.63246 | 0.0105784 | 7.85852 | 7.8733 | 7.00302 | 7.3147 |
| C9orf91 | −1.42897 | 0.00073664 | 8.26075 | 8.34645 | 7.79379 | 7.78347 |
| CALHM2 | −1.64518 | 0.00017881 | 8.24546 | 8.20452 | 7.44934 | 7.56415 |
| CARS | −1.40356 | 0.00086423 | 7.53935 | 7.54806 | 6.98705 | 7.12219 |
| CBS | −1.68784 | 0.00541567 | 8.08606 | 7.6982 | 7.12502 | 7.14888 |
| CD109 | 3.03259 | 0.00024728 | 5.77752 | 5.69929 | 7.44997 | 7.22794 |
| CD180 | −1.91119 | 0.00083066 | 10.0145 | 9.90411 | 8.93814 | 9.11152 |
| CD244 | −1.41359 | 0.00114907 | 7.91903 | 7.97857 | 7.38197 | 7.51689 |
| CD33 | −2.65766 | 6.95E−05 | 9.86459 | 9.8736 | 8.54315 | 8.37473 |
| CD69 | 3.06524 | 0.00104659 | 6.29279 | 5.89638 | 7.68366 | 7.73752 |
| CD74 | −1.62141 | 0.0125951 | 7.65329 | 7.30356 | 6.64808 | 6.91427 |
| CDC42EP3 | 1.85125 | 0.00014582 | 6.48032 | 6.42218 | 7.29244 | 7.38706 |
| CEP68 | 1.94736 | 7.74E−05 | 6.7979 | 6.86005 | 7.7768 | 7.8042 |
| CHAC1 | −2.24194 | 0.00201898 | 7.22332 | 6.97528 | 5.7691 | 6.10001 |
| CHRDL1 | 1.5696 | 0.0114694 | 5.21536 | 5.34217 | 5.81455 | 6.04377 |
| CIDEB | −2.30501 | 0.00019259 | 8.47756 | 8.42801 | 7.24889 | 7.24712 |
| CNKSR3 | 1.4015 | 0.00671442 | 6.55067 | 6.6916 | 7.09157 | 7.12463 |
| COMMD6 | −1.61937 | 0.00236046 | 9.47295 | 9.34712 | 8.76718 | 8.66202 |
| CORO6 | 1.40765 | 0.0111591 | 5.82259 | 5.92465 | 6.40981 | 6.32399 |
| CSF2RB | −1.41855 | 0.00358142 | 7.19019 | 7.05435 | 6.67804 | 6.55767 |
| CST3 | −1.42551 | 3.54E−05 | 9.7683 | 9.78097 | 9.25578 | 9.27054 |
| CTTN | 2.37764 | 0.00018889 | 5.95312 | 5.82921 | 7.03662 | 7.24477 |
| CXorf21 | −1.43817 | 0.00017057 | 8.74319 | 8.78746 | 8.28445 | 8.19773 |
| CXXC5 | −1.47338 | 0.00012636 | 8.33717 | 8.29311 | 7.78304 | 7.72898 |
| CYTIP | −1.55512 | 0.0204221 | 7.7037 | 7.94563 | 7.16243 | 7.21284 |
| CYTL1 | 1.46757 | 0.0418745 | 7.25056 | 7.32615 | 7.94357 | 7.74 |
| CYTSB | −1.62794 | 0.00077137 | 6.80891 | 6.78963 | 6.0626 | 6.12984 |
| DAGLB | 1.42507 | 0.00356511 | 9.10645 | 9.17871 | 9.6302 | 9.67702 |
| DHRS9 | 1.73879 | 0.00092002 | 5.60445 | 5.81331 | 6.4642 | 6.54974 |
| DKFZp686O24166 | −1.60285 | 2.72E−05 | 10.7782 | 10.7969 | 10.0929 | 10.1209 |
| DNAJB4 | 1.41258 | 0.00077884 | 8.41491 | 8.30589 | 8.8128 | 8.90466 |
| DNAJC12 | −1.55325 | 0.00046721 | 10.056 | 10.0934 | 9.37451 | 9.50422 |
| DOK4 | 1.48092 | 0.00079621 | 7.4038 | 7.36613 | 7.9663 | 7.93662 |
| DPEP1 | 6.93097 | 9.42E−06 | 5.92918 | 6.03924 | 8.70835 | 8.84618 |
| DPYD | 1.77894 | 0.00033472 | 5.57614 | 5.58173 | 6.39864 | 6.42127 |
| DUSP5P | −1.51769 | 0.0208686 | 6.94855 | 6.51907 | 6.11546 | 6.1484 |
| EDIL3 | 1.79102 | 0.00017759 | 9.7096 | 9.71488 | 10.6067 | 10.4994 |
| EEF1A1 | 1.55556 | 0.0430797 | 9.39752 | 8.99468 | 9.75257 | 9.91449 |
| EIF4A2 | 1.59418 | 0.00102093 | 7.77606 | 7.75977 | 8.43901 | 8.44245 |
| ELAC1 | 1.53513 | 0.0344856 | 6.43532 | 6.40835 | 7.04182 | 7.03858 |
| ELOVL7 | 1.53571 | 0.00632569 | 7.16829 | 7.31014 | 7.89759 | 7.81866 |
| ENTPD1 | 1.68754 | 0.00158751 | 9.02469 | 8.91301 | 9.72263 | 9.72491 |
| ERG | 1.47994 | 0.00142434 | 9.53569 | 9.42513 | 9.97294 | 10.119 |
| ERVFRDE1 | 1.41568 | 0.00580833 | 6.15088 | 6.06195 | 6.53532 | 6.6805 |
| ESAM | 1.44088 | 0.0346154 | 6.26189 | 5.93874 | 6.68321 | 6.57132 |
| FAM101B | −2.03632 | 7.63E−07 | 9.07205 | 9.04233 | 8.03283 | 8.02964 |
| FAM105A | 1.56419 | 0.00261185 | 9.82829 | 9.6652 | 10.3316 | 10.4527 |
| FAM27E3 | −1.4189 | 0.0275738 | 7.44669 | 7.22639 | 6.91906 | 6.74447 |
| FAM58A | 1.4225 | 0.00866111 | 6.99315 | 7.02642 | 7.57616 | 7.46026 |
| FAM84B | −1.46689 | 0.00985356 | 7.3079 | 7.04219 | 6.66856 | 6.57599 |
| FAR2 | 1.43315 | 0.00018106 | 9.19919 | 9.15727 | 9.74102 | 9.65381 |
| FBXW7 | 2.07025 | 0.00079637 | 7.83907 | 7.91197 | 8.79281 | 9.05783 |
| FGD4 | 1.48653 | 0.0127819 | 5.29459 | 5.44009 | 6.06289 | 5.8157 |
| FGF16 | 3.13879 | 0.00205912 | 5.07283 | 5.01509 | 7.01225 | 6.37609 |
| FHL1 | −1.42982 | 0.00351559 | 8.14492 | 7.97001 | 7.47026 | 7.61301 |
| FLJ38379 | 1.80736 | 0.0103363 | 5.89958 | 5.99468 | 6.86929 | 6.73274 |
| FLNA | −1.45959 | 0.00056288 | 8.72303 | 8.72737 | 8.1908 | 8.16848 |
| FLRT2 | 1.5062 | 0.00066276 | 7.50603 | 7.40353 | 7.98197 | 8.10941 |
| FMNL1 | −1.5298 | 0.00142968 | 8.57443 | 8.50695 | 7.99377 | 7.86092 |
| FOLH1 | 1.92811 | 0.00624121 | 6.806 | 6.68802 | 7.51004 | 7.87835 |
| FOLH1B | 2.0107 | 0.00286568 | 6.50824 | 6.34838 | 7.24759 | 7.62444 |
| FOSB | 1.904 | 0.0452988 | 6.59521 | 5.77372 | 7.28202 | 6.94498 |
| FRRS1 | −1.64358 | 0.00786712 | 6.92891 | 6.81696 | 5.99686 | 6.31534 |
| FRY | 2.18681 | 0.00018485 | 5.20401 | 5.17305 | 6.33979 | 6.29493 |
| FYB | −1.8869 | 0.00019855 | 7.17028 | 7.03615 | 6.14144 | 6.23296 |
| GABARAPL2 | 1.41294 | 0.00211298 | 10.1194 | 10.1653 | 10.657 | 10.6251 |
| GAFA3 | −1.53919 | 0.00794211 | 6.3121 | 6.40293 | 5.82018 | 5.6505 |
| GALNT3 | −1.63857 | 0.00146529 | 6.99701 | 6.92973 | 6.26353 | 6.23832 |
| GATS | 1.62398 | 0.00015884 | 6.08871 | 6.0859 | 6.78125 | 6.79242 |
| GATSL1 | 1.52119 | 0.00111185 | 7.26486 | 7.18208 | 7.75588 | 7.90145 |
| GCH1 | 1.5649 | 0.00117967 | 7.27854 | 7.35322 | 7.91618 | 8.00771 |
| GGTA1 | 2.33719 | 0.00028642 | 5.99986 | 5.79644 | 7.14014 | 7.1057 |
| GNAI1 | 1.46127 | 0.00758511 | 6.9393 | 7.05205 | 7.63209 | 7.45371 |
| GPA33 | 2.39008 | 5.47E−06 | 5.39214 | 5.33677 | 6.60551 | 6.63752 |
| GPR141 | −1.92989 | 6.37E−05 | 8.77847 | 8.62655 | 7.76486 | 7.74311 |
| GPT2 | −1.54053 | 0.0118405 | 6.86666 | 6.53173 | 5.99369 | 6.15785 |
| GRPEL2 | −1.46312 | 0.00422298 | 10.4764 | 10.2354 | 9.772 | 9.84177 |
| GSDMB | 1.59604 | 0.00636467 | 6.37398 | 6.44543 | 7.25457 | 6.91383 |
| GSN | 1.65075 | 7.39E−07 | 10.8639 | 10.8585 | 11.6011 | 11.5675 |
| GUCY1A3 | 1.84401 | 0.0139154 | 6.59148 | 6.60219 | 7.69719 | 7.26217 |
| GUCY1B3 | 1.89583 | 0.00098286 | 6.52225 | 6.40008 | 7.43194 | 7.33605 |
| H3F3B | 1.43627 | 0.0249651 | 9.75494 | 9.66876 | 10.2499 | 10.2185 |
| HCG27 | −1.44551 | 0.00824483 | 6.85673 | 6.60277 | 6.23057 | 6.16577 |
| HCST | −1.46775 | 0.00933771 | 6.41596 | 6.43636 | 5.94508 | 5.80002 |
| HIVEP3 | −1.51411 | 0.00159331 | 6.17837 | 6.1408 | 5.58288 | 5.53936 |
| HMHA1 | −1.46147 | 0.00534458 | 7.53563 | 7.41809 | 6.80511 | 7.05378 |
| HOXA5 | 1.8874 | 0.00237887 | 6.65475 | 6.36123 | 7.50717 | 7.34161 |
| HPSE | 1.64133 | 0.00015647 | 7.9396 | 7.86925 | 8.63504 | 8.60353 |
| HSPG2 | 1.40013 | 0.00103275 | 8.10889 | 8.12453 | 8.63685 | 8.5677 |
| ICA1 | 1.42909 | 0.00735713 | 7.81902 | 7.82122 | 8.27304 | 8.3974 |
| ICAM3 | 1.48337 | 0.00032259 | 7.7987 | 7.7984 | 8.39294 | 8.34191 |
| ID1 | 1.50535 | 0.00015177 | 7.78096 | 7.88022 | 8.42938 | 8.41199 |
| IGFBP7 | −1.52179 | 0.00178795 | 11.034 | 11.0058 | 10.3728 | 10.4554 |
| IGSF10 | −1.45766 | 0.00127409 | 9.64134 | 9.64051 | 9.1345 | 9.06003 |
| IKZF1 | −1.40619 | 0.00108906 | 10.1186 | 10.0434 | 9.59001 | 9.5885 |
| IL1RAP | 1.40196 | 0.00027566 | 8.52319 | 8.52241 | 8.95298 | 9.06751 |
| IL3RA | 1.45206 | 0.00168427 | 5.69986 | 5.58665 | 6.20813 | 6.15458 |
| IL6ST | 1.63758 | 0.00162213 | 9.29156 | 9.27043 | 9.98403 | 10.0011 |
| IL8 | 1.48792 | 0.0219222 | 8.40636 | 8.30829 | 8.96781 | 8.89345 |
| INSIG2 | 1.60679 | 0.0014475 | 7.14409 | 6.99815 | 7.79684 | 7.71376 |
| IRF1 | 1.5309 | 0.0090653 | 6.35067 | 6.08084 | 6.87938 | 6.78088 |
| IRX3 | −1.59261 | 0.00044021 | 9.02088 | 9.01868 | 8.33882 | 8.35795 |
| ITGA3 | 1.72909 | 0.00012552 | 5.25553 | 5.13812 | 5.9583 | 6.01538 |
| ITGA6 | 1.47635 | 0.00612225 | 7.22148 | 7.21273 | 7.71408 | 7.84419 |
| ITGA9 | 1.51085 | 0.00371871 | 8.67461 | 8.74723 | 9.34832 | 9.26424 |
| ITM2A | 1.62889 | 0.0185877 | 6.61226 | 6.96081 | 7.53752 | 7.44333 |
| JDP2 | −1.55948 | 0.00049749 | 8.51115 | 8.46739 | 7.86298 | 7.83344 |
| JMJD1C | 1.4024 | 1.53E−05 | 11.0243 | 11.0225 | 11.5179 | 11.5047 |
| KCNAB2 | −1.96282 | 0.00023314 | 9.80415 | 9.85277 | 8.95349 | 8.75757 |
| KIAA0182 | −1.97479 | 0.00789046 | 7.95768 | 7.84384 | 7.19014 | 6.64799 |
| KIAA1324L | 2.12255 | 0.0006249 | 7.24715 | 7.14024 | 8.14925 | 8.40973 |
| KIAA1370 | 1.71736 | 0.00105491 | 7.15128 | 7.20117 | 7.86202 | 8.05081 |
| KIAA1462 | 1.56214 | 0.00122691 | 8.13687 | 8.07859 | 8.67055 | 8.83195 |
| KLHDC2 | 1.4289 | 0.0114115 | 9.11855 | 9.13531 | 9.6246 | 9.65906 |
| KLHL24 | 1.54546 | 0.00700477 | 8.4005 | 8.55283 | 9.07215 | 9.13724 |
| LAPTM5 | −1.72158 | 0.00023642 | 10.6506 | 10.5529 | 9.89009 | 9.74592 |
| LBH | 1.6314 | 0.0148177 | 6.71671 | 6.97468 | 7.35168 | 7.75193 |
| LCP1 | −2.58614 | 5.69E−05 | 10.3324 | 10.1786 | 8.93077 | 8.83867 |
| LGALS12 | −1.92575 | 0.00216789 | 10.0531 | 9.98228 | 8.9555 | 9.18901 |
| LGALS9 | −1.43113 | 0.0379031 | 7.93237 | 7.62495 | 7.31913 | 7.20389 |
| LGALS9B | −1.46412 | 0.00655844 | 9.90142 | 9.83993 | 9.36492 | 9.27638 |
| LGALS9C | −1.45607 | 0.00729234 | 9.6366 | 9.48558 | 9.06904 | 8.96899 |
| LIN7A | −1.58159 | 0.00724763 | 7.15709 | 6.99282 | 6.45216 | 6.37501 |
| LOC100130100 | 1.48338 | 0.00543639 | 5.95032 | 5.87795 | 6.39415 | 6.5719 |
| LOC100131860 | −1.5393 | 0.00398473 | 8.72327 | 8.75875 | 7.99728 | 8.24019 |
| LOC100133280 | −1.44619 | 0.0023109 | 7.48707 | 7.28182 | 6.85055 | 6.85381 |
| LOC284232 | 1.44875 | 0.00884096 | 5.51381 | 5.68851 | 6.04931 | 6.22263 |
| LOC284757 | −1.72907 | 0.00047036 | 6.79275 | 6.82709 | 6.039 | 6.00085 |
| LOC387790 | −1.42261 | 0.00903046 | 7.35528 | 7.58134 | 7.01823 | 6.90132 |
| LOC388955 | −1.41139 | 0.00122255 | 10.2698 | 10.2572 | 9.79261 | 9.74019 |
| LOC643332 | −3.53288 | 0.00018853 | 11.6276 | 11.5484 | 9.73768 | 9.7967 |
| LPAR1 | −1.52498 | 0.00015007 | 7.68992 | 7.61883 | 7.02893 | 7.06225 |
| LPAR6 | 2.88705 | 0.0001059 | 6.39219 | 6.3156 | 7.87803 | 7.88895 |
| LPIN2 | 1.46782 | 0.0040745 | 8.16193 | 8.33245 | 8.7126 | 8.88913 |
| LPXN | −1.87525 | 0.00022788 | 8.03042 | 8.18261 | 7.23029 | 7.16856 |
| LRG1 | 1.56826 | 0.00265232 | 6.6612 | 6.68372 | 7.21607 | 7.42717 |
| LRRC34 | −1.55799 | 0.00045494 | 9.16777 | 9.1414 | 8.54729 | 8.48251 |
| LRRC4 | 2.47144 | 2.76E−05 | 5.63986 | 5.78847 | 6.98297 | 7.05606 |
| LRRC70 | 2.68075 | 6.00E−05 | 6.99385 | 6.84065 | 8.37832 | 8.30146 |
| LST1 | −1.56791 | 0.001024 | 8.43812 | 8.37188 | 7.6594 | 7.85292 |
| MACF1 | 1.52231 | 0.00084184 | 7.55296 | 7.6978 | 8.2443 | 8.21898 |
| MAGED2 | 1.85262 | 9.69E−06 | 8.03016 | 8.06373 | 8.92489 | 8.94814 |
| MAP1B | −1.47318 | 0.00044691 | 7.51396 | 7.56799 | 6.98214 | 6.98194 |
| MBD5 | 1.42492 | 6.12E−05 | 5.83993 | 5.76768 | 6.3201 | 6.30928 |
| MERTK | 1.53009 | 0.0014471 | 6.89526 | 6.87359 | 7.57493 | 7.42116 |
| MFSD2A | −1.45351 | 0.00040918 | 7.55611 | 7.55861 | 7.06182 | 6.97383 |
| MIR142 | 1.76286 | 0.00884504 | 7.20727 | 7.13914 | 8.21202 | 7.77022 |
| MIR21 | 4.60366 | 0.00281494 | 5.58325 | 4.87285 | 7.66421 | 7.19745 |
| MIR221 | −1.45999 | 0.00053971 | 9.59685 | 9.60175 | 9.04295 | 9.06375 |
| MIR223 | −1.93127 | 0.00112156 | 9.06133 | 8.92388 | 7.92117 | 8.16494 |
| MIRLET7F1 | 1.54435 | 0.0278768 | 5.64951 | 5.53827 | 6.05237 | 6.3894 |
| MMP28 | 2.38415 | 0.0001198 | 5.89656 | 5.70676 | 7.00222 | 7.10804 |
| MNS1 | −1.66242 | 0.00221421 | 8.25471 | 8.31623 | 7.43193 | 7.67243 |
| MOCS2 | −1.4163 | 0.0270215 | 9.48487 | 9.4758 | 8.84103 | 9.11537 |
| MRC2 | −1.48323 | 0.00056963 | 7.8808 | 7.85396 | 7.23484 | 7.36243 |
| MRPL15 | 1.44243 | 0.0063755 | 9.32915 | 9.41228 | 9.95826 | 9.84018 |
| MTSS1 | 2.77072 | 0.00078385 | 6.53083 | 6.39145 | 8.14413 | 7.71867 |
| MYO1F | −1.63975 | 0.00103308 | 8.07989 | 8.0906 | 7.44746 | 7.29607 |
| NCKAP1 | 1.64177 | 0.00376162 | 7.86804 | 7.74819 | 8.44039 | 8.60634 |
| NDUFAF1 | 1.73415 | 0.0354393 | 6.66374 | 6.71977 | 7.4571 | 7.51487 |
| NEK6 | −1.54421 | 0.0002169 | 9.36085 | 9.38644 | 8.77722 | 8.71632 |
| NFKBIZ | 1.4548 | 0.00669358 | 6.60089 | 6.31397 | 7.0088 | 6.9877 |
| NHSL1 | −2.82473 | 0.00127304 | 7.82996 | 7.34056 | 6.03747 | 6.13683 |
| NIPAL2 | −1.51135 | 0.00090116 | 7.99539 | 7.8813 | 7.30864 | 7.37638 |
| NLRP3 | −1.66024 | 0.00491272 | 6.10926 | 5.86378 | 5.2142 | 5.29606 |
| NOG | 1.50432 | 0.00246815 | 9.22846 | 9.18981 | 9.75163 | 9.84488 |
| NPR3 | 1.75898 | 0.021036 | 6.78515 | 6.98607 | 7.97093 | 7.42977 |
| NPW | −1.55153 | 0.00833411 | 9.86147 | 9.74106 | 9.12866 | 9.2065 |
| NRM | −1.52075 | 0.0203813 | 9.52996 | 9.31496 | 8.79796 | 8.83741 |
| NTSR1 | −2.38698 | 0.00106978 | 8.37578 | 8.13935 | 6.94003 | 7.06472 |
| NUDT7 | −1.87111 | 0.00108895 | 8.04585 | 8.05686 | 7.12473 | 7.17019 |
| OBFC2B | −1.46938 | 0.00089058 | 9.99524 | 9.91024 | 9.38722 | 9.40785 |
| OLFM4 | −3.47052 | 2.71E−05 | 6.28397 | 6.23941 | 4.57339 | 4.35969 |
| OR52K1 | −1.45427 | 0.00940184 | 6.3382 | 6.2199 | 5.64148 | 5.83602 |
| OR52K3P | −1.7698 | 0.0117197 | 7.06851 | 6.62056 | 6.06624 | 5.97566 |
| OSBPL10 | 2.03065 | 0.00032795 | 6.00812 | 6.057 | 6.95276 | 7.15624 |
| OTUD1 | 1.50505 | 0.00578198 | 6.62874 | 6.36027 | 7.1564 | 7.01222 |
| OVOS | −1.5647 | 0.0380273 | 7.33085 | 7.46776 | 6.66589 | 6.84096 |
| P2RX7 | −1.76879 | 0.00034316 | 7.0694 | 7.0587 | 6.20912 | 6.27346 |
| P2RY13 | −1.57021 | 0.0169957 | 10.0813 | 9.95414 | 9.20576 | 9.52775 |
| P2RY14 | 1.57451 | 0.00188397 | 6.64525 | 6.75007 | 7.37408 | 7.33104 |
| PAFAH2 | 1.46865 | 0.00156279 | 7.46425 | 7.48392 | 7.99597 | 8.06119 |
| PARVG | −1.49454 | 0.00028192 | 10.0466 | 10.0673 | 9.48921 | 9.46533 |
| PCCA | −1.41433 | 0.00116671 | 6.78246 | 6.82146 | 6.24623 | 6.35745 |
| PCDHB14 | 1.70552 | 0.0138083 | 5.03575 | 5.15837 | 6.00576 | 5.72877 |
| PCK2 | −1.54997 | 0.00198758 | 9.6459 | 9.41347 | 8.89357 | 8.90131 |
| PDE1A | 2.99588 | 6.27E−05 | 5.72176 | 5.5025 | 7.16827 | 7.22196 |
| PDE4B | 1.40625 | 0.00808613 | 6.3144 | 6.32916 | 6.81913 | 6.80813 |
| PDGFC | 1.69247 | 0.0199002 | 5.67365 | 5.63357 | 6.35852 | 6.46696 |
| PDPK1 | −1.65977 | 0.00353248 | 7.64434 | 7.73792 | 6.9275 | 6.9928 |
| PHACTR1 | −1.45682 | 0.00116395 | 7.34778 | 7.29077 | 6.71895 | 6.83395 |
| PHGDH | −1.61269 | 0.0014797 | 11.5635 | 11.3965 | 10.8069 | 10.7742 |
| PITPNC1 | −1.41836 | 0.00094156 | 7.23707 | 7.37031 | 6.79978 | 6.79916 |
| PLA2G4A | −1.53497 | 0.00148934 | 7.46883 | 7.52063 | 6.83304 | 6.92001 |
| PLAC8 | 1.44716 | 0.00016139 | 8.92673 | 8.90371 | 9.49201 | 9.40489 |
| PLCB2 | −1.72318 | 0.00027767 | 7.86602 | 7.90113 | 7.04632 | 7.15068 |
| PLEKHA3 | 1.46514 | 0.0241521 | 7.76021 | 8.08659 | 8.36732 | 8.58156 |
| PLEKHA5 | 1.70616 | 0.00059964 | 6.34143 | 6.24092 | 7.15109 | 6.97277 |
| PLIN2 | 2.00538 | 0.00268828 | 8.80292 | 8.89821 | 9.84182 | 9.86706 |
| PLP2 | −1.72774 | 0.00055895 | 9.55374 | 9.4804 | 8.71362 | 8.74275 |
| PLXDC2 | −1.62853 | 0.00049623 | 10.3113 | 10.1686 | 9.56617 | 9.5066 |
| PLXNA4 | 1.80806 | 0.00054013 | 9.38622 | 9.39651 | 10.2235 | 10.2681 |
| PODXL | −1.60542 | 0.00044875 | 8.9375 | 8.80827 | 8.22973 | 8.15014 |
| POLR3G | −1.53877 | 0.00227875 | 9.41959 | 9.51091 | 8.90428 | 8.78266 |
| PORCN | −1.48579 | 0.0118005 | 7.88061 | 7.78211 | 7.30876 | 7.2115 |
| PPAPDC1B | 1.45005 | 0.000971 | 8.09096 | 8.09648 | 8.57266 | 8.68698 |
| PPIC | 1.93173 | 0.00269593 | 7.55005 | 7.31333 | 8.32543 | 8.43774 |
| PPP1R15A | 1.52981 | 0.0294095 | 7.2673 | 6.76342 | 7.65703 | 7.60041 |
| PRKCH | 2.24422 | 4.11E−05 | 8.69894 | 8.60848 | 9.78697 | 9.85287 |
| PRSSL1 | −1.45078 | 0.00614818 | 10.8077 | 10.7107 | 10.2533 | 10.1914 |
| PSAT1 | −1.88708 | 0.00039517 | 10.6061 | 10.4064 | 9.52857 | 9.65162 |
| PSD4 | −1.68871 | 0.00235741 | 6.9634 | 6.71089 | 6.15766 | 6.0048 |
| PTGER4 | −1.43041 | 0.0103623 | 7.07435 | 7.04082 | 6.40259 | 6.67974 |
| PTGS1 | −1.73578 | 0.0003314 | 9.06611 | 8.88795 | 8.1408 | 8.22209 |
| PTGS2 | 1.99529 | 0.00795463 | 6.26885 | 5.86319 | 6.86913 | 7.2561 |
| PTK2B | −1.48392 | 0.00190753 | 9.52076 | 9.53916 | 9.03728 | 8.88381 |
| PTPLAD2 | −1.47203 | 0.00175221 | 9.26853 | 9.22923 | 8.70468 | 8.67748 |
| PTPN13 | −2.22563 | 0.00038015 | 6.93937 | 6.71095 | 5.7148 | 5.6271 |
| PTPN22 | −2.16729 | 0.00198768 | 6.72627 | 6.5463 | 5.59661 | 5.44417 |
| PTPN6 | −1.71555 | 9.54E−05 | 8.00707 | 7.92738 | 7.21109 | 7.16601 |
| PTPRC | −1.98181 | 0.00031002 | 8.93072 | 8.83237 | 7.99965 | 7.7898 |
| PTPRM | 1.65787 | 1.62E−05 | 9.89288 | 9.94774 | 10.6695 | 10.6298 |
| PYCARD | −1.44728 | 0.00060886 | 9.08504 | 8.96803 | 8.46941 | 8.51696 |
| RAB37 | −1.44888 | 0.00131214 | 9.63612 | 9.46464 | 9.03096 | 8.99991 |
| RAB3A | −1.54292 | 0.0005281 | 8.23033 | 8.21804 | 7.65707 | 7.53998 |
| RAC2 | −1.5858 | 0.00345588 | 11.134 | 11.0058 | 10.4302 | 10.3792 |
| RASGRP2 | −1.75273 | 0.00209072 | 7.49731 | 7.42091 | 6.61169 | 6.68731 |
| RASGRP4 | −1.77185 | 0.00124465 | 7.13805 | 6.9259 | 6.15121 | 6.26223 |
| RASSF2 | −1.76002 | 0.00020987 | 6.73841 | 6.58177 | 5.86255 | 5.82645 |
| RASSF4 | −2.23587 | 0.00108008 | 7.39048 | 7.57598 | 6.44953 | 6.19526 |
| RBPMS | 1.73555 | 0.00118125 | 5.37146 | 5.31727 | 6.17905 | 6.10047 |
| RCBTB2 | 1.74279 | 0.00055819 | 7.95633 | 7.96919 | 8.76084 | 8.76748 |
| RECK | 1.57411 | 0.00248861 | 7.87818 | 7.81622 | 8.53438 | 8.4691 |
| RHOF | −1.40029 | 0.00261363 | 9.85731 | 9.81413 | 9.35062 | 9.34936 |
| RHOJ | 1.83202 | 0.00194677 | 5.25277 | 5.25121 | 6.03547 | 6.21537 |
| RLTPR | 1.51103 | 0.00031123 | 5.6641 | 5.60478 | 6.25225 | 6.20769 |
| RNASE2 | −1.98712 | 0.00017118 | 11.3694 | 11.3634 | 10.3648 | 10.3865 |
| RNASE3 | −2.8382 | 0.00023613 | 8.43908 | 8.31515 | 7.01291 | 6.73137 |
| RNF165 | 1.58763 | 0.00170644 | 6.12865 | 6.22112 | 6.74638 | 6.93714 |
| RNF41 | −1.43367 | 0.00318619 | 9.19008 | 9.06294 | 8.64937 | 8.56422 |
| RNU11 | 2.20963 | 0.00169914 | 7.00756 | 6.91611 | 8.3126 | 7.89867 |
| RNU2-1 | 1.41895 | 0.0181497 | 10.1492 | 10.2427 | 10.5266 | 10.875 |
| ROPN1L | −2.74075 | 0.00047618 | 7.3314 | 7.09846 | 5.72239 | 5.79833 |
| RPL21 | 1.41328 | 0.00222291 | 10.8264 | 10.8505 | 11.2921 | 11.3829 |
| RPL21P28 | 1.40035 | 0.00105646 | 10.7857 | 10.8346 | 11.2748 | 11.3171 |
| RUNX2 | 1.44456 | 0.0007675 | 8.41617 | 8.5084 | 9.00583 | 8.97999 |
| SAMHD1 | −1.74353 | 0.00087872 | 8.73976 | 8.53267 | 7.83365 | 7.83476 |
| SAMSN1 | 1.80375 | 0.0116773 | 8.74761 | 8.64411 | 9.50965 | 9.58407 |
| SAT1 | 1.60132 | 0.00678324 | 9.30976 | 9.26132 | 9.93141 | 9.99819 |
| SCARNA9 | 1.49904 | 0.00224259 | 6.92725 | 6.85675 | 7.3818 | 7.5703 |
| SCD5 | −1.45566 | 0.00626377 | 7.02225 | 7.05442 | 6.59771 | 6.39562 |
| SCUBE1 | 1.42835 | 5.50E−05 | 6.73739 | 6.70378 | 7.22198 | 7.24789 |
| SELK | 1.50221 | 0.0142079 | 8.2229 | 8.31714 | 8.81304 | 8.90118 |
| SELL | 1.55679 | 0.00218559 | 6.5756 | 6.60695 | 7.21854 | 7.24116 |
| SEMA4A | −2.51591 | 0.00027798 | 8.60918 | 8.44195 | 7.29496 | 7.09401 |
| SEMA4D | −1.68978 | 3.37E−05 | 9.211 | 9.25961 | 8.46656 | 8.49038 |
| SENP7 | 1.44003 | 0.00029925 | 7.30351 | 7.39347 | 7.84634 | 7.90284 |
| SERPINB8 | −2.59308 | 0.0002306 | 9.27931 | 9.27551 | 7.90627 | 7.89922 |
| SERPINB9 | 2.74786 | 1.91E−05 | 8.41058 | 8.32742 | 9.81552 | 9.8391 |
| SERTAD3 | 1.41569 | 0.00076721 | 6.08413 | 6.03011 | 6.57258 | 6.54467 |
| SESN1 | 1.46288 | 0.0022672 | 6.836 | 6.91283 | 7.39835 | 7.44811 |
| SESN3 | −1.77087 | 0.00196559 | 9.11352 | 9.12778 | 8.42549 | 8.16688 |
| SH3RF3 | −1.43075 | 0.00399602 | 7.96399 | 7.9301 | 7.44434 | 7.41622 |
| SH3TC2 | 2.45085 | 0.00131364 | 6.92729 | 6.74935 | 7.9373 | 8.3259 |
| SIGLEC12 | −1.98673 | 7.89E−05 | 9.04549 | 8.96409 | 7.95274 | 8.07605 |
| SIPA1L2 | 1.55855 | 0.00442378 | 6.88913 | 7.13629 | 7.71018 | 7.59565 |
| SIRPB1 | −1.92613 | 0.00034286 | 8.57607 | 8.41532 | 7.46807 | 7.63191 |
| SLC1A4 | −1.82485 | 0.0159047 | 7.43107 | 6.82545 | 6.26165 | 6.2593 |
| SLC25A30 | 1.55817 | 0.00053791 | 7.45159 | 7.34568 | 8.02175 | 8.05522 |
| SLC28A3 | −1.70163 | 0.00392992 | 9.20575 | 9.22276 | 8.40279 | 8.49187 |
| SLC2A3 | 1.69356 | 0.00070408 | 7.81099 | 7.92069 | 8.58465 | 8.66714 |
| SLC2A5 | −1.74414 | 0.00575877 | 6.97231 | 7.1733 | 6.23878 | 6.30181 |
| SLC35D2 | 1.46355 | 0.0241019 | 6.94297 | 6.76288 | 7.2466 | 7.55819 |
| SLC38A1 | −1.9281 | 0.00148997 | 10.1118 | 9.77227 | 9.00121 | 8.98848 |
| SLC40A1 | 1.54549 | 0.00692202 | 6.15527 | 6.32715 | 6.87339 | 6.86516 |
| SLC43A3 | −2.61219 | 0.00065411 | 10.543 | 10.348 | 8.94912 | 9.17138 |
| SLC44A2 | 1.88817 | 0.00035993 | 8.35746 | 8.30231 | 9.20945 | 9.28429 |
| SLC6A9 | −1.55246 | 0.00561523 | 7.32384 | 7.15151 | 6.59674 | 6.6095 |
| SLC7A1 | −1.43512 | 0.00169715 | 9.62802 | 9.53905 | 9.07371 | 9.05102 |
| SLC7A11 | −2.54646 | 0.00124848 | 8.26869 | 7.82324 | 6.69832 | 6.69662 |
| SLC7A5 | −2.31081 | 0.00052179 | 10.4848 | 10.2405 | 9.16646 | 9.14207 |
| SLIT2 | 1.80802 | 4.12E−05 | 5.60218 | 5.52275 | 6.4221 | 6.41164 |
| SLPI | 2.23237 | 9.60E−05 | 6.73148 | 6.57965 | 7.81386 | 7.81442 |
| SMAGP | 1.71317 | 0.00012282 | 7.46267 | 7.45851 | 8.29638 | 8.17815 |
| SNORA13 | 1.49405 | 0.0343166 | 6.36175 | 6.49522 | 6.92632 | 7.08911 |
| SNORA14B | 1.59947 | 0.0411248 | 7.44046 | 7.37755 | 8.18038 | 7.99282 |
| SNORA27 | 1.41154 | 0.00530613 | 7.42798 | 7.19541 | 7.76515 | 7.85278 |
| SNORA31 | 1.48337 | 0.00044583 | 7.135 | 7.0202 | 7.60042 | 7.69253 |
| SNORA33 | 1.44444 | 0.0111568 | 6.55316 | 6.48244 | 7.20403 | 6.8926 |
| SNORA37 | 1.43773 | 0.0405851 | 7.35581 | 7.76649 | 8.05342 | 8.11646 |
| SNORA38 | 1.56714 | 0.0324707 | 7.62469 | 7.5238 | 8.40695 | 8.0378 |
| SNORA54 | 1.59354 | 0.0406215 | 7.01491 | 7.05343 | 7.94994 | 7.46288 |
| SNORA75 | 1.44414 | 0.00949418 | 8.99269 | 8.93208 | 9.47128 | 9.51391 |
| SNORA7B | 1.5076 | 0.0185003 | 9.11188 | 9.15038 | 9.83921 | 9.60756 |
| SNORD12C | 1.42939 | 0.00064755 | 10.5753 | 10.6016 | 11.1085 | 11.0992 |
| SNORD14E | 2.33051 | 0.00528492 | 10.0837 | 9.70118 | 11.0629 | 11.1632 |
| SNORD20 | 1.40731 | 0.00145372 | 7.17187 | 7.19835 | 7.69079 | 7.66532 |
| SNORD31 | 1.40068 | 0.00593135 | 9.19453 | 8.96439 | 9.55739 | 9.5738 |
| SNORD45B | 1.47479 | 0.033261 | 10.4226 | 10.6277 | 10.9866 | 11.1847 |
| SNORD50A | 1.4588 | 0.00170386 | 9.9171 | 9.89154 | 10.4053 | 10.4929 |
| SNORD54 | 1.66197 | 0.0179825 | 8.95734 | 9.05593 | 9.61625 | 9.86281 |
| SNORD60 | 1.51269 | 0.00831843 | 7.473 | 7.72923 | 8.23065 | 8.16582 |
| SNORD61 | 1.69187 | 0.00038472 | 7.28954 | 7.176 | 7.96662 | 8.01616 |
| SNORD82 | 1.40219 | 0.0195924 | 7.88528 | 8.11548 | 8.41214 | 8.56398 |
| SNRPD3 | 1.44847 | 0.0347101 | 10.4044 | 10.6319 | 11.0247 | 11.0807 |
| SNTB1 | −1.45256 | 0.00432741 | 9.9702 | 9.9211 | 9.38993 | 9.42417 |
| SNX10 | 2.2974 | 0.00141506 | 6.25931 | 6.10603 | 7.28329 | 7.48205 |
| SPARC | 1.43409 | 9.54E−05 | 11.3079 | 11.3134 | 11.7967 | 11.8649 |
| SPN | −1.5103 | 4.30E−05 | 10.3123 | 10.2605 | 9.71987 | 9.66328 |
| SQRDL | 1.63343 | 0.00830629 | 7.13739 | 7.49405 | 8.06823 | 7.97902 |
| ST6GALNAC3 | −1.40443 | 0.0154111 | 6.28857 | 6.02078 | 5.6004 | 5.72898 |
| STAP1 | 1.88867 | 0.00025764 | 7.75513 | 7.73247 | 8.66602 | 8.65631 |
| STC2 | −1.88434 | 0.00404189 | 7.27632 | 6.91552 | 6.16112 | 6.20259 |
| STK32B | −1.52096 | 0.00207098 | 8.10402 | 8.09532 | 7.55365 | 7.43573 |
| TAGAP | −1.58787 | 0.00072607 | 7.25138 | 7.17548 | 6.63728 | 6.4554 |
| TARP | −1.42852 | 0.00755633 | 7.35207 | 7.25589 | 6.72864 | 6.85028 |
| TBC1D2B | −1.60741 | 0.00014826 | 9.02583 | 9.00134 | 8.30753 | 8.35016 |
| TBXA2R | −1.4191 | 0.0104676 | 6.69736 | 6.4712 | 5.98795 | 6.17066 |
| TFAP2A | 1.62883 | 0.0053541 | 5.38978 | 5.66587 | 6.14327 | 6.32003 |
| TGM5 | −1.46994 | 4.80E−05 | 6.25719 | 6.2056 | 5.70042 | 5.65087 |
| THRA | −1.46579 | 0.00164533 | 8.92448 | 8.88764 | 8.35769 | 8.35107 |
| THSD7A | 2.3935 | 0.00056131 | 5.24897 | 5.415 | 6.53423 | 6.64799 |
| TM2D2 | 1.55887 | 0.00032736 | 7.89805 | 8.00922 | 8.54665 | 8.64161 |
| TM4SF1 | 6.73532 | 1.22E−05 | 6.8766 | 7.06571 | 9.68307 | 9.76274 |
| TMEM111 | 1.4649 | 0.00311738 | 9.66341 | 9.79916 | 10.2408 | 10.3234 |
| TMEM173 | −1.8503 | 0.00017896 | 9.28623 | 9.21397 | 8.36416 | 8.36051 |
| TNNT1 | −1.50316 | 0.00038425 | 7.72757 | 7.68675 | 7.09877 | 7.13955 |
| TP53I3 | 1.45867 | 0.00164883 | 7.23851 | 7.30212 | 7.83075 | 7.79919 |
| TP53INP1 | 2.27233 | 0.00017836 | 5.67676 | 5.55433 | 6.78003 | 6.8194 |
| TPSAB1 | 1.41273 | 0.00178824 | 5.67544 | 5.60897 | 6.17843 | 6.10295 |
| TRAF3IP3 | −1.48756 | 0.00614293 | 6.93605 | 6.80699 | 6.3825 | 6.21465 |
| TRH | 2.21999 | 0.00044497 | 6.35226 | 6.5181 | 7.61809 | 7.55337 |
| TSPAN18 | 3.16877 | 0.00099272 | 8.89073 | 8.72307 | 10.2964 | 10.6452 |
| TSPAN7 | 1.47714 | 0.0001712 | 11.4816 | 11.423 | 11.9864 | 12.0438 |
| TSPYL1 | 1.48553 | 0.0224455 | 6.17481 | 5.99591 | 6.70049 | 6.61218 |
| TUBE1 | −1.42766 | 0.0373022 | 7.61116 | 7.37473 | 6.93785 | 7.02073 |
| UBA7 | 1.51499 | 0.0009708 | 8.02996 | 8.05386 | 8.67547 | 8.60697 |
| ULBP1 | −2.10944 | 0.00612045 | 8.49609 | 7.96418 | 7.05764 | 7.24891 |
| UNC93B1 | −1.48575 | 0.00190831 | 9.29596 | 9.20827 | 8.66241 | 8.69945 |
| USP2 | −1.42235 | 0.0080148 | 7.69952 | 7.5402 | 7.00416 | 7.219 |
| USP53 | 1.54323 | 0.00019289 | 7.85177 | 7.84769 | 8.42369 | 8.52768 |
| UTRN | 1.60698 | 0.00031493 | 8.59699 | 8.56925 | 9.25028 | 9.28467 |
| VAV3 | 1.65451 | 0.00017615 | 10.2206 | 10.2187 | 10.9233 | 10.9688 |
| VEPH1 | 2.93049 | 0.0006913 | 4.503 | 4.7895 | 6.37632 | 6.01847 |
| VNN1 | −2.3348 | 0.00136865 | 8.62855 | 8.37226 | 7.12039 | 7.43382 |
| VSTM1 | 1.5931 | 0.00193699 | 9.59818 | 9.58651 | 10.2346 | 10.2938 |
| WARS | −1.58393 | 0.00256649 | 10.6431 | 10.4029 | 9.92513 | 9.79381 |
| WDFY4 | −1.53272 | 0.00147207 | 8.07067 | 8.05114 | 7.41719 | 7.47243 |
| WIPI1 | 1.69209 | 0.00405428 | 5.77898 | 5.48946 | 6.37529 | 6.41077 |
| XRCC6BP1 | −1.55134 | 0.00119824 | 8.77991 | 8.82817 | 8.19326 | 8.1478 |
| YPEL1 | 1.40616 | 0.0173771 | 5.79062 | 5.5854 | 6.06791 | 6.29163 |
| YPEL5 | 1.55947 | 0.00025389 | 8.81046 | 8.80567 | 9.43995 | 9.45831 |
| ZBTB8B | 1.65062 | 0.0065805 | 6.46247 | 6.55366 | 7.41199 | 7.05015 |
| ZC3H12C | 1.67042 | 0.00206458 | 6.38775 | 6.56565 | 7.2275 | 7.20632 |
| ZC3H6 | 1.49675 | 0.00084272 | 6.31966 | 6.25633 | 6.82901 | 6.91064 |
| ZEB1 | 1.53945 | 0.0124442 | 8.58793 | 8.51194 | 9.26199 | 9.08271 |
| ZFP36 | 1.4111 | 0.0315186 | 7.02378 | 6.88908 | 7.47454 | 7.43198 |
| ZFYVE16 | 1.44479 | 0.0159096 | 7.52163 | 7.62791 | 8.08648 | 8.12479 |
| ZNF436 | 1.79221 | 3.74E−05 | 8.22347 | 8.14638 | 8.98771 | 9.06563 |
| ZNF589 | −1.41746 | 0.00198715 | 7.85462 | 7.98797 | 7.37807 | 7.45792 |
| ZNF608 | 1.53931 | 0.00034861 | 8.41011 | 8.45372 | 9.02072 | 9.08769 |
| ZNF774 | −1.41058 | 0.0171807 | 6.82401 | 6.57346 | 6.29703 | 6.10786 |
| ZNF792 | 1.56199 | 0.00188229 | 6.12848 | 6.211 | 6.82599 | 6.80025 |
| ZNF804A | −1.43811 | 0.00362115 | 8.89413 | 8.73899 | 8.35032 | 8.23444 |

TABLE 3
Genes showing differential expression in Kasumi-1^{A-E-KD} compared to
Kasumi-1^{Cont} cells, as measured by expression arrays (listed are genes that showed
fold-change of at least 1.4 and p-value <0.05)

| | Fold | | | | | |
|---|---|---|---|---|---|---|
| | Change | | | | | |
| | relative to | | | | | |
| | Non- | | Non- | Non- | A-E | |
| Gene Symbol | targeting | p-value | taregting_1 | taregting_2 | KD_1 | A-E KD_2 |
| ABCA2 | 3.22579 | 0.0106287 | 5.60138 | 5.89519 | 7.34175 | 7.53414 |
| ABHD4 | 1.60641 | 0.00021758 | 7.77885 | 7.79443 | 8.47689 | 8.46407 |
| ABR | 1.64715 | 0.0245918 | 6.23433 | 6.11019 | 6.79538 | 6.98909 |
| ACCN2 | −2.30037 | 0.0033559 | 7.55301 | 7.63793 | 6.449 | 6.3382 |
| ADA | 2.09758 | 0.0045242 | 8.36548 | 8.38007 | 9.36974 | 9.51326 |
| ADAM12 | −3.77458 | 0.0128419 | 7.03393 | 6.89176 | 5.25396 | 4.83909 |
| ADAMTS3 | −2.50908 | 0.011999 | 8.08672 | 7.79702 | 6.59149 | 6.63794 |
| ADAMTSL4 | −1.44478 | 0.0122621 | 7.49773 | 7.61536 | 7.03348 | 7.01792 |
| AHNAK | 2.61791 | 0.00936406 | 5.79737 | 5.81387 | 7.05898 | 7.32909 |
| AIF1 | 1.57987 | 0.00703134 | 9.89453 | 9.99977 | 10.625 | 10.5889 |
| AIF1L | −2.98901 | 0.00032707 | 8.15487 | 8.145 | 6.54213 | 6.59842 |
| ALCAM | 2.10958 | 0.00674851 | 8.48958 | 8.50734 | 9.48694 | 9.66389 |
| ALDOC | −1.79333 | 0.012676 | 8.66731 | 8.59236 | 7.87534 | 7.69904 |
| AMPD3 | 1.66096 | 0.00393566 | 6.22951 | 6.19136 | 6.90053 | 6.98438 |
| ANKRD22 | −3.5661 | 0.0145255 | 8.36292 | 8.29759 | 6.27479 | 6.71703 |
| ANPEP | 1.77194 | 0.0145152 | 6.32979 | 6.53011 | 7.26393 | 7.24663 |
| ANTXR1 | −3.00031 | 0.0039484 | 7.92082 | 7.8681 | 6.40571 | 6.21299 |
| ANTXR2 | −1.4842 | 0.0103784 | 10.775 | 10.6602 | 10.1591 | 10.1367 |
| ANXA2 | −1.69268 | 0.00796573 | 8.13701 | 8.08716 | 7.41624 | 7.28932 |
| AOAH | 9.17443 | 0.0016805 | 4.4676 | 4.72 | 7.75538 | 7.82746 |
| APBB2 | −1.64474 | 0.0157227 | 8.3168 | 8.23899 | 7.6424 | 7.47767 |
| AR | −1.72145 | 0.0276036 | 7.94909 | 7.88795 | 7.2643 | 7.0055 |
| ARC | −1.47043 | 0.0164605 | 7.94388 | 7.83021 | 7.37543 | 7.28618 |
| ARG2 | −2.83944 | 0.00732863 | 6.64577 | 6.65921 | 5.01745 | 5.27631 |
| ARHGAP1 | 1.47172 | 0.00122829 | 9.43773 | 9.42073 | 9.96912 | 10.0043 |
| ARHGAP24 | −1.40125 | 0.0485635 | 6.95108 | 6.78501 | 6.30713 | 6.45553 |
| ARHGEF12 | −3.10993 | 0.00406653 | 8.20928 | 8.15849 | 6.44543 | 6.64858 |
| ARHGEF3 | −1.84677 | 0.00836351 | 8.06732 | 7.90653 | 7.11499 | 7.08885 |
| ARPP19 | −1.40481 | 0.00710545 | 8.99832 | 8.96721 | 8.45385 | 8.53092 |
| ASPH | −1.42357 | 0.00563362 | 8.98426 | 8.93219 | 8.42048 | 8.47694 |
| ASPHD1 | −1.40178 | 0.0306078 | 7.09599 | 7.15003 | 6.55278 | 6.71873 |
| ASS1 | −1.47774 | 0.0116525 | 9.65604 | 9.6543 | 9.03043 | 9.15313 |
| ATG4C | 1.48652 | 0.00609358 | 7.92263 | 7.83357 | 8.44466 | 8.45541 |
| ATP2B4 | 3.09853 | 0.00029622 | 8.65744 | 8.60134 | 10.2625 | 10.2595 |
| ATP8B2 | −1.59384 | 0.00688137 | 8.56947 | 8.50123 | 7.81834 | 7.90734 |
| ATP8B4 | 1.94061 | 0.0321972 | 5.90849 | 5.71152 | 6.62075 | 6.91227 |
| BAHCC1 | 1.68568 | 0.00445379 | 7.65467 | 7.60147 | 8.33854 | 8.42426 |
| BASP1 | −1.73105 | 0.00078258 | 8.92547 | 8.93655 | 8.16082 | 8.11791 |
| BBS2 | 1.4281 | 7.50E−06 | 7.18006 | 7.17851 | 7.6922 | 7.69456 |
| BCL2 | 2.34125 | 0.00274287 | 7.71712 | 7.71419 | 8.87854 | 9.00733 |
| BIN2 | 2.2258 | 0.026482 | 5.6236 | 5.72072 | 6.64106 | 7.0119 |
| BMP4 | −1.70175 | 0.013341 | 7.4485 | 7.27309 | 6.576 | 6.61155 |
| BPI | 5.38983 | 0.0155851 | 5.1399 | 4.76821 | 7.13995 | 7.62864 |
| BRI3BP | 1.42706 | 0.0455507 | 10.3018 | 10.2928 | 10.9237 | 10.697 |
| C10orf114 | −2.18976 | 0.00266178 | 6.97244 | 6.8799 | 5.83112 | 5.75967 |
| C10orf54 | 1.99676 | 0.00610902 | 5.75553 | 5.91175 | 6.83725 | 6.82536 |
| C11orf17 | −1.53165 | 0.0260087 | 8.68148 | 8.49488 | 7.93394 | 8.01224 |
| C12orf75 | 1.45866 | 0.00565643 | 6.57977 | 6.65362 | 7.14321 | 7.17947 |
| C13orf15 | −2.33275 | 0.00130682 | 8.85284 | 8.93473 | 7.65505 | 7.68846 |
| C15orf39 | 1.90794 | 0.00090763 | 7.14217 | 7.12236 | 8.09057 | 8.03798 |
| C16orf54 | 1.58535 | 0.00431697 | 6.67364 | 6.75577 | 7.36421 | 7.3948 |
| C17orf60 | −2.84686 | 0.00204468 | 8.26272 | 8.18591 | 6.77149 | 6.6584 |
| C1orf186 | −1.44338 | 0.00520194 | 7.9613 | 7.89651 | 7.41995 | 7.37895 |
| C1orf57 | −1.45677 | 0.0141236 | 8.1505 | 8.18301 | 7.56084 | 7.68712 |
| C1orf71 | −1.67535 | 0.00444062 | 8.34748 | 8.26692 | 7.5335 | 7.59198 |
| C1S | −1.85757 | 0.0267009 | 6.99595 | 6.83137 | 5.89604 | 6.14445 |
| C3AR1 | −4.86022 | 0.00341898 | 8.49739 | 8.25978 | 6.03619 | 6.15894 |
| C8orf73 | 1.79625 | 0.00739747 | 7.88711 | 7.8175 | 8.63303 | 8.76156 |
| C9orf89 | 1.93896 | 0.00660485 | 7.23042 | 7.07787 | 8.12584 | 8.09302 |
| CACNB3 | −1.61061 | 0.00625528 | 9.02559 | 8.97448 | 8.26414 | 8.36072 |
| CACNB4 | −1.41705 | 0.0203426 | 9.57637 | 9.43511 | 9.02066 | 8.98503 |
| CAMK2G | 1.43731 | 0.00090043 | 9.91138 | 9.93187 | 10.4569 | 10.4331 |
| CBFA2T3 | 1.60052 | 0.0317118 | 6.9373 | 7.13537 | 7.78916 | 7.64059 |
| CC2D2A | −2.18462 | 0.0153816 | 6.69088 | 6.53631 | 5.36773 | 5.60469 |
| CCDC109B | −2.44763 | 0.00716672 | 8.3313 | 8.29757 | 6.91444 | 7.13167 |
| CCDC59 | −1.51949 | 0.0237407 | 8.44961 | 8.29128 | 7.71489 | 7.81882 |
| CCDC88A | 1.62841 | 0.00210477 | 9.08543 | 9.11864 | 9.77777 | 9.83323 |
| CCND3 | 1.46823 | 0.00623941 | 7.9079 | 7.92184 | 8.42553 | 8.51236 |
| CD244 | 1.58494 | 0.0133803 | 7.65369 | 7.75425 | 8.30924 | 8.42756 |
| CD300A | 1.4111 | 0.015837 | 7.20292 | 7.282 | 7.78868 | 7.68988 |
| CD300C | 1.40946 | 0.00459639 | 6.7035 | 6.64017 | 7.17846 | 7.15549 |
| CD33 | 2.8113 | 0.00209305 | 9.53172 | 9.45385 | 10.9279 | 11.0402 |
| CD38 | 3.04391 | 0.00204021 | 5.91844 | 5.92472 | 7.45493 | 7.60009 |
| CD44 | 1.51382 | 0.0019417 | 11.4655 | 11.4397 | 12.0278 | 12.0738 |
| CD48 | 1.84898 | 0.00607603 | 6.0553 | 5.93076 | 6.84904 | 6.91047 |
| CD53 | −2.86635 | 0.00471833 | 11.3567 | 11.2281 | 9.85589 | 9.69055 |
| CD58 | −1.55288 | 0.0351731 | 9.01056 | 9.0062 | 8.25112 | 8.49574 |
| CD82 | 2.11199 | 0.00051451 | 6.2271 | 6.1782 | 7.28235 | 7.28016 |
| CD84 | 4.07055 | 0.00282319 | 4.664 | 4.87828 | 6.80858 | 6.78415 |
| CD96 | −2.13474 | 0.00202891 | 7.76515 | 7.76347 | 6.7196 | 6.6209 |
| CECR6 | 1.64305 | 0.0322643 | 6.7146 | 6.94409 | 7.61075 | 7.48069 |
| CEP55 | 1.50196 | 0.00058596 | 7.90836 | 7.92845 | 8.51531 | 8.49519 |
| CEP70 | −1.527 | 0.0241981 | 7.8992 | 7.85289 | 7.1714 | 7.3593 |
| CHCHD10 | −1.42839 | 0.0427504 | 9.86231 | 9.94106 | 9.28468 | 9.48992 |
| CHST12 | 1.73465 | 0.0223944 | 6.73937 | 6.85834 | 7.69882 | 7.48817 |
| CIB3 | 4.34255 | 0.00885089 | 4.85917 | 5.03923 | 6.88843 | 7.24706 |
| CKB | −2.67134 | 0.00414705 | 8.79861 | 8.7384 | 7.26446 | 7.43743 |
| CLDN15 | −1.72236 | 0.0205093 | 8.8884 | 8.82177 | 8.17983 | 7.96158 |
| CLEC5A | −1.98045 | 0.00651321 | 10.5852 | 10.4909 | 9.48765 | 9.6168 |
| CLIP2 | 2.96118 | 6.37E−05 | 5.56079 | 5.55183 | 7.11081 | 7.13415 |
| CNKSR3 | −1.47617 | 0.048038 | 6.6272 | 6.51046 | 5.89328 | 6.12066 |
| CRIP1 | 1.69539 | 0.00771564 | 6.36348 | 6.41333 | 7.21253 | 7.08753 |
| CSF1R | 3.67655 | 0.00058276 | 6.07699 | 6.02526 | 7.89221 | 7.96674 |
| CSRNP2 | −1.43952 | 0.0173946 | 8.20524 | 8.25967 | 7.64211 | 7.77162 |
| CSRP1 | −1.67154 | 0.00283856 | 10.4733 | 10.4383 | 9.75013 | 9.67912 |
| CST3 | 1.44168 | 0.0472593 | 9.69506 | 9.63635 | 10.0781 | 10.3088 |
| CST7 | 5.25192 | 0.00842437 | 6.28022 | 6.69047 | 8.79588 | 8.9605 |
| CTDSPL | −1.55761 | 0.00240069 | 8.95344 | 8.92227 | 8.27129 | 8.32576 |
| CTNNBIP1 | 1.45872 | 0.0334584 | 7.45297 | 7.61199 | 8.01294 | 8.14142 |
| CTSD | 1.84392 | 0.00444818 | 10.8269 | 10.9046 | 11.704 | 11.7931 |
| CTSG | 1.98309 | 0.00395794 | 9.77812 | 9.8056 | 10.7188 | 10.8404 |
| CXCR3 | 1.55007 | 0.00709649 | 6.04533 | 6.14528 | 6.7469 | 6.70838 |
| CYLD | 1.44137 | 0.00631902 | 6.94819 | 6.89746 | 7.41663 | 7.48391 |
| CYP2S1 | −1.85104 | 0.0179063 | 8.55178 | 8.42157 | 7.69973 | 7.49694 |
| CYP46A1 | −2.13931 | 0.0175102 | 8.13852 | 8.03069 | 6.85057 | 7.12434 |
| CYTL1 | −1.63553 | 0.0143672 | 7.62044 | 7.54185 | 6.94789 | 6.79489 |
| CYTSB | 1.51526 | 0.0258878 | 7.13296 | 7.06292 | 7.60556 | 7.78945 |
| DAPP1 | −1.51727 | 0.00267773 | 7.26626 | 7.20933 | 6.62358 | 6.64907 |
| DCLRE1C | 1.67629 | 0.00871457 | 7.75291 | 7.67321 | 8.40074 | 8.51592 |
| DCPS | 1.74634 | 0.00053195 | 7.08443 | 7.05337 | 7.86308 | 7.88339 |
| DCTN6 | −1.41618 | 0.0337131 | 7.98004 | 8.15396 | 7.60219 | 7.5278 |
| DDB2 | 1.52102 | 0.0168308 | 7.69918 | 7.66245 | 8.36321 | 8.20851 |
| DHRS3 | −1.84091 | 0.00749322 | 8.36164 | 8.21537 | 7.38517 | 7.43102 |
| DOCK10 | −1.43623 | 0.0244365 | 9.89552 | 9.77584 | 9.37117 | 9.25563 |
| DOCK6 | −1.54049 | 0.0451325 | 6.86695 | 6.84746 | 6.37059 | 6.09704 |
| DPEP1 | −2.09025 | 0.0357737 | 7.16869 | 6.76687 | 5.85521 | 5.95299 |
| DPYSL2 | −1.40994 | 0.027827 | 9.08757 | 8.9241 | 8.48897 | 8.53145 |
| DRAM1 | −2.99562 | 0.00144511 | 10.2038 | 10.1161 | 8.53584 | 8.61837 |
| DUSP1 | −1.55765 | 0.0491714 | 7.23439 | 6.94198 | 6.46645 | 6.43118 |
| DUSP10 | 1.9949 | 0.0426742 | 6.02187 | 6.17948 | 6.89944 | 7.29454 |
| DUSP6 | −1.6602 | 0.00705827 | 9.85134 | 9.78054 | 9.03396 | 9.13521 |
| E2F5 | −1.44771 | 0.0428232 | 8.24794 | 8.13649 | 7.55881 | 7.75807 |
| ECM1 | −1.46936 | 0.00466868 | 8.7451 | 8.69823 | 8.19648 | 8.13648 |
| EDIL3 | −7.595 | 0.0012061 | 8.12011 | 8.06663 | 5.07023 | 5.26642 |
| EFHD2 | 1.79663 | 0.0275199 | 7.08882 | 7.14026 | 7.81896 | 8.1007 |
| ELF4 | 2.19463 | 0.00524816 | 7.22832 | 7.38226 | 8.40964 | 8.4689 |
| EMID1 | 1.74498 | 0.00383423 | 6.84922 | 6.75428 | 7.58964 | 7.62028 |
| EMP2 | −1.9657 | 0.00985025 | 7.8168 | 7.68681 | 6.84942 | 6.70409 |
| EMR2 | 3.81563 | 0.0151171 | 5.94 | 6.07131 | 7.70645 | 8.16869 |
| ENAH | −1.57043 | 0.0111527 | 6.97914 | 6.89008 | 6.33661 | 6.23028 |
| ENTPD1 | −3.1966 | 0.0142729 | 8.0885 | 7.81981 | 6.12615 | 6.42909 |
| EPHX1 | −1.4409 | 0.00724958 | 7.94553 | 7.8743 | 7.35526 | 7.41064 |
| ERAP2 | 1.54037 | 0.0199047 | 8.10297 | 8.16706 | 8.67497 | 8.84161 |
| ERBB2IP | 1.5894 | 0.0217548 | 9.60905 | 9.41416 | 10.1566 | 10.2036 |
| ERG | −1.51218 | 0.00023528 | 9.16914 | 9.17677 | 8.58465 | 8.56801 |
| ERLIN2 | −1.56482 | 0.00304301 | 7.90618 | 7.94853 | 7.31012 | 7.25259 |
| ESAM | −2.13135 | 0.0346365 | 6.51673 | 6.54453 | 5.64705 | 5.23068 |
| ETS2 | 1.57062 | 0.0142829 | 9.24668 | 9.20889 | 9.80273 | 9.9555 |
| ETV5 | −1.91752 | 0.0199868 | 7.10776 | 6.86611 | 5.98789 | 6.10749 |
| EVI2A | −1.61923 | 0.0186643 | 8.61376 | 8.67452 | 7.85741 | 8.04027 |
| FAM101B | 2.48533 | 0.00107311 | 8.90976 | 8.89603 | 10.1738 | 10.2588 |
| FAM105A | −1.67693 | 0.0109472 | 9.57808 | 9.43157 | 8.78772 | 8.73028 |
| FAM107B | −1.41515 | 0.0116847 | 10.376 | 10.3694 | 9.81718 | 9.92624 |
| FAM46A | 1.42464 | 0.00925174 | 10.3456 | 10.3063 | 10.7912 | 10.8819 |
| FARP2 | −1.44615 | 0.0130883 | 7.48316 | 7.36233 | 6.87904 | 6.90202 |
| FCGR1A | −1.96709 | 0.00244591 | 10.0199 | 9.98033 | 9.06817 | 8.9799 |
| FCGR1B | −1.92085 | 0.00541143 | 9.88311 | 9.79018 | 8.94667 | 8.84314 |
| FCGRT | −1.54353 | 0.0112469 | 9.55229 | 9.54909 | 8.8575 | 8.99142 |
| FES | 1.5307 | 0.0267797 | 7.86518 | 7.86813 | 8.37828 | 8.58342 |
| FHL1 | −1.45463 | 0.0397166 | 8.28682 | 8.09704 | 7.70904 | 7.59353 |
| FLJ38379 | −1.63497 | 0.0295302 | 7.19393 | 6.97216 | 6.31682 | 6.43075 |
| FLNA | 1.69643 | 0.0256105 | 8.53491 | 8.41941 | 9.12944 | 9.34987 |
| FLOT1 | −1.81739 | 0.00023971 | 7.60708 | 7.61736 | 6.73803 | 6.76266 |
| FLOT2 | 2.13966 | 0.022266 | 6.052 | 6.38336 | 7.29811 | 7.33202 |
| FLVCR2 | −1.65116 | 0.0174131 | 7.32168 | 7.12888 | 6.49377 | 6.50983 |
| FMNL2 | −1.7304 | 0.00072629 | 7.52453 | 7.48503 | 6.70561 | 6.72173 |
| FNBP1L | −1.59717 | 0.032213 | 8.10555 | 7.97416 | 7.25885 | 7.46982 |
| FOSL2 | −1.71815 | 0.00381856 | 10.0594 | 9.96317 | 9.22531 | 9.23555 |
| FOXN3 | −1.42377 | 0.0158352 | 10.6798 | 10.5797 | 10.1614 | 10.0787 |
| FRMD6 | 1.42671 | 0.0122943 | 6.06501 | 6.0439 | 6.51075 | 6.62355 |
| FSD1 | −1.71379 | 0.0183468 | 6.47064 | 6.64325 | 5.84257 | 5.71694 |
| FYB | 1.77638 | 0.0156958 | 6.25258 | 6.04273 | 6.98258 | 6.9706 |
| FZD2 | −1.54117 | 0.0209939 | 8.15078 | 8.22019 | 7.64652 | 7.47639 |
| GAA | −1.44184 | 0.0339549 | 8.4927 | 8.56564 | 7.90831 | 8.09419 |
| GALNT1 | −1.50834 | 0.0480601 | 10.3261 | 10.2929 | 9.58266 | 9.85042 |
| GALNT3 | 1.648 | 0.00959683 | 6.76957 | 6.6784 | 7.49929 | 7.39012 |
| GBGT1 | 1.62268 | 0.047343 | 7.42448 | 7.32441 | 7.92337 | 8.22228 |
| GCH1 | −1.71904 | 0.00285163 | 6.9493 | 6.8673 | 6.11842 | 6.13497 |
| GFI1 | −1.4051 | 0.0107238 | 11.0028 | 10.944 | 10.4408 | 10.5246 |
| GGTA1 | −1.83601 | 0.0038889 | 6.80416 | 6.75766 | 5.85469 | 5.95399 |
| GHRL | 1.67214 | 0.0307915 | 6.43634 | 6.55599 | 7.1188 | 7.35692 |
| GLI3 | −1.97163 | 0.00026941 | 7.13867 | 7.16192 | 6.1598 | 6.18202 |
| GLIPR1 | −1.40742 | 0.0281877 | 9.11732 | 8.9837 | 8.5056 | 8.60932 |
| GLMN | −1.4827 | 0.0306161 | 8.7851 | 8.61961 | 8.19339 | 8.07486 |
| GNA15 | 1.50664 | 0.00385142 | 8.16527 | 8.11006 | 8.70466 | 8.75334 |
| GNG12 | −1.58944 | 0.0152364 | 8.42175 | 8.33384 | 7.63831 | 7.78024 |
| GPN3 | −1.40576 | 0.00787251 | 8.8249 | 8.75976 | 8.27161 | 8.33035 |
| GPR114 | 4.0541 | 0.00551373 | 5.7761 | 5.96826 | 7.77563 | 8.00749 |
| GPR124 | −1.96531 | 0.00547148 | 8.81216 | 8.68976 | 7.73752 | 7.81488 |
| GSTM3 | −1.49231 | 0.011553 | 8.33092 | 8.36008 | 7.70705 | 7.82885 |
| GUCY1B3 | −2.00133 | 0.00727896 | 7.03336 | 6.90498 | 6.02525 | 5.91117 |
| GYPC | −1.53715 | 0.00598809 | 9.93974 | 9.89825 | 9.25522 | 9.34227 |
| HACE1 | −1.52599 | 0.0129726 | 9.23456 | 9.13861 | 8.52568 | 8.628 |
| HCST | 4.12679 | 0.0071136 | 7.01833 | 7.14161 | 8.9629 | 9.28707 |
| HIST1H2BK | −1.75295 | 0.040443 | 9.67179 | 9.62435 | 9.00459 | 8.67198 |
| HIVEP1 | −1.46296 | 0.0292035 | 8.75657 | 8.5744 | 8.08655 | 8.14664 |
| HIVEP3 | 1.55497 | 0.0426467 | 6.62492 | 6.4519 | 7.28012 | 7.07048 |
| HLA-E | −1.43285 | 0.0219225 | 9.8611 | 9.72238 | 9.3088 | 9.23691 |
| HOMER3 | −1.5602 | 0.0344719 | 8.70556 | 8.77069 | 7.97847 | 8.21431 |
| HOOK3 | −1.56771 | 0.0131507 | 8.88611 | 8.89738 | 8.318 | 8.16817 |
| HPCAL1 | 1.45353 | 0.0307554 | 8.28301 | 8.24555 | 8.7088 | 8.89889 |
| HPSE | −2.99899 | 0.00216524 | 7.77004 | 7.68895 | 6.20675 | 6.08329 |
| HSP90AA6P | −1.66305 | 0.00437652 | 6.82846 | 6.90837 | 6.16244 | 6.10673 |
| ICAM1 | −1.84351 | 0.0183012 | 7.90749 | 7.75867 | 6.85515 | 7.0461 |
| ICAM3 | 3.12967 | 0.00374428 | 6.84157 | 6.8638 | 8.3983 | 8.59908 |
| ID2 | 2.15689 | 0.00518688 | 6.86071 | 6.86876 | 8.05376 | 7.89361 |
| IGFBP4 | −2.05871 | 0.00228656 | 10.4444 | 10.4386 | 9.44957 | 9.34994 |
| IGFBP7 | 2.12108 | 0.00321563 | 11.3499 | 11.3235 | 12.3612 | 12.4817 |
| IL13RA1 | −1.82213 | 0.0202167 | 7.99747 | 7.9609 | 6.98992 | 7.2372 |
| IL17RA | 2.28466 | 0.0002917 | 8.08367 | 8.12423 | 9.2941 | 9.29777 |
| IL1RAP | −1.42113 | 0.00044592 | 7.429 | 7.43635 | 6.93569 | 6.91557 |
| IL6R | 6.52337 | 0.00111527 | 6.523 | 6.39947 | 9.1008 | 9.2329 |
| IL8 | −3.45852 | 0.00171094 | 7.65107 | 7.53008 | 5.75756 | 5.84328 |
| INA | −3.34966 | 0.00012083 | 7.35042 | 7.3811 | 5.63324 | 5.61025 |
| INO80C | −1.55198 | 0.0325772 | 7.79491 | 7.75464 | 7.02507 | 7.25626 |
| INPP4B | −2.15244 | 0.0165779 | 7.29393 | 7.01697 | 6.08969 | 6.00926 |
| INPP5A | 1.61626 | 0.00728413 | 8.42774 | 8.31653 | 9.08581 | 9.04379 |
| IPCEF1 | −1.9627 | 0.00279954 | 9.93561 | 9.87065 | 8.89022 | 8.97036 |
| IQGAP2 | 1.84548 | 0.00227523 | 8.79049 | 8.74504 | 9.68736 | 9.61616 |
| IRF1 | 1.50265 | 0.0346606 | 6.61854 | 6.60581 | 7.08754 | 7.31182 |
| IRF8 | 2.44763 | 0.0186267 | 6.81625 | 7.05218 | 8.09129 | 8.3599 |
| ISCA1 | −1.42816 | 0.00125013 | 9.41524 | 9.38468 | 8.89568 | 8.87592 |
| ISYNA1 | −1.51459 | 0.0259532 | 9.08033 | 8.99878 | 8.35106 | 8.5302 |
| ITGA6 | 1.53794 | 0.0414066 | 7.46144 | 7.45658 | 7.94958 | 8.21044 |
| ITGA9 | −3.30571 | 0.0100109 | 8.14741 | 7.82289 | 6.19765 | 6.32273 |
| ITGB2 | 2.04244 | 0.0272229 | 8.58132 | 8.60466 | 9.45012 | 9.79645 |
| ITM2C | 1.99419 | 0.0045239 | 8.75464 | 8.82159 | 9.84219 | 9.72565 |
| JAG1 | 1.41019 | 0.00220125 | 7.95584 | 7.91022 | 8.42414 | 8.43369 |
| JMJD1C | −1.49937 | 0.0189358 | 10.5865 | 10.5386 | 9.90019 | 10.0561 |
| KAT2B | 1.44911 | 0.00072803 | 7.56693 | 7.59536 | 8.11372 | 8.1189 |
| KCNAB2 | 1.88467 | 0.00949454 | 9.16635 | 9.14873 | 9.98255 | 10.1611 |
| KDELC1 | −1.48375 | 0.0430143 | 6.7838 | 6.61723 | 6.22047 | 6.04207 |
| KIAA0125 | 1.81753 | 0.0248589 | 5.99223 | 6.05708 | 7.02129 | 6.75198 |
| KIAA0182 | 2.46251 | 0.00330428 | 7.83712 | 7.9815 | 9.22949 | 9.1894 |
| KIAA1462 | −2.33329 | 0.00852749 | 8.14183 | 7.98281 | 6.75881 | 6.9211 |
| KIF3C | −1.74973 | 0.0319704 | 8.08905 | 8.02525 | 7.10561 | 7.39442 |
| KLF10 | 1.65782 | 0.0246188 | 6.69341 | 6.52332 | 7.41741 | 7.2579 |
| KLF7 | 1.5273 | 0.0092751 | 7.66097 | 7.56774 | 8.26191 | 8.18876 |
| LAMB1 | −2.68517 | 0.00085612 | 7.76901 | 7.71743 | 6.28541 | 6.35101 |
| LAMC1 | −1.5832 | 0.00997807 | 7.38242 | 7.38779 | 6.6556 | 6.78892 |
| LAPTM5 | 2.45311 | 0.00085 | 10.2507 | 10.2159 | 11.5614 | 11.4944 |
| LBR | −1.72987 | 0.019082 | 10.5676 | 10.3947 | 9.62122 | 9.7598 |
| LCP1 | 5.87542 | 0.00027953 | 8.61241 | 8.56489 | 11.1078 | 11.1788 |
| LGALS1 | 2.99361 | 0.0146487 | 7.04245 | 6.84028 | 8.68836 | 8.35815 |
| LGALS12 | 1.4229 | 0.00984938 | 10.0132 | 9.95298 | 10.5329 | 10.4509 |
| LHFP | −1.51413 | 0.0263108 | 7.99262 | 8.07584 | 7.52562 | 7.34586 |
| LILRA2 | −1.65388 | 0.0134284 | 9.39459 | 9.24147 | 8.55532 | 8.62904 |
| LOC100008589 | −1.46542 | 0.00158655 | 9.56097 | 9.57454 | 8.99553 | 9.03736 |
| LOC153684 | 1.45527 | 0.0191028 | 7.31792 | 7.31766 | 7.78318 | 7.93499 |
| LOC284422 | 1.57794 | 0.00495706 | 6.69179 | 6.59967 | 7.29738 | 7.31017 |
| LOC284757 | 1.69515 | 0.00593536 | 6.57326 | 6.5467 | 7.3788 | 7.26399 |
| LPAR1 | −1.97878 | 0.00465133 | 7.51713 | 7.47787 | 6.44842 | 6.57735 |
| LPAR6 | −2.84085 | 0.00401906 | 6.93901 | 6.74921 | 5.32482 | 5.35076 |
| LPHN1 | −1.6231 | 0.0165528 | 8.97583 | 8.8967 | 8.3195 | 8.15552 |
| LPHN3 | −1.78268 | 0.00187404 | 6.78008 | 6.72226 | 5.89541 | 5.93884 |
| LPXN | 1.46785 | 0.0158884 | 8.64144 | 8.69825 | 9.28823 | 9.15888 |
| LRFN4 | 1.67035 | 0.00037654 | 6.52188 | 6.49383 | 7.25112 | 7.2449 |
| LRRC17 | −2.70002 | 0.0248323 | 7.42026 | 7.13307 | 6.02352 | 5.66388 |
| LRRC33 | 1.58748 | 0.00187951 | 9.27849 | 9.2368 | 9.94446 | 9.90429 |
| LST1 | 2.39606 | 3.17E−05 | 8.38062 | 8.38604 | 9.65055 | 9.63743 |
| LYZ | 1.65369 | 0.0230671 | 7.94714 | 8.08972 | 8.65752 | 8.83071 |
| MAFG | 1.4665 | 0.0102248 | 8.92367 | 8.93076 | 9.53577 | 9.42342 |
| MAGED2 | −1.58979 | 0.0205719 | 8.53729 | 8.35312 | 7.7445 | 7.80823 |
| MAGED4 | −1.56631 | 0.00034043 | 8.14395 | 8.1212 | 7.48154 | 7.48886 |
| MARCH3 | 1.69894 | 0.00360018 | 7.1792 | 7.16918 | 7.98455 | 7.89309 |
| MBP | 1.69894 | 0.00051942 | 6.08863 | 6.10515 | 6.84618 | 6.87688 |
| MCTP1 | −1.55788 | 0.022314 | 9.34648 | 9.1659 | 8.65253 | 8.58069 |
| MCTP2 | 2.27459 | 0.00363376 | 6.96932 | 6.87764 | 8.16417 | 8.054 |
| ME3 | 1.51606 | 0.00711818 | 7.68134 | 7.64906 | 8.31382 | 8.21723 |
| MGAT4B | 1.40055 | 0.0136265 | 7.90525 | 7.8261 | 8.39314 | 8.31021 |
| MIER3 | −1.44966 | 0.0308558 | 9.02801 | 8.95881 | 8.54761 | 8.36778 |
| MMP28 | −1.6536 | 0.0224007 | 7.07334 | 7.03207 | 6.21857 | 6.43562 |
| MRC2 | 1.40018 | 0.017738 | 7.50325 | 7.50424 | 7.92381 | 8.05491 |
| MT1G | −1.51367 | 0.00602206 | 9.67842 | 9.7492 | 9.14611 | 9.08541 |
| MYCBP2 | 1.40056 | 0.0046572 | 9.435 | 9.38942 | 9.87396 | 9.92247 |
| MYO10 | −2.41211 | 0.00545769 | 8.86619 | 8.76649 | 7.46608 | 7.62601 |
| MYO1B | −1.53675 | 0.0450056 | 7.17692 | 6.9156 | 6.46463 | 6.38813 |
| MYO1F | 2.14191 | 0.0120161 | 7.86721 | 7.84255 | 8.83285 | 9.07471 |
| MYO1G | 1.41628 | 0.0279426 | 9.45693 | 9.51626 | 9.90826 | 10.0691 |
| NAV1 | −1.88229 | 0.00483673 | 9.26193 | 9.14458 | 8.26598 | 8.31555 |
| NCF4 | −1.44568 | 0.00778436 | 9.36095 | 9.36738 | 8.78534 | 8.8795 |
| NCKAP1 | −2.84747 | 0.010182 | 6.60093 | 6.39155 | 4.87429 | 5.09883 |
| NDST2 | −1.4935 | 0.00098203 | 9.48305 | 9.44875 | 8.89314 | 8.88127 |
| NFATC2 | 1.85839 | 0.0023322 | 7.57016 | 7.62091 | 8.45457 | 8.52462 |
| NFE2 | 4.54911 | 0.00047378 | 4.94641 | 5.0371 | 7.16291 | 7.19177 |
| NINJ2 | 1.47535 | 0.0430839 | 6.99552 | 7.03594 | 7.69545 | 7.45811 |
| NIPAL2 | 1.89383 | 0.00760288 | 8.08426 | 8.01033 | 9.04045 | 8.89676 |
| NKG7 | 6.75135 | 0.0045181 | 6.18961 | 6.51648 | 9.01979 | 9.19665 |
| NKX2-4 | 1.40284 | 0.0240006 | 7.51707 | 7.65065 | 8.11063 | 8.0338 |
| NOTCH2 | 2.28149 | 0.00217254 | 6.59406 | 6.5259 | 7.70609 | 7.79384 |
| NOV | −1.95016 | 0.00773441 | 9.19192 | 9.26427 | 8.34168 | 8.18732 |
| NRP1 | 1.47559 | 0.0459439 | 6.47742 | 6.55048 | 6.95606 | 7.19441 |
| NRXN2 | 1.65397 | 0.0135785 | 6.90857 | 6.99206 | 7.75082 | 7.60167 |
| NUP210 | 1.5071 | 0.00115763 | 9.88638 | 9.89496 | 10.4628 | 10.5021 |
| OGDHL | −1.49474 | 0.0200105 | 7.13065 | 6.98163 | 6.43903 | 6.51347 |
| OGG1 | 2.06209 | 0.00569102 | 5.57958 | 5.71515 | 6.6507 | 6.73225 |
| OSBPL11 | −1.75786 | 0.0200452 | 6.76887 | 6.82038 | 5.86669 | 6.09492 |
| P2RY2 | 1.80401 | 0.00502009 | 7.20336 | 7.15758 | 7.97563 | 8.08772 |
| PAPSS2 | −2.06903 | 0.00793989 | 9.66988 | 9.4865 | 8.50839 | 8.55008 |
| PARVG | 2.15136 | 0.00096149 | 9.76366 | 9.73782 | 10.8242 | 10.8878 |
| PCSK6 | −1.45385 | 0.00574141 | 7.29728 | 7.2177 | 6.72785 | 6.70738 |
| PDE1C | −2.37387 | 0.00278728 | 9.03478 | 8.96393 | 7.80779 | 7.69645 |
| PGAP1 | −1.57568 | 0.0388637 | 7.70886 | 7.55698 | 6.86747 | 7.08641 |
| PHF1 | −1.44006 | 0.00652771 | 8.14077 | 8.07241 | 7.55484 | 7.60608 |
| PHLPP2 | −1.45152 | 0.0167115 | 7.36227 | 7.25826 | 6.82013 | 6.72529 |
| PIK3C2A | −1.41114 | 0.00735676 | 9.73952 | 9.73081 | 9.28094 | 9.19568 |
| PIM1 | 2.45921 | 0.0293301 | 6.93062 | 7.10835 | 8.10841 | 8.52695 |
| PLAC8 | 2.03312 | 0.00190724 | 9.91304 | 9.83439 | 10.876 | 10.9188 |
| PLCB2 | 1.61902 | 0.00054942 | 7.58774 | 7.56641 | 8.25987 | 8.28453 |
| PLD4 | 1.63815 | 0.00822393 | 8.80943 | 8.7328 | 9.53566 | 9.4307 |
| PLEKHG2 | −1.5276 | 0.00217806 | 7.35 | 7.36801 | 6.72062 | 6.77486 |
| PLK3 | −1.99704 | 0.0129465 | 7.14016 | 6.98698 | 6.15102 | 5.98038 |
| PLP2 | 2.23552 | 0.00265799 | 9.34734 | 9.43831 | 10.5925 | 10.5144 |
| PLXNA4 | −6.90498 | 0.00090276 | 8.73364 | 8.71006 | 6.01719 | 5.85123 |
| PLXNB2 | 1.82759 | 0.00558761 | 7.3647 | 7.30891 | 8.1477 | 8.26579 |
| PODNL1 | −1.61781 | 0.00729779 | 6.64858 | 6.74913 | 5.97277 | 6.03685 |
| PPFIBP1 | −1.72181 | 0.00365635 | 7.2004 | 7.10632 | 6.37625 | 6.36262 |
| PRAM1 | 2.93779 | 0.00164442 | 7.24237 | 7.20881 | 8.84117 | 8.71947 |
| PRDM8 | −2.01877 | 0.00181122 | 10.0327 | 9.96258 | 9.00938 | 8.95896 |
| PREX1 | 1.58069 | 0.0217098 | 7.39487 | 7.32409 | 7.92764 | 8.11244 |
| PRICKLE1 | −1.60798 | 0.0324006 | 8.58173 | 8.43959 | 7.72084 | 7.92999 |
| PRKCD | 4.09554 | 0.00418317 | 6.08582 | 6.03468 | 7.96483 | 8.22377 |
| PRKCH | −1.52651 | 4.54E−05 | 9.47366 | 9.46575 | 8.86059 | 8.85834 |
| PRTFDC1 | −2.11816 | 0.00094202 | 6.86609 | 6.8512 | 5.80825 | 5.74342 |
| PRTN3 | −1.4279 | 0.00390801 | 11.1429 | 11.1747 | 10.6729 | 10.6169 |
| PTK2 | −3.06866 | 0.0156324 | 10.1054 | 9.99529 | 8.23564 | 8.62986 |
| PTPLAD1 | −1.44438 | 0.0412168 | 10.8908 | 10.7879 | 10.4074 | 10.2103 |
| PTPN12 | 3.35541 | 0.0261914 | 5.99087 | 5.5863 | 7.32959 | 7.74055 |
| PTPN22 | 2.35751 | 0.0194793 | 5.91057 | 5.6322 | 7.11515 | 6.90215 |
| PTPN6 | 2.03521 | 0.0126452 | 7.54888 | 7.38179 | 8.57156 | 8.40948 |
| PTPRE | −1.62913 | 0.0187807 | 9.17493 | 9.01785 | 8.33388 | 8.45069 |
| PTPRK | −1.40028 | 0.0372634 | 7.19801 | 7.15681 | 6.59744 | 6.78594 |
| PTPRM | −1.68158 | 0.000782 | 9.32578 | 9.30165 | 8.58106 | 8.54673 |
| PYCARD | 1.44215 | 0.0496332 | 9.33884 | 9.30004 | 9.9684 | 9.72693 |
| RAB27B | −1.76167 | 0.0021797 | 7.93905 | 7.87053 | 7.07094 | 7.10474 |
| RAB31 | 1.48743 | 4.88E−05 | 9.54607 | 9.55294 | 10.1203 | 10.1244 |
| RAB9A | −1.40746 | 0.020837 | 7.95573 | 7.81369 | 7.37801 | 7.40522 |
| RAC2 | 1.63112 | 0.00065174 | 10.6496 | 10.6813 | 11.3799 | 11.3627 |
| RAG1AP1 | 1.82586 | 0.0111553 | 7.19343 | 7.32785 | 8.06565 | 8.1928 |
| RASA3 | 1.6683 | 0.011278 | 6.36291 | 6.4161 | 7.05341 | 7.20236 |
| RASAL3 | 1.42574 | 0.00849327 | 6.89066 | 6.92538 | 7.37556 | 7.46391 |
| RASGRP2 | 2.73832 | 0.00185262 | 7.75523 | 7.85385 | 9.29646 | 9.2192 |
| RASSF2 | 8.33815 | 0.00061574 | 6.0415 | 5.89337 | 9.04403 | 9.0103 |
| RASSF3 | −1.45201 | 0.0152342 | 8.30718 | 8.34828 | 7.72571 | 7.85364 |
| RAVER2 | 2.88394 | 0.00096899 | 6.5962 | 6.69028 | 8.16402 | 8.17854 |
| RBAK | −1.45025 | 0.0444864 | 6.96396 | 6.8255 | 6.26404 | 6.45282 |
| RBKS | −1.99286 | 0.00768158 | 7.03579 | 6.87102 | 5.98864 | 5.92849 |
| RCBTB2 | −2.0888 | 0.0386819 | 7.59385 | 7.375 | 6.23633 | 6.60717 |
| RCN3 | −1.80639 | 0.0445956 | 7.06948 | 6.72205 | 5.97493 | 6.11038 |
| RET | −1.46169 | 0.00726256 | 8.79404 | 8.83985 | 8.22835 | 8.31027 |
| RETN | 1.8044 | 0.0408048 | 6.11393 | 6.25141 | 7.19783 | 6.87055 |
| RFC2 | 1.40554 | 0.0396667 | 8.87034 | 8.82286 | 9.23972 | 9.43574 |
| RIMBP3 | 1.70765 | 0.0125689 | 5.70911 | 5.7932 | 6.44657 | 6.59976 |
| RNASE2 | 2.23271 | 0.0127308 | 11.0353 | 10.7718 | 12.0538 | 12.0708 |
| RNASE3 | 3.38734 | 0.00161517 | 7.80602 | 7.67933 | 9.47115 | 9.53451 |
| RNF144A | 1.44149 | 0.0033695 | 7.46221 | 7.4344 | 7.94849 | 8.00324 |
| RORC | −1.97157 | 0.0116999 | 7.67534 | 7.85167 | 6.72375 | 6.84456 |
| RPS6KA1 | 3.89877 | 0.00405083 | 7.28962 | 7.31032 | 9.1381 | 9.38788 |
| RUNX1T1 | −1.47398 | 0.00991176 | 10.8751 | 10.8191 | 10.336 | 10.2388 |
| RUNX3 | 1.64053 | 0.0253863 | 7.92869 | 7.78439 | 8.66154 | 8.47986 |
| RXRA | 1.46584 | 0.0395469 | 7.01234 | 6.8211 | 7.52886 | 7.40804 |
| S100A4 | 2.03649 | 0.0397444 | 6.3825 | 6.77414 | 7.52609 | 7.68272 |
| SAMD9L | 1.53847 | 0.00924018 | 7.715 | 7.65682 | 8.36007 | 8.25475 |
| SAMSN1 | −1.95929 | 0.0153479 | 7.59314 | 7.6698 | 6.54572 | 6.77656 |
| SCPEP1 | 1.85889 | 0.00744408 | 7.70728 | 7.74638 | 8.54617 | 8.69638 |
| SELL | 2.32738 | 0.0101508 | 6.89377 | 6.65064 | 8.01396 | 7.96786 |
| SELPLG | 15.7743 | 0.00122238 | 5.84449 | 5.61429 | 9.78729 | 9.63049 |
| SEMA4A | 1.40453 | 0.0312877 | 8.24212 | 8.31571 | 8.6882 | 8.8498 |
| SEMA4D | 1.5826 | 0.0224418 | 5.71048 | 5.90107 | 6.50129 | 6.43485 |
| SERPINA1 | −2.17559 | 0.00710327 | 8.31085 | 8.24384 | 7.06702 | 7.24486 |
| SERPINB1 | −1.45852 | 0.00103241 | 11.7341 | 11.7459 | 11.179 | 11.212 |
| SERPINB9 | −7.51782 | 0.00708342 | 8.46638 | 8.43422 | 5.29426 | 5.78571 |
| SETD7 | −1.42715 | 0.0266475 | 8.65608 | 8.60664 | 8.03639 | 8.20005 |
| SEZ6L2 | −1.41002 | 0.00219142 | 6.86856 | 6.91462 | 6.39272 | 6.39903 |
| SFXN3 | 1.83943 | 0.00176995 | 7.31076 | 7.38304 | 8.23426 | 8.21806 |
| SH2D3C | 1.96766 | 0.00298305 | 6.55424 | 6.65829 | 7.59502 | 7.57048 |
| SH3BP2 | 1.53941 | 0.0219585 | 6.11778 | 5.95498 | 6.61219 | 6.70532 |
| SH3TC2 | −2.59589 | 0.0462089 | 6.92662 | 6.33749 | 5.17089 | 5.34076 |
| SHANK3 | −1.74174 | 0.00531482 | 8.02176 | 7.91032 | 7.14739 | 7.18364 |
| SIPA1L2 | −2.18596 | 0.0152291 | 6.72203 | 6.54828 | 5.61776 | 5.39603 |
| SIRPB1 | 1.71825 | 0.0182969 | 8.2868 | 8.27535 | 9.16897 | 8.95506 |
| SIRPB2 | −3.17242 | 0.00541968 | 6.96891 | 7.20849 | 5.45155 | 5.39468 |
| SLA | 4.70632 | 0.00046355 | 6.39034 | 6.45121 | 8.61809 | 8.69266 |
| SLC12A7 | 5.60081 | 0.00072376 | 4.94701 | 5.04951 | 7.44088 | 7.52691 |
| SLC18A2 | −1.58483 | 0.014895 | 10.5898 | 10.4802 | 9.80964 | 9.93165 |
| SLC22A4 | −1.6147 | 0.021269 | 8.22157 | 8.13097 | 7.39311 | 7.57691 |
| SLC25A23 | −1.47963 | 0.0198254 | 7.94097 | 7.77947 | 7.29762 | 7.29235 |
| SLC29A3 | 1.65444 | 0.0197729 | 7.89231 | 7.91758 | 8.7342 | 8.52839 |
| SLC2A3 | −2.74706 | 0.00189074 | 7.2523 | 7.14346 | 5.77267 | 5.7073 |
| SLC2A5 | −1.42815 | 0.00443299 | 7.16202 | 7.20242 | 6.69584 | 6.64029 |
| SLC31A2 | 1.63472 | 0.0328005 | 7.05516 | 7.00429 | 7.60957 | 7.86796 |
| SLC35D2 | −1.49152 | 0.0297688 | 6.89463 | 6.74087 | 6.17423 | 6.30769 |
| SLC36A1 | 1.41778 | 0.00549752 | 7.49466 | 7.42055 | 7.96696 | 7.95552 |
| SLC37A3 | −1.81757 | 0.0383161 | 7.66536 | 7.65301 | 6.62352 | 6.97083 |
| SLC39A11 | 1.40245 | 0.0115888 | 8.49634 | 8.39068 | 8.92735 | 8.93556 |
| SLC41A1 | −1.56541 | 0.00525033 | 8.76565 | 8.67175 | 8.06941 | 8.07491 |
| SLC43A3 | 1.70592 | 0.0123381 | 10.4035 | 10.2974 | 11.1892 | 11.0528 |
| SLC44A2 | −2.18682 | 0.0252705 | 8.44874 | 8.19508 | 7.06125 | 7.3249 |
| SLC48A1 | 1.67073 | 0.00233385 | 6.74241 | 6.72984 | 7.51188 | 7.44132 |
| SLC7A11 | −1.95395 | 0.00333316 | 6.89539 | 6.7879 | 5.89075 | 5.85975 |
| SLC9A7 | −1.91798 | 0.00630631 | 7.94482 | 7.93253 | 6.92437 | 7.0738 |
| SLCO3A1 | 1.48542 | 0.00109562 | 6.12239 | 6.12537 | 6.7136 | 6.6759 |
| SLCO4C1 | 1.55249 | 0.00310615 | 6.25938 | 6.27095 | 6.86477 | 6.93472 |
| SMAGP | −1.58494 | 0.0135723 | 7.15955 | 7.01672 | 6.39183 | 6.45558 |
| SNAP23 | −1.57976 | 0.0293823 | 10.1532 | 10.1578 | 9.3802 | 9.61145 |
| SNED1 | −1.98401 | 0.0142971 | 6.96728 | 6.74453 | 5.91072 | 5.82425 |
| SNORA62 | −1.47045 | 0.0389545 | 8.18127 | 8.29032 | 7.77865 | 7.58042 |
| SORT1 | −1.55294 | 0.00821496 | 10.4467 | 10.4576 | 9.75945 | 9.87476 |
| SP100 | 1.46436 | 0.00539879 | 7.13134 | 7.2094 | 7.7318 | 7.70948 |
| SPARC | −1.49017 | 0.00452232 | 11.2691 | 11.2003 | 10.6772 | 10.6412 |
| SRPX | −1.86251 | 0.00299052 | 9.36034 | 9.35893 | 8.41322 | 8.51156 |
| SSBP2 | −1.85194 | 0.0190344 | 9.38485 | 9.47354 | 8.65643 | 8.42388 |
| ST3GAL4 | 1.67265 | 0.0411673 | 6.17463 | 6.13939 | 6.74473 | 7.05356 |
| ST3GAL5 | −2.20173 | 0.003339 | 8.73156 | 8.74475 | 7.53389 | 7.66515 |
| ST3GAL6 | −1.51838 | 0.00655571 | 7.68722 | 7.69859 | 7.13907 | 7.04168 |
| STEAP3 | 1.40758 | 0.0246432 | 7.91297 | 7.82266 | 8.42572 | 8.29634 |
| STK10 | 1.70255 | 0.00329377 | 8.02229 | 8.01611 | 8.74284 | 8.83096 |
| STK17B | 2.17894 | 0.0034891 | 7.96802 | 7.8356 | 9.03213 | 9.01874 |
| STX3 | −1.4182 | 0.0165896 | 8.5653 | 8.43671 | 8.01066 | 7.98323 |
| STX7 | −1.57568 | 0.0186168 | 8.41503 | 8.39201 | 7.6575 | 7.83758 |
| SUSD1 | 1.59738 | 0.010961 | 7.00573 | 7.14053 | 7.72549 | 7.77219 |
| SV2A | −1.53105 | 0.0180398 | 9.72374 | 9.60443 | 9.10824 | 8.99089 |
| SYTL1 | 1.64932 | 0.00908906 | 6.44238 | 6.35736 | 7.17646 | 7.06702 |
| TARP | −1.42447 | 0.00743181 | 7.62945 | 7.71643 | 7.17068 | 7.15436 |
| TBC1D10C | 1.41643 | 0.0481788 | 7.12224 | 7.0123 | 7.46918 | 7.66988 |
| TBC1D19 | 1.57843 | 0.0142813 | 6.62056 | 6.60984 | 7.19432 | 7.35306 |
| TBC1D4 | −1.63444 | 0.0251372 | 9.04355 | 8.89524 | 8.34791 | 8.17329 |
| TCTEX1D1 | −1.75934 | 0.0101971 | 9.98158 | 9.82197 | 9.06415 | 9.10932 |
| TDRD7 | 1.41069 | 0.0196963 | 6.54397 | 6.61545 | 7.13713 | 7.0151 |
| TDRKH | −1.69119 | 0.00194939 | 6.8419 | 6.79191 | 6.03653 | 6.08119 |
| TEAD2 | −1.97289 | 0.0261732 | 7.08104 | 6.8481 | 5.87196 | 6.09656 |
| TESC | 4.08994 | 0.00183732 | 7.91689 | 7.88633 | 9.84781 | 10.0196 |
| TET1 | −2.00914 | 0.00108498 | 10.3032 | 10.2962 | 9.32613 | 9.26014 |
| TLE4 | 2.02418 | 0.00709843 | 8.37733 | 8.20551 | 9.30205 | 9.31548 |
| TM4SF1 | −5.64728 | 0.00284349 | 7.31757 | 7.11403 | 4.80459 | 4.63189 |
| TMEM150A | −1.45744 | 0.012521 | 7.333 | 7.38726 | 6.87177 | 6.76163 |
| TMEM173 | 2.27915 | 0.00315267 | 9.04411 | 9.07648 | 10.1839 | 10.3137 |
| TMEM53 | 1.41177 | 0.0358153 | 7.20728 | 7.3898 | 7.76386 | 7.82824 |
| TMEM87B | −1.66125 | 0.00621387 | 8.81806 | 8.76741 | 8.11264 | 8.0083 |
| TNFRSF10D | −1.86882 | 0.0188731 | 8.26295 | 8.01299 | 7.24949 | 7.22219 |
| TNFRSF21 | −1.76304 | 0.0269031 | 7.55475 | 7.281 | 6.59495 | 6.60466 |
| TNFSF10 | 1.82131 | 0.0136717 | 6.13842 | 5.96431 | 6.96986 | 6.86283 |
| TNFSF13B | −1.63759 | 0.00988704 | 10.5078 | 10.4918 | 9.71737 | 9.85904 |
| TOM1L1 | −1.92011 | 0.0292009 | 7.96445 | 7.70641 | 6.99622 | 6.79225 |
| TPSAB1 | 1.65718 | 0.0163079 | 6.7992 | 6.98256 | 7.5979 | 7.64133 |
| TRAF3IP3 | 2.72485 | 0.00054725 | 6.27628 | 6.32781 | 7.72628 | 7.77017 |
| TRGV3 | −1.69318 | 0.0109879 | 8.35491 | 8.41856 | 7.70072 | 7.55327 |
| TRH | −1.84356 | 0.0201983 | 8.23729 | 8.31584 | 7.27292 | 7.51522 |
| TRPC2 | 2.71476 | 0.00557693 | 6.3381 | 6.53021 | 7.92446 | 7.8255 |
| TSNAX | −1.46818 | 0.0221232 | 9.41086 | 9.26863 | 8.83005 | 8.74139 |
| TSPAN18 | −7.34871 | 0.00314444 | 9.26589 | 9.32416 | 6.57663 | 6.25844 |
| TSPAN32 | 1.84628 | 0.00014941 | 5.76603 | 5.78759 | 6.66061 | 6.66226 |
| TSPAN7 | −2.39258 | 0.00515445 | 11.3935 | 11.2464 | 10.1144 | 10.0084 |
| TTC28 | −1.41638 | 0.00014881 | 9.77322 | 9.77632 | 9.27849 | 9.26664 |
| TTC7B | −1.53173 | 0.0248505 | 8.92576 | 8.80842 | 8.33146 | 8.17241 |
| TUBB4 | −1.93865 | 0.0162217 | 7.68861 | 7.44453 | 6.62797 | 6.59507 |
| TYROBP | 1.68495 | 0.02683 | 6.97583 | 7.11169 | 7.90239 | 7.69054 |
| UNC84A | −1.86097 | 0.0408217 | 6.75483 | 6.56224 | 5.6024 | 5.92256 |
| USP28 | 1.56805 | 0.0244105 | 7.78649 | 7.68051 | 8.47114 | 8.2938 |
| USP53 | −1.61842 | 0.0289739 | 7.93207 | 7.71461 | 7.07595 | 7.18155 |
| UST | −1.94086 | 0.0289672 | 7.17255 | 7.03298 | 6.2972 | 5.99494 |
| VASH1 | −1.43496 | 0.015783 | 7.68094 | 7.71416 | 7.11242 | 7.24067 |
| VAV3 | −1.86056 | 0.00927838 | 10.2068 | 10.057 | 9.19211 | 9.28017 |
| VOPP1 | 1.6287 | 0.0150565 | 7.84845 | 7.68319 | 8.44125 | 8.49784 |
| VSIG4 | −3.86148 | 0.00423695 | 8.64016 | 8.56848 | 6.53304 | 6.77729 |
| WDFY3 | −1.47072 | 0.00363639 | 9.50066 | 9.44381 | 8.93373 | 8.89769 |
| WDFY4 | 2.40477 | 0.00411016 | 8.19521 | 8.17403 | 9.3698 | 9.53124 |
| XYLT1 | 3.144 | 0.0122212 | 7.44 | 7.12166 | 8.84036 | 9.02651 |
| ZBTB8B | −1.43004 | 0.00163407 | 7.15818 | 7.12088 | 6.63287 | 6.61407 |
| ZEB1 | −1.8639 | 0.00341994 | 6.6122 | 6.56999 | 5.74103 | 5.64451 |
| ZFP106 | −1.40715 | 0.0116856 | 8.20338 | 8.0965 | 7.65147 | 7.66286 |
| ZFP36L2 | 1.52448 | 0.0004219 | 11.2265 | 11.2015 | 11.8233 | 11.8213 |
| ZNF438 | −1.52332 | 0.0209287 | 6.69559 | 6.53234 | 5.97064 | 6.04285 |
| ZNF792 | −1.69795 | 0.0106 | 6.68574 | 6.54118 | 5.88222 | 5.81713 |

Tables 4A-4B: Genes showing inverse expression pattern in Kasumi1^{RX1-KD} cells (Table 4A) and Kasumi1^{A-E-KD} cells (Table 4B), both compared to Kasumi1^{l Cont} cells, as measured by expression arrays (listed are genes that showed fold-change of at least 1.4 and p-value<0.05).

TABLE 4A

| | Fold | | | | | |
|---|---|---|---|---|---|---|
| | Change | | | | | |
| | relative to | | | | | |
| Gene | non- | | Non- | Non- | RUNX1 | RUNX1 |
| Symbol | targeting | p-value | taregting_1 | taregting_2 | KD_1 | KD_2 |
| ADAMTS3 | 1.80447 | 0.00025414 | 7.5457 | 7.66217 | 8.45669 | 8.45433 |
| AIF1 | −1.50136 | 0.00178403 | 10.0478 | 10.0881 | 9.39191 | 9.57138 |
| ALDOC | 2.117 | 0.00182717 | 10.0601 | 10.0109 | 11.0603 | 11.1747 |
| ARHGEF3 | 2.08274 | 0.000045 | 7.5762 | 7.52328 | 8.64067 | 8.57577 |
| ATP2B4 | −1.42073 | 0.0006802 | 8.37919 | 8.26548 | 7.83943 | 7.79198 |
| BCL2 | −1.72227 | 0.00154257 | 7.71963 | 7.61849 | 6.97341 | 6.7961 |
| BRI3BP | −1.81913 | 8.29E−07 | 10.1605 | 10.2048 | 9.32354 | 9.31531 |
| C10orf114 | 1.7224 | 0.00170023 | 7.61435 | 7.69044 | 8.29985 | 8.57378 |
| C11orf17 | 1.47968 | 0.0009167 | 9.51608 | 9.59567 | 10.0999 | 10.1424 |
| C16orf54 | −1.47511 | 0.0104102 | 6.72924 | 6.51036 | 6.1588 | 5.95915 |
| C8orf73 | −1.63246 | 0.0105784 | 7.85852 | 7.8733 | 7.00302 | 7.3147 |
| CD244 | −1.41359 | 0.00114907 | 7.91903 | 7.97857 | 7.38197 | 7.51689 |
| CD33 | −2.65766 | 0.0000695 | 9.86459 | 9.8736 | 8.54315 | 8.37473 |
| CNKSR3 | 1.4015 | 0.00671442 | 6.55067 | 6.6916 | 7.09157 | 7.12463 |
| CST3 | −1.42551 | 0.0000354 | 9.7683 | 9.78097 | 9.25578 | 9.27054 |
| CYTL1 | 1.46757 | 0.0418745 | 7.25056 | 7.32615 | 7.94357 | 7.74 |
| CYTSB | −1.62794 | 0.00077137 | 6.80891 | 6.78963 | 6.0626 | 6.12984 |
| DPEP1 | 6.93097 | 0.00000942 | 5.92918 | 6.03924 | 8.70835 | 8.84618 |
| EDIL3 | 1.79102 | 0.00017759 | 9.7096 | 9.71488 | 10.6067 | 10.4994 |
| ENTPD1 | 1.68754 | 0.00158751 | 9.02469 | 8.91301 | 9.72263 | 9.72491 |
| ERG | 1.47994 | 0.00142434 | 9.53569 | 9.42513 | 9.97294 | 10.119 |
| ESAM | 1.44088 | 0.0346154 | 6.26189 | 5.93874 | 6.68321 | 6.57132 |
| FAM101B | −2.03632 | 7.63E−07 | 9.07205 | 9.04233 | 8.03283 | 8.02964 |
| FAM105A | 1.56419 | 0.00261185 | 9.82829 | 9.6652 | 10.3316 | 10.4527 |
| FLJ38379 | 1.80736 | 0.0103363 | 5.89958 | 5.99468 | 6.86929 | 6.73274 |
| FLNA | −1.45959 | 0.00056288 | 8.72303 | 8.72737 | 8.1908 | 8.16848 |
| FYB | −1.8869 | 0.00019855 | 7.17028 | 7.03615 | 6.14144 | 6.23296 |
| GALNT3 | −1.63857 | 0.00146529 | 6.99701 | 6.92973 | 6.26353 | 6.23832 |
| GCH1 | 1.5649 | 0.00117967 | 7.27854 | 7.35322 | 7.91618 | 8.00771 |
| GGTA1 | 2.33719 | 0.00028642 | 5.99986 | 5.79644 | 7.14014 | 7.1057 |
| GUCY1B3 | 1.89583 | 0.00098286 | 6.52225 | 6.40008 | 7.43194 | 7.33605 |
| HCST | −1.46775 | 0.00933771 | 6.41596 | 6.43636 | 5.94508 | 5.80002 |
| HIVEP3 | −1.51411 | 0.00159331 | 6.17837 | 6.1408 | 5.58288 | 5.53936 |
| HPSE | 1.64133 | 0.00015647 | 7.9396 | 7.86925 | 8.63504 | 8.60353 |
| IGFBP7 | −1.52179 | 0.00178795 | 11.034 | 11.0058 | 10.3728 | 10.4554 |
| IL1RAP | 1.40196 | 0.00027566 | 8.52319 | 8.52241 | 8.95298 | 9.06751 |
| IL8 | 1.48792 | 0.0219222 | 8.40636 | 8.30829 | 8.96781 | 8.89345 |
| ITGA9 | 1.51085 | 0.00371871 | 8.67461 | 8.74723 | 9.34832 | 9.26424 |
| JMJD1C | 1.4024 | 0.0000153 | 11.0243 | 11.0225 | 11.5179 | 11.5047 |
| KCNAB2 | −1.96282 | 0.00023314 | 9.80415 | 9.85277 | 8.95349 | 8.75757 |
| KIAA0182 | −1.97479 | 0.00789046 | 7.95768 | 7.84384 | 7.19014 | 6.64799 |
| KIAA1462 | 1.56214 | 0.00122691 | 8.13687 | 8.07859 | 8.67055 | 8.83195 |
| LAPTM5 | −1.72158 | 0.00023642 | 10.6506 | 10.5529 | 9.89009 | 9.74592 |
| LCP1 | −2.58614 | 0.0000569 | 10.3324 | 10.1786 | 8.93077 | 8.83867 |
| LGALS12 | −1.92575 | 0.00216789 | 10.0531 | 9.98228 | 8.9555 | 9.18901 |
| LOC284757 | −1.72907 | 0.00047036 | 6.79275 | 6.82709 | 6.039 | 6.00085 |
| LPAR6 | 2.88705 | 0.0001059 | 6.39219 | 6.3156 | 7.87803 | 7.88895 |
| LPXN | −1.87525 | 0.00022788 | 8.03042 | 8.18261 | 7.23029 | 7.16856 |
| LST1 | −1.56791 | 0.001024 | 8.43812 | 8.37188 | 7.6594 | 7.85292 |
| MAGED2 | 1.85262 | 0.00000969 | 8.03016 | 8.06373 | 8.92489 | 8.94814 |
| MMP28 | 2.38415 | 0.0001198 | 5.89656 | 5.70676 | 7.00222 | 7.10804 |
| MRC2 | −1.48323 | 0.00056963 | 7.8808 | 7.85396 | 7.23484 | 7.36243 |
| MYO1F | −1.63975 | 0.00103308 | 8.07989 | 8.0906 | 7.44746 | 7.29607 |
| NCKAP1 | 1.64177 | 0.00376162 | 7.86804 | 7.74819 | 8.44039 | 8.60634 |
| NIPAL2 | −1.51135 | 0.00090116 | 7.99539 | 7.8813 | 7.30864 | 7.37638 |
| PARVG | −1.49454 | 0.00028192 | 10.0466 | 10.0673 | 9.48921 | 9.46533 |
| PLCB2 | −1.72318 | 0.00027767 | 7.86602 | 7.90113 | 7.04632 | 7.15068 |
| PLP2 | −1.72774 | 0.00055895 | 9.55374 | 9.4804 | 8.71362 | 8.74275 |
| PLXNA4 | 1.80806 | 0.00054013 | 9.38622 | 9.39651 | 10.2235 | 10.2681 |
| PRKCH | 2.24422 | 0.0000411 | 8.69894 | 8.60848 | 9.78697 | 9.85287 |
| PTPN22 | −2.16729 | 0.00198768 | 6.72627 | 6.5463 | 5.59661 | 5.44417 |
| PTPN6 | −1.71555 | 0.0000954 | 8.00707 | 7.92738 | 7.21109 | 7.16601 |
| PTPRM | 1.65787 | 0.0000162 | 9.89288 | 9.94774 | 10.6695 | 10.6298 |
| PYCARD | −1.44728 | 0.00060886 | 9.08504 | 8.96803 | 8.46941 | 8.51696 |
| RAC2 | −1.5858 | 0.00345588 | 11.134 | 11.0058 | 10.4302 | 10.3792 |
| RASGRP2 | −1.75273 | 0.00209072 | 7.49731 | 7.42091 | 6.61169 | 6.68731 |
| RASSF2 | −1.76002 | 0.00020987 | 6.73841 | 6.58177 | 5.86255 | 5.82645 |
| RCBTB2 | 1.74279 | 0.00055819 | 7.95633 | 7.96919 | 8.76084 | 8.76748 |
| RNASE2 | −1.98712 | 0.00017118 | 11.3694 | 11.3634 | 10.3648 | 10.3865 |
| RNASE3 | −2.8382 | 0.00023613 | 8.43908 | 8.31515 | 7.01291 | 6.73137 |
| SAMSN1 | 1.80375 | 0.0116773 | 8.74761 | 8.64411 | 9.50965 | 9.58407 |
| SEMA4A | −2.51591 | 0.00027798 | 8.60918 | 8.44195 | 7.29496 | 7.09401 |
| SEMA4D | −1.68978 | 0.0000337 | 9.211 | 9.25961 | 8.46656 | 8.49038 |
| SERPINB9 | 2.74786 | 0.0000191 | 8.41058 | 8.32742 | 9.81552 | 9.8391 |
| SH3TC2 | 2.45085 | 0.00131364 | 6.92729 | 6.74935 | 7.9373 | 8.3259 |
| SIPA1L2 | 1.55855 | 0.00442378 | 6.88913 | 7.13629 | 7.71018 | 7.59565 |
| SIRPB1 | −1.92613 | 0.00034286 | 8.57607 | 8.41532 | 7.46807 | 7.63191 |
| SLC2A3 | 1.69356 | 0.00070408 | 7.81099 | 7.92069 | 8.58465 | 8.66714 |
| SLC35D2 | 1.46355 | 0.0241019 | 6.94297 | 6.76288 | 7.2466 | 7.55819 |
| SLC43A3 | −2.61219 | 0.00065411 | 10.543 | 10.348 | 8.94912 | 9.17138 |
| SLC44A2 | 1.88817 | 0.00035993 | 8.35746 | 8.30231 | 9.20945 | 9.28429 |
| SMAGP | 1.71317 | 0.00012282 | 7.46267 | 7.45851 | 8.29638 | 8.17815 |
| SPARC | 1.43409 | 0.0000954 | 11.3079 | 11.3134 | 11.7967 | 11.8649 |
| TM4SF1 | 6.73532 | 0.0000122 | 6.8766 | 7.06571 | 9.68307 | 9.76274 |
| TMEM173 | −1.8503 | 0.00017896 | 9.28623 | 9.21397 | 8.36416 | 8.36051 |
| TRAF3IP3 | −1.48756 | 0.00614293 | 6.93605 | 6.80699 | 6.3825 | 6.21465 |
| TRH | 2.21999 | 0.00044497 | 6.35226 | 6.5181 | 7.61809 | 7.55337 |
| TSPAN18 | 3.16877 | 0.00099272 | 8.89073 | 8.72307 | 10.2964 | 10.6452 |
| TSPAN7 | 1.47714 | 0.0001712 | 11.4816 | 11.423 | 11.9864 | 12.0438 |
| USP53 | 1.54323 | 0.00019289 | 7.85177 | 7.84769 | 8.42369 | 8.52768 |
| VAV3 | 1.65451 | 0.00017615 | 10.2206 | 10.2187 | 10.9233 | 10.9688 |
| WDFY4 | −1.53272 | 0.00147207 | 8.07067 | 8.05114 | 7.41719 | 7.47243 |
| ZBTB8B | 1.65062 | 0.0065805 | 6.46247 | 6.55366 | 7.41199 | 7.05015 |
| ZEB1 | 1.53945 | 0.0124442 | 8.58793 | 8.51194 | 9.26199 | 9.08271 |
| ZNF792 | 1.56199 | 0.00188229 | 6.12848 | 6.211 | 6.82599 | 6.80025 |

TABLE 4B

| | Fold | | | | | |
|---|---|---|---|---|---|---|
| | Change | | | | | |
| | relative to | | | | | |
| Gene | non- | | Non- | Non- | A-E | A-E |
| Symbol | targeting | p-value | taregting_1 | taregting_2 | KD_1 | KD_2 |
| ADAMTS3 | −2.50908 | 0.011999 | 8.08672 | 7.79702 | 6.59149 | 6.63794 |
| AIF1 | 1.57987 | 0.00703134 | 9.89453 | 9.99977 | 10.625 | 10.5889 |
| ALDOC | −1.79333 | 0.012676 | 8.66731 | 8.59236 | 7.87534 | 7.69904 |
| ARHGEF3 | −1.84677 | 0.00836351 | 8.06732 | 7.90653 | 7.11499 | 7.08885 |
| ATP2B4 | 3.09853 | 0.00029622 | 8.65744 | 8.60134 | 10.2625 | 10.2595 |
| BCL2 | 2.34125 | 0.00274287 | 7.71712 | 7.71419 | 8.87854 | 9.00733 |
| BRI3BP | 1.42706 | 0.0455507 | 10.3018 | 10.2928 | 10.9237 | 10.697 |
| C10orf114 | −2.18976 | 0.00266178 | 6.97244 | 6.8799 | 5.83112 | 5.75967 |
| C11orf17 | −1.53165 | 0.0260087 | 8.68148 | 8.49488 | 7.93394 | 8.01224 |
| C16orf54 | 1.58535 | 0.00431697 | 6.67364 | 6.75577 | 7.36421 | 7.3948 |
| C8orf73 | 1.79625 | 0.00739747 | 7.88711 | 7.8175 | 8.63303 | 8.76156 |
| CD244 | 1.58494 | 0.0133803 | 7.65369 | 7.75425 | 8.30924 | 8.42756 |
| CD33 | 2.8113 | 0.00209305 | 9.53172 | 9.45385 | 10.9279 | 11.0402 |
| CNKSR3 | −1.47617 | 0.048038 | 6.6272 | 6.51046 | 5.89328 | 6.12066 |
| CST3 | 1.44168 | 0.0472593 | 9.69506 | 9.63635 | 10.0781 | 10.3088 |
| CYTL1 | −1.63553 | 0.0143672 | 7.62044 | 7.54185 | 6.94789 | 6.79489 |
| CYTSB | 1.51526 | 0.0258878 | 7.13296 | 7.06292 | 7.60556 | 7.78945 |
| DPEP1 | −2.09025 | 0.0357737 | 7.16869 | 6.76687 | 5.85521 | 5.95299 |
| EDIL3 | −7.595 | 0.0012061 | 8.12011 | 8.06663 | 5.07023 | 5.26642 |
| ENTPD1 | −3.1966 | 0.0142729 | 8.0885 | 7.81981 | 6.12615 | 6.42909 |
| ERG | −1.51218 | 0.00023528 | 9.16914 | 9.17677 | 8.58465 | 8.56801 |
| ESAM | −2.13135 | 0.0346365 | 6.51673 | 6.54453 | 5.64705 | 5.23068 |
| FAM101B | 2.48533 | 0.00107311 | 8.90976 | 8.89603 | 10.1738 | 10.2588 |
| FAM105A | −1.67693 | 0.0109472 | 9.57808 | 9.43157 | 8.78772 | 8.73028 |
| FLJ38379 | −1.63497 | 0.0295302 | 7.19393 | 6.97216 | 6.31682 | 6.43075 |
| FLNA | 1.69643 | 0.0256105 | 8.53491 | 8.41941 | 9.12944 | 9.34987 |
| FYB | 1.77638 | 0.0156958 | 6.25258 | 6.04273 | 6.98258 | 6.9706 |
| GALNT3 | 1.648 | 0.00959683 | 6.76957 | 6.6784 | 7.49929 | 7.39012 |
| GCH1 | −1.71904 | 0.00285163 | 6.9493 | 6.8673 | 6.11842 | 6.13497 |
| GGTA1 | −1.83601 | 0.0038889 | 6.80416 | 6.75766 | 5.85469 | 5.95399 |
| GUCY1B3 | −2.00133 | 0.00727896 | 7.03336 | 6.90498 | 6.02525 | 5.91117 |
| HCST | 4.12679 | 0.0071136 | 7.01833 | 7.14161 | 8.9629 | 9.28707 |
| HIVEP3 | 1.55497 | 0.0426467 | 6.62492 | 6.4519 | 7.28012 | 7.07048 |
| HPSE | −2.99899 | 0.00216524 | 7.77004 | 7.68895 | 6.20675 | 6.08329 |
| IGFBP7 | 2.12108 | 0.00321563 | 11.3499 | 11.3235 | 12.3612 | 12.4817 |
| IL1RAP | −1.42113 | 0.00044592 | 7.429 | 7.43635 | 6.93569 | 6.91557 |
| IL8 | −3.45852 | 0.00171094 | 7.65107 | 7.53008 | 5.75756 | 5.84328 |
| ITGA9 | −3.30571 | 0.0100109 | 8.14741 | 7.82289 | 6.19765 | 6.32273 |
| JMJD1C | −1.49937 | 0.0189358 | 10.5865 | 10.5386 | 9.90019 | 10.0561 |
| KCNAB2 | 1.88467 | 0.00949454 | 9.16635 | 9.14873 | 9.98255 | 10.1611 |
| KIAA0182 | 2.46251 | 0.00330428 | 7.83712 | 7.9815 | 9.22949 | 9.1894 |
| KIAA1462 | −2.33329 | 0.00852749 | 8.14183 | 7.98281 | 6.75881 | 6.9211 |
| LAPTM5 | 2.45311 | 0.00085 | 10.2507 | 10.2159 | 11.5614 | 11.4944 |
| LCP1 | 5.87542 | 0.00027953 | 8.61241 | 8.56489 | 11.1078 | 11.1788 |
| LGALS12 | 1.4229 | 0.00984938 | 10.0132 | 9.95298 | 10.5329 | 10.4509 |
| LOC284757 | 1.69515 | 0.00593536 | 6.57326 | 6.5467 | 7.3788 | 7.26399 |
| LPAR6 | −2.84085 | 0.00401906 | 6.93901 | 6.74921 | 5.32482 | 5.35076 |
| LPXN | 1.46785 | 0.0158884 | 8.64144 | 8.69825 | 9.28823 | 9.15888 |
| LST1 | 2.39606 | 0.0000317 | 8.38062 | 8.38604 | 9.65055 | 9.63743 |
| MAGED2 | −1.58979 | 0.0205719 | 8.53729 | 8.35312 | 7.7445 | 7.80823 |
| MMP28 | −1.6536 | 0.0224007 | 7.07334 | 7.03207 | 6.21857 | 6.43562 |
| MRC2 | 1.40018 | 0.017738 | 7.50325 | 7.50424 | 7.92381 | 8.05491 |
| MYO1F | 2.14191 | 0.0120161 | 7.86721 | 7.84255 | 8.83285 | 9.07471 |
| NCKAP1 | −2.84747 | 0.010182 | 6.60093 | 6.39155 | 4.87429 | 5.09883 |
| NIPAL2 | 1.89383 | 0.00760288 | 8.08426 | 8.01033 | 9.04045 | 8.89676 |
| PARVG | 2.15136 | 0.00096149 | 9.76366 | 9.73782 | 10.8242 | 10.8878 |
| PLCB2 | 1.61902 | 0.00054942 | 7.58774 | 7.56641 | 8.25987 | 8.28453 |
| PLP2 | 2.23552 | 0.00265799 | 9.34734 | 9.43831 | 10.5925 | 10.5144 |
| PLXNA4 | −6.90498 | 0.00090276 | 8.73364 | 8.71006 | 6.01719 | 5.85123 |
| PRKCH | −1.52651 | 0.0000454 | 9.47366 | 9.46575 | 8.86059 | 8.85834 |
| PTPN22 | 2.35751 | 0.0194793 | 5.91057 | 5.6322 | 7.11515 | 6.90215 |
| PTPN6 | 2.03521 | 0.0126452 | 7.54888 | 7.38179 | 8.57156 | 8.40948 |
| PTPRM | −1.68158 | 0.000782 | 9.32578 | 9.30165 | 8.58106 | 8.54673 |
| PYCARD | 1.44215 | 0.0496332 | 9.33884 | 9.30004 | 9.9684 | 9.72693 |
| RAC2 | 1.63112 | 0.00065174 | 10.6496 | 10.6813 | 11.3799 | 11.3627 |
| RASGRP2 | 2.73832 | 0.00185262 | 7.75523 | 7.85385 | 9.29646 | 9.2192 |
| RASSF2 | 8.33815 | 0.00061574 | 6.0415 | 5.89337 | 9.04403 | 9.0103 |
| RCBTB2 | −2.0888 | 0.0386819 | 7.59385 | 7.375 | 6.23633 | 6.60717 |
| RNASE2 | 2.23271 | 0.0127308 | 11.0353 | 10.7718 | 12.0538 | 12.0708 |
| RNASE3 | 3.38734 | 0.00161517 | 7.80602 | 7.67933 | 9.47115 | 9.53451 |
| SAMSN1 | −1.95929 | 0.0153479 | 7.59314 | 7.6698 | 6.54572 | 6.77656 |
| SEMA4A | 1.40453 | 0.0312877 | 8.24212 | 8.31571 | 8.6882 | 8.8498 |
| SEMA4D | 1.5826 | 0.0224418 | 5.71048 | 5.90107 | 6.50129 | 6.43485 |
| SERPINB9 | −7.51782 | 0.00708342 | 8.46638 | 8.43422 | 5.29426 | 5.78571 |
| SH3TC2 | −2.59589 | 0.0462089 | 6.92662 | 6.33749 | 5.17089 | 5.34076 |
| SIPA1L2 | −2.18596 | 0.0152291 | 6.72203 | 6.54828 | 5.61776 | 5.39603 |
| SIRPB1 | 1.71825 | 0.0182969 | 8.2868 | 8.27535 | 9.16897 | 8.95506 |
| SLC2A3 | −2.74706 | 0.00189074 | 7.2523 | 7.14346 | 5.77267 | 5.7073 |
| SLC35D2 | −1.49152 | 0.0297688 | 6.89463 | 6.74087 | 6.17423 | 6.30769 |
| SLC43A3 | 1.70592 | 0.0123381 | 10.4035 | 10.2974 | 11.1892 | 11.0528 |
| SLC44A2 | −2.18682 | 0.0252705 | 8.44874 | 8.19508 | 7.06125 | 7.3249 |
| SMAGP | −1.58494 | 0.0135723 | 7.15955 | 7.01672 | 6.39183 | 6.45558 |
| SPARC | −1.49017 | 0.00452232 | 11.2691 | 11.2003 | 10.6772 | 10.6412 |
| TM4SF1 | −5.64728 | 0.00284349 | 7.31757 | 7.11403 | 4.80459 | 4.63189 |
| TMEM173 | 2.27915 | 0.00315267 | 9.04411 | 9.07648 | 10.1839 | 10.3137 |
| TRAF3IP3 | 2.72485 | 0.00054725 | 6.27628 | 6.32781 | 7.72628 | 7.77017 |
| TRH | −1.84356 | 0.0201983 | 8.23729 | 8.31584 | 7.27292 | 7.51522 |
| TSPAN18 | −7.34871 | 0.00314444 | 9.26589 | 9.32416 | 6.57663 | 6.25844 |
| TSPAN7 | −2.39258 | 0.00515445 | 11.3935 | 11.2464 | 10.1144 | 10.0084 |
| USP53 | −1.61842 | 0.0289739 | 7.93207 | 7.71461 | 7.07595 | 7.18155 |
| VAV3 | −1.86056 | 0.00927838 | 10.2068 | 10.057 | 9.19211 | 9.28017 |
| WDFY4 | 2.40477 | 0.00411016 | 8.19521 | 8.17403 | 9.3698 | 9.53124 |
| ZBTB8B | −1.43004 | 0.00163407 | 7.15818 | 7.12088 | 6.63287 | 6.61407 |
| ZEB1 | −1.8639 | 0.00341994 | 6.6122 | 6.56999 | 5.74103 | 5.64451 |
| ZNF792 | −1.69795 | 0.0106 | 6.68574 | 6.54118 | 5.88222 | 5.81713 |

TABLE 5
Genes showing inverse expression pattern in Kasumi-1^{RX1-KD} and
Kasumi-1^{A-E-KD} cells (listed in Tables 4A-4B, above) are functionally
enriched for cell death and apoptosis as revealed by IPA analysis.

| | | Predicted | | | |
|---|---|---|---|---|---|
| Functional | | Activation | Activation | | # Mole- |
| Annotation | p-Value | State | z-score | Molecules | cules |
| cell death | 6.69E−07 | Increased | 3.176 | AIF1, ALDOC, ANPEP, | 53 |
| | | | | ATP2B4, BCL2, BCL6, | |
| | | | | BMP4, BPI, CD244, | |
| | | | | CD33, CD69, CST3, | |
| | | | | EDIL3, ENTPD1, ERG, | |
| | | | | FLNA, GALNT3, GPR65, | |
| | | | | GUCY1B3, HCST, HPSE, | |
| | | | | ICAM3, IGFBP7, IL8, | |
| | | | | IRF1, ITGA6, LGALS12, | |
| | | | | LPAR1, NCKAP1, PLAC8, | |
| | | | | PRKCH, PTGS2, PTPN22, | |
| | | | | PTPN6, PYCARD, RAC2, | |
| | | | | RASGRP2, RASSF2, RNASE2, | |
| | | | | RNASE3, SELL, SEMA4A, | |
| | | | | SEMA4D, SERPINB9, SLC2A3, | |
| | | | | SLC7A11, SLIT2, SPARC, | |
| | | | | TMEM173, TPSAB1/TPSB2, | |
| | | | | TRH, VAV3, ZEB1 | |
| apoptosis | 1.37E−05 | Increased | 2.857 | AIF1, ALDOC, ANPEP, | 43 |
| | | | | ATP2B4, BCL2, BCL6, | |
| | | | | BMP4, BPI, CD33, | |
| | | | | CD69, CST3, EDIL3, | |
| | | | | ENTPD1, ERG, GALNT3, | |
| | | | | GPR65, HPSE, ICAM3, | |
| | | | | IGFBP7, IL8, IRF1, | |
| | | | | ITGA6, LGALS12, LPAR1, | |
| | | | | NCKAP1, PLAC8, PRKCH, | |
| | | | | PTGS2, PTPN22, PTPN6, | |
| | | | | PYCARD, RAC2, RASSF2, | |
| | | | | SELL, SEMA4A, SERPINB9, | |
| | | | | SLC2A3, SLIT2, SPARC, | |
| | | | | TPSAB1/TPSB2, TRH, | |
| | | | | VAV3, ZEB1 | |
| necrosis | 5.11E−05 | Increased | 3.308 | ANPEP, ATP2B4, BCL2, | 40 |
| | | | | BCL6, BMP4, BPI, | |
| | | | | CD33, CD69, CST3, | |
| | | | | EDIL3, ERG, FLNA, | |
| | | | | GALNT3, GPR65, GUCY1B3, | |
| | | | | IGFBP7, IL8, IRF1, | |
| | | | | ITGA6, LGALS12, LPAR1, | |
| | | | | PLAC8, PRKCH, PTGS2, | |
| | | | | PTPN22, PTPN6, PYCARD, | |
| | | | | RAC2, RNASE2, RNASE3, | |
| | | | | SELL, SEMA4D, SERPINB9, | |
| | | | | SLC7A11, SPARC, TMEM173, | |
| | | | | TPSAB1/TPSB2, TRH, VAV3, | |
| | | | | ZEB1 | |
| proliferation of | 4.61E−03 | Decreased | −2.213 | BCL2, BCL6, BMP4, | 22 |
| tumor cell lines | | | | ENTPD1, ERG, FLNA, | |
| | | | | HPSE, IGFBP7, IL8, | |
| | | | | IRF1, ITGA6, LCP1, | |
| | | | | PLXNA4, PRKCH, PTGS2, | |
| | | | | PTPN22, PTPN6, SEMA4D, | |
| | | | | SLC7A11, SPARC, TRH, | |
| | | | | ZEB1 | |
| proliferation of | 4.61E−03 | Decreased | −2.213 | BCL2, BCL6, BMP4, | 22 |
| tumor cell lines | | | | ENTPD1, ERG, FLNA, | |
| | | | | HPSE, IGFBP7, IL8, | |
| | | | | IRF1, ITGA6, LCP1, | |
| | | | | PLXNA4, PRKCH, PTGS2, | |
| | | | | PTPN22, PTPN6, SEMA4D, | |
| | | | | SLC7A11, SPARC, TRH, | |
| | | | | ZEB1 | |
| homing of cells | 1.87E−08 | Increased | 2.145 | AIF1, BCL2, BMP4, | 20 |
| | | | | C3AR1, CD69, CST3, | |
| | | | | FYB, IL8, ITGA6, | |
| | | | | LCP1, MYO1F, PTGS2, | |
| | | | | PTPN6, RAC2, RNASE2, | |
| | | | | RNASE3, SELL, SELPLG, | |
| | | | | SEMA4D, SLIT2 | |
| quantity of | 1.23E−04 | Increased | 2.413 | C3AR1, GALNT3, HPSE, | 14 |
| protein in blood | | | | IRF1, LGALS12, MTSS1, | |
| | | | | NPR3, PTGS2, PYCARD, | |
| | | | | SAMSN1, SELL,TMEM173, | |
| | | | | TRH, VAV3 | |
| migration of | 2.77E−05 | Decreased | −2.778 | BMP4, EDIL3, ERG, | 11 |
| endothelial cells | | | | ESAM, FLNA, HPSE, | |
| | | | | IL8, ITGA9, PTGS2, | |
| | | | | SLIT2, VAV3 | |
| migration of | 2.77E−05 | Decreased | −2.778 | BMP4, EDIL3, ERG, | 11 |
| endothelial cells | | | | ESAM, FLNA, HPSE, | |
| | | | | IL8, ITGA9, PTGS2, | |
| | | | | SLIT2, VAV3 | |
| hypersensitive | 1.20E−03 | Increased | 2.236 | BCL2, C3AR1, CST3, | 11 |
| reaction | | | | FLNA, IL8, ITGA6, | |
| | | | | LAPTM5, PTGS2, PYCARD, | |
| | | | | SELL, SEMA4A | |
| quantity of | 8.10E−04 | Decreased | −2.376 | ATP2B4, ENTPD1, GUCY1B3, | 7 |
| cyclic | | | | IL8, LGALS12, PTGS2, TRH | |
| nucleotides | | | | | |
| quantity of | 8.10E−04 | Decreased | −2.376 | ATP2B4, ENTPD1, GUCY1B3, | 7 |
| cyclic | | | | IL8, LGALS12, PTGS2, TRH | |
| nucleotides | | | | | |
| quantity of | 8.10E−04 | Decreased | −2.376 | ATP2B4, ENTPD1, GUCY1B3, | 7 |
| cyclic | | | | IL8, LGALS12, PTGS2, TRH | |
| nucleotides | | | | | |
| quantity of | 4.85E−04 | Increased | 2.219 | BCL2, CD244, HCST, | 5 |
| natural killer | | | | IRF1, PYCARD | |
| cells | | | | | |
| quantity of | 4.85E−04 | Increased | 2.219 | BCL2, CD244, HCST, | 5 |
| natural killer | | | | IRF1, PYCARD | |
| cells | | | | | |
| delayed | 2.44E−03 | Increased | 2 | BCL2, LAPTM5, PYCARD, | 5 |
| hypersensitive | | | | SELL ,SEMA4A | |
| reaction | | | | | |
| movement of | 3.95E−03 | Decreased | −2.161 | IL8, ITGA9, PTGS2, | 5 |
| vascular | | | | SLIT2, VAV3 | |
| endothelial cells | | | | | |
| movement of | 3.95E−03 | Decreased | −2.161 | IL8, ITGA9, PTGS2, | 5 |
| vascular | | | | SLIT2, VAV3 | |
| endothelial cells | | | | | |
| proliferation of | 4.76E−03 | Decreased | −2.219 | BCL6, IGFBP7, IRF1, | 5 |
| lymphoma cell | | | | ITGA6, PTPN6 | |
| lines | | | | | |
| proliferation of | 4.76E−03 | Decreased | −2.219 | BCL6, IGFBP7, IRF1, | 5 |
| lymphoma cell | | | | ITGA6, PTPN6 | |
| lines | | | | | |

### Example 4

### RUNX1- and A-E Genomic-Occupancy Patterns

To selectively map the genomic occupancy of either RUNX1 or A-E, ChIP-seq using anti-RUNX1 C-terminus or anti-ETO specific antibodies (FIG. 3C) was conducted. Data analysis revealed 14,247 RUNX1-bound genomic regions and a comparable number (13,070) of A-E bound regions. As could have been predicted from their common DNA binding RD, genomic occupancy of RUNX1 and A-E was highly correlated (FIGS. 3D and 3E). Despite this strong quantitative correlation, the present inventors also noted a spectrum of differential A-E/RUNX1 binding (FIG. 3E), suggesting variable binding affinities of the two TFs at loci with different genomic contexts.

To study the impact of binding patterns on the transcriptional response to KD of either RUNX1 or A-E, the ChIP-Seq and gene expression datasets were integrated. Significant numbers of genes proximal to RUNX1/A-E shared regions were downregulated following RUNX1 KD and upregulated in response to A-E KD (FIGS. 3F and 3G), supporting the notion that direct competition between the two TFs is the underlying mechanism driving the leukemogenic transcriptional program. Specifically, the inherent similarities in binding preferences of RUNX1 and A-E resulted in an opposing regulatory response that explained the different cellular phenotypes resulting from KD of either RUNX1 or A-E. However, the differential A-E/RUNX1 binding (FIG. 3E) also manifested in inverse regulation of their uniquely occupied genes (FIGS. 3F and 3G). This observation suggests that the two TFs might also compete indirectly, due to distinct sequence affinities and/or interaction with cooperating TFs.

### Example 5

### Comparative Sequence Analysis of Uniquely Occupied RUNX1/A-E Genomic Regions

The opposing transcriptional response of RUNX1 and A-E shared and unique target genes prompted the inventors to further characterize the properties of A-E- and/or RUNX1-bound regions. In comparison to uniquely A-E bound regions, a significantly higher proportion of RUNX1-unique peaks were localized at the vicinity of transcription start site (TSS) (FIG. 4A), suggesting that RUNX1 has an advantage over A-E in binding to promoter regions in Kasumi-1 cells. Sequence analysis of genomic regions uniquely bound by either A-E or RUNX1 revealed significantly lower frequency of the canonical RUNX motif within A-E-bound regions (FIG. 4B, left). On the other hand, these A-E-occupied regions exhibited higher frequency of a variant RUNX motif, compared to the uniquely bound RUNX1 peaks (FIG. 4B, right). Interestingly, the ratio between the two motifs quantitatively predicted the ratio between RUNX1 and A-E ChIP-seq enrichment (FIG. 4C, p<2.2e⁻¹⁶). The enrichment of promoter occupancy by RUNX1 and differential affinities of A-E and RUNX1 to the variant and canonical RUNX motifs suggest that subtle sequence preferences contributed to differential binding and consequent biological activity of the two TFs.

Further sequence analysis of RUNX1- and A-E-occupied regions revealed that while both bound regions were enriched for the ETS TF motif (FIG. 4D, upper), only A-E unique regions were specifically enriched for the palindromic motif CAGCTG, bound by the E-Box TF AP4 (FIG. 4D, lower). This latter observation is consistent with previous studies [Gardini, A. et al., PLoS (2008) Genet 4] indicating that A-E interactions with E-Box proteins facilitate its binding to the DNA and with more recent finding of enrichment in E-Box-binding proteins among A-E-unique peaks [Ptasinska et al. (2012), supra]. Given that AP4 is highly expressed in Kasumi-1 cells (FIG. 4E), a AP4 ChIP-seq was performed and a comparison was made to the distribution of AP4 binding sites to A-E and RUNX1 occupancy profiles. Although significant numbers of ChIP-seq peaks were common to the three TFs there was no preference for A-E/AP4 co-occupancy compared to that of RUNX1/AP4 (FIG. 4F). This finding would argue that AP4 is unlikely the only E-Box TF that preferentially interacts with A-E. Nevertheless, the possibility that A-E and AP4 regulate a common subset of genes in Kasumi-1 cells cannot be ruled out, potentially through protein-protein interaction as recently reported for AP4 and RUNX1 [Egawa, T. and Littman D R, Proc Natl Acad Sci USA (2011) 108, 14873-14878]. The ChIP-seq sequence analysis possibly explains the mechanism underlying the opposing regulatory effects of RUNX1 and A-E, suggesting that sequence context and protein-protein interactions play role in their overall impact on the cell-transcriptional program.

### Example 6

### Gene-Expression Analysis of Apoptosis-Inhibited Kasumi-1^{RX1-KD} Cells Revealed Altered Expression of Critical Mitotic Progression Genes

Because RUNX1 KD in Kasumi-1 cells triggered extensive caspase-dependent apoptosis (FIGS. 1A-1L), the present inventors sought to identify the molecular pathways involved in this process. Differential gene expression was measured in Z-VAD-FMK-treated Kasumi-1^{RX1-KD} cells (Kasumi-1^{RX1-KD+Z}) compared to Z-VAD-FMK-treated control cells (Kasumi-1^{Cont+Z}) (see FIGS. 1H and 1I).

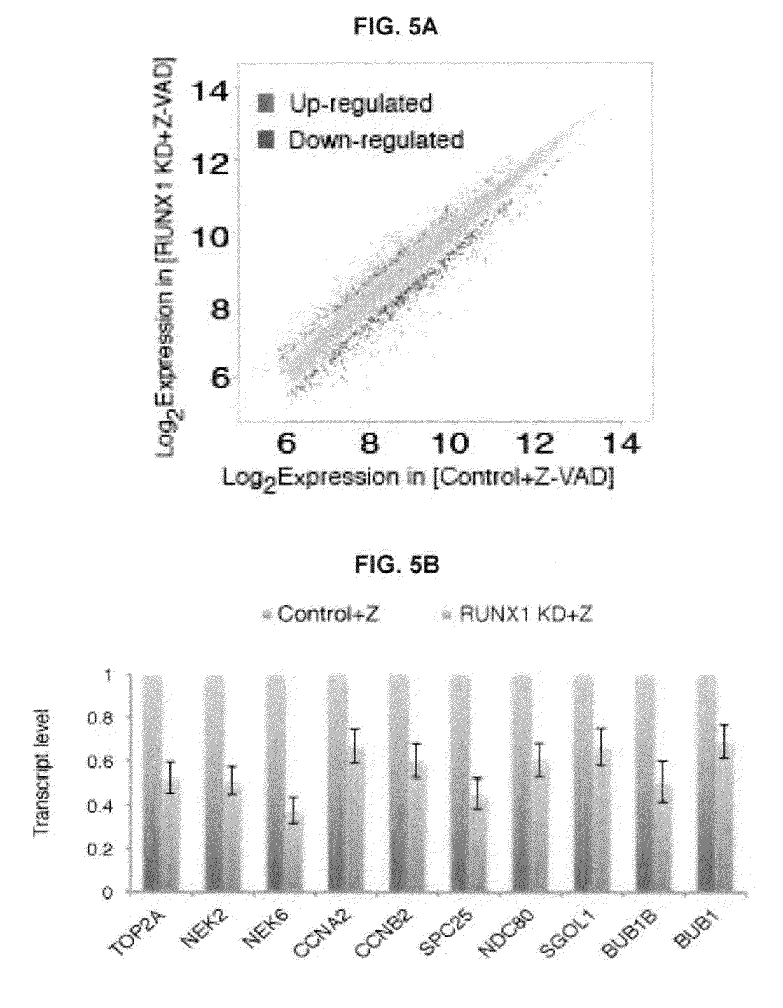

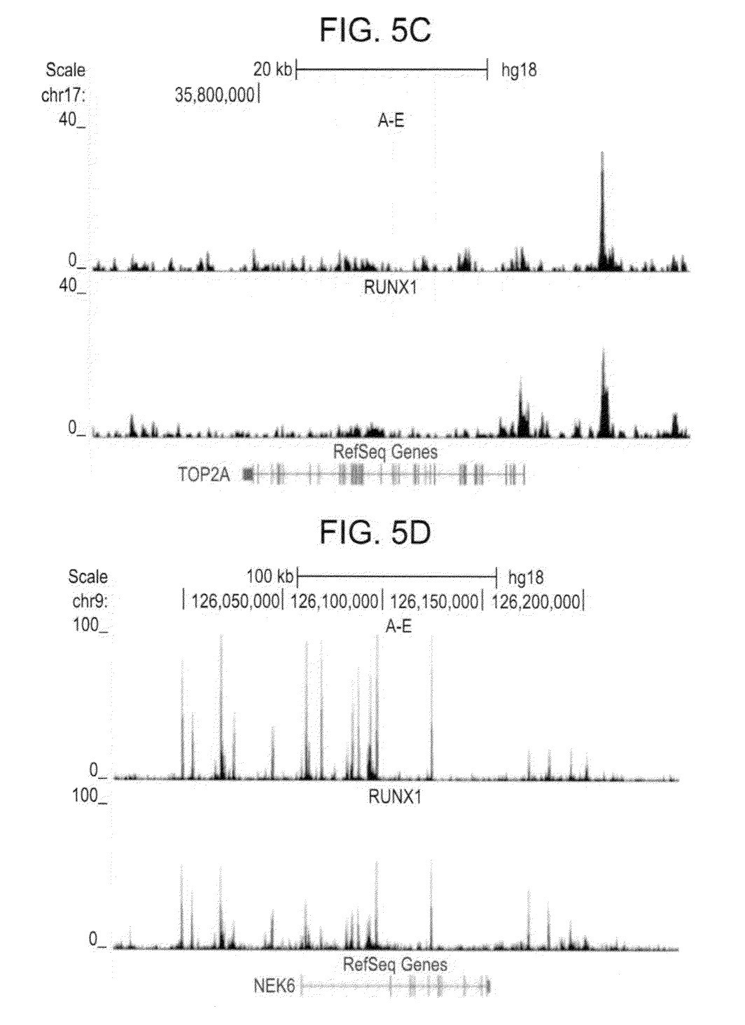

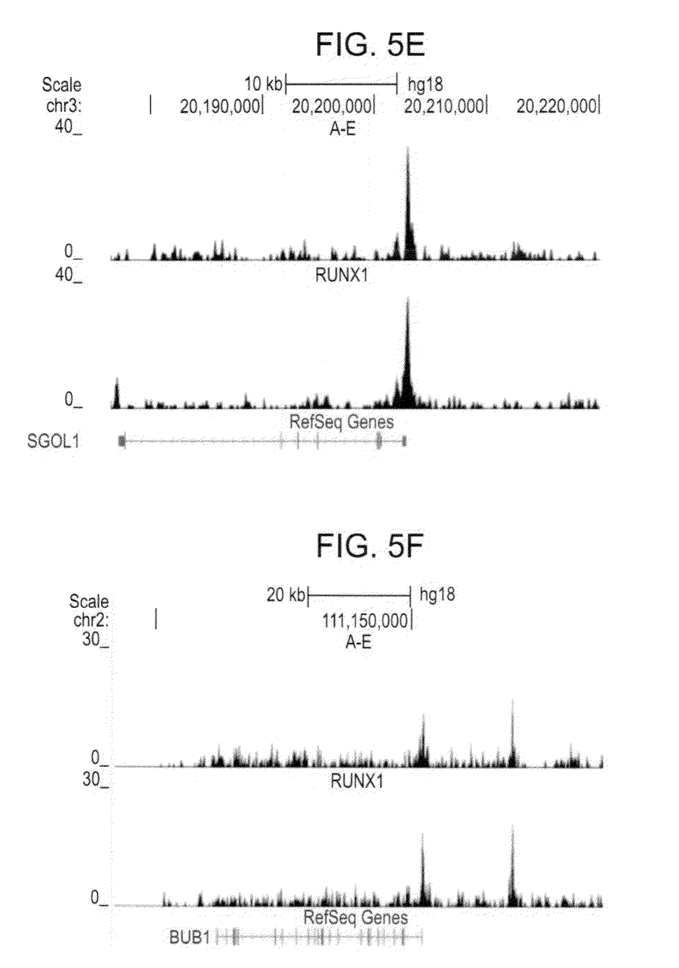

Gene-expression analysis revealed that 920 genes were differentially expressed in Kasumi-1^{KX1-KD+Z} compared to Kasumi-1^{Cont+Z} cells (FIG. 5A and Table 6, hereinbelow). Out of these RUNX1-responsive genes, 485 and 435 genes were respectively up- or downregulated. Functional annotation analysis indicated that Kasumi-1^{RUNX1-KD+Z} differentially expressed genes were highly enriched for genes with critical functions in mitosis (Table 7, hereinbelow). This unique RUNX1-responsive mitotic subset included genes involved in regulation of the mitotic checkpoint, also known as the spindle-assembly checkpoint (SAC) [Lam-Gonzalez P. et al., Curr Biol (2012) 22, R966-980]. Expression of several key mitotic- and SAC-genes downregulated in Kasumi-1^{KX1-KD+Z} was validated by RT-qPCR (FIG. 5B). Interestingly, among these responsive genes, the genomic loci of TOP2A, NEK6, SGOL1 and BUB1 exhibited similar ChIP-Seq occupancy of RUNX1 and A-E (FIGS. 5C-5F). Collectively, the data is compatible with the possibility that RUNX1 positively regulates these mitotic-critical genes, but its KD in Kasumi-1^{RX1-KD} cells enables A-E to bind and repress their expression resulting in mitotic impairment and apoptosis.

TABLE 6
Genes showing differential expression in Kasumi-1^{RX1-KD+Z} compared to
Kasumi-1^{Cont+Z}, as measured by expression arrays (listed are genes that showed
fold-change of at least 1.4 and p-value <0.05)

| | Fold Change | | | | | RUNX1 |
|---|---|---|---|---|---|---|
| | relative to | | Non- | Non- | RUNX1 | KD + |
| | Non- | | taregting + Z- | taregting + Z- | KD + Z- | Z- |
| Gene | targeting + Z- | | VAD- | VAD- | VAD- | VAD- |
| Symbol | VAD-FMK | p-value | FMK_1 | FMK_2 | FMK_1 | FMK_2 |
| ABCA3 | 1.68838 | 0.00642162 | 7.60624 | 7.66603 | 8.44477 | 8.33878 |
| ABCB1 | 1.43113 | 0.00759986 | 9.39382 | 9.36446 | 9.85339 | 9.93919 |
| ABCC5 | 1.46338 | 0.0132069 | 8.10178 | 7.97857 | 8.60592 | 8.57304 |
| ABHD10 | −1.48045 | 0.0321601 | 8.28813 | 8.47844 | 7.7752 | 7.8593 |
| ABHD11 | −1.43827 | 0.0308679 | 7.77597 | 7.91621 | 7.25867 | 7.38483 |
| ACACB | −1.8964 | 0.00605261 | 7.83346 | 7.96296 | 6.9431 | 7.00679 |
| ACVR1 | 1.74864 | 0.0081516 | 9.92527 | 9.78395 | 10.6801 | 10.6416 |
| ACVR1B | 1.94453 | 0.00452033 | 8.00496 | 8.08232 | 8.95116 | 9.05495 |
| ACVR1C | 1.48818 | 0.0259556 | 6.67803 | 6.68764 | 7.35051 | 7.16226 |
| ADAM9 | 1.43626 | 0.0240265 | 11.458 | 11.3521 | 11.9906 | 11.8642 |
| ADCY9 | −1.61738 | 0.0385962 | 7.18099 | 7.45069 | 6.66117 | 6.58319 |
| ADRB2 | −1.45277 | 0.0431812 | 7.47429 | 7.48055 | 6.82292 | 7.05432 |
| ADRBK1 | −1.80246 | 0.0209537 | 9.06982 | 9.29013 | 8.27091 | 8.38911 |
| ADRBK2 | −1.78514 | 0.00491627 | 9.56011 | 9.66911 | 8.80074 | 8.7564 |
| AGPAT4 | 2.00077 | 0.00763304 | 6.04056 | 6.09518 | 6.98486 | 7.152 |
| AHI1 | 1.71592 | 0.0136172 | 8.17738 | 8.05515 | 8.96381 | 8.82669 |
| AKR1CL1 | 2.36169 | 0.00025325 | 6.66337 | 6.65481 | 7.91817 | 7.87964 |
| ALOX5AP | −1.59121 | 0.0303644 | 6.4035 | 6.59635 | 5.7592 | 5.90041 |
| AMN1 | 1.4661 | 0.0401368 | 8.77376 | 8.5609 | 9.26029 | 9.17833 |
| AMOTL1 | 1.40995 | 0.0292506 | 8.34749 | 8.47392 | 8.96566 | 8.84704 |
| ANK2 | 1.66713 | 0.00316409 | 6.27076 | 6.2252 | 6.95057 | 7.02012 |
| ANKDD1A | 1.69711 | 0.0129572 | 7.23469 | 7.07369 | 7.9521 | 7.88242 |
| ANKRD10 | 1.59855 | 0.0099722 | 11.0762 | 10.9554 | 11.6611 | 11.7241 |
| ANKRD22 | −2.78863 | 0.0247621 | 7.80631 | 8.27992 | 6.57806 | 6.54906 |
| ANKRD32 | −1.4984 | 0.0123624 | 8.35386 | 8.42219 | 7.86046 | 7.74874 |
| ANKRD36B | −1.40151 | 0.0452008 | 10.7245 | 10.8656 | 10.2274 | 10.3888 |
| ANKRD6 | 1.70561 | 0.0471443 | 7.63169 | 7.43091 | 8.44303 | 8.16015 |
| ANLN | −1.62786 | 0.00833155 | 10.9859 | 10.895 | 10.2834 | 10.1916 |
| ANO10 | 1.47799 | 0.00073012 | 10.6829 | 10.6958 | 11.2391 | 11.2667 |
| ANXA2 | 1.55372 | 0.0112568 | 10.5446 | 10.4291 | 11.1585 | 11.0867 |
| ANXA3 | 1.86899 | 0.018269 | 6.01917 | 5.83607 | 6.91299 | 6.74676 |
| ANXA4 | 1.60861 | 0.00474848 | 9.48151 | 9.57031 | 10.195 | 10.2284 |
| AOC2 | 1.44587 | 0.0207243 | 7.38825 | 7.23268 | 7.8415 | 7.84331 |
| ARHGAP10 | 2.09716 | 0.00089925 | 7.99357 | 8.0489 | 9.07347 | 9.10588 |
| ARHGAP11B | −1.52597 | 0.0206746 | 10.0082 | 9.83162 | 9.29858 | 9.32181 |
| ARHGAP15 | −1.58158 | 0.0219894 | 11.1092 | 11.278 | 10.4791 | 10.5854 |
| ARHGAP25 | −1.78556 | 0.0339246 | 9.47569 | 9.78347 | 8.75694 | 8.82946 |
| ARHGAP30 | −1.54841 | 0.0219038 | 7.39852 | 7.42655 | 6.68787 | 6.87563 |
| ARHGAP32 | 1.45693 | 0.00509872 | 7.43036 | 7.43608 | 7.93734 | 8.01496 |
| ARHGAP4 | −2.68494 | 0.00721656 | 8.52215 | 8.67331 | 7.07745 | 7.26823 |
| ARHGAP9 | −1.54147 | 0.0114699 | 7.74852 | 7.86933 | 7.15461 | 7.21462 |
| ARHGDIB | −2.05233 | 0.0369881 | 11.8699 | 12.135 | 10.8086 | 11.1219 |
| ARHGEF6 | −1.70547 | 0.0201039 | 10.2572 | 10.4785 | 9.60473 | 9.59062 |
| ARID5A | 1.55305 | 0.0056084 | 9.51701 | 9.42956 | 10.1276 | 10.0892 |
| ARL4A | −1.41571 | 0.04585 | 8.10243 | 7.95255 | 7.44375 | 7.6082 |
| ARMCX1 | 1.64683 | 0.0109503 | 9.05298 | 8.93099 | 9.66644 | 9.7569 |
| ARMCX3 | 1.4797 | 0.0288882 | 8.3592 | 8.16383 | 8.83713 | 8.81651 |
| ARRDC4 | −1.46764 | 0.0242641 | 8.40074 | 8.57164 | 7.95297 | 7.91241 |
| ARSB | −1.59848 | 0.0427819 | 9.31123 | 9.59723 | 8.79935 | 8.75571 |
| ARVCF | 1.86915 | 0.001994 | 6.01353 | 6.05516 | 6.9713 | 6.90215 |
| ASF1B | −1.57793 | 0.0198142 | 10.3538 | 10.4461 | 9.82382 | 9.65999 |
| ASPH | 1.41404 | 0.0165936 | 10.0359 | 9.92068 | 10.5086 | 10.4476 |
| ASPHD1 | 1.42009 | 0.0124538 | 8.85651 | 8.75458 | 9.33706 | 9.286 |
| ATAD2 | −1.63731 | 0.0104043 | 10.6404 | 10.7223 | 10.0306 | 9.90944 |
| ATAD5 | −1.5136 | 0.00994822 | 9.70751 | 9.76348 | 9.19069 | 9.08433 |
| ATG9A | 1.42865 | 0.0359304 | 8.16251 | 7.96734 | 8.60266 | 8.55649 |
| ATN1 | 1.49882 | 0.00951713 | 8.60164 | 8.69131 | 9.26609 | 9.19452 |
| ATOH1 | −2.23311 | 0.0113562 | 7.48868 | 7.32831 | 6.1541 | 6.34478 |
| ATP2A3 | −1.85368 | 0.0494331 | 8.26167 | 8.66757 | 7.54088 | 7.60758 |
| ATP8B4 | −2.2534 | 0.00916169 | 6.57174 | 6.35242 | 5.26284 | 5.31712 |
| ATXN1 | 1.58358 | 0.00762933 | 8.91224 | 9.00185 | 9.65747 | 9.58299 |
| AXIN1 | 1.47629 | 0.0248497 | 7.73734 | 7.74937 | 8.39541 | 8.21525 |
| AZU1 | −2.66918 | 0.0198062 | 7.03924 | 7.34355 | 5.6416 | 5.90841 |
| B3GAT3 | 1.45449 | 0.0108208 | 7.88503 | 7.78192 | 8.35042 | 8.39754 |
| BAALC | −1.81934 | 0.0178316 | 9.16479 | 9.37786 | 8.35987 | 8.45594 |
| BACH2 | 2.34988 | 0.00430046 | 7.27257 | 7.35082 | 8.61531 | 8.47325 |
| BAI1 | 2.10815 | 0.00430633 | 10.6102 | 10.4971 | 11.6723 | 11.5869 |
| BCAR1 | 1.70601 | 0.00107564 | 6.36034 | 6.41092 | 7.15678 | 7.15574 |
| BCAR3 | 1.73513 | 0.0122063 | 7.64291 | 7.5649 | 8.47856 | 8.31934 |
| BCAT1 | 1.9501 | 0.00059749 | 9.62644 | 9.58094 | 10.5734 | 10.5611 |
| BCAT2 | 1.66273 | 0.00951068 | 7.81254 | 7.94876 | 8.5907 | 8.63771 |
| BCL11A | −1.97225 | 0.0414952 | 9.34352 | 9.71879 | 8.46613 | 8.6365 |
| BCL2L1 | 1.52586 | 0.0142836 | 9.70667 | 9.84255 | 10.3558 | 10.4127 |
| BCL2L11 | 1.83296 | 0.013406 | 10.4312 | 10.2303 | 11.2241 | 11.1858 |
| BHLHE41 | 1.98341 | 0.0206355 | 7.39157 | 7.11893 | 8.19632 | 8.29014 |
| BLM | −1.96298 | 0.0190012 | 9.32895 | 9.57548 | 8.53682 | 8.42153 |
| BLVRB | 1.44557 | 0.038828 | 7.7149 | 7.74604 | 8.36891 | 8.15531 |
| BMF | 1.4235 | 0.00515638 | 7.62228 | 7.54903 | 8.09778 | 8.09241 |
| BMPR1A | 1.75866 | 0.00062741 | 6.50327 | 6.48941 | 7.29162 | 7.33001 |
| BRCA2 | −1.82558 | 0.0188477 | 8.37871 | 8.58246 | 7.67739 | 7.54707 |
| BRI3BP | −2.29365 | 0.0253903 | 7.86503 | 8.25379 | 6.87009 | 6.85343 |
| BRPF3 | 1.46796 | 1.32E−05 | 9.91659 | 9.92041 | 10.4729 | 10.4717 |
| BST1 | −1.65832 | 0.0253343 | 9.00593 | 9.24132 | 8.38089 | 8.40692 |
| BUB1 | −1.62681 | 0.0398247 | 10.8553 | 11.0104 | 10.3527 | 10.1089 |
| BUB1B | −1.58166 | 0.0431265 | 8.64329 | 8.91783 | 8.0828 | 8.15545 |
| BZRAP1 | −1.70175 | 0.0194185 | 7.94208 | 8.10794 | 7.18808 | 7.32791 |
| C10orf10 | 1.48399 | 0.0452145 | 6.35988 | 6.28591 | 6.77257 | 7.01219 |
| C10orf26 | 1.43505 | 0.00291375 | 9.66905 | 9.72517 | 10.2209 | 10.2155 |
| C10orf78 | −1.53579 | 0.0304918 | 9.47348 | 9.33717 | 8.69919 | 8.87349 |
| C11orf80 | 1.5176 | 0.0390836 | 7.87632 | 7.63155 | 8.36288 | 8.34857 |
| C11orf82 | −1.59699 | 0.0317716 | 8.50204 | 8.64572 | 7.99878 | 7.79826 |
| C11orf9 | 1.78581 | 0.00820202 | 6.57059 | 6.43293 | 7.37111 | 7.30557 |
| C12orf48 | −1.47063 | 0.014423 | 8.8977 | 8.99509 | 8.43679 | 8.34314 |
| C12orf76 | 1.40683 | 0.0103035 | 6.455 | 6.55423 | 7.00579 | 6.98834 |
| C14orf49 | −1.40669 | 0.0342644 | 6.985 | 7.11528 | 6.49069 | 6.62498 |
| C15orf29 | 1.44799 | 0.0260201 | 10.0104 | 9.9521 | 10.4324 | 10.5982 |
| C15orf42 | −1.70198 | 0.00609177 | 10.3765 | 10.4458 | 9.69311 | 9.59473 |
| C16orf93 | −1.56555 | 0.00696769 | 7.89997 | 8.00825 | 7.30379 | 7.31109 |
| C17orf60 | −2.20505 | 0.0264907 | 10.4857 | 10.7705 | 9.36236 | 9.61221 |
| C1orf107 | 1.55867 | 0.00838311 | 8.29075 | 8.33207 | 9.00699 | 8.89646 |
| C1orf163 | 1.47448 | 0.0212919 | 7.65915 | 7.82356 | 8.28955 | 8.31359 |
| C1orf228 | −2.08748 | 0.00086002 | 7.81848 | 7.82995 | 6.73183 | 6.79308 |
| C1orf96 | −1.44814 | 0.00902432 | 9.97389 | 9.9482 | 9.37739 | 9.4763 |
| C20orf197 | −3.81561 | 0.0292058 | 7.56495 | 8.23996 | 5.96276 | 5.97833 |
| C21orf70 | 1.45189 | 0.0183593 | 6.65423 | 6.57259 | 7.08973 | 7.21296 |
| C2orf65 | −2.17475 | 0.0453281 | 7.44206 | 7.92603 | 6.51309 | 6.61329 |
| C2orf66 | 1.43834 | 0.00348469 | 6.61211 | 6.65322 | 7.13382 | 7.18033 |
| C3AR1 | −1.71796 | 0.0232573 | 10.2208 | 10.0671 | 9.45694 | 9.26951 |
| C3orf35 | 1.67836 | 0.0397743 | 7.38823 | 7.08557 | 8.01032 | 7.95759 |
| C4orf21 | −1.72128 | 0.0106804 | 9.91064 | 9.9497 | 9.22594 | 9.06743 |
| C4orf46 | −1.70746 | 0.00987994 | 8.84139 | 8.98753 | 8.1678 | 8.1174 |
| C5 | −3.64401 | 0.00894334 | 7.50985 | 7.82271 | 5.88488 | 5.71663 |
| C5orf13 | −2.12653 | 0.0158469 | 11.2686 | 11.5074 | 10.229 | 10.3701 |
| C5orf32 | 1.51394 | 0.0280002 | 8.86701 | 8.67671 | 9.40767 | 9.33267 |
| C5orf39 | −1.45299 | 0.0200125 | 8.50234 | 8.64026 | 8.06746 | 7.99708 |
| C6orf145 | 1.83885 | 0.0203454 | 6.84455 | 7.06769 | 7.89622 | 7.77363 |
| C6orf167 | −1.43086 | 0.0414216 | 8.93849 | 8.94623 | 8.53401 | 8.31695 |
| C9orf100 | −1.5418 | 0.0258381 | 7.83822 | 7.97921 | 7.35837 | 7.20983 |
| C9orf30 | 2.08314 | 0.0144895 | 8.76595 | 8.53777 | 9.77049 | 9.65075 |
| C9orf93 | −1.59032 | 0.0139463 | 6.48326 | 6.6251 | 5.8481 | 5.92162 |
| CAMK2N1 | 1.61805 | 0.0135537 | 6.75369 | 6.85383 | 7.43352 | 7.56252 |
| CAMSAP1L1 | 2.3167 | 0.0303805 | 7.33672 | 6.90736 | 8.30834 | 8.35989 |
| CAP2 | 2.99769 | 3.89E−05 | 7.71926 | 7.70359 | 9.28926 | 9.3013 |
| CAPN2 | 1.55552 | 0.00737276 | 9.15369 | 9.20166 | 9.8646 | 9.76553 |
| CAPNS2 | −1.40181 | 0.00070586 | 6.50369 | 6.47783 | 6.00269 | 6.00425 |
| CARM1 | 1.41497 | 0.00880953 | 8.52815 | 8.50136 | 9.06091 | 8.97015 |
| CASC5 | −1.59077 | 0.016391 | 9.78937 | 9.76137 | 9.19132 | 9.01997 |
| CASK | 2.04088 | 0.00635951 | 8.75762 | 8.67764 | 9.81895 | 9.6747 |
| CAV2 | 1.72307 | 0.0464103 | 6.10776 | 5.7666 | 6.68188 | 6.76244 |
| CCDC18 | −1.66897 | 0.00496064 | 9.1349 | 9.10652 | 8.43203 | 8.33148 |
| CCDC50 | 1.48528 | 0.00658589 | 8.61423 | 8.68001 | 9.25079 | 9.18491 |
| CCDC92 | 1.41934 | 0.0313482 | 8.82815 | 9.00453 | 9.39671 | 9.44641 |
| CCL20 | 1.80088 | 0.00362861 | 6.74903 | 6.85134 | 7.65221 | 7.64556 |
| CCNA2 | −1.53108 | 0.0243774 | 10.276 | 10.2433 | 9.74146 | 9.54873 |
| CCNB2 | −1.72686 | 0.0013067 | 10.5758 | 10.5798 | 9.81809 | 9.7612 |
| CCNE2 | −1.43112 | 0.0133435 | 10.2415 | 10.1638 | 9.73169 | 9.6393 |
| CD109 | 1.42138 | 0.00214032 | 9.14205 | 9.15629 | 9.67886 | 9.63406 |
| CD151 | 1.63973 | 0.00180853 | 9.77957 | 9.80054 | 10.475 | 10.532 |
| CD22 | 2.57322 | 0.00299271 | 6.31472 | 6.44993 | 7.71398 | 7.77783 |
| CD244 | −5.32907 | 0.00041959 | 7.52879 | 7.59965 | 5.18485 | 5.11582 |
| CD300LF | −2.35359 | 0.0287485 | 7.91721 | 8.32633 | 6.82399 | 6.94983 |
| CD302 | −2.42708 | 0.0152498 | 10.7778 | 11.0834 | 9.6047 | 9.69803 |
| CD33 | −1.85053 | 0.0017117 | 8.65953 | 8.71178 | 7.77182 | 7.82361 |
| CD34 | −4.039 | 0.00708597 | 9.08344 | 9.35319 | 7.10011 | 7.30853 |
| CD70 | 1.43916 | 0.00715947 | 7.54954 | 7.63564 | 8.12978 | 8.10586 |
| CD74 | −2.00524 | 0.00094091 | 8.49435 | 8.53512 | 7.48786 | 7.53407 |
| CD82 | −1.89005 | 0.00816652 | 6.65327 | 6.81522 | 5.83624 | 5.79539 |
| CD96 | −3.95335 | 0.00098759 | 8.25034 | 8.32943 | 6.35503 | 6.25858 |
| CD97 | 1.74289 | 0.00407429 | 9.88471 | 9.98709 | 10.741 | 10.7338 |
| CDC20 | −1.54751 | 0.0135276 | 10.5414 | 10.4007 | 9.81804 | 9.8642 |
| CDC34 | 1.42216 | 0.00432322 | 9.63078 | 9.57028 | 10.0942 | 10.123 |
| CDC42BPA | 1.55886 | 0.0073192 | 7.52113 | 7.49949 | 8.20482 | 8.09678 |
| CDC45 | −1.79645 | 0.01755 | 8.4808 | 8.57941 | 7.78715 | 7.58277 |
| CDC6 | −1.77092 | 0.00467358 | 9.92097 | 9.80962 | 9.05081 | 9.03078 |
| CDC7 | −1.90484 | 0.0261977 | 7.60236 | 7.89429 | 6.86618 | 6.77114 |
| CDCA2 | −1.50393 | 0.038804 | 8.96033 | 9.02136 | 8.51762 | 8.28659 |
| CDCA3 | −1.63381 | 0.00320515 | 7.10324 | 7.17024 | 6.45071 | 6.40628 |
| CDCA5 | −1.82764 | 0.0179625 | 7.61779 | 7.64578 | 6.87917 | 6.64444 |
| CDCA7L | −1.45324 | 0.0208383 | 10.5102 | 10.6594 | 10.0718 | 10.0192 |
| CDCA8 | −1.40881 | 0.0121329 | 9.37282 | 9.47623 | 8.9487 | 8.91138 |
| CDKN3 | −1.6799 | 0.00489685 | 9.53651 | 9.57669 | 8.8568 | 8.75966 |
| CDT1 | −1.45086 | 0.0145413 | 7.42603 | 7.53739 | 6.97923 | 6.91038 |
| CDYL2 | 1.59712 | 0.00801213 | 9.04996 | 9.07668 | 9.79814 | 9.67945 |
| CECR1 | −1.86682 | 0.033361 | 8.42527 | 8.75079 | 7.64292 | 7.73198 |
| CELF2 | −2.31485 | 0.0283209 | 10.3737 | 10.733 | 9.23718 | 9.44768 |
| CELF6 | 1.53654 | 0.0385073 | 6.33891 | 6.53988 | 7.13384 | 6.98431 |
| CENPE | −1.49753 | 0.00760892 | 10.0058 | 9.96289 | 9.44814 | 9.35534 |
| CENPF | −1.56409 | 0.0473694 | 9.59506 | 9.73292 | 9.14701 | 8.89033 |
| CENPI | −1.59016 | 0.0292138 | 9.53967 | 9.66338 | 9.0316 | 8.83309 |
| CENPM | −1.63156 | 0.0344145 | 8.11062 | 8.3629 | 7.48378 | 7.57723 |
| CENPW | −2.13676 | 0.00106516 | 9.53309 | 9.48123 | 8.38708 | 8.43639 |
| CEP110 | −1.70281 | 0.0103945 | 9.20401 | 9.25012 | 8.53461 | 8.38368 |
| CFL2 | 1.4791 | 0.00061403 | 8.91457 | 8.89993 | 9.46004 | 9.4839 |
| CHD7 | 1.67789 | 0.00045587 | 8.97136 | 8.9877 | 9.73987 | 9.71248 |
| CIT | −1.82765 | 0.0248491 | 9.24476 | 9.45146 | 8.5722 | 8.38404 |
| CLCN6 | 1.43949 | 0.00210225 | 9.79316 | 9.74727 | 10.2883 | 10.3033 |
| CLDN12 | 1.57406 | 0.0264362 | 9.80428 | 9.60171 | 10.3966 | 10.3184 |
| CLEC11A | −2.18594 | 0.0375655 | 8.41768 | 8.84634 | 7.43508 | 7.57243 |
| CLEC5A | −3.71904 | 0.00369756 | 9.45101 | 9.64831 | 7.59457 | 7.71488 |
| CLIP1 | 1.67523 | 0.00884142 | 10.0073 | 9.86645 | 10.6834 | 10.6791 |
| CLIP4 | 2.06577 | 0.00734604 | 6.80572 | 6.7455 | 7.90732 | 7.73726 |
| CLN8 | 1.56613 | 0.0178169 | 9.70273 | 9.74821 | 10.2881 | 10.4572 |
| CLSPN | −1.54393 | 0.0383285 | 7.93876 | 8.18779 | 7.45803 | 7.41529 |
| CNKSR3 | 1.47911 | 0.0203172 | 8.51197 | 8.51362 | 9.15926 | 8.99578 |
| CNRIP1 | 1.61195 | 0.0448715 | 6.72606 | 6.64376 | 7.2284 | 7.51905 |
| COLEC12 | 1.60131 | 0.0180591 | 7.13739 | 7.20762 | 7.76614 | 7.93738 |
| CORO1A | −3.27657 | 0.0110112 | 10.9876 | 11.3374 | 9.40303 | 9.49754 |
| CRIM1 | 1.43619 | 0.010228 | 7.19959 | 7.10765 | 7.70269 | 7.64904 |
| CRTC3 | 1.43062 | 0.00076193 | 10.2233 | 10.2226 | 10.7539 | 10.7253 |
| CSF1 | 2.52498 | 0.00155017 | 8.52838 | 8.48575 | 9.79516 | 9.8915 |
| CSNK1E | 1.42185 | 0.00275543 | 10.1051 | 10.1045 | 10.5859 | 10.6393 |
| CSNK1G1 | −1.67605 | 0.00156624 | 9.57504 | 9.63242 | 8.85172 | 8.86561 |
| CST3 | −1.53356 | 0.0406487 | 9.01225 | 9.25286 | 8.56031 | 8.47103 |
| CTH | −1.42752 | 0.0399853 | 8.75329 | 8.5465 | 8.11357 | 8.1592 |
| CTSB | 1.5821 | 0.00975787 | 10.7981 | 10.8706 | 11.4412 | 11.5511 |
| CTSL1 | 1.87251 | 0.00484245 | 6.40096 | 6.49062 | 7.39532 | 7.30621 |
| CTTN | 3.12138 | 0.00530477 | 8.17549 | 7.94935 | 9.74505 | 9.66416 |
| CTTNBP2NL | 1.45513 | 0.0483855 | 9.50258 | 9.268 | 9.9653 | 9.88757 |
| CXADR | 1.88598 | 0.0169516 | 8.11941 | 7.88041 | 8.89816 | 8.93229 |
| CXCL10 | 1.64684 | 0.0146677 | 12.0106 | 11.87 | 12.6069 | 12.7131 |
| CXorf21 | −5.83016 | 0.00400915 | 10.1821 | 10.4604 | 7.69562 | 7.85976 |
| CXorf23 | 1.4609 | 0.011174 | 8.17661 | 8.27209 | 8.73774 | 8.80467 |
| CYBA | −1.46139 | 0.00125203 | 6.89797 | 6.86772 | 6.34763 | 6.32338 |
| CYHR1 | 1.41941 | 0.0118091 | 9.20757 | 9.23645 | 9.67382 | 9.78079 |
| CYSLTR1 | −3.11147 | 0.0200145 | 10.5707 | 10.9606 | 8.99649 | 9.25969 |
| CYTH1 | 1.4627 | 0.0176504 | 11.2219 | 11.1264 | 11.6664 | 11.7791 |
| DAB2 | 1.42014 | 0.0141317 | 10.9217 | 10.8955 | 11.3552 | 11.474 |
| DAPK3 | 1.46998 | 0.0459062 | 8.51859 | 8.27341 | 8.93794 | 8.96565 |
| DAPP1 | −1.94026 | 0.00259002 | 9.82754 | 9.86319 | 8.9345 | 8.84373 |
| DBN1 | 1.51715 | 0.00433032 | 7.97534 | 8.05432 | 8.62029 | 8.6121 |
| DCBLD2 | 1.65783 | 0.0131984 | 9.83583 | 9.75389 | 10.5982 | 10.4501 |
| DCK | −1.56465 | 0.0345703 | 9.69189 | 9.90774 | 9.09435 | 9.2136 |
| DDIT3 | 1.41873 | 0.0236644 | 11.2236 | 11.0667 | 11.6595 | 11.6401 |
| DEF8 | 1.46488 | 0.0226577 | 7.47447 | 7.48372 | 8.11411 | 7.94566 |
| DENND1C | −1.59177 | 0.00521921 | 8.16073 | 8.24545 | 7.50855 | 7.55637 |
| DENND3 | −1.46344 | 0.0301948 | 10.426 | 10.6188 | 9.95722 | 9.98881 |
| DENND5B | 1.5476 | 0.0105547 | 8.52593 | 8.44612 | 9.16768 | 9.06444 |
| DEPDC1 | −1.45552 | 0.0164572 | 9.92415 | 9.80575 | 9.36141 | 9.28542 |
| DEPDC7 | −1.47716 | 0.0229791 | 8.42256 | 8.3729 | 7.9181 | 7.75171 |
| DFNA5 | 2.42904 | 0.00355753 | 8.6466 | 8.49381 | 9.84531 | 9.85587 |
| DHFR | −1.85464 | 0.0169358 | 9.7186 | 9.88898 | 8.83177 | 8.99353 |
| DIAPH3 | −1.4096 | 0.00036986 | 8.80702 | 8.79475 | 8.29831 | 8.31289 |
| DLG5 | 1.67741 | 0.00216333 | 8.7153 | 8.71193 | 9.42513 | 9.49457 |
| DLGAP5 | −1.57223 | 0.0226721 | 9.46823 | 9.49758 | 8.92901 | 8.73117 |
| DLL1 | −1.61847 | 0.0316749 | 9.31462 | 9.07269 | 8.53656 | 8.46149 |
| DMPK | 2.11411 | 0.0129612 | 7.46758 | 7.2596 | 8.37578 | 8.51149 |
| DMWD | 2.0928 | 0.00795865 | 8.15845 | 8.06658 | 9.26181 | 9.09408 |
| DNAH17 | 2.36305 | 0.036841 | 5.82786 | 5.81698 | 7.30796 | 6.81818 |
| DNAJB2 | 1.58641 | 0.0311359 | 6.82497 | 6.61248 | 7.32805 | 7.44093 |
| DOCK4 | 1.46508 | 0.0353235 | 8.01212 | 7.87165 | 8.57278 | 8.41295 |
| DOCK6 | 1.80473 | 0.0100223 | 7.24797 | 7.41738 | 8.17007 | 8.19885 |
| DPY19L2P1 | −1.67934 | 0.0199286 | 6.47293 | 6.37236 | 5.58009 | 5.76941 |
| DPY19L2P2 | −1.50672 | 0.0166945 | 6.2184 | 6.34259 | 5.6429 | 5.73526 |
| DPYD | 1.44645 | 0.00120303 | 8.5161 | 8.48241 | 9.02415 | 9.03938 |
| DSCC1 | −1.96474 | 0.00260775 | 7.98531 | 8.08476 | 7.05711 | 7.06428 |
| DSCR3 | 1.44339 | 0.0318984 | 10.7871 | 10.9161 | 11.3088 | 11.4534 |
| DTL | −1.72251 | 0.0178139 | 11.0061 | 11.1493 | 10.3715 | 10.2149 |
| DUSP3 | 1.59347 | 0.0118162 | 9.38722 | 9.36578 | 10.1216 | 9.97573 |
| DUSP6 | −1.51948 | 0.0017958 | 9.73736 | 9.77017 | 9.16986 | 9.13052 |
| DUSP8 | 1.61055 | 0.036776 | 8.21748 | 8.01774 | 8.89691 | 8.71341 |
| DYNLT3 | 1.44723 | 0.039026 | 9.25569 | 9.40036 | 9.94227 | 9.78038 |
| EAF2 | −1.58243 | 0.0024889 | 11.1052 | 11.1573 | 10.4487 | 10.4896 |
| ECE1 | 1.52766 | 0.0134032 | 8.15337 | 8.22829 | 8.86304 | 8.74126 |
| ELANE | −1.79069 | 0.0148988 | 8.62124 | 8.82474 | 7.86217 | 7.90279 |
| ELMO1 | −1.46156 | 0.023502 | 11.0131 | 11.1688 | 10.5786 | 10.5083 |
| EMB | −1.44485 | 0.0373041 | 10.7286 | 10.8479 | 10.1703 | 10.3443 |
| EMR2 | −1.66661 | 0.0433776 | 6.99634 | 6.89417 | 6.0581 | 6.35859 |
| ENAH | 1.46167 | 0.0119809 | 8.63941 | 8.7602 | 9.24417 | 9.25068 |
| ENO2 | 1.66999 | 0.0115132 | 7.34141 | 7.182 | 7.99386 | 8.00924 |
| EPAG | 1.4997 | 0.0210597 | 6.02702 | 5.87661 | 6.57864 | 6.49433 |
| EPS8 | 1.55807 | 0.00676562 | 8.75854 | 8.8595 | 9.46458 | 9.43299 |
| ERLIN1 | −1.44551 | 0.00971246 | 11.7274 | 11.8326 | 11.2525 | 11.2442 |
| ERLIN2 | −1.50523 | 0.0205718 | 8.05882 | 8.19988 | 7.49024 | 7.5885 |
| ESCO2 | −1.75712 | 0.0327761 | 9.72213 | 10.0015 | 9.10589 | 8.99127 |
| ESPL1 | −1.56163 | 0.00275673 | 8.44458 | 8.46143 | 7.84272 | 7.77718 |
| EVL | −1.43238 | 0.0264246 | 7.33471 | 7.4559 | 6.9379 | 6.81589 |
| EXO1 | −1.91586 | 0.00438161 | 8.41679 | 8.51512 | 7.56622 | 7.48972 |
| F2RL2 | 4.95343 | 0.00049156 | 5.93659 | 5.96233 | 8.30744 | 8.20833 |
| FABP3 | 2.21143 | 0.00783839 | 5.74529 | 5.85335 | 6.85781 | 7.03078 |
| FAM101B | −1.41905 | 0.0188538 | 8.49069 | 8.61467 | 8.08097 | 8.01455 |
| FAM105A | −1.96287 | 0.0106932 | 8.46357 | 8.66018 | 7.61388 | 7.56394 |
| FAM128A | 1.53001 | 0.0142235 | 7.89551 | 8.03117 | 8.54739 | 8.60636 |
| FAM177A1 | 1.60277 | 0.00614392 | 7.94938 | 7.84939 | 8.59926 | 8.56064 |
| FAM40B | 1.70763 | 0.0251925 | 9.15907 | 8.9093 | 9.80403 | 9.80832 |
| FAM50A | 1.6703 | 0.00145634 | 9.99816 | 9.96814 | 10.7472 | 10.6993 |
| FAM65A | 1.65066 | 0.0035686 | 8.34898 | 8.36356 | 9.122 | 9.03662 |
| FAM69A | 1.48567 | 0.0481257 | 8.4546 | 8.22721 | 8.97512 | 8.84892 |
| FAM72D | −1.84404 | 0.00253997 | 10.7387 | 10.6496 | 9.8113 | 9.81129 |
| FAM84B | −1.56818 | 0.0139859 | 7.67024 | 7.75461 | 6.99823 | 7.12844 |
| FANCB | −1.5225 | 0.0211439 | 8.9191 | 8.83397 | 8.34895 | 8.19123 |
| FANCD2 | −1.67472 | 0.0159819 | 10.2677 | 10.3958 | 9.65827 | 9.51744 |
| FANCI | −1.53228 | 0.00906314 | 10.0978 | 10.1626 | 9.56386 | 9.46518 |
| FBXL2 | 2.72777 | 0.00319154 | 7.22782 | 7.15938 | 8.56682 | 8.71583 |
| FBXO31 | 1.48626 | 0.0147463 | 7.15568 | 7.2536 | 7.82663 | 7.72602 |
| FBXO48 | −1.44267 | 0.0343641 | 8.03235 | 8.04241 | 7.60914 | 7.40814 |
| FBXW8 | 1.60931 | 0.0016779 | 8.51919 | 8.57166 | 9.24208 | 9.22166 |
| FERMT2 | 2.15877 | 0.0403661 | 7.38522 | 6.9256 | 8.25455 | 8.27669 |
| FES | −1.74312 | 0.0469137 | 7.23701 | 7.59492 | 6.63402 | 6.59457 |
| FGD6 | 1.83582 | 0.00467181 | 8.16485 | 8.08362 | 9.04498 | 8.95634 |
| FHL1 | 1.66892 | 0.00899992 | 7.84747 | 7.75726 | 8.487 | 8.59556 |
| FJX1 | 1.41388 | 0.00580564 | 8.04815 | 7.98507 | 8.49465 | 8.53788 |
| FLCN | 1.60961 | 0.013688 | 8.22313 | 8.06458 | 8.84805 | 8.81308 |
| FLJ13224 | −1.47978 | 0.0177762 | 6.79631 | 6.72673 | 6.12812 | 6.26417 |
| FLJ35776 | 2.18046 | 0.013413 | 8.35002 | 8.09053 | 9.36679 | 9.32303 |
| FLNB | 1.57632 | 0.0117737 | 9.55929 | 9.68153 | 10.2391 | 10.3148 |
| FLNC | 2.15644 | 0.0271818 | 8.16255 | 7.79068 | 9.10109 | 9.06944 |
| FLOT1 | 1.52552 | 0.0138048 | 8.57952 | 8.46305 | 9.08768 | 9.1735 |
| FLRT2 | 2.71578 | 0.00519632 | 8.4864 | 8.34351 | 9.78032 | 9.93232 |
| FMNL2 | 1.63731 | 0.00923415 | 10.778 | 10.6408 | 11.4264 | 11.4151 |
| FMNL3 | 2.32647 | 0.00557609 | 8.73284 | 8.55133 | 9.87062 | 9.84984 |
| FNIP2 | 1.5062 | 0.0308666 | 10.871 | 10.6783 | 11.4105 | 11.3206 |
| FOSL2 | 1.59776 | 0.00499941 | 9.78368 | 9.68859 | 10.4057 | 10.4186 |
| FOXP2 | 1.42145 | 0.0382597 | 7.91421 | 8.10598 | 8.55282 | 8.48211 |
| FRAT1 | −1.46682 | 0.0283782 | 9.05447 | 9.1228 | 8.44715 | 8.62474 |
| FRMD8 | 1.51943 | 0.00236386 | 7.0533 | 7.03784 | 7.67746 | 7.62073 |
| FYB | −3.37167 | 0.00609035 | 8.67282 | 8.91488 | 6.9752 | 7.10558 |
| G6PD | 1.75165 | 0.00239574 | 10.1993 | 10.2714 | 11.0275 | 11.0606 |
| GAB2 | 1.42348 | 0.0143873 | 9.19701 | 9.1252 | 9.72079 | 9.62026 |
| GABARAPL1 | 1.54779 | 0.0496508 | 9.41347 | 9.16343 | 9.99391 | 9.84341 |
| GABBR1 | 1.56508 | 0.00550968 | 8.90576 | 8.81332 | 9.51933 | 9.49222 |
| GABPA | −1.45655 | 0.0337662 | 7.62116 | 7.54215 | 7.13347 | 6.94473 |
| GALNT3 | −1.75598 | 0.00395922 | 7.79895 | 7.80245 | 7.03966 | 6.93719 |
| GAPT | −3.94184 | 0.00325587 | 9.57985 | 9.77401 | 7.75626 | 7.63986 |
| GAS2L3 | 1.74984 | 0.0122076 | 7.03846 | 6.88315 | 7.81355 | 7.72251 |
| GATA2 | 1.75159 | 0.0288731 | 7.20751 | 6.97487 | 7.8211 | 7.97861 |
| GATS | 1.51605 | 0.00046119 | 8.24898 | 8.25803 | 8.8659 | 8.84175 |
| GBE1 | 1.7071 | 0.00207811 | 9.30057 | 9.28524 | 10.0301 | 10.0988 |
| GCLM | 1.41189 | 0.0100914 | 8.40631 | 8.35223 | 8.9194 | 8.8344 |
| GDI1 | 1.43389 | 0.00052674 | 11.3658 | 11.3546 | 11.8696 | 11.8906 |
| GDPD3 | 1.78555 | 0.0379092 | 6.44039 | 6.12273 | 7.06428 | 7.17157 |
| GGA1 | 1.53896 | 0.00171966 | 8.80635 | 8.80531 | 9.4536 | 9.40196 |
| GINS1 | −1.52461 | 0.0492762 | 9.18572 | 9.44827 | 8.75807 | 8.65905 |
| GINS2 | −2.00572 | 0.0277102 | 9.63532 | 9.85467 | 8.8717 | 8.61004 |
| GK | 1.43568 | 0.0163906 | 11.2989 | 11.2359 | 11.849 | 11.7293 |
| GLIPR2 | −1.56173 | 0.0186405 | 8.74104 | 8.91042 | 8.15503 | 8.21014 |
| GLRX | −1.50137 | 0.024815 | 10.0614 | 10.2459 | 9.54875 | 9.58607 |
| GMNN | −1.43317 | 0.0147264 | 9.12675 | 9.00484 | 8.52805 | 8.56514 |
| GNA11 | 1.51593 | 0.0027379 | 6.63168 | 6.63232 | 7.20074 | 7.26367 |
| GNA15 | −1.42383 | 0.0178657 | 8.34341 | 8.43036 | 7.93078 | 7.82345 |
| GNG12 | 1.61709 | 0.00736217 | 10.2121 | 10.1022 | 10.8269 | 10.8742 |
| GNPDA1 | 1.43447 | 0.00252801 | 10.2705 | 10.2621 | 10.7609 | 10.8127 |
| GNPTAB | −1.41442 | 0.026436 | 11.0335 | 11.1943 | 10.5933 | 10.6341 |
| GPAM | −2.91804 | 0.0210567 | 10.7149 | 10.8303 | 9.00721 | 9.44796 |
| GPNMB | 1.56943 | 0.0176807 | 8.82226 | 8.99705 | 9.56631 | 9.5535 |
| GPR141 | −4.74125 | 0.01317 | 7.71215 | 8.11328 | 5.83329 | 5.50161 |
| GPR160 | −1.70063 | 0.040828 | 8.64653 | 8.96596 | 8.04198 | 8.03836 |
| GRIP1 | 1.4619 | 0.0341369 | 6.36315 | 6.23967 | 6.93281 | 6.76568 |
| GSDMB | 1.73912 | 0.0015269 | 7.70535 | 7.73309 | 8.54556 | 8.48959 |
| GSG2 | −1.41558 | 0.0250819 | 7.71202 | 7.68788 | 7.27859 | 7.11852 |
| GSTM2 | 1.64246 | 0.0242566 | 5.75734 | 5.87851 | 6.62985 | 6.43773 |
| GTF2IRD1 | 1.63342 | 0.0050005 | 7.42536 | 7.33518 | 8.06599 | 8.11035 |
| GTPBP2 | 1.41685 | 0.00119928 | 10.7622 | 10.7912 | 11.2697 | 11.2891 |
| GTPBP6 | 1.42193 | 0.0198864 | 5.97867 | 6.01029 | 6.43137 | 6.5733 |
| GYPC | −1.64824 | 0.0472527 | 9.61688 | 9.94152 | 9.06654 | 9.05002 |
| HABP4 | 1.40327 | 0.049633 | 6.59026 | 6.78043 | 7.23547 | 7.11281 |
| HAT1 | −1.41107 | 4.15E−05 | 11.2575 | 11.2569 | 10.7572 | 10.7636 |
| HAUS4 | −1.59877 | 0.0300275 | 8.94518 | 9.17594 | 8.41668 | 8.35051 |
| HCST | −1.67707 | 0.00405127 | 7.31418 | 7.28463 | 6.50819 | 6.59873 |
| HEATR7A | 1.43731 | 0.00904456 | 8.5403 | 8.56907 | 9.12606 | 9.03005 |
| HEBP2 | 1.63798 | 0.00051245 | 8.85313 | 8.83533 | 9.5427 | 9.56959 |
| HECTD3 | 1.56567 | 0.00454839 | 8.8044 | 8.88324 | 9.50963 | 9.47159 |
| HEG1 | 2.27213 | 0.00363489 | 8.41526 | 8.27771 | 9.55037 | 9.51069 |
| HELLS | −1.59774 | 0.00445694 | 9.37748 | 9.29662 | 8.68142 | 8.64062 |
| HESX1 | −1.66513 | 0.00036651 | 8.65353 | 8.62973 | 7.89846 | 7.91354 |
| HGS | 1.49238 | 0.00577794 | 9.23806 | 9.18818 | 9.8271 | 9.75436 |
| HIST1H1B | −1.61174 | 0.00736168 | 9.52683 | 9.55018 | 8.90814 | 8.79163 |
| HIST1H1C | −1.74837 | 0.0179203 | 8.79463 | 8.77357 | 7.86923 | 8.08696 |
| HIST1H2AB | −2.2358 | 0.0176132 | 9.07163 | 8.77992 | 7.7093 | 7.82068 |
| HIST1H2AE | −1.7777 | 0.00876292 | 10.9074 | 10.8058 | 10.086 | 9.96714 |
| HIST1H2AG | −1.62619 | 0.0039456 | 9.3088 | 9.26124 | 8.54627 | 8.62078 |
| HIST1H2AH | −1.79043 | 0.0422158 | 8.68939 | 8.45402 | 7.59739 | 7.86541 |
| HIST1H2AI | −1.85449 | 0.014543 | 8.86228 | 8.77029 | 7.82684 | 8.02369 |
| HIST1H2AK | −1.59338 | 0.0247314 | 10.1917 | 9.97642 | 9.40784 | 9.41607 |
| HIST1H2AL | −1.52872 | 0.0126615 | 10.0961 | 9.96178 | 9.43472 | 9.39852 |
| HIST1H2AM | −2.29281 | 0.0489673 | 10.5821 | 10.0748 | 9.02483 | 9.2379 |
| HIST1H2BB | −2.13655 | 0.00648044 | 7.58857 | 7.71822 | 6.49771 | 6.61851 |
| HIST1H2BF | −2.06596 | 0.0182003 | 11.7207 | 11.7485 | 10.5453 | 10.8303 |
| HIST1H2BG | −1.71312 | 0.0498994 | 10.6125 | 10.2692 | 9.6091 | 9.71943 |
| HIST1H2BH | −1.40754 | 0.00600705 | 11.4861 | 11.5624 | 11.0355 | 11.0268 |
| HIST1H2BI | −1.97142 | 0.0352404 | 7.20852 | 6.83088 | 6.03679 | 6.04413 |
| HIST1H2BL | −1.78689 | 0.00102608 | 9.07019 | 9.09419 | 8.26875 | 8.22072 |
| HIST1H2BO | −1.63117 | 0.00827054 | 10.1101 | 9.99633 | 9.3167 | 9.37792 |
| HIST1H3A | −2.11337 | 0.0171165 | 9.83528 | 9.56575 | 8.57289 | 8.66903 |
| HIST1H3B | −1.9643 | 0.00440469 | 11.7921 | 11.6703 | 10.7795 | 10.7349 |
| HIST1H3C | −1.92206 | 0.0190309 | 9.27425 | 9.03339 | 8.26504 | 8.1573 |
| HIST1H3F | −1.76703 | 0.0266108 | 10.1216 | 9.96907 | 9.33749 | 9.11056 |
| HIST1H3G | −2.03931 | 0.00121875 | 8.93594 | 8.99859 | 7.9216 | 7.95676 |
| HIST1H3H | −1.54604 | 0.0116685 | 11.3881 | 11.3712 | 10.6831 | 10.8191 |
| HIST1H3I | −1.48711 | 0.0107409 | 12.7048 | 12.8233 | 12.1834 | 12.1996 |
| HIST1H3J | −1.95052 | 0.0132831 | 11.02 | 10.8137 | 9.90874 | 9.99722 |
| HIST1H4A | −2.23604 | 0.0290602 | 8.91232 | 8.56561 | 7.47366 | 7.68238 |
| HIST1H4B | −1.77034 | 0.00424384 | 8.97265 | 8.8768 | 8.12526 | 8.07613 |
| HIST1H4C | −1.53494 | 0.00580171 | 12.1838 | 12.1856 | 11.6138 | 11.5192 |
| HIST1H4D | −1.67323 | 0.00309479 | 10.3829 | 10.4601 | 9.69382 | 9.66384 |
| HIST1H4E | −1.44383 | 0.00282839 | 11.2103 | 11.2078 | 10.6509 | 10.7074 |
| HIST1H4I | −2.02631 | 0.0103848 | 8.22799 | 8.27713 | 7.13199 | 7.33542 |
| HIST1H4K | −1.42979 | 0.0119069 | 10.3315 | 10.4005 | 9.80505 | 9.89528 |
| HIST2H2AB | −1.50199 | 0.0165304 | 11.1917 | 11.2573 | 10.7066 | 10.5686 |
| HIST2H3A | −1.48365 | 0.00400811 | 10.2168 | 10.2807 | 9.66273 | 9.69648 |
| HJURP | −1.4537 | 0.0434436 | 8.40645 | 8.56201 | 8.03099 | 7.85801 |
| HMGA1 | 1.43774 | 0.00799842 | 8.79023 | 8.87953 | 9.37376 | 9.34359 |
| HMGXB3 | 1.44075 | 0.0134559 | 9.61407 | 9.50384 | 10.1136 | 10.058 |
| HMOX1 | 2.29665 | 0.0321679 | 9.62834 | 9.19647 | 10.6566 | 10.5673 |
| HPDL | −1.57455 | 0.0422713 | 6.65103 | 6.59731 | 5.83274 | 6.10572 |
| HRH2 | −1.76397 | 0.00493041 | 6.54187 | 6.62996 | 5.7298 | 5.80438 |
| HRK | 1.40542 | 0.00264513 | 8.64697 | 8.67697 | 9.17335 | 9.13259 |
| HSD17B14 | 1.84321 | 0.0115818 | 7.19105 | 7.01246 | 7.94933 | 8.01861 |
| HSP90AA6P | −1.54877 | 0.0168227 | 10.3527 | 10.2524 | 9.60543 | 9.73741 |
| ICAM1 | 1.66103 | 0.00877074 | 9.90009 | 9.84392 | 10.541 | 10.6671 |
| ICAM3 | −1.6636 | 0.0356959 | 7.03608 | 7.31494 | 6.41141 | 6.471 |
| IDH2 | −1.61004 | 0.0203562 | 9.41315 | 9.59825 | 8.85529 | 8.78191 |
| IGFBP7 | −2.13967 | 0.0373923 | 8.88083 | 9.29638 | 7.92402 | 8.05843 |
| IKBKE | −1.40705 | 0.0446704 | 8.16463 | 8.33358 | 7.68952 | 7.82337 |
| IKZF1 | −1.77916 | 0.0428475 | 9.12656 | 9.47982 | 8.49257 | 8.45142 |
| IL16 | −1.40538 | 0.0129577 | 6.48587 | 6.53742 | 5.97048 | 6.07089 |
| IL20RB | 1.47916 | 0.0207015 | 6.19346 | 6.25056 | 6.86424 | 6.70933 |
| IL4R | 1.42185 | 0.0430731 | 7.15418 | 7.28516 | 7.81449 | 7.6404 |
| IL6R | 1.96875 | 0.00759972 | 7.55499 | 7.71054 | 8.57408 | 8.646 |
| IMP3 | −1.50862 | 0.0253646 | 8.17163 | 7.97899 | 7.48186 | 7.4823 |
| INPP5D | −2.04502 | 0.0280531 | 10.3405 | 10.6624 | 9.3966 | 9.54204 |
| IQGAP2 | −1.78638 | 0.0300514 | 8.10246 | 8.39719 | 7.43086 | 7.39471 |
| IRAK1 | 1.55414 | 0.016544 | 8.22588 | 8.11867 | 8.87157 | 8.74522 |
| IRAK2 | 2.10495 | 0.0168094 | 10.3981 | 10.133 | 11.3875 | 11.2912 |
| IRX3 | −1.73786 | 0.00210937 | 9.30112 | 9.3739 | 8.54476 | 8.53563 |
| ITGA3 | 2.76051 | 0.0013076 | 8.45404 | 8.3503 | 9.85609 | 9.87812 |
| ITGA4 | −2.10771 | 0.0324682 | 10.2668 | 10.644 | 9.44228 | 9.31717 |
| ITGA6 | 2.13504 | 0.00190445 | 9.03422 | 9.12779 | 10.1852 | 10.1654 |
| ITGAX | 1.49515 | 0.0302874 | 7.64335 | 7.43802 | 8.13286 | 8.10908 |
| ITIH4 | −1.4176 | 0.0117155 | 7.15022 | 7.16889 | 6.71028 | 6.60193 |
| ITPKB | 1.43416 | 0.0336962 | 6.17943 | 6.16177 | 6.59322 | 6.7884 |
| ITPRIPL2 | 1.42137 | 0.0373484 | 7.99045 | 7.79895 | 8.43376 | 8.3702 |
| ITSN1 | 1.45848 | 0.0222962 | 7.97569 | 7.81953 | 8.4693 | 8.41485 |
| JPH1 | 1.52345 | 0.00324406 | 6.83945 | 6.77086 | 7.40738 | 7.41762 |
| KCTD7 | 1.65245 | 0.00568492 | 9.78216 | 9.72041 | 10.4305 | 10.5212 |
| KDM1B | −1.61577 | 0.0394344 | 8.93936 | 9.10367 | 8.44473 | 8.21385 |
| KIAA0101 | −1.95523 | 0.0134896 | 10.5697 | 10.7499 | 9.76151 | 9.62347 |
| KIAA0182 | −1.64489 | 0.03928 | 7.91794 | 8.11772 | 7.19247 | 7.40722 |
| KIAA0319 | 1.45215 | 0.0334423 | 5.89121 | 5.9645 | 6.37196 | 6.56013 |
| KIAA0355 | 1.62784 | 0.0107007 | 8.02276 | 8.14509 | 8.82729 | 8.74648 |
| KIAA0427 | 1.72413 | 0.0005832 | 7.4522 | 7.41888 | 8.23051 | 8.21231 |
| KIAA0913 | 1.41496 | 0.0282426 | 8.62273 | 8.50379 | 9.12613 | 9.00192 |
| KIAA1045 | 1.72494 | 0.0303154 | 5.55202 | 5.6393 | 6.24902 | 6.5154 |
| KIAA1147 | −1.83769 | 0.0165887 | 7.83678 | 8.03857 | 7.11392 | 7.00564 |
| KIAA1524 | −1.55325 | 0.00308745 | 10.4925 | 10.4983 | 9.89534 | 9.82482 |
| KIAA1737 | 1.48012 | 0.00217238 | 9.09482 | 9.07537 | 9.62626 | 9.67536 |
| KIF11 | −1.61906 | 0.00366675 | 10.427 | 10.5105 | 9.77993 | 9.76728 |
| KIF14 | −1.66075 | 0.039046 | 8.92683 | 8.76401 | 8.2384 | 7.98878 |
| KIF15 | −1.9636 | 0.00863028 | 9.90226 | 10.0724 | 9.0462 | 8.98148 |
| KIF1B | 1.57 | 0.00084415 | 8.35971 | 8.35087 | 9.02445 | 8.98766 |
| KIF20A | −1.89243 | 0.00872583 | 9.15741 | 9.32703 | 8.33914 | 8.30482 |
| KIF20B | −1.61317 | 0.0200026 | 10.1958 | 10.2908 | 9.64031 | 9.46643 |
| KIF23 | −1.57541 | 0.0207533 | 9.52189 | 9.58814 | 8.98936 | 8.80923 |
| KIF2C | −1.5394 | 0.024526 | 8.69454 | 8.84806 | 8.21194 | 8.08593 |
| KIFC1 | −1.41907 | 0.0156577 | 8.73047 | 8.61815 | 8.19992 | 8.1388 |
| KIRREL | 1.73361 | 0.0111158 | 6.73041 | 6.77752 | 7.4667 | 7.62878 |
| KIT | −2.63519 | 0.0189673 | 11.5531 | 11.9028 | 10.243 | 10.4171 |
| KITLG | 1.81375 | 0.00418232 | 7.85898 | 7.79307 | 8.64006 | 8.72994 |
| KLC2 | 1.49365 | 0.0158524 | 8.01362 | 8.15389 | 8.68543 | 8.63976 |
| KLF3 | 1.47834 | 0.00224379 | 10.8679 | 10.9212 | 11.4562 | 11.4609 |
| KLF6 | 1.54513 | 0.010831 | 10.419 | 10.3076 | 11.0262 | 10.9559 |
| KLHDC8B | 2.933 | 0.00397205 | 8.97652 | 8.81883 | 10.3916 | 10.5085 |
| KNTC1 | −1.58655 | 0.0159123 | 9.53379 | 9.54525 | 8.95845 | 8.7888 |
| KRTCAP3 | 1.40331 | 0.00603734 | 6.81997 | 6.88545 | 7.36114 | 7.32196 |
| LAIR1 | −3.42817 | 0.0201276 | 10.1797 | 10.5965 | 8.46191 | 8.75939 |
| LAMP3 | −1.7791 | 0.0187771 | 8.77622 | 8.91329 | 8.10661 | 7.92061 |
| LAPTM5 | −3.21846 | 0.0104405 | 10.0224 | 10.367 | 8.53025 | 8.48647 |
| LAT | 1.72408 | 0.00154847 | 6.99137 | 7.03836 | 7.78053 | 7.82085 |
| LCP1 | −2.32931 | 0.0066119 | 9.65024 | 9.77766 | 8.57073 | 8.41737 |
| LDLRAD3 | 1.58485 | 0.00135869 | 9.22044 | 9.2659 | 9.9167 | 9.89833 |
| LGALS3 | 2.03072 | 0.00379386 | 7.92963 | 7.81263 | 8.91684 | 8.86939 |
| LGALS9B | −1.41097 | 0.0490126 | 10.9793 | 11.2058 | 10.5813 | 10.6105 |
| LGALS9C | −1.47142 | 0.0320502 | 10.4752 | 10.6541 | 9.95797 | 10.0569 |
| LIFR | 1.55405 | 0.0224055 | 9.38005 | 9.27665 | 10.0463 | 9.8825 |
| LIMK1 | 1.66746 | 0.00174397 | 8.21138 | 8.2726 | 8.98346 | 8.97581 |
| LIN7A | −2.01588 | 0.0223737 | 7.27567 | 7.44444 | 6.47732 | 6.21997 |
| LIPH | 1.92801 | 0.00958804 | 5.2071 | 5.34921 | 6.16463 | 6.28591 |
| LITAF | 2.78565 | 0.00260853 | 7.2508 | 7.22672 | 8.79144 | 8.6421 |
| LMAN2L | 1.41511 | 0.00250644 | 8.44085 | 8.3906 | 8.91698 | 8.91631 |
| LMLN | 1.41983 | 0.0284185 | 8.21735 | 8.34363 | 8.84624 | 8.72617 |
| LMNA | 1.66478 | 0.00494141 | 7.47962 | 7.45281 | 8.25167 | 8.15142 |
| LOC100129503 | 1.5249 | 0.023562 | 6.89364 | 7.02185 | 7.63675 | 7.49618 |
| LOC100131541 | 1.92987 | 0.0149642 | 8.51292 | 8.29337 | 9.39313 | 9.31017 |
| LOC100131826 | 1.56768 | 0.0307517 | 7.05073 | 6.84803 | 7.65535 | 7.54067 |
| LOC100133299 | 1.46218 | 0.0236862 | 10.0501 | 9.87837 | 10.5141 | 10.5107 |
| LOC1720 | −2.0665 | 0.0275212 | 9.83251 | 9.84313 | 8.6133 | 8.96796 |
| LOC388022 | 2.1336 | 0.00967847 | 7.99662 | 8.09308 | 9.23516 | 9.04112 |
| LOC389787 | −1.46216 | 0.0422067 | 11.2049 | 11.0657 | 10.494 | 10.6804 |
| LOC442421 | −1.51377 | 0.0032321 | 9.18813 | 9.16257 | 8.5456 | 8.60881 |
| LOC643332 | −5.0515 | 0.012118 | 7.80417 | 8.15052 | 5.83402 | 5.44724 |
| LOC643837 | 1.42083 | 0.00594654 | 7.74097 | 7.66393 | 8.21672 | 8.20165 |
| LOC652904 | −1.44747 | 0.00782941 | 7.68389 | 7.64867 | 7.08864 | 7.17685 |
| LOC654433 | 1.76237 | 0.0154498 | 9.00951 | 8.81161 | 9.70017 | 9.75598 |
| LOC729595 | −1.42786 | 7.87E−05 | 6.71649 | 6.72538 | 6.20608 | 6.20809 |
| LONP1 | 1.51926 | 0.0306458 | 7.19685 | 7.08764 | 7.83893 | 7.65228 |
| LPCAT2 | −2.19387 | 0.00376152 | 11.5272 | 11.6434 | 10.4133 | 10.4903 |
| LPHN3 | −2.52041 | 0.0421648 | 6.40447 | 6.96652 | 5.38416 | 5.3195 |
| LPP | 1.79979 | 0.0055835 | 9.91762 | 9.80501 | 10.6795 | 10.7388 |
| LPXN | −1.72512 | 0.00595409 | 9.89083 | 9.76969 | 9.03652 | 9.05061 |
| LRMP | −2.73222 | 0.00448602 | 8.68719 | 8.7896 | 7.37123 | 7.2054 |
| LRP1 | 1.56438 | 0.00287873 | 6.08726 | 6.15665 | 6.76656 | 6.76853 |
| LRRC17 | −1.58725 | 0.012116 | 6.16943 | 6.25037 | 5.48136 | 5.60537 |
| LRRC39 | 1.88368 | 0.0252766 | 6.8289 | 6.63041 | 7.53333 | 7.75309 |
| LRRC70 | 1.7066 | 0.0230498 | 6.59014 | 6.82637 | 7.49499 | 7.46377 |
| LRSAM1 | 1.53172 | 0.0132575 | 7.49594 | 7.35298 | 8.04261 | 8.03661 |
| LSS | 1.45355 | 0.00266624 | 8.80384 | 8.78154 | 9.30667 | 9.35786 |
| LST1 | −1.59939 | 0.0203629 | 6.70133 | 6.84475 | 6.16259 | 6.02844 |
| LTBR | 1.50036 | 0.00088878 | 8.21137 | 8.20376 | 8.80992 | 8.77583 |
| LY6G5B | 1.42799 | 0.00141338 | 9.85223 | 9.88987 | 10.3895 | 10.3806 |
| LY86 | −1.91951 | 0.0248674 | 6.76001 | 6.64103 | 5.89877 | 5.6208 |
| LY96 | 2.12651 | 0.0314053 | 8.77758 | 8.46636 | 9.5887 | 9.83221 |
| LYZ | −5.64795 | 0.00487345 | 9.90665 | 10.0407 | 7.63762 | 7.3143 |
| MAFG | 1.58095 | 0.00644391 | 8.03141 | 8.01438 | 8.63107 | 8.73631 |
| MAML2 | 1.7221 | 0.0113099 | 9.25446 | 9.08762 | 9.94445 | 9.96596 |
| MAMLD1 | 1.72496 | 0.00995345 | 6.21154 | 6.08004 | 6.88844 | 6.97627 |
| MAN2B1 | −1.48102 | 0.0291848 | 10.027 | 10.2086 | 9.5906 | 9.51181 |
| MAP1B | 1.69191 | 0.0228093 | 9.40303 | 9.17129 | 10.0587 | 10.0329 |
| MAP2 | 3.80734 | 0.0159313 | 5.61837 | 5.21035 | 7.20497 | 7.48132 |
| MAST2 | 1.43705 | 0.00856726 | 9.18631 | 9.14777 | 9.73491 | 9.64539 |
| MBD5 | 1.50042 | 0.00650347 | 8.0489 | 7.95569 | 8.59651 | 8.57882 |
| MBNL2 | 1.43563 | 0.0182534 | 9.15339 | 9.03068 | 9.65036 | 9.57707 |
| MBNL3 | −1.94261 | 0.0493249 | 8.11481 | 8.52773 | 7.44219 | 7.28437 |
| MCM10 | −1.67398 | 0.0374031 | 8.98793 | 9.28271 | 8.40467 | 8.37941 |
| MCM6 | −1.63932 | 0.012584 | 9.20421 | 9.36515 | 8.56479 | 8.57837 |
| MCOLN3 | 1.48229 | 0.00090656 | 8.59438 | 8.62386 | 9.16826 | 9.18563 |
| MED27 | 1.43556 | 0.0117414 | 6.45939 | 6.40014 | 7.0001 | 6.90265 |
| MEGF9 | −2.15765 | 0.035545 | 9.01474 | 8.84075 | 8.01483 | 7.62173 |
| METTL7A | −1.58946 | 0.0279834 | 7.11574 | 7.30938 | 6.48339 | 6.60467 |
| MFAP4 | −5.29889 | 0.0059783 | 8.79751 | 9.15045 | 6.5069 | 6.62968 |
| MFI2 | 1.62183 | 0.00100572 | 8.75517 | 8.71596 | 9.44348 | 9.4229 |
| MGAT4B | 1.63429 | 0.0110134 | 8.87057 | 8.77959 | 9.47412 | 9.59336 |
| MICALL1 | 1.41952 | 0.00505972 | 7.75988 | 7.78738 | 8.3124 | 8.24567 |
| MID2 | 1.58197 | 0.0282417 | 7.91141 | 7.77678 | 8.59735 | 8.41427 |
| MINA | 1.58945 | 0.0164435 | 7.55377 | 7.54591 | 8.13165 | 8.30507 |
| MIR181B1 | −1.62752 | 0.0401535 | 8.02738 | 8.27563 | 7.5242 | 7.37347 |
| MIR1977 | −1.65588 | 0.0349719 | 11.8248 | 11.7499 | 11.1944 | 10.9251 |
| MIR221 | −2.06135 | 0.0247509 | 8.98441 | 9.29354 | 8.15942 | 8.03136 |
| MIR223 | −7.9485 | 0.0116947 | 9.27984 | 9.74065 | 6.28853 | 6.7506 |
| MKI67 | −2.16208 | 0.00514335 | 10.2893 | 10.4206 | 9.28838 | 9.19663 |
| MLC1 | −1.63097 | 0.0170238 | 7.20678 | 7.38732 | 6.6148 | 6.56784 |
| MLF1IP | −1.5393 | 0.0198863 | 8.60954 | 8.61713 | 8.08007 | 7.90206 |
| MLH3 | 1.59297 | 0.00466083 | 8.90439 | 8.97579 | 9.64085 | 9.58277 |
| MLLT4 | 1.42775 | 0.00316833 | 8.0261 | 8.06888 | 8.58079 | 8.54167 |
| MMP10 | 2.01599 | 0.0265975 | 5.69589 | 5.49172 | 6.73915 | 6.47145 |
| MNS1 | −1.81679 | 0.0356056 | 7.52687 | 7.57214 | 6.8536 | 6.52262 |
| MPO | −2.82986 | 0.0278869 | 13.2263 | 13.4227 | 11.5873 | 12.0602 |
| MPZL1 | 1.56578 | 0.015269 | 8.36101 | 8.20932 | 8.904 | 8.96011 |
| MRAS | 1.72224 | 0.00335391 | 8.60317 | 8.56028 | 9.32584 | 9.40617 |
| MSH2 | −1.4659 | 0.0468928 | 8.66443 | 8.91005 | 8.21913 | 8.25179 |
| MSH5 | −1.63676 | 0.00089937 | 8.53382 | 8.57589 | 7.84048 | 7.84754 |
| MSRA | 1.53117 | 0.0285025 | 8.33836 | 8.43263 | 9.09512 | 8.90513 |
| MT1G | −1.41068 | 0.0274885 | 10.2352 | 10.3626 | 9.85735 | 9.74768 |
| MT1X | −1.50248 | 0.0112484 | 11.69 | 11.6521 | 11.1436 | 11.0238 |
| MTBP | −1.54822 | 0.0126736 | 8.83061 | 8.94946 | 8.21935 | 8.2995 |
| MTL5 | −1.51913 | 0.0480313 | 7.24681 | 7.4207 | 6.62438 | 6.83665 |
| MTMR11 | 1.77856 | 0.0242953 | 6.73655 | 6.95344 | 7.75078 | 7.60063 |
| MTSS1 | 2.48796 | 8.68E−05 | 8.66825 | 8.68347 | 9.98121 | 10.0004 |
| MXD3 | −1.41982 | 0.00524989 | 8.36701 | 8.43975 | 7.89215 | 7.90319 |
| MYB | −2.43756 | 0.0257242 | 11.9467 | 12.1811 | 10.6039 | 10.9531 |
| MYO18B | 1.60338 | 0.00241168 | 7.69717 | 7.73432 | 8.42475 | 8.36897 |
| MYO1B | 1.66376 | 0.00914318 | 8.55746 | 8.43294 | 9.19611 | 9.26317 |
| MYO1F | −2.37032 | 0.0115766 | 7.85721 | 7.91628 | 6.50979 | 6.77353 |
| MYO1G | −2.96333 | 0.00665311 | 8.65465 | 8.90517 | 7.18413 | 7.24125 |
| MYO6 | 1.48835 | 0.016983 | 8.80839 | 8.75924 | 9.42916 | 9.28589 |
| N4BP2L1 | 1.46449 | 0.00399111 | 6.58852 | 6.58016 | 7.16936 | 7.10011 |
| NAGA | −1.40071 | 0.0408898 | 8.6562 | 8.84847 | 8.29858 | 8.23378 |
| NANOS1 | −1.77966 | 0.0381862 | 10.3497 | 10.0244 | 9.31615 | 9.39469 |
| NAV1 | 1.84065 | 0.00168169 | 8.81231 | 8.74319 | 9.64738 | 9.66854 |
| NBEAL1 | 1.44134 | 0.0363138 | 10.5017 | 10.3005 | 10.9048 | 10.9522 |
| NCAPD3 | −1.4922 | 0.0318572 | 10.1187 | 10.226 | 9.68589 | 9.50396 |
| NCAPG | −1.55619 | 0.0352234 | 10.6611 | 10.8333 | 10.1971 | 10.0213 |
| NCAPG2 | −1.47458 | 0.0226182 | 9.09511 | 9.18708 | 8.65314 | 8.50844 |
| NCAPH | −1.4773 | 0.0315798 | 9.76096 | 9.85567 | 9.33625 | 9.15447 |
| NCKAP1 | 1.63407 | 0.0159789 | 10.8437 | 10.667 | 11.4842 | 11.4435 |
| NCRNA00152 | 1.51901 | 0.00059297 | 10.9333 | 10.9573 | 11.5399 | 11.557 |
| NDC80 | −1.51753 | 0.0364663 | 9.27251 | 9.19788 | 8.74559 | 8.52135 |
| NDFIP2 | 1.55259 | 0.0196019 | 7.43169 | 7.27699 | 7.94263 | 8.0354 |
| NDRG1 | 1.45894 | 0.00173155 | 10.3585 | 10.4037 | 10.9243 | 10.9278 |
| NEIL3 | −1.60035 | 0.02118 | 7.3358 | 7.51081 | 6.794 | 6.69583 |
| NEK2 | −1.93609 | 0.00326934 | 7.99666 | 8.02944 | 7.11202 | 7.00778 |
| NEK3 | 1.42494 | 0.0133275 | 8.469 | 8.37479 | 8.89632 | 8.96928 |
| NEK6 | −2.26374 | 0.00652532 | 8.1495 | 8.33718 | 7.04594 | 7.08333 |
| NELF | 1.52369 | 0.00552683 | 8.06638 | 8.13176 | 8.73808 | 8.67519 |
| NEURL3 | 1.60844 | 0.0457799 | 7.3388 | 7.55228 | 8.23939 | 8.02302 |
| NFATC2 | −1.87309 | 0.0194948 | 7.97468 | 8.2196 | 7.15346 | 7.22999 |
| NHEJ1 | 1.49782 | 0.0297062 | 7.70593 | 7.90316 | 8.41631 | 8.35852 |
| NLN | 1.563 | 0.00293491 | 8.16009 | 8.098 | 8.78948 | 8.75724 |
| NLRP3 | −1.71504 | 0.0306226 | 6.64196 | 6.57925 | 5.69653 | 5.9682 |
| NMNAT3 | −1.40112 | 0.0141928 | 7.27187 | 7.38739 | 6.85289 | 6.83322 |
| NPFF | 1.45144 | 0.00210164 | 7.37841 | 7.33562 | 7.90679 | 7.8822 |
| NQO1 | 2.48424 | 0.00149931 | 9.21768 | 9.11597 | 10.4778 | 10.4814 |
| NR1D1 | 1.61577 | 0.00029701 | 7.85569 | 7.85306 | 8.53473 | 8.55845 |
| NRM | −2.08185 | 0.0369433 | 7.49901 | 7.91718 | 6.64425 | 6.65621 |
| NRP2 | 2.19973 | 0.00955787 | 7.70995 | 7.4906 | 8.76028 | 8.71493 |
| NSA2 | −1.40305 | 0.013489 | 11.1861 | 11.272 | 10.7025 | 10.7785 |
| NSUN6 | −1.40748 | 0.0103586 | 9.40166 | 9.36146 | 8.84202 | 8.93486 |
| NTAN1 | 1.51885 | 0.0255594 | 8.67116 | 8.47773 | 9.19496 | 9.15988 |
| NUCB2 | −1.66156 | 0.0103202 | 10.6054 | 10.7424 | 9.97189 | 9.91087 |
| NUDT6 | −1.4778 | 0.00355642 | 7.07661 | 7.03794 | 6.46622 | 6.52141 |
| NUDT7 | −1.79518 | 0.00993054 | 8.65637 | 8.77726 | 7.81327 | 7.9321 |
| NUF2 | −1.80027 | 0.0080204 | 9.08126 | 9.23004 | 8.32496 | 8.28992 |
| NUPR1 | 1.92123 | 0.00298838 | 8.0185 | 8.06052 | 8.9344 | 9.02868 |
| NUSAP1 | −1.47767 | 0.00094264 | 10.3278 | 10.3458 | 9.78825 | 9.75867 |
| ODZ3 | 1.46753 | 0.0159652 | 5.90506 | 5.8575 | 6.36801 | 6.50133 |
| OPTN | 1.71209 | 0.0239693 | 9.28378 | 9.11434 | 10.063 | 9.88659 |
| OR52K1 | −1.70577 | 0.0131279 | 7.07183 | 7.19241 | 6.29602 | 6.42737 |
| OR52K3P | −2.14747 | 0.00335664 | 7.57005 | 7.44377 | 6.39356 | 6.41498 |
| ORC1L | −1.93182 | 0.00468482 | 7.89514 | 7.99264 | 7.03729 | 6.95056 |
| OTUD7B | 1.70566 | 0.0174211 | 8.25583 | 8.07606 | 8.98663 | 8.88593 |
| OVGP1 | 1.51731 | 0.0350212 | 6.2367 | 6.01588 | 6.76215 | 6.69346 |
| OVOS | −1.60842 | 0.0293486 | 6.80753 | 6.5674 | 6.0055 | 5.99814 |
| P2RX4 | 1.50607 | 0.0115426 | 10.0975 | 9.97765 | 10.6058 | 10.6509 |
| P2RX7 | 1.70701 | 0.00790378 | 10.6502 | 10.5144 | 11.366 | 11.3415 |
| P2RY13 | −2.58807 | 0.0340791 | 7.14446 | 7.53825 | 6.1392 | 5.79977 |
| P2RY8 | −3.12493 | 0.0294121 | 8.54081 | 9.02021 | 6.97648 | 7.2969 |
| P4HA2 | 1.50261 | 0.012355 | 7.71479 | 7.67689 | 8.22018 | 8.34643 |
| PALLD | 1.58118 | 0.00197707 | 6.51667 | 6.45876 | 7.14342 | 7.15401 |
| PAM | 1.43793 | 0.0164715 | 9.84892 | 9.72877 | 10.3449 | 10.2808 |
| PARP1 | −1.40096 | 0.0367997 | 9.12306 | 9.30315 | 8.69347 | 8.75992 |
| PARVG | −1.72716 | 0.00061819 | 9.22243 | 9.23617 | 8.42253 | 8.45927 |
| PAX8 | 1.57109 | 0.0271169 | 6.5512 | 6.33357 | 7.10699 | 7.08132 |
| PCBP4 | 1.49443 | 0.0163355 | 7.36396 | 7.50743 | 7.99342 | 8.03716 |
| PCGF2 | 1.41073 | 0.00339703 | 7.41467 | 7.38358 | 7.87108 | 7.92006 |
| PCNA | −1.76845 | 0.020131 | 10.8897 | 10.6899 | 9.90363 | 10.031 |
| PCSK6 | 2.59163 | 0.00264096 | 8.3903 | 8.29137 | 9.76527 | 9.66412 |
| PDE8A | 1.4387 | 0.00600579 | 10.0518 | 10.0434 | 10.613 | 10.5318 |
| PDE8B | 1.44169 | 0.049821 | 6.03404 | 5.79853 | 6.41057 | 6.47754 |
| PDGFA | 1.67901 | 0.00494448 | 6.68401 | 6.66447 | 7.36999 | 7.4737 |
| PDGFC | 1.42886 | 0.0174174 | 7.17116 | 7.3079 | 7.76254 | 7.74624 |
| PECAM1 | −2.08603 | 0.0175265 | 6.70794 | 6.97895 | 5.7392 | 5.82617 |
| PGBD1 | 1.47537 | 0.0378877 | 8.81411 | 8.60732 | 9.31598 | 9.22762 |
| PHKA1 | 1.59941 | 0.00563987 | 9.37929 | 9.3386 | 10.0834 | 9.98961 |
| PHLPP2 | 1.41893 | 0.0101391 | 9.93393 | 9.95072 | 10.4977 | 10.3966 |
| PI4K2A | 1.41068 | 0.0338581 | 8.79249 | 8.61375 | 9.22779 | 9.17124 |
| PIGK | −1.7762 | 0.0313823 | 9.54138 | 9.83292 | 8.8214 | 8.89531 |
| PIK3IP1 | 1.45939 | 0.00355658 | 9.04383 | 9.02675 | 9.54919 | 9.61213 |
| PITPNM2 | 2.03557 | 0.0226518 | 5.99405 | 6.29911 | 7.20926 | 7.13476 |
| PLA2G4C | 2.08882 | 0.00362526 | 9.63184 | 9.5123 | 10.6114 | 10.6581 |
| PLAC8 | −2.09015 | 0.0223159 | 7.04339 | 7.36656 | 6.14403 | 6.13871 |
| PLAC8L1 | 1.79238 | 0.0131375 | 6.50265 | 6.47495 | 7.23421 | 7.42715 |
| PLCB2 | −1.7792 | 0.0099066 | 7.31785 | 7.39263 | 6.44951 | 6.59851 |
| PLD4 | −1.84216 | 0.00913997 | 7.56712 | 7.73554 | 6.78032 | 6.75955 |
| PLEK | −2.24054 | 0.0154966 | 11.2741 | 11.5185 | 10.1515 | 10.3134 |
| PLEKHA1 | 1.641 | 0.0104185 | 7.53108 | 7.51852 | 8.31261 | 8.16613 |
| PLEKHM1 | 1.79647 | 0.0020174 | 9.78633 | 9.81987 | 10.6824 | 10.6141 |
| PLEKHO1 | 1.43063 | 0.00325042 | 9.29388 | 9.32432 | 9.80045 | 9.85105 |
| PLIN2 | 1.53164 | 0.0130722 | 11.435 | 11.3591 | 11.9521 | 12.0721 |
| PLK1 | −1.48749 | 0.030332 | 10.1471 | 10.3505 | 9.68491 | 9.66689 |
| PLOD3 | 1.42879 | 0.00127205 | 8.61101 | 8.62172 | 9.14874 | 9.11357 |
| PLXDC2 | −2.34334 | 0.0185468 | 10.3912 | 10.7291 | 9.31558 | 9.34753 |
| PLXNA1 | 1.57334 | 0.0167638 | 7.1272 | 7.29375 | 7.88471 | 7.8439 |
| PLXNA3 | 1.76096 | 0.00183377 | 8.78207 | 8.76524 | 9.624 | 9.55604 |
| PMP22 | 1.75091 | 0.0398973 | 6.66365 | 6.64295 | 7.29529 | 7.62751 |
| POLA2 | −1.42448 | 0.0402988 | 9.24194 | 9.38459 | 8.88083 | 8.72484 |
| POLE | −1.55351 | 0.0135115 | 9.32466 | 9.47265 | 8.77285 | 8.75338 |
| POLR3G | 1.40852 | 0.017332 | 8.9083 | 8.80867 | 9.30949 | 9.39583 |
| POR | 1.45575 | 0.00560787 | 8.72081 | 8.6394 | 9.22357 | 9.22016 |
| PPFIBP1 | 1.78304 | 0.00317123 | 8.77233 | 8.74123 | 9.63557 | 9.54667 |
| PPP1R16A | 1.48334 | 0.0202538 | 7.7129 | 7.79516 | 8.39406 | 8.2517 |
| PPP2R5B | 1.65999 | 0.021205 | 8.20084 | 8.00005 | 8.87197 | 8.79126 |
| PRIM1 | −2.28462 | 0.0173814 | 9.23964 | 9.55722 | 8.21823 | 8.19472 |
| PRIM2 | −1.5055 | 0.0390312 | 9.15886 | 9.39296 | 8.6585 | 8.71283 |
| PRKCH | 2.1464 | 0.0171894 | 8.0656 | 8.33574 | 9.2462 | 9.35897 |
| PRO2012 | 1.53876 | 0.0131536 | 8.04838 | 8.06623 | 8.75055 | 8.60761 |
| PRR11 | −2.14417 | 0.0137121 | 9.38535 | 9.626 | 8.35551 | 8.45501 |
| PRSSL1 | −3.32705 | 0.0352073 | 8.24258 | 8.88478 | 6.73642 | 6.92245 |
| PSD3 | −1.92428 | 0.0238468 | 6.48185 | 6.75695 | 5.73103 | 5.61913 |
| PSEN2 | 1.60261 | 0.0178105 | 6.88008 | 6.91437 | 7.48722 | 7.66808 |
| PTGER2 | −2.07283 | 0.00309672 | 9.10524 | 9.01371 | 8.04456 | 7.97118 |
| PTGER4 | −1.71944 | 0.0181812 | 11.3223 | 11.1142 | 10.4608 | 10.4118 |
| PTK2 | 1.4417 | 0.0100163 | 11.3945 | 11.3887 | 11.9725 | 11.8662 |
| PTK2B | −1.53365 | 0.0179527 | 8.99929 | 9.16628 | 8.47297 | 8.45867 |
| PTPDC1 | 1.44855 | 0.00830492 | 7.53659 | 7.47325 | 8.07694 | 8.00211 |
| PTPN22 | −2.67113 | 0.00671586 | 7.07096 | 7.18072 | 5.81143 | 5.60534 |
| PTPN6 | −1.46116 | 0.00481497 | 7.57657 | 7.51513 | 6.9762 | 7.02128 |
| PTPRC | −1.82144 | 0.0106636 | 10.4834 | 10.657 | 9.7291 | 9.68106 |
| PTPRCAP | −1.73145 | 0.0378529 | 7.85942 | 8.16964 | 7.25575 | 7.18935 |
| PTPRE | −2.04142 | 0.0148033 | 9.86951 | 10.1227 | 8.97095 | 8.96213 |
| PTPRM | 1.44212 | 0.00167887 | 10.3219 | 10.3639 | 10.8658 | 10.8764 |
| PTX3 | −2.35612 | 0.0161602 | 8.33684 | 8.03627 | 6.89787 | 7.0024 |
| PXK | −1.57323 | 0.0236801 | 12.1166 | 12.3072 | 11.5207 | 11.5956 |
| PXN | 1.45406 | 0.00017984 | 7.73801 | 7.75018 | 8.28025 | 8.28812 |
| PYCARD | −1.55544 | 0.0399294 | 8.26789 | 8.52188 | 7.72413 | 7.79099 |
| QRICH2 | 1.48028 | 0.00942879 | 7.62749 | 7.738 | 8.24552 | 8.2517 |
| RAB27A | 1.49448 | 0.00065797 | 8.54986 | 8.56355 | 9.14955 | 9.12314 |
| RAB37 | −4.56254 | 0.0142379 | 9.16017 | 9.4592 | 6.90211 | 7.33758 |
| RAB44 | −1.89157 | 0.00019893 | 6.50033 | 6.5147 | 5.57713 | 5.59873 |
| RABL3 | 1.42603 | 9.33E−05 | 9.19762 | 9.19234 | 9.7028 | 9.71117 |
| RAC2 | −3.15965 | 0.00269209 | 8.90888 | 9.06939 | 7.29766 | 7.36108 |
| RAD51AP1 | −1.79915 | 0.0396676 | 8.84761 | 8.95474 | 8.21939 | 7.88834 |
| RAD51L1 | −1.46432 | 0.0133861 | 8.67233 | 8.79359 | 8.20417 | 8.16129 |
| RAD54L | −1.61188 | 0.0160982 | 8.50972 | 8.65319 | 7.94447 | 7.84096 |
| RAI14 | 1.90257 | 0.0069305 | 8.82736 | 8.67528 | 9.69504 | 9.6635 |
| RANBP3L | −3.18532 | 0.0333239 | 9.45117 | 9.2267 | 7.95967 | 7.37531 |
| RAP2B | 1.41799 | 0.0345403 | 9.7463 | 9.56566 | 10.1928 | 10.1269 |
| RAPH1 | 1.79148 | 0.0353597 | 6.14075 | 5.82495 | 6.86246 | 6.78554 |
| RASAL3 | 1.54602 | 0.0256804 | 7.93178 | 7.79521 | 8.41532 | 8.56879 |
| RASGEF1B | 1.64879 | 0.00221551 | 8.76791 | 8.75564 | 9.51664 | 9.44973 |
| RASGRP2 | −1.45631 | 0.0303892 | 6.31123 | 6.44706 | 5.76791 | 5.90575 |
| RASGRP4 | −2.02443 | 0.0100999 | 6.74819 | 6.9165 | 5.75536 | 5.87429 |
| RASSF2 | −2.90343 | 0.00580215 | 7.74547 | 7.86033 | 6.16246 | 6.36782 |
| RASSF4 | −1.50089 | 0.0009775 | 7.46841 | 7.43335 | 6.87041 | 6.85972 |
| RASSF8 | 1.76966 | 0.00369381 | 7.66445 | 7.62332 | 8.51313 | 8.42157 |
| RBM43 | −1.4445 | 0.031564 | 9.56586 | 9.7537 | 9.15165 | 9.10677 |
| RCAN2 | 1.52653 | 0.0132711 | 6.86464 | 6.77471 | 7.37497 | 7.48489 |
| RENBP | 1.44072 | 0.0478159 | 5.86421 | 5.96061 | 6.32982 | 6.54857 |
| RET | −3.07437 | 0.0115723 | 8.0428 | 8.38851 | 6.62757 | 6.56315 |
| RFC1 | −2.4026 | 0.0198229 | 12.1315 | 12.0081 | 10.6353 | 10.9751 |
| RFC4 | −1.4195 | 0.0351322 | 10.9943 | 11.1762 | 10.6144 | 10.5452 |
| RGS2 | −1.51149 | 0.046949 | 12.2552 | 12.1345 | 11.4794 | 11.7184 |
| RGS9 | 1.60611 | 0.00923014 | 9.89843 | 9.79509 | 10.5716 | 10.4891 |
| RHCG | 1.96362 | 0.0376795 | 7.06607 | 6.68436 | 7.88628 | 7.81118 |
| RHOBTB1 | 1.44397 | 0.0223816 | 7.47561 | 7.52028 | 8.10549 | 7.95048 |
| RHOC | 1.46744 | 0.00064615 | 11.0867 | 11.0647 | 11.6202 | 11.6378 |
| RILPL1 | 2.50381 | 0.00237194 | 6.18392 | 6.05608 | 7.43476 | 7.45349 |
| RMI1 | −1.8298 | 0.0118047 | 8.71982 | 8.75492 | 7.95962 | 7.77175 |
| RNASE2 | −3.82597 | 0.0008118 | 8.62332 | 8.69575 | 6.68207 | 6.76536 |
| RNASEH2B | −1.64378 | 0.0170498 | 10.4591 | 10.6343 | 9.86604 | 9.7933 |
| RNASET2 | −1.52142 | 0.0231269 | 8.79212 | 8.93846 | 8.20133 | 8.3184 |
| RNF130 | −1.44014 | 0.0434946 | 11.2016 | 11.4252 | 10.7677 | 10.8067 |
| RNF145 | 1.49565 | 0.0162958 | 9.00761 | 8.8706 | 9.55056 | 9.48921 |
| RNF185 | 1.44745 | 0.00049386 | 9.39466 | 9.37995 | 9.91152 | 9.93012 |
| RNF19B | 1.53782 | 0.013732 | 9.81778 | 9.69438 | 10.4169 | 10.337 |
| RNU105C | 1.6442 | 0.0245255 | 8.48693 | 8.28742 | 9.16069 | 9.04843 |
| ROPN1L | −1.47023 | 0.0323621 | 8.36555 | 8.17777 | 7.67442 | 7.75683 |
| RPL15 | −1.41741 | 0.027349 | 12.3726 | 12.3146 | 11.7605 | 11.9202 |
| RPL21P44 | 1.45833 | 0.00206337 | 8.32309 | 8.29055 | 8.86981 | 8.83247 |
| RRAGC | 1.45647 | 0.0244544 | 10.692 | 10.5233 | 11.1691 | 11.1312 |
| RRAS | 1.72076 | 0.00948465 | 10.8715 | 11.0232 | 11.7184 | 11.7425 |
| RRM2 | −1.80915 | 0.0130438 | 8.90738 | 8.7142 | 7.93541 | 7.97554 |
| RRP12 | 1.41831 | 0.0390384 | 9.4163 | 9.3839 | 10.0056 | 9.80291 |
| RTN4IP1 | −1.58969 | 0.0125155 | 6.94977 | 7.02658 | 6.25439 | 6.38446 |
| RUFY3 | 1.43446 | 0.00576368 | 9.9187 | 9.95341 | 10.4923 | 10.4209 |
| RUNDC2A | 1.53845 | 0.0420193 | 7.14273 | 7.39551 | 7.92719 | 7.854 |
| RUNDC2C | 1.53891 | 0.00309823 | 8.52304 | 8.5268 | 9.11219 | 9.18148 |
| RUSC2 | 1.76175 | 0.00081655 | 8.34706 | 8.30697 | 9.15602 | 9.13203 |
| SAMD4A | 1.64271 | 0.017816 | 8.00569 | 7.81198 | 8.62701 | 8.62282 |
| SAMHD1 | −1.6183 | 0.032799 | 11.1502 | 11.3774 | 10.5083 | 10.6304 |
| SASH1 | 1.72952 | 0.0151054 | 5.82142 | 5.92478 | 6.74704 | 6.5799 |
| SAV1 | 1.43005 | 0.0366382 | 10.3868 | 10.1913 | 10.8328 | 10.7774 |
| SCARNA9L | −1.70262 | 0.0481905 | 10.3449 | 10.2189 | 9.35097 | 9.67733 |
| SEL1L3 | 2.06829 | 0.0328709 | 8.82267 | 8.45681 | 9.75548 | 9.62087 |
| SEMA4C | 1.43374 | 0.0367722 | 7.25941 | 7.21766 | 7.85869 | 7.65794 |
| SEMA6A | 4.853 | 0.00911326 | 6.41137 | 5.97354 | 8.46364 | 8.47901 |
| SERPINA1 | −1.54795 | 0.028598 | 7.28207 | 7.13318 | 6.65681 | 6.49772 |
| SERPINB1 | −1.59703 | 0.0490884 | 12.1247 | 12.3845 | 11.4939 | 11.6646 |
| SERPINB9 | 2.01973 | 0.00525659 | 7.38969 | 7.25749 | 8.30488 | 8.37062 |
| SESN3 | −1.59036 | 0.011375 | 9.73878 | 9.73433 | 9.13917 | 8.99522 |
| SGCB | 1.61457 | 0.0379507 | 9.49194 | 9.21512 | 10.0523 | 10.0371 |
| SGOL1 | −1.80832 | 0.00799473 | 9.81139 | 9.84403 | 9.04819 | 8.89794 |
| SGPL1 | 1.55574 | 0.0211275 | 9.61757 | 9.45144 | 10.2165 | 10.1277 |
| SGSH | 1.82209 | 0.016226 | 8.37353 | 8.16464 | 9.17406 | 9.09529 |
| SGSM3 | 1.58347 | 0.00867932 | 7.29419 | 7.17371 | 7.88163 | 7.91244 |
| SH3BP5 | 2.00833 | 0.00792231 | 6.71891 | 6.71833 | 7.81469 | 7.63454 |
| SH3KBP1 | −1.59181 | 0.0010513 | 13.7782 | 13.7655 | 13.0804 | 13.122 |
| SH3PXD2B | 2.0348 | 0.00141223 | 8.93786 | 8.97468 | 9.94728 | 10.015 |
| SHB | 1.54045 | 0.0114247 | 8.20457 | 8.30378 | 8.83219 | 8.92287 |
| SHCBP1 | −1.52708 | 0.0230609 | 9.42944 | 9.38948 | 8.89093 | 8.70643 |
| SIGLEC1 | −3.45533 | 0.00649817 | 10.0261 | 10.2786 | 8.29245 | 8.43462 |
| SIGLEC12 | −1.8668 | 0.0224889 | 7.07121 | 7.2977 | 6.36166 | 6.20612 |
| SKA1 | −1.60186 | 0.00748458 | 8.67753 | 8.76154 | 8.08142 | 7.99815 |
| SKA2 | −1.81253 | 0.021289 | 11.8345 | 12.0885 | 11.096 | 11.111 |
| SKA3 | −1.6007 | 0.0143581 | 8.5884 | 8.75073 | 8.00397 | 7.97776 |
| SLA2 | −1.53369 | 0.0270947 | 6.2799 | 6.4524 | 5.80668 | 5.69162 |
| SLAMF7 | 1.75068 | 0.040108 | 8.34773 | 8.04674 | 9.07722 | 8.93308 |
| SLC15A2 | −1.50247 | 0.0323198 | 9.08569 | 9.30145 | 8.6149 | 8.59757 |
| SLC15A3 | 1.90224 | 8.20E−05 | 6.90529 | 6.8929 | 7.83247 | 7.82112 |
| SLC15A4 | 1.49112 | 2.10E−05 | 10.5963 | 10.5964 | 11.1754 | 11.1701 |
| SLC17A5 | 1.59913 | 0.00179495 | 11.1921 | 11.1485 | 11.8289 | 11.8663 |
| SLC18A2 | −2.31165 | 0.0215525 | 9.32805 | 9.65319 | 8.20348 | 8.35992 |
| SLC25A35 | −1.40629 | 0.00891169 | 6.66246 | 6.74301 | 6.18711 | 6.23457 |
| SLC27A3 | −1.42207 | 0.0401604 | 6.42007 | 6.57998 | 5.92399 | 6.06008 |
| SLC27A4 | 1.50982 | 0.00349384 | 8.3861 | 8.39962 | 9.0218 | 8.95266 |
| SLC2A1 | 1.53009 | 0.00597657 | 10.5632 | 10.5023 | 11.1831 | 11.1097 |
| SLC35D2 | 1.41758 | 0.00826938 | 9.80488 | 9.83913 | 10.3682 | 10.2827 |
| SLC38A1 | 1.51409 | 0.00857112 | 10.5625 | 10.5572 | 11.1026 | 11.214 |
| SLC38A6 | 1.72148 | 0.00785169 | 9.63372 | 9.50136 | 10.3288 | 10.3735 |
| SLC38A7 | 1.68752 | 0.0004205 | 8.36097 | 8.35568 | 9.12849 | 9.09797 |
| SLC43A3 | −1.45776 | 0.0319343 | 9.83307 | 9.91161 | 9.42009 | 9.23709 |
| SLC44A5 | 1.49178 | 0.00127534 | 5.64022 | 5.62486 | 6.19043 | 6.22872 |
| SLC4A5 | 1.71559 | 0.00248722 | 6.85974 | 6.78251 | 7.60461 | 7.59506 |
| SLC6A6 | 1.54212 | 0.0147487 | 10.9 | 10.7476 | 11.4576 | 11.4398 |
| SLC7A11 | 1.6742 | 0.00417429 | 11.3276 | 11.2418 | 12.0501 | 12.0062 |
| SLC9A3R1 | −1.6209 | 0.0265336 | 10.5945 | 10.7692 | 9.90897 | 10.0611 |
| SLCO2B1 | 4.78102 | 0.00666086 | 7.14378 | 6.82384 | 9.14789 | 9.33437 |
| SMAD1 | 1.42941 | 0.0152356 | 8.62114 | 8.71825 | 9.22736 | 9.14288 |
| SMAD7 | 1.46374 | 0.00203687 | 5.94315 | 5.99097 | 6.52346 | 6.50997 |
| SMC2 | −1.80251 | 0.0340813 | 8.80795 | 9.01239 | 8.18464 | 7.93569 |
| SNAI2 | −1.41519 | 0.0462892 | 9.1829 | 9.05226 | 8.52597 | 8.7072 |
| SNORD14E | −1.48296 | 0.0332366 | 12.6083 | 12.4434 | 11.8902 | 12.0244 |
| SNORD50B | −2.13392 | 0.0472444 | 11.7691 | 11.388 | 10.3286 | 10.6414 |
| SNTB1 | −1.70647 | 0.00550762 | 9.80377 | 9.81148 | 9.09394 | 8.97928 |
| SNTB2 | 1.4232 | 0.0158561 | 7.87126 | 7.82312 | 8.41658 | 8.29607 |
| SNX10 | −1.5693 | 0.0378534 | 7.35401 | 7.26186 | 6.77961 | 6.53603 |
| SNX24 | 1.88329 | 0.00173068 | 6.88125 | 6.94247 | 7.80253 | 7.84771 |
| SNX25 | 1.84724 | 0.00845265 | 7.50055 | 7.43341 | 8.42707 | 8.27762 |
| SNX29 | 1.82068 | 0.0193714 | 7.77153 | 8.00432 | 8.71552 | 8.78929 |
| SOCS6 | 1.40908 | 0.0341009 | 7.43402 | 7.54641 | 8.06004 | 7.90989 |
| SORL1 | −2.54481 | 0.0365749 | 9.5184 | 9.9874 | 8.28182 | 8.52886 |
| SPC24 | −1.40855 | 0.00703209 | 7.58504 | 7.65954 | 7.10942 | 7.14675 |
| SPC25 | −2.04969 | 0.0221541 | 9.60809 | 9.85709 | 8.60198 | 8.79239 |
| SPDYA | 1.75308 | 0.00819136 | 5.835 | 5.9453 | 6.65106 | 6.74901 |
| SPHK1 | 1.67842 | 0.00304194 | 9.53456 | 9.45224 | 10.2371 | 10.2439 |
| SPIN4 | −1.5982 | 0.0445967 | 9.45543 | 9.65955 | 8.77409 | 8.98801 |
| SPIRE1 | 1.81922 | 0.00998545 | 8.98524 | 8.84907 | 9.72644 | 9.83451 |
| SPN | −2.96784 | 0.00793895 | 9.10752 | 9.3703 | 7.61923 | 7.71976 |
| SPNS1 | 1.43264 | 0.00323831 | 9.2596 | 9.21804 | 9.77856 | 9.73643 |
| SPTBN1 | 1.69593 | 0.00913187 | 10.0791 | 10.2237 | 10.9257 | 10.9012 |
| SRGAP1 | 1.41314 | 0.0452016 | 6.2357 | 6.18811 | 6.81803 | 6.60359 |
| SRXN1 | 1.44778 | 0.0291611 | 9.7957 | 9.6114 | 10.2514 | 10.2233 |
| ST3GAL6 | 1.63437 | 0.0266589 | 8.9201 | 9.06357 | 9.60676 | 9.79438 |
| STAP1 | −2.23618 | 0.00860978 | 10.3215 | 10.5379 | 9.26134 | 9.27601 |
| STARD10 | 1.54201 | 0.0181637 | 7.97325 | 7.83703 | 8.58143 | 8.47847 |
| STARD5 | −1.57655 | 0.0309711 | 10.5347 | 10.4994 | 9.74322 | 9.97727 |
| STARD9 | 1.4815 | 0.00247245 | 7.98508 | 7.95197 | 8.51269 | 8.55847 |
| STK10 | 1.63565 | 0.00840099 | 8.98938 | 9.10732 | 9.72975 | 9.78667 |
| STK40 | 1.44729 | 0.0356824 | 9.15029 | 9.24578 | 9.63952 | 9.82327 |
| STX2 | 1.62628 | 0.00288396 | 9.90617 | 9.83452 | 10.56 | 10.5838 |
| STX3 | 1.42126 | 0.00226919 | 10.8537 | 10.8146 | 11.3556 | 11.3271 |
| SUPT3H | 1.55639 | 0.00578027 | 8.7406 | 8.64498 | 9.32154 | 9.34045 |
| SVIL | 1.85417 | 0.00955355 | 8.2304 | 8.4057 | 9.21166 | 9.20598 |
| SYNJ2 | 2.77414 | 0.00250254 | 5.89756 | 5.76067 | 7.27361 | 7.3287 |
| TAGAP | −1.8081 | 0.0215543 | 8.2425 | 8.45938 | 7.56356 | 7.42938 |
| TANC2 | 2.10076 | 0.0110782 | 8.66987 | 8.88345 | 9.80863 | 9.8865 |
| TARP | −1.7872 | 0.0474214 | 6.28537 | 6.27972 | 5.63404 | 5.25564 |
| TBC1D22B | 1.42593 | 7.73E−05 | 9.74469 | 9.73619 | 10.2509 | 10.2538 |
| TBC1D25 | 1.45026 | 0.0114321 | 9.32796 | 9.24543 | 9.86354 | 9.78247 |
| TBC1D7 | 1.49173 | 0.00792583 | 8.36196 | 8.32662 | 8.96984 | 8.87271 |
| TBC1D8 | 1.69405 | 0.00027315 | 9.08905 | 9.06903 | 9.83191 | 9.84712 |
| TBC1D9 | 1.95767 | 0.00415961 | 7.76492 | 7.64065 | 8.66352 | 8.68033 |
| TBXAS1 | 1.44215 | 0.0242217 | 5.90313 | 6.07005 | 6.52158 | 6.50803 |
| TCF19 | −1.5777 | 0.0118911 | 7.05614 | 7.14825 | 6.38853 | 6.50021 |
| TCP11L2 | 1.60785 | 0.00777168 | 8.77535 | 8.80762 | 9.41805 | 9.53519 |
| TCTEX1D2 | 1.89146 | 0.0180506 | 8.02938 | 7.87428 | 8.77299 | 8.96967 |
| TDP2 | 1.42207 | 0.00471897 | 11.8008 | 11.7309 | 12.2721 | 12.2756 |
| TEP1 | 1.40825 | 0.0131831 | 9.55575 | 9.51686 | 10.0841 | 9.97633 |
| TESK2 | 1.66991 | 0.00220982 | 10.2287 | 10.2191 | 10.9291 | 10.9981 |
| TFE3 | 1.70774 | 0.0110829 | 9.90192 | 9.76822 | 10.6546 | 10.5597 |
| TFPI | 2.28473 | 0.00012845 | 8.36512 | 8.35967 | 9.54119 | 9.56765 |
| TICAM1 | 1.66266 | 0.00483304 | 7.50968 | 7.41246 | 8.17854 | 8.21057 |
| TK1 | −1.75003 | 0.00552166 | 9.23803 | 9.27401 | 8.50614 | 8.39115 |
| TLR6 | 1.75702 | 0.014572 | 6.99321 | 6.89734 | 7.8453 | 7.6715 |
| TM4SF1 | 3.00116 | 0.00854377 | 6.27123 | 6.04912 | 7.84276 | 7.64863 |
| TM4SF19 | 2.51509 | 0.00449844 | 10.8802 | 10.7092 | 12.1518 | 12.0988 |
| TM7SF3 | −2.36855 | 0.0175569 | 11.5675 | 11.8765 | 10.4144 | 10.5415 |
| TMBIM1 | 1.54094 | 0.00204242 | 11.0146 | 11.0659 | 11.6759 | 11.6522 |
| TMC7 | 1.65526 | 0.012673 | 6.07598 | 5.94334 | 6.78602 | 6.68742 |
| TMC8 | −1.90625 | 0.0413225 | 8.13707 | 8.52538 | 7.42166 | 7.37932 |
| TMCO3 | 1.52831 | 0.00210259 | 8.57812 | 8.634 | 9.21495 | 9.22103 |
| TMEFF1 | 1.54085 | 0.00484795 | 8.45571 | 8.36967 | 9.04342 | 9.02941 |
| TMEM106C | −1.42222 | 0.00443781 | 9.70741 | 9.68358 | 9.21916 | 9.15554 |
| TMEM110 | −1.41565 | 0.0294765 | 9.90642 | 10.0308 | 9.40482 | 9.52948 |
| TMEM120B | 1.78073 | 0.0124592 | 8.39106 | 8.52065 | 9.35615 | 9.2205 |
| TMEM140 | 2.16114 | 0.00543136 | 10.1205 | 9.9576 | 11.1392 | 11.1624 |
| TMEM149 | −1.44412 | 0.00136613 | 6.69495 | 6.71507 | 6.15798 | 6.19166 |
| TMEM191A | −1.57092 | 0.0409108 | 8.8259 | 8.60787 | 7.98394 | 8.14659 |
| TMEM194B | −1.52441 | 0.0128869 | 8.86108 | 8.97775 | 8.27298 | 8.34935 |
| TMEM22 | 1.51637 | 0.0354856 | 7.35895 | 7.25782 | 8.01369 | 7.80432 |
| TMEM43 | 1.44363 | 0.0022034 | 10.3852 | 10.4317 | 10.9292 | 10.947 |
| TMEM63C | −1.6374 | 0.0290751 | 6.95639 | 7.20281 | 6.35398 | 6.38241 |
| TMEM65 | 1.50125 | 0.00021554 | 8.8517 | 8.85383 | 9.44747 | 9.43039 |
| TMOD1 | 2.30161 | 0.03145 | 8.73244 | 8.3299 | 9.81876 | 9.64886 |
| TNFAIP1 | 1.43733 | 0.0122013 | 9.4638 | 9.36058 | 9.9628 | 9.90836 |
| TNFRSF10A | 1.40439 | 0.0206661 | 8.04118 | 7.91666 | 8.43362 | 8.50411 |
| TNFRSF10B | 1.61124 | 0.0140741 | 9.33748 | 9.22261 | 10.0275 | 9.90898 |
| TNFRSF12A | 1.49685 | 0.0310912 | 9.38853 | 9.24207 | 9.97259 | 9.82187 |
| TNFRSF14 | 1.98576 | 0.0130443 | 7.41141 | 7.19567 | 8.33059 | 8.25588 |
| TNFSF10 | −2.14027 | 0.00445255 | 11.544 | 11.5769 | 10.5343 | 10.391 |
| TNFSF13B | −1.84685 | 0.0328412 | 10.7086 | 10.9076 | 10.054 | 9.79203 |
| TOP2A | −1.84284 | 0.00854576 | 11.2292 | 11.3812 | 10.4542 | 10.3924 |
| TOR1B | −1.50034 | 0.0127338 | 9.67427 | 9.80506 | 9.16744 | 9.14131 |
| TPI1P2 | −1.50153 | 0.0163448 | 7.405 | 7.35307 | 6.72128 | 6.86393 |
| TRAF3IP3 | −3.01563 | 0.00158178 | 7.42251 | 7.44814 | 5.78076 | 5.90497 |
| TRAIP | −1.54204 | 0.0343968 | 8.56091 | 8.77838 | 8.09309 | 7.99652 |
| TRAM2 | 1.60189 | 0.01209 | 9.48794 | 9.55023 | 10.1302 | 10.2676 |
| TREM1 | 1.65455 | 0.0157758 | 7.53744 | 7.60988 | 8.38504 | 8.21516 |
| TRIB3 | 1.82797 | 0.00251597 | 7.77282 | 7.72652 | 8.65702 | 8.58281 |
| TRIM16 | 1.4975 | 0.0222924 | 7.25227 | 7.21358 | 7.90181 | 7.72916 |
| TRIM16L | 1.78009 | 0.00155693 | 7.42241 | 7.46263 | 8.24848 | 8.30046 |
| TRIP10 | 1.61952 | 0.0280379 | 8.74948 | 8.60839 | 9.27868 | 9.47031 |
| TRPM2 | −1.68798 | 0.0115846 | 6.84478 | 6.9871 | 6.11987 | 6.20141 |
| TRPS1 | 1.88723 | 0.0008061 | 7.86834 | 7.89907 | 8.82099 | 8.77896 |
| TRPV2 | 1.63957 | 0.022986 | 6.18235 | 6.30752 | 6.86773 | 7.04878 |
| TSC1 | 1.47491 | 0.00366181 | 9.83953 | 9.89577 | 10.4474 | 10.4091 |
| TSKU | 1.75022 | 0.00820831 | 7.38636 | 7.34302 | 8.10188 | 8.24259 |
| TSNARE1 | 1.6089 | 0.0138628 | 8.33187 | 8.33108 | 9.09918 | 8.93592 |
| TTC7B | 1.40786 | 0.0101544 | 9.8366 | 9.93138 | 10.3612 | 10.3938 |
| TTK | −1.6809 | 0.0154038 | 9.70908 | 9.64667 | 9.01739 | 8.83989 |
| TTLL1 | −1.42848 | 0.027128 | 7.29437 | 7.46572 | 6.87753 | 6.8536 |
| TTLL7 | 1.52374 | 0.0138475 | 7.14226 | 7.27619 | 7.78971 | 7.84398 |
| TULP4 | 1.71269 | 0.00891566 | 8.30937 | 8.37822 | 9.05479 | 9.18533 |
| TXNIP | −1.60419 | 0.016655 | 13.0205 | 13.1465 | 12.3386 | 12.4646 |
| TXNRD1 | 1.63793 | 0.0271101 | 11.4581 | 11.2458 | 12.119 | 12.0086 |
| TYMS | −1.753 | 0.0118026 | 10.1732 | 10.2546 | 9.48293 | 9.32516 |
| UAP1L1 | 1.56397 | 0.00403831 | 8.06939 | 8.03915 | 8.73772 | 8.66123 |
| UBE2H | 1.54222 | 0.0122286 | 10.9898 | 10.8568 | 11.5694 | 11.5272 |
| UBE2T | −1.57051 | 0.0353166 | 10.0492 | 9.99422 | 9.24777 | 9.49317 |
| UCP2 | −1.44291 | 0.0489591 | 10.954 | 11.0264 | 10.3452 | 10.5772 |
| UHRF1 | −1.80279 | 0.00580912 | 6.79184 | 6.91361 | 6.0255 | 5.97948 |
| UIMC1 | −1.76222 | 0.0489812 | 10.0793 | 10.0102 | 9.41202 | 9.04268 |
| UNC93B1 | −1.47747 | 0.0473739 | 8.92092 | 9.17239 | 8.46475 | 8.5023 |
| UQCRH | −1.45347 | 0.0115706 | 7.16718 | 7.25488 | 6.71031 | 6.63274 |
| USP17 | −1.9499 | 0.0449593 | 7.5898 | 7.18815 | 6.49185 | 6.3593 |
| USP17L6P | −2.33903 | 0.0186768 | 7.98545 | 7.64602 | 6.59828 | 6.58137 |
| USP31 | 1.5557 | 0.0169764 | 8.6916 | 8.75012 | 9.43731 | 9.27952 |
| USP35 | 1.43767 | 0.0132918 | 6.10309 | 5.9919 | 6.54614 | 6.59631 |
| USP53 | 1.45062 | 0.00467158 | 10.9628 | 10.9367 | 11.5208 | 11.452 |
| USP54 | 1.68289 | 0.00051094 | 8.69614 | 8.6806 | 9.45442 | 9.42422 |
| VAC14 | 1.44712 | 0.0393538 | 9.20023 | 9.4175 | 9.83298 | 9.85113 |
| VAMP8 | −1.54865 | 0.0269179 | 11.7341 | 11.945 | 11.2157 | 11.2014 |
| VAT1 | 1.85995 | 0.00366324 | 10.089 | 9.98168 | 10.9391 | 10.9221 |
| VAV1 | −1.93243 | 0.035353 | 8.46097 | 8.71758 | 7.50753 | 7.77019 |
| VDR | −1.46164 | 0.0158719 | 7.13761 | 7.2133 | 6.68654 | 6.56919 |
| VRK1 | −1.5175 | 0.0101822 | 10.0307 | 10.0923 | 9.51266 | 9.40696 |
| WDR19 | 1.58088 | 0.0155719 | 9.28353 | 9.17741 | 9.95558 | 9.82682 |
| WDR76 | −1.68386 | 0.0285046 | 9.1099 | 9.35319 | 8.5248 | 8.43475 |
| WEE1 | −1.43879 | 0.0165958 | 10.7675 | 10.6787 | 10.2504 | 10.1461 |
| WIPF3 | −3.46629 | 0.0163805 | 8.2735 | 8.7115 | 6.62135 | 6.77686 |
| WWC2 | 1.5746 | 0.00627344 | 8.31924 | 8.255 | 8.98315 | 8.90106 |
| WWTR1 | 1.40489 | 0.00218077 | 8.71742 | 8.67161 | 9.18368 | 9.18626 |
| XIAP | 1.61936 | 0.00457977 | 8.74439 | 8.66619 | 9.42719 | 9.37423 |
| XRCC2 | −1.44428 | 0.0183644 | 10.1575 | 10.2588 | 9.73021 | 9.62543 |
| XRCC6BP1 | −1.7268 | 0.00156151 | 9.16238 | 9.22473 | 8.4049 | 8.406 |
| YBX1P2 | −1.45693 | 0.0101959 | 10.6443 | 10.6337 | 10.0411 | 10.1511 |
| ZBTB38 | 1.77585 | 0.0272737 | 8.12519 | 8.30463 | 9.1505 | 8.93633 |
| ZC3H12C | 2.0913 | 0.00351807 | 8.74843 | 8.64939 | 9.80274 | 9.72388 |
| ZCCHC12 | −1.47322 | 0.0239141 | 6.72802 | 6.55345 | 6.07038 | 6.09315 |
| ZDHHC14 | 1.64146 | 0.0313795 | 7.45952 | 7.29372 | 8.19138 | 7.99182 |
| ZFP36L2 | −1.41492 | 0.00429031 | 11.3799 | 11.3889 | 10.9163 | 10.8511 |
| ZNF521 | 1.77677 | 0.0436116 | 5.17123 | 5.34414 | 5.9301 | 6.24378 |
| ZNF529 | 1.46144 | 0.00815283 | 8.11388 | 8.20162 | 8.72855 | 8.68172 |
| ZNF589 | 1.46839 | 0.027631 | 9.52734 | 9.36789 | 9.95188 | 10.0518 |
| ZNF609 | 1.42586 | 0.00351117 | 9.54718 | 9.55125 | 10.0914 | 10.0307 |
| ZNF675 | −1.41471 | 0.0167937 | 10.8592 | 10.7335 | 10.3151 | 10.2766 |
| ZNF724P | −1.4874 | 0.0251815 | 10.1183 | 10.1043 | 9.44611 | 9.63089 |
| ZNF730 | −1.53005 | 0.012227 | 7.82789 | 7.94853 | 7.24222 | 7.30704 |
| ZNF749 | 1.49302 | 0.00250312 | 7.62476 | 7.66793 | 8.24392 | 8.20523 |
| ZNF76 | 1.425 | 0.00347843 | 8.54245 | 8.4927 | 9.04568 | 9.01139 |
| ZNF774 | 1.61962 | 0.00098069 | 8.03433 | 7.99923 | 8.72537 | 8.6995 |
| ZNF850P | −1.42489 | 0.0373898 | 8.31313 | 8.38502 | 7.93332 | 7.74314 |
| ZSCAN20 | 1.56562 | 0.00062514 | 6.99349 | 6.97119 | 7.6408 | 7.61736 |

TABLE 7
Genes showing differential expression in Kasumi-1^{RX1-KD+Z} (listed in
Table 6, above) are functionally enriched for cell cycle and mitotic functions
as indicated by DAVID (p-value = 3.31E−10, FDR = 3.16E 07).
Similar results were obtained using IPA and GSEA (unpublished analyses).

| Gene Symbol | Transcript ID | Gene Name |
|---|---|---|
| BUB1 | 8054580 | budding uninhibited by benzimidazoles 1 homolog (ye |
| BUB1B | 7982663 | budding uninhibited by benzimidazoles 1 homolog bet |
| CASC5 | 7982757 | cancer susceptibility candidate 5 |
| CCNA2 | 8102643 | cyclin A2 |
| CCNB2 | 7983969 | cyclin B2 |
| Ccne2 | 8151871 | cyclin E2 |
| cdc20 | 7900699 | cell division cycle 20 homolog (S. cerevisiae) |
| Cdc45 | 8071212 | CDC45 cell division cycle 45-like (S. cerevisiae) |
| cdc6 | 8007071 | cell division cycle 6 homolog (S. cerevisiae) |
| CDC7 | 7902913 | cell division cycle 7 homolog (S. cerevisiae) |
| CDCA8 | 7900167 | cell division cycle associated 8 |
| Cdt1 | 7997839 | chromatin licensing and DNA replication factor 1 |
| CENPE | 8102076 | centromere protein E, 312 kDa |
| CENPF | 7909708 | centromere protein F, 350/400ka (mitosin) |
| CENPI | 8168794 | centromere protein I |
| cenpm | 8076393 | centromere protein M |
| Cep110 | 8157534 | centrosomal protein 110 kDa |
| CLIP1 | 7967255 | CAP-GLY domain containing linker protein 1 |
| CSNK1E | 8076056 | casein kinase 1, epsilon |
| Dhfr | 8112902 | dihydrofolate reductase |
| Dhfr | 8022640 | dihydrofolate reductase |
| Dhfr | 8112914 | dihydrofolate reductase |
| Gins1 | 8061471 | GINS complex subunit 1 (Psf1 homolog) |
| GINS2 | 8003204 | GINS complex subunit 2 (Psf2 homolog) |
| gmnn | 8117225 | geminin, DNA replication inhibitor |
| HSP90AA1 | 8103722 | heat shock protein 90 kDa alpha (cytosolic), |
| HSP90AA2 | 8103722 | heat shock protein 90 kDa alpha (cytosolic), |
| kif20a | 8108301 | kinesin family member 20A |
| KIF23 | 7984540 | kinesin family member 23 |
| KIF2C | 7901010 | kinesin family member 2C |
| KNTC1 | 7959408 | kinetochore associated 1 |
| Mcm10 | 7926259 | minichromosome maintenance complex component 10 |
| MCM6 | 8055426 | minichromosome maintenance complex component 6 |
| MLF1IP | 8103932 | MLF1 interacting protein |
| NDC80 | 8019857 | NDC80 homolog, kinetochore complex component (S. ce |
| NEK2 | 7924096 | NIMA (never in mitosis gene a)-related kinase 2 |
| NUF2 | 7906930 | NUF2, NDC80 kinetochore complex component, homolog |
| Ore11 | 7916167 | origin recognition complex, subunit 1-like (yeast) |
| pcnA | 8064844 | proliferating cell nuclear antigen |
| PLK1 | 7994109 | polo-like kinase 1 (Drosophila) |
| pola2 | 7941214 | polymerase (DNA directed), alpha 2 (70 kD subunit) |
| Pole | 7967736 | polymerase (DNA directed), epsilon |
| PPP2R5B | 7941087 | protein phosphatase 2, regulatory subunit B′, beta |
| PRIM1 | 7964271 | primase, DNA, polypeptide 1 (49 kDa) |
| PRIM2 | 8120411 | primase, DNA, polypeptide 2 (58 kDa) |
| RfC4 | 8092640 | replication factor C (activator 1) 4, 37 kDa |
| rrm2 | 8040223 | ribonucleotide reductase M2 polypeptide |
| sgol1 | 8085754 | shugoshin-like 1 (S. pombe) |
| Ska1 | 8021187 | chromosome 18 open reading frame 24 |
| SKA2 | 8157691 | family with sequence similarity 33, member A; simil |
| Spc24 | 8034122 | SPC24, NDC80 kinetochore complex component, homolog |
| spc25 | 8056572 | SPC25, NDC80 kinetochore complex component, homolog |
| Tyms | 8019842 | thymidylate synthetase |

### Example 7

### Opposing Effects of RUNX1 and A-E on SAC Signaling

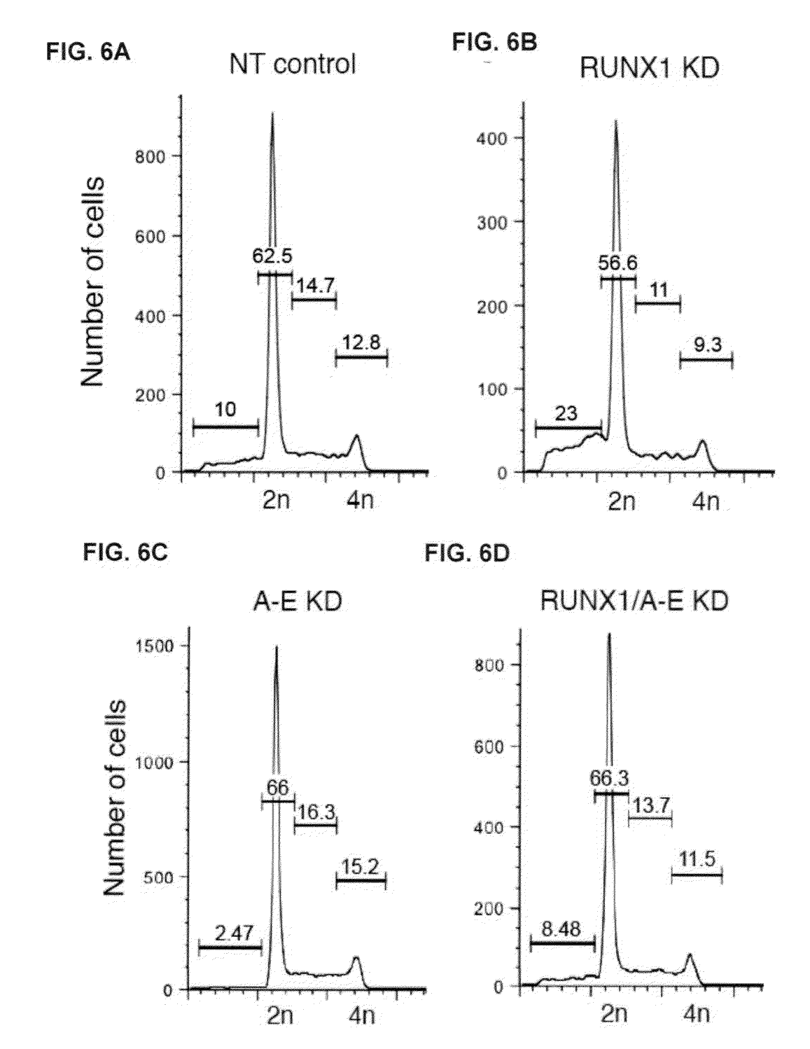

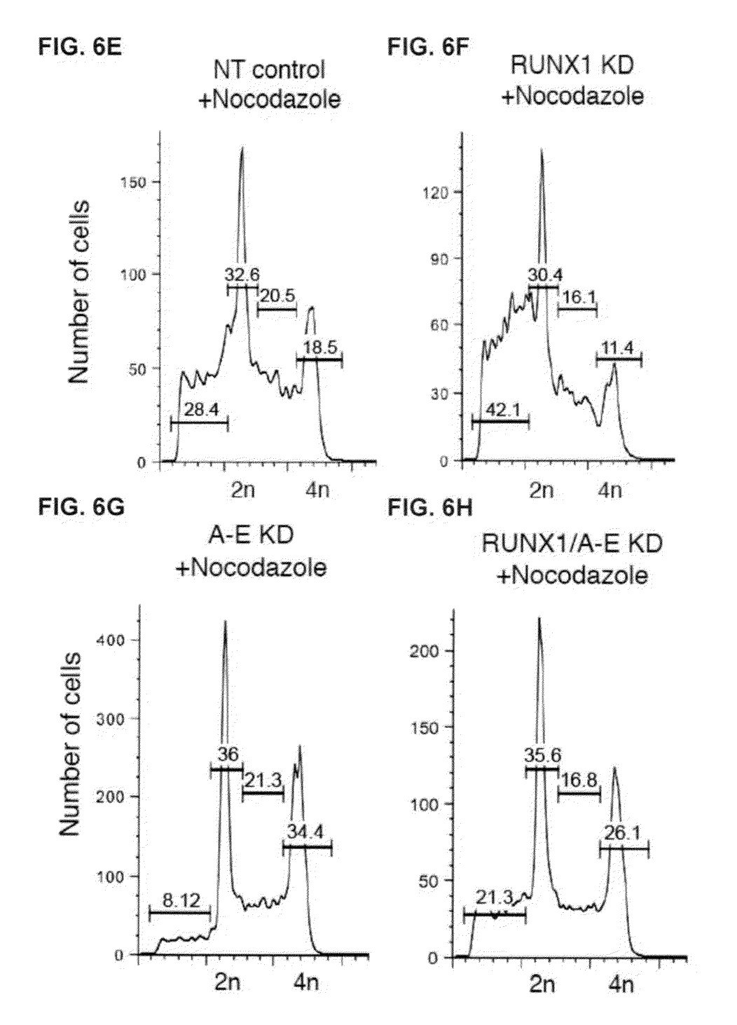

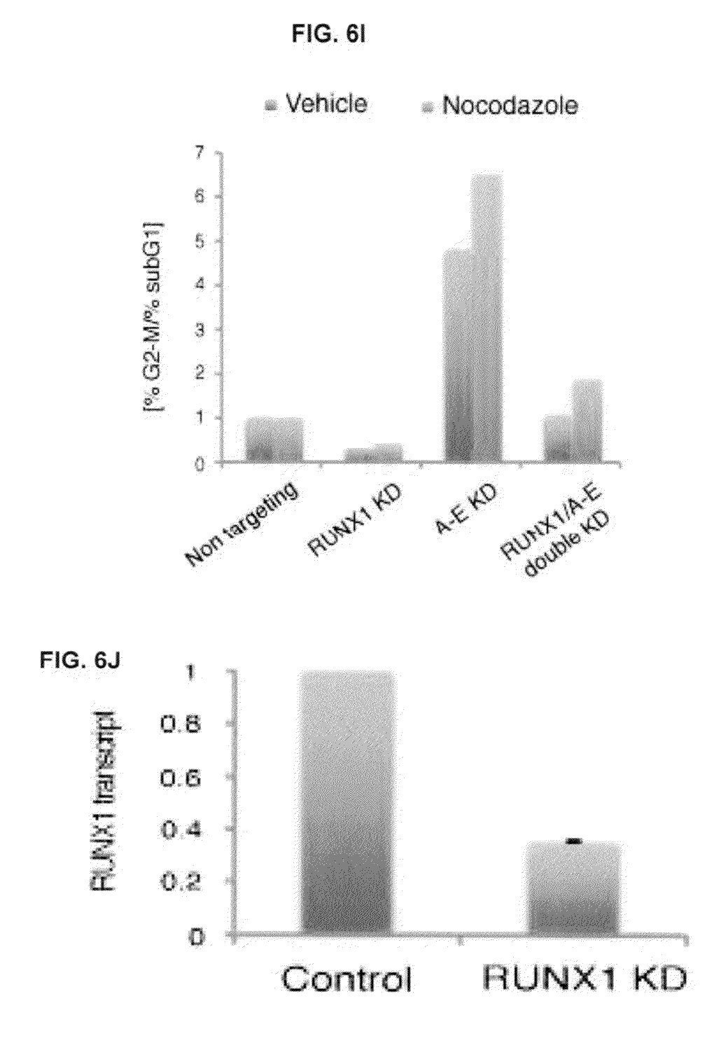

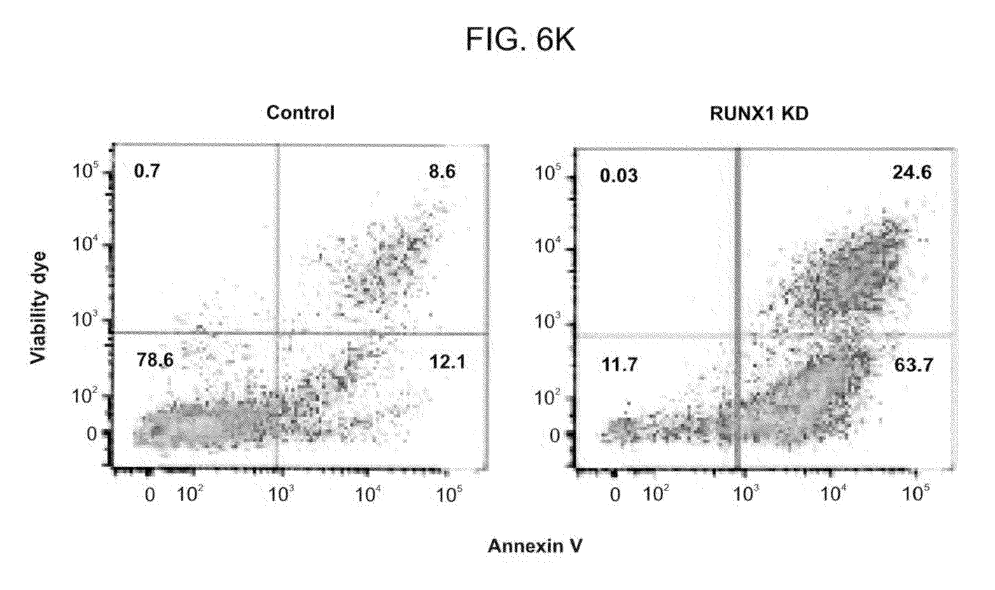

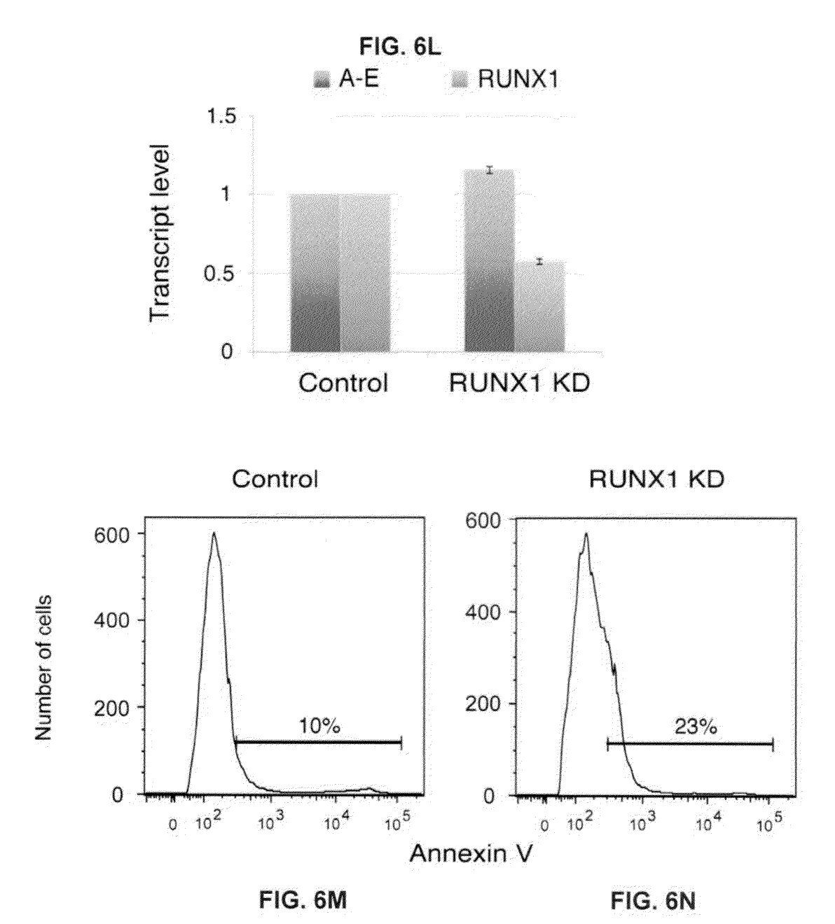

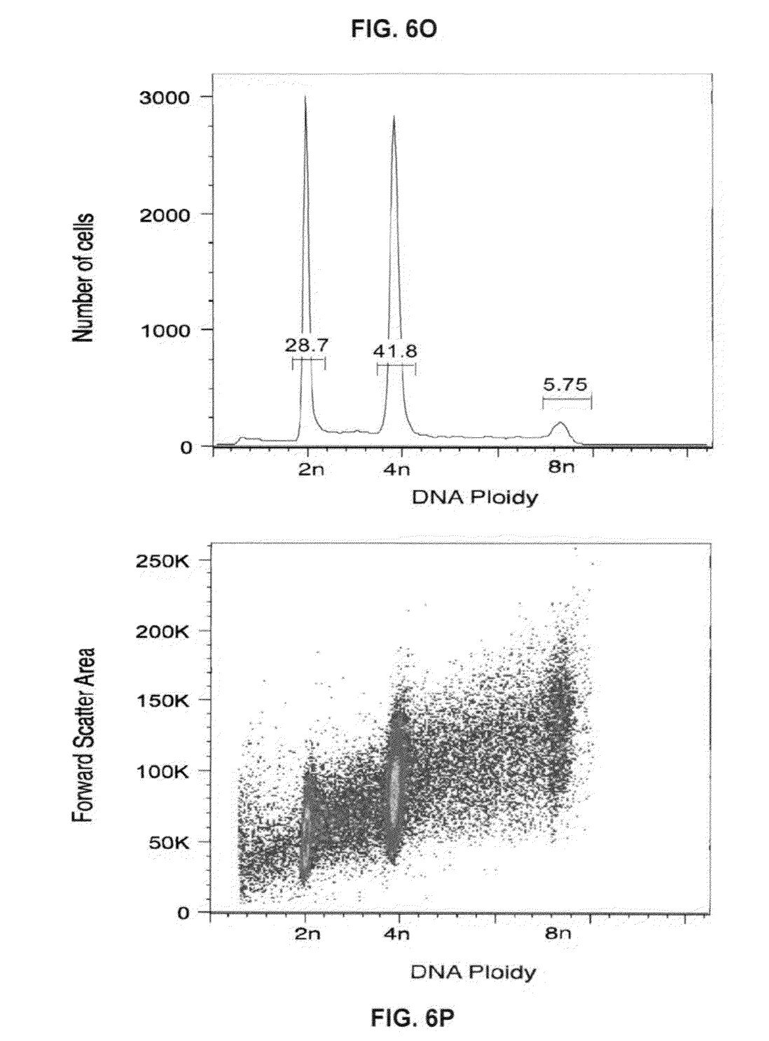

As a sensitive measurement of SAC activity, the microtubule-depolarizing agent Nocodazole (NOC) was used, which induces SAC causing cell arrest at M phase. The question asked was how the induced change in RUNX1 and A-E levels affects SAC signaling. Accordingly, the cell cycle of NOC-treated Kasumi-1^{Cont}, Kasumi-1^{RX1-KD}, Kasumi-1^{A-E-KD} and double-KD Kasumi-1^{RX1/A-E-KD} cells was characterized compared to cells treated with vehicle (FIGS. 6A-6H).

Overall, the ability of NOC-treated cells to arrest cell cycle at M phase was inversely correlated with the proportion of dead/apoptotic cells accumulated in subG1 (FIGS. 6E-6H). Specifically, NOC-treated Kasumi-1^{RX1-KD} and Kasumi-1^{A-E-KD} cells respectively displayed diminished or elevated capacity to arrest at M-phase, compared to NOC-treated Kasumi-1^{Cont} cells. Consequently, the proportion of their subG1 populations was increased (Kasumi-1^{RX1-KD}) or decreased (Kasumi-1^{A-E-KD}) (FIGS. 6E-6H). Of particular relevance to this finding is the observation that cells expressing a C-terminal truncated isoform of A-E, designated A-Etr, display enhanced mitotic progression upon NOC treatment [Boyapati, A. et al., Blood (2007) 109, 3963-3971].

The complementary outcomes of these experiments suggest that while RUNX1 positively regulates SAC activity, A-E represses it. The findings that KD of A-E in Kasumi-1^{RX1-KD} cells (Kasumi-1^{RX1/AE-KD}) restored SAC activity (FIGS. 6E-6H) and rescued cells from apoptosis (FIGS. 2C-2G) support this conclusion. Significantly, the opposing regulatory effects of A-E and RUNX1 on cellular gene expression noted above is reflected here in their impact on cell capacity to arrest at M-phase and avoid cell death (FIG. 6I). Thus, a threshold of WT RUNX1 activity is essential in t(8;21) AML cells to counter A-E-mediated inhibition of SAC signaling to prevent complete disruption of SAC and subsequent apoptosis, possibly due to mitotic catastrophe.

### Example 8

### RUNX1 Activity is Also Required for Survival of Inv(16) ME-1 Cell Line

Next, the present inventors addressed whether the addiction of t(8;21) Kasumi-1 cell line to RUNX1 constitutes a common phenomenon in an additional sub-type of human acute myeloid leukemia also associated with partial loss of RUNX1 function. This AML sub-type known as inv(16)⁺ is characterized by an inversion of chromosome 16 consequently leading both to decreased expression and reduced activity of CBFβ, a protein factor critical for RUNX1 function.

Using the inv(16) AML cell line ME-1 [Yanagisawa, K. et al., Blood (1991) 78, 451-457], the impact of RUNX1 KD on cell survival was examined Significantly, RUNX1 KD (FIG. 6J) produced a marked increase in Annexin-V staining of both viable and nonviable cells (FIG. 6K), indicating RUNX1 KD-mediated enhancement of apoptosis.

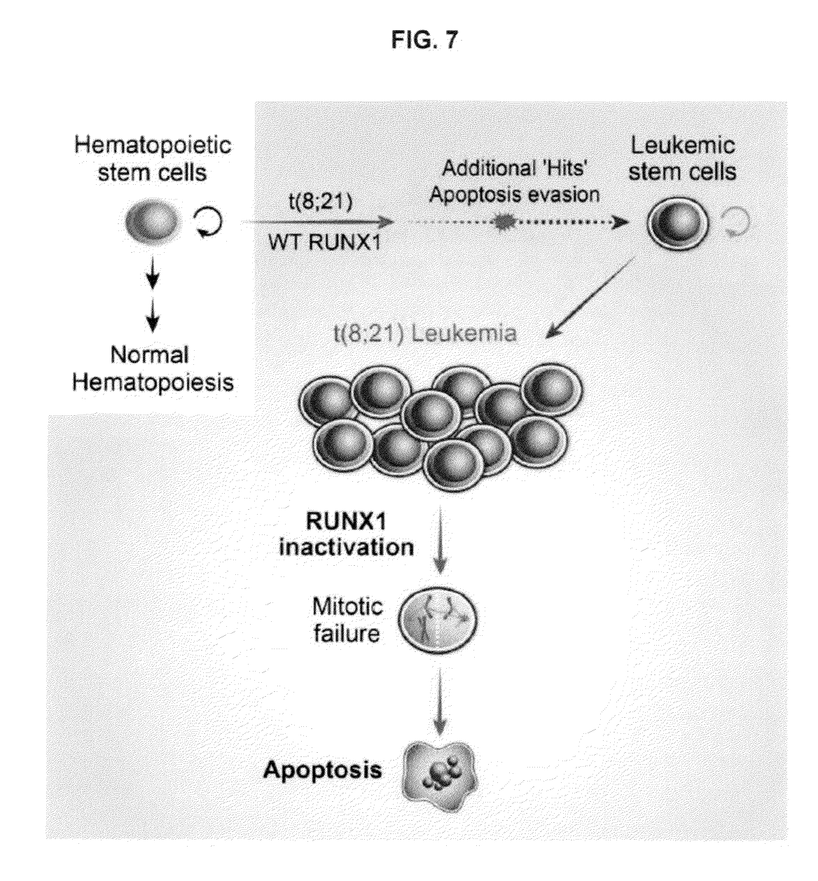

To evaluate the involvement of WT RUNX1 in the development of A-E-mediated preleukemic cell phenotype, a preleukemic cell model was used of human CD34⁺ progenitor cells transduced by A-E expressing lentivirus (as described in detail in the ‘materials and experimental procedures’ section above). Transfection of CD34⁺/A-E cells with siRNA against RUNX1 (FIG. 7) resulted in reduced expression of RUNX1 associated with an increased proportion of Annexin-V⁺ positive cells as compared with cells transfected with control NT siRNA (FIGS. 6L-6N). This finding indicates that RUNX1 activity is required for the preleukemic CD34⁺/A-E cells viability and underscores the critical importance of the RUNX1/A-E balance for the leukemogenic process.

Cell-cycle analysis of untreated ME-1 cells identified a mixed population of diploid and tetraploid cells (FIG. 6O-6P), characteristic of cells with attenuated mitotic functions. This abnormal ME-1 cell-cycle profile rendered recording cell death using DNA content analysis unfeasible. The data suggest that the inv(16) ME-1 cell viability, similarly to that of t(8;21) Kasumi-1 cell line, physiologically depends on RUNX1 activity. The observations that inv(16) AML patients have no inactivating mutations in RUNX1 [Goyama, S. and Mulloy J C. (2011), supra] or in CBFβ [Heilman, S. et al., Cancer Res (2006) 66, 11214-11218] support this conclusion.

### DISCUSSION

The evolvement of cancer cells involves acquisition of several hallmark capabilities, including accelerated proliferation, self-renewal and evasion of apoptosis. The prevailing notion is that t(8;21) AML is initiated by chromosomal translocation that occurs in bone marrow (BM) hematopoietic stem cells (HSCs). The resulting pre-leukemic stem cells (Pre-LSC) that express the oncogenic fusion protein self-renew and persist in BM. During AML development, these Pre-LSC undergo clonal transformation in a multistep process involving additional genetic alterations that abrogate cell-growth regulations. The role of the chimeric A-E protein in the etiology of t(8;21) AML has been widely studied. However, the importance of native RUNX1 for the development of t(8;21) or inv(16) AML subtypes remained obscure.

In the present study it was shown that expression of native RUNX1 is crucial for the survival of t(8;21) Kasumi-1 and inv(16) ME-1 AML leukemic cell-lines, so that RUNX1 KD evoked apoptotic cell death. The medical significance of this leukemic-cell addiction to native RUNX1 is underscored by clinical data [Goyama, S. and Mulloy J C. (2011), supra] showing that active RUNX1 is usually maintained in t(8;21) and inv(16) AML patients whereas, the gene is frequently inactivated in other forms of AML [Schnittger, S. et al., Blood (2011) 117, 2348-2357]. Furthermore, WT RUNX1 is not only preserved, but frequently amplified among patients with t(12;21) B-cell acute lymphoblastic leukemia (ALL), suggesting that WT RUNX1 is also instrumental in t(12;21) ALL development. Yet a different mechanism underlies the requirement of RUNX1 expression for cell growth of the t(4;11) mixed lineage leukemia (MLL) MV4-11 and SEM cell-lines.

Using Z-VAD-FMK and ImageStream© System analysis, it was demonstrated herein that RUNX1 KD-induced Kasumi-1 cell death is caspase-dependent and associated with mitochondrial membrane depolarization. Significantly, this cell death involves A-E gain-of-function activity shown by the complete rescue from apoptosis upon A-E KD in Kasumi-1^{RX1-KD} cells. Consistent with the involvement of A-E in Kasumi-1^{RX1-KD} cell death, ChIP-seq and gene expression data demonstrated opposing effects of RUNX1 and A-E on their common target genes. Moreover, it was shown herein that RUNX1 can modulate the expression of A-E uniquely regulated genes, suggesting that RUNX1 and A-E compete for common cooperating TFs. Upon RUNX1 KD, these TFs might be recruited by A-E leading to aberrant expression of RUNX1 uniquely regulated genes. This regulatory mechanism drives the overall alterations in gene expression characterizing Kasumi-1^{RX1-KD} cells. Compatible with this interpretation, uniquely bound A-E and RUNX1 regions are enriched for the motif of ETS TF family members that interact with the common DNA-binding domain of RUNX1 and A-E.

The notion of A-E involvement in Kasumi-1^{RX1-KD} cell death corresponds with the findings that A-E has inherent pro-apoptotic activity [Lu, Y. et al., Leukemia (2006) 20, 987-993], that opposes its leukemogenicity. The present data suggests that WT RUNX1 counters this pro-apoptotic activity and thereby contributes to long-term survival of t(8;21) pre-leukemic HSCs and consequently to leukemia development. Indeed, RUNX1 is highly expressed in CD34⁺ long-term HSCs where it transcriptionally regulates CD34 expression [Levantini, E. et al., EMBO J (2011) 30, 4059-4070]. Moreover, A-E-transduced CD34⁺ hematopoietic cells yield highly proliferative cytokine-dependent cultures [Mulloy, J. et al., Blood (2003) 102, 4369-4376], suggesting that the pro-apoptotic activity of A-E in CD34⁺ HSCs is attenuated. Similarly, ectopic expression of C-S in cultured CD34⁺ hematopoietic cells produced long-term cell lines [Wunderlich, M. et al., Blood (2006) 108, 1690-1697]. This finding is compatible with the present observation that RUNX1 is also required for survival of inv(16) leukemic cell line ME-1. It also supports the conclusion that development of A-E- or C-S-mediated leukemia (CBF-leukemias) depends on a delicate balance between the oncogenic impact of the chimeric A-E and C-S proteins and anti-apoptotic activity of RUNX1. Accordingly, the two deletion mutants, A-E9a and CBFβ-SMMHC_{d179-221}, which accelerate leukemia development in mice, have a lower capacity to inhibit RUNX1 activity [Kamikubo, Y. et al., Cancer Cell (2010) 17, 455-468], attests to the crucial role of WT RUNX1 in the etiology of CBF-leukemia. Collectively, the data indicates that RUNX1 effectively inhibits the chimeric protein-mediated apoptosis in leukemic cell lines, but at which step?

A large number of studies have reported that RUNX1 plays an important role in cell-cycle control by promoting G1 to S progression [reviewed in Friedman, A. J Cell Physiol (2009) 219, 520-524]. The present study revealed that RUNX1 KD in Kasumi-1 cell-line caused enhanced A-E activity, resulting in decreased expression of key mitosis-regulatory genes. The aberrant expression of these RUNX1-regulated genes compromises mitotic functions including SAC activity leading to apoptosis. This finding uncovers a previously unknown role of RUNX1 as regulator of SAC functions and explains its importance for the viability of Kasumi-1, and likely ME-1, leukemic cell lines. Of note, RUNX1 activity increases during G2/M due to Cdk-mediated phosphorylation of the protein [Friedman (2009), supra]. During M phase, the SAC maintains genomic stability by delaying cell division until accurate chromosome segregation is achieved. Defects in SAC function generate aneuploidy that could facilitate tumorigenesis. Therefore, it is possible that the initial reduction of RUNX1 activity in BM HSCs by t(8;21) translocation contributes to the accumulation of additional genetic alterations required for onset of leukemia (FIG. 7).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a hematological malignancy associated with an altered RUNX1 activity or expression, the method comprising administering to a subject in need thereof a therapeutically effective amount of an agent which directly downregulates an activity or expression of RUNX1, thereby treating the hematological malignancy associated with the altered RUNX1 activity or expression.

2. The method of claim 1, wherein said RUNX1 is as set forth in SEQ ID NO: 44, 56 or 58.

3. The method of claim 1, wherein said agent which downregulates said activity or expression of RUNX1 does not substantially affect an activity or expression of the altered RUNX1.

4. The method of claim 1, wherein said hematological malignancy is a leukemia or lymphoma.

5. The method of claim 4, wherein said leukemia is an acute myeloid leukemia (AML) or an acute lymphoblastic leukemia (ALL).

6. The method of claim 5, wherein said AML is selected from the group consisting of type t(8;21), type inv(16) and type t(3;21).

7-9. (canceled)

10. The method of claim 5, wherein said ALL is type t(12;21).

11. The method of claim 1, wherein said agent is a polynucleotide agent or a small molecule.

12. (canceled)

13. The method of claim 11, wherein said polynucleotide agent is directed to a nucleic acid region selected from the group consisting of SEQ ID NO: 43, SEQ ID NO: 45, SEQ ID NO: 47, SEQ ID NO: 49, SEQ ID NO: 50, SEQ ID NO: 55 and SEQ ID NO: 57.

14. The method of claim 11, wherein said polynucleotide agent comprises 15-25 nucleotides.

15. The method of claim 11, wherein said polynucleotide agent is selected from the group consisting of SEQ ID NO: 52 and SEQ ID NO: 53.

16. (canceled)

17. The method of claim 1, wherein said RUNX1 is a wild-type RUNX1.

18. The method of claim 1, wherein said therapeutically effective amount initiates apoptosis of hematopoietic cells of said hematological malignancy.

19. The method of claim 18, wherein said apoptosis is caspase dependent.

20. (canceled)

21. The method of claim 1, further comprising administering to the subject a pro-apoptotic agent for targeted killing of the hematological malignancy.

22. The method of claim 21, wherein said pro-apoptotic agent is caspase dependent.

23-24. (canceled)

25. A method of inducing apoptosis of hematopoietic cells associated with an altered RUNX1 activity or expression, the method comprising administering to the hematopoietic cells a therapeutically effective amount of an agent which directly downregulates an activity or expression of RUNX1, thereby inducing the apoptosis of the hematopoietic cells.

26-30. (canceled)

31. An isolated polynucleotide which directly downregulates RUNX1 but not AML1-ETO (A-E), AML1-EVI1 or ETV6-RUNX1 (TEL/AML1).

32. The isolated polynucleotide of claim 31, wherein said polynucleotide comprises a nucleic acid sequence as set forth in SEQ ID NO: 52 or SEQ ID NO: 53.

33. A nucleic acid construct comprising the isolated polynucleotide of claim 31.

34. A pharmaceutical composition comprising the isolated polynucleotide of claim 31 and a pharmaceutically acceptable carrier.

35-37. (canceled)

38. A pharmaceutical composition comprising the isolated polynucleotide of claim 31, a pro-apoptotic agent and a pharmaceutically acceptable carrier.


SEQUENCE LISTING (73 sequences)

SEQ ID NO: 1 - DNA, 20 bp, Artificial sequence
  Single strand DNA oligonucleotide
tctgcagaactttccagtcg

SEQ ID NO: 2 - DNA, 20 bp, Artificial sequence
  Single strand DNA oligonucleotide
aaggcgcctggatagtgcat

SEQ ID NO: 3 - DNA, 22 bp, Artificial sequence
  Single strand DNA oligonucleotide
cacctaccacagagccatcaaa

SEQ ID NO: 4 - DNA, 22 bp, Artificial sequence
  Single strand DNA oligonucleotide
atccacaggtgagtctggcatt

SEQ ID NO: 5 - DNA, 20 bp, Artificial sequence
  Single strand DNA oligonucleotide
tgtatacccctggtgggaga

SEQ ID NO: 6 - DNA, 20 bp, Artificial sequence
  Single strand DNA oligonucleotide
tcataactccggtccctctg

SEQ ID NO: 7 - DNA, 19 bp, Artificial sequence
  Single strand DNA oligonucleotide
ctgcccagatcgtgtgctc

SEQ ID NO: 8 - DNA, 22 bp, Artificial sequence
  Single strand DNA oligonucleotide
aggttgagggtcaggacttttt

SEQ ID NO: 9 - DNA, 22 bp, Artificial sequence
  Single strand DNA oligonucleotide
tttacctgggctcaatggtttg

SEQ ID NO: 10 - DNA, 21 bp, Artificial sequence
  Single strand DNA oligonucleotide
tgcatcgccgttgataattgt

SEQ ID NO: 11 - DNA, 20 bp, Artificial sequence
  Single strand DNA oligonucleotide
gcagacagaccaggaaggag

SEQ ID NO: 12 - DNA, 20 bp, Artificial sequence
  Single strand DNA oligonucleotide
aggtgaactggaaccacagg

SEQ ID NO: 13 - DNA, 22 bp, Artificial sequence
  Single strand DNA oligonucleotide
ccaccctcaatataatcagcgg

SEQ ID NO: 14 - DNA, 23 bp, Artificial sequence
  Single strand DNA oligonucleotide
gtttcgattccgtctgactcttc

SEQ ID NO: 15 - DNA, 20 bp, Artificial sequence
  Single strand DNA oligonucleotide
tgcgtcctctctcaggagtg

SEQ ID NO: 16 - DNA, 20 bp, Artificial sequence
  Single strand DNA oligonucleotide
ggtccatgatgtcgtcagcc

SEQ ID NO: 17 - DNA, 22 bp, Artificial sequence
  Single strand DNA oligonucleotide
ctttcaaccactagcactagcc

SEQ ID NO: 18 - DNA, 22 bp, Artificial sequence
  Single strand DNA oligonucleotide
tgccctgagtcaatttcacttc

SEQ ID NO: 19 - DNA, 21 bp, Artificial sequence
  Single strand DNA oligonucleotide
agactgccaaagtgtatggga

SEQ ID NO: 20 - DNA, 21 bp, Artificial sequence
  Single strand DNA oligonucleotide
gcaaggtacggtctgagttcc

SEQ ID NO: 21 - DNA, 22 bp, Artificial sequence
  Single strand DNA oligonucleotide
caggactgtgtcaaggagatcg

SEQ ID NO: 22 - DNA, 21 bp, Artificial sequence
  Single strand DNA oligonucleotide
atgttcagctcgttgtcttcg

SEQ ID NO: 23 - DNA, 23 bp, Artificial sequence
  Single strand DNA oligonucleotide
tggaaagcaaacagtaaacagcc

SEQ ID NO: 24 - DNA, 21 bp, Artificial sequence
  Single strand DNA oligonucleotide
gggcatcttcacgctctattt

SEQ ID NO: 25 - DNA, 20 bp, Artificial sequence
  Single strand DNA oligonucleotide
ccgacggtgtccagtgattt

SEQ ID NO: 26 - DNA, 22 bp, Artificial sequence
  Single strand DNA oligonucleotide
tgttgttttggtgggttgaact

SEQ ID NO: 27 - DNA, 22 bp, Artificial sequence
  Single strand DNA oligonucleotide
gaccctaagaatcctgagagcc

SEQ ID NO: 28 - DNA, 23 bp, Artificial sequence
  Single strand DNA oligonucleotide
ggggcactatctgacacttcata

SEQ ID NO: 29 - DNA, 21 bp, Artificial sequence
  Single strand DNA oligonucleotide
gtgcccctcatacgaacttcc

SEQ ID NO: 30 - DNA, 21 bp, Artificial sequence
  Single strand DNA oligonucleotide
gtgcaaaaggatacccaaggt

SEQ ID NO: 31 - DNA, 21 bp, Artificial sequence
  Single strand DNA oligonucleotide
aactcagcagtcacctcatct

SEQ ID NO: 32 - DNA, 21 bp, Artificial sequence
  Single strand DNA oligonucleotide
tgcacctacgtttaggcagag

SEQ ID NO: 33 - DNA, 20 bp, Artificial sequence
  Single strand DNA oligonucleotide
gcaccgacaattccaagctc

SEQ ID NO: 34 - DNA, 21 bp, Artificial sequence
  Single strand DNA oligonucleotide
tgtgcttcgttgtggtacaga

SEQ ID NO: 35 - DNA, 22 bp, Artificial sequence
  Single strand DNA oligonucleotide
acaatcaacggagaaagcatga

SEQ ID NO: 36 - DNA, 20 bp, Artificial sequence
  Single strand DNA oligonucleotide
ctccaccacctgatgcaact

SEQ ID NO: 37 - DNA, 22 bp, Artificial sequence
  Single strand DNA oligonucleotide
tggctgtggtattgtagaaagc

SEQ ID NO: 38 - DNA, 21 bp, Artificial sequence
  Single strand DNA oligonucleotide
ttggcatcatcgagtttggga

SEQ ID NO: 39 - DNA, 22 bp, Artificial sequence
  Single strand DNA oligonucleotide
tgcttcgtgaactgaaacatcc

SEQ ID NO: 40 - DNA, 23 bp, Artificial sequence
  Single strand DNA oligonucleotide
ccagagtcaactgagtcatcact

SEQ ID NO: 41 - DNA, 20 bp, Artificial sequence
  Single strand DNA oligonucleotide
ggacttcgagcaagagatgg

SEQ ID NO: 42 - DNA, 20 bp, Artificial sequence
  Single strand DNA oligonucleotide
agcactgtgttggcgtacag

SEQ ID NO: 43 - DNA, 6212 bp, Homo sapiens
ggaaaggggaacagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtgagcatcaccaacccacagccaaggcggcgctggcttttttttttttttaatctttaacaatttgaatatttgtttttacaaaggtgcattttttaatagggcttggggagtcccagaggtatccagcagaggggagaagaaagagagatgtagggctagaggggtgaggctgaaacagtgacctgtcttggttttcgctccgaaggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccacgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcgggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaacttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaatgcttctttctaactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacata

SEQ ID NO: 44 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 45 - DNA, 502 bp, Homo sapiens
  misc_feature: RUNX1 target sequence - Target mRNA sequence harbored specifically by the wild-type (non-translocated) RUNX1 transcript in t(12;21) ALL cells
ggaaaggggaacagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtgagcatcaccaacccacagccaaggcggcgctggcttttttttttttttaatctttaacaatttgaatatttgtttttacaaaggtgcattttttaatagggcttggggagtcccagaggtatccagcagaggggagaagaaagagagatgtagggctagaggggtgaggctgaaacagtgacctgtcttggttttcgctccgaaggtaaaagaaatcattgagtcccccgccttcagaagagggtgcattttcaggaggaagcgatggcttcagacagcatatttgagtcatttccttcgtacccacagtgcttcatgagag

SEQ ID NO: 46 - protein, 20 aa, Homo sapiens
  misc_feature: Amino acid of RUNX1 target sequence
MASDSIFESFPSYPQCFMRE

SEQ ID NO: 47 - DNA, 5155 bp, Homo sapiens
  misc_feature: RUNX1 target sequence - Target mRNA sequence harbored specifically by the wild-type (non-translocated) RUNX1 transcript in t(8;21) AML cells
gacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccacgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcgggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaacttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaatgcttctttctaactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacata

SEQ ID NO: 48 - protein, 276 aa, Homo sapiens
  misc_feature: Amino acid of RUNX1 target sequence
RHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 49 - RNA, 19 nt, Artificial sequence
  RUNX1 siRNA target sequence
gacaucggcagaaacuaga

SEQ ID NO: 50 - RNA, 19 nt, Artificial sequence
  RUNX1 siRNA target sequence
ggcgauaggucucacgcaa

SEQ ID NO: 51 - RNA, 19 nt, Artificial sequence
  A-E siRNA target sequence
ccucgaaaucguacugaga

SEQ ID NO: 52 - RNA, 21 nt, Artificial sequence
  SiRNA used for KD of RUNX1 (Targets seq id no 49)
ucuaguuucugccgaugucuu

SEQ ID NO: 53 - RNA, 21 nt, Artificial sequence
  SiRNA used for KD of RUNX1 (Targets seq id no 50)
uugcgugagaccuaucgccuu

SEQ ID NO: 54 - RNA, 21 nt, Artificial sequence
  SiRNA used for KD of A-E(Targets seq id no 51)
ucucaguacgauuucgagguu

SEQ ID NO: 55 - DNA, 2722 bp, Homo sapiens
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggaggaagacacagcaccctggagatgttaaggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggattaaatgagttagcctgcagaaatcacttgatgcacagcacatgggaagcattgtgtgtatttattaatccttcacaaagtctttgagatatatttttatcaaatatttagcatggatcccggtacactttcaatacttaataaatggtcaatgttattctttttcactatt

SEQ ID NO: 56 - protein, 250 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQEEDTAPWRC

SEQ ID NO: 57 - DNA, 7274 bp, Homo sapiens
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 58 - protein, 453 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 59 - protein, 752 aa, Homo sapiens
  misc_feature: AML1-ETO fusion protein (BAA03089.1 originates from t(8;21) genomic locus)
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRNRTEKHSTMPDSPVDVKTQSRLTPPTMPPPPTTQGAPRTSSFTPTTLTNGTSHSPTALNGAPSPPNGFSNGPSSSSSSSLANQQLPPACGARQLSKLKRFLTTLQQFGNDISPEIGERVRTLVLGLVNSTLTIEEFHSKLQEATNFPLRPFVIPFLKANLPLLQRELLHCARLAKQNPAQYLAQHEQLLLDASTTSPVDSSELLLDVNENGKRRTPDRTKENGFDREPLHSEHPSKRPCTISPGQRYSPNNGLSYQPNGLPHPTPPPPQHYRLDDMAIAHHYRDSYRHPSHRDLRDRNRPMGLHGTRQEEMIDHRLTDREWAEEWKHLDHLLNCIMDMVEKTRRSLTVLRRCQEADREELNYWIRRYSDAEDLKKGGGSSSSHSRQQSPVNPDPVALDAHREFLHRPASGYVPEEIWKKAEEAVNEVKRQAMTELQKAVSEAERKAHDMITTERAKMERTVAEAKRQAAEDALAVINQQEDSSESCWNCGRKASETCSGCNTARYCGSFCQHKDWEKHHHICGQTLQAQQQGDTPAVSSSVTPNSGAGSPMDTPPAATPRSTTPGTPSTIETTPR

SEQ ID NO: 60 - protein, 1395 aa, Homo sapiens
  misc_feature: Amino acid SEQUENCE of AML1-EVI1 fusion protein (AAB29907.1, originates from t(3;21) genomic locus)
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRNNECVYGNYPEIPLEEMPDADGVASTPSLNIQEPCSPATSSEAFTPKEGSPYKAPIYIPDDIPIPAEFELRESNMPGAGLGIWTKRKIEVGEKFGPYVGEQRSNLKDPSYGWEILDEFYNVKFCIDASQPDVGSWLKYIRFAGCYDQHNLVACQINDQIFYRVVADIAPGEELLLFMKSEDYPHETMAPDIHEERQYRCEDCDQLFESKAELADHQKFPCSTPHSAFSMVEEDFQQKLESENDLQEIHTIQECKECDQVFPDLQSLEKHMLSHTEEREYKCDQCPKAFNWKSNLIRHQMSHDSGKHYECENCAKVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGANRHPAGLTFPTAPGFSFSFPGLFPSGLYHRPPLIPASSPVKGLSSTEQTNKSQSPLMTHPQILPATQDILKALSKHPSVGDNKPVELQPERSSEERPFEKISDQSESSDLDDVSTPSGSDLETTSGSDLESDIESDKEKFKENGKMFKDKVSPLQNLASINNKKEYSNHSIFSPSLEEQTAVSGAVNDSIKAIASIAEKYFGSTGLVGLQDKKVGALPYPSMFPLPFFPAFSQSMYPFPDRDLRSLPLKMEPQSPGEVKKLQKGSSESPFDLTTKRKDEKPLTPVPSKPPVTPATSQDQPLDLSMGSRSRASGTKLTEPRKNHVFGGKKGSNVESRPASDGSLQHARPTPFFMDPIYRVEKRKLTDPLEALKEKYLRPSPGFLFHPQMSAIENMAEKLESFSALKPEASELLQSVPSMFNFRAPPNALPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESTGAILDDKEDAYFTEIRNFIGNSNHGSQSPRNVEERMNGSHFKDEKALVTSQNSDLLDDEEVEDEVLLDEEDEDNDITGKTGKEPVTSNLHEGNPEDDYEETSALEMSCKTSPVRYKEEEYKSGLSALDHIRHFTDSLKMRKMEDNQYSEAELSSFSTSHVPEELKQPLHRKSKSQAYAMMLSLSDKESLHSTSHSSSNVWHSMARAAAESSAIQSISHV

SEQ ID NO: 61 - DNA, 5155 bp, Homo sapiens; identical to SEQ ID NO: 47
  misc_feature: Wild-type RUNX1 Unique Target Sequences (mRNA) in the context of t(3;21) (AML1-EVI1) Leukemia

SEQ ID NO: 62 - protein, 276 aa, Homo sapiens; identical to SEQ ID NO: 48
  misc_feature: Wild-type RUNX1 Unique Amino acid Target Sequences (Protein) in the context of t(3;21) (AML1-EVI1) Leukemia

SEQ ID NO: 63 - DNA, 2110 bp, Homo sapiens
  misc_feature: RUNX1 portion in AML1-ETO fusion transcript [t(8;21) leukemia, D13979]
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaa

SEQ ID NO: 64 - DNA, 5710 bp, Homo sapiens
  misc_feature: RUNX1 portion in ETV6-RUNX1 (also known as TEL-AML1) fusion transcript [t(12;21) leukemia]
aatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccacgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcgggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaacttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaatgcttctttctaactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacata

SEQ ID NO: 65 - DNA, 2134 bp, Homo sapiens
  misc_feature: RUNX1 portion in AML1-EVI1 fusion transcript [t(3;21) leukemia, S69002.1]
tttccaggcactctcattcatagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaactttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgcttggcggttacgcggcgctcgtgggtgttcgtgccttcggagcagctaaccggcgggtgctgggcgacggtggaggagtatcgttctcgctgcttgcccgagtcagggctgagtcaccccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgtttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaa

SEQ ID NO: 66 - DNA, 19 bp, Artificial sequence
  SiRNA target sequence against RUNX1 transcript in t(12;21) ALL cells
cctcataaacaaccacaga

SEQ ID NO: 67 - DNA, 19 bp, Artificial sequence
  SiRNA target sequence against RUNX1 transcript in t(12;21) ALL cells
accacaagttgggtagcct

SEQ ID NO: 68 - DNA, 19 bp, Artificial sequence
  SiRNA target sequence against RUNX1 transcript in t(12;21) ALL cells
cgaatcacactgaatgcaa

SEQ ID NO: 69 - DNA, 19 bp, Artificial sequence
  SiRNA target sequence against RUNX1 transcript in t(12;21) ALL cells
ggctgaaacagtgacctgt

SEQ ID NO: 70 - DNA, 19 bp, Artificial sequence
  SiRNA target sequence against RUNX1 transcript in t(8;21) AML cells
ggatacaaggcagatccaa

SEQ ID NO: 71 - DNA, 19 bp, Artificial sequence
  SiRNA target sequence against RUNX1 transcript in t(8;21) AML cells
ggatccattgcctctcctt

SEQ ID NO: 72 - DNA, 19 bp, Artificial sequence
  SiRNA target sequence against RUNX1 transcript in t(8;21) AML cells
cctcctaccacctgtacta

SEQ ID NO: 73 - DNA, 19 bp, Artificial sequence
  SiRNA target sequence against RUNX1 transcript in t(8;21) AML cells
ggaaagcaaacaggaagat